(19) United States
(12) Patent Application Publication
(10) Pub. No.: US 2011/0269761 A1
(43) Pub. Date: Nov. 3, 2011

(54) ARYLSULPHONYLGLYCINE DERIVATIVES, THE PREPARATION THEREOF AND THEIR USE AS MEDICAMENTS

(75) Inventors: Elke Langkopf, Warthausen (DE); Frank Himmelsbach, Mittelbiberach (DE); Juergen Mack, Biberach (DE); Alexander Pautsch, Ulm (DE); Corinna Schoelch, Mittelbiberach (DE); Annette Schuler-Metz, Ulm (DE); Ruediger Streicher, Biberach (DE); Holger Wagner, Mettenberg (DE)

(73) Assignee: BOEHRINGER INGELHEIM INTERNATIONAL GMBH, Ingelheim am Rhein (DE)

(21) Appl. No.: 12/988,452

(22) PCT Filed: Apr. 17, 2009
(86) PCT No.: PCT/EP09/54593

§ 371 Date: Mar. 3, 2011

(30) Foreign Application Priority Data
Apr. 19, 2008 (DE) 102008019838.2

(51) Int. Cl. A61K 31/5377; A61K 31/277; C07C 271/12; A61K 31/27; C07C 309/65; C07D 271/06; A61K 31/4245; C07D 271/10; C07D 239/02; A61K 31/505; C07D 213/55; A61K 31/44; C07D 295/00; A61K 31/5375; C07D 241/04; A61K 31/495; C07D 215/38; A61K 31/47; C07D 403/04; A61K 31/506; C07D 413/04; A61P 3/10; C07C 255/57

(52) U.S. Cl. 514/236.5; 558/413; 514/522; 560/28; 514/487; 558/54; 548/131; 514/364; 548/143; 544/335; 514/256; 546/335; 514/357; 544/159; 514/237.5; 544/388; 514/255.01; 546/171; 514/311; 544/295; 514/252.14; 544/122; 514/235.8; 544/114

(57) ABSTRACT

The invention relates to substituted aryl-sulphonylglycine derivatives of general formula (I) wherein the groups R^{a} to R^{f}, A and Z are defined as in the specification and claims, which are suitable for preparing a pharmaceutical composition for the treatment of metabolic disorders, particularly type 1 or type 2 diabetes mellitus.

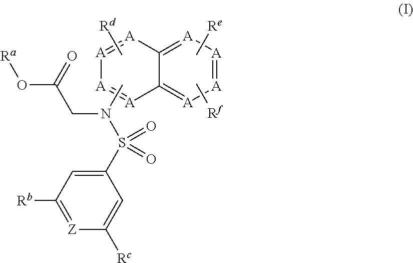

The present invention relates to new substituted arylsulphonylglycine derivatives of general formula

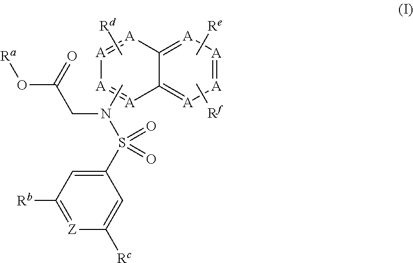

wherein the groups R^{a} to R^{f}, A and Z are defined as hereinafter, including the tautomers, stereoisomers, mixtures thereof and salts thereof. This invention further relates to medicaments containing a compound of formula (I) according to the invention as well as the use of a compound according to the invention for preparing a medicament for the treatment of metabolic disorders, particularly type 1 or type 2 diabetes mellitus. The invention also relates to processes for preparing a medicament as well as a compound according to the invention.

Compounds of formula (I) are suitable for preventing the inhibiting effect of glycogen phosphorylase on the activity of glycogen synthase by stopping the interaction of glycogen phosphorylase a with the G_{L} subunit of glycogen-associated protein phosphatase 1 (PP1). Compounds with these properties stimulate glycogen synthesis and are proposed for the treatment of metabolic disorders, particularly diabetes (P. Cohen, Nature Reviews Molecular Cell Biology 2006, 7, 867-874).

### AIM OF THE INVENTION

The aim of the present invention is to provide new arylsulphonylamino-methylphosphonic acid derivatives that suppress the interaction of glycogen phosphorylase a with the G_{L} subunit of glycogen-associated protein phosphatase 1 (PP1).

A further aim of the present invention is to provide new pharmaceutical compositions that are suitable for the prevention and/or treatment of metabolic disorders, particularly diabetes.

Another aim of this invention is to provide a process for preparing the compounds according to the invention.

Other aims of the present invention will become directly apparent to the skilled man from the foregoing remarks and those that follow.

### OBJECT OF THE INVENTION

In a first aspect the present invention relates to new substituted arylsulphonylglycine derivatives of general formula:

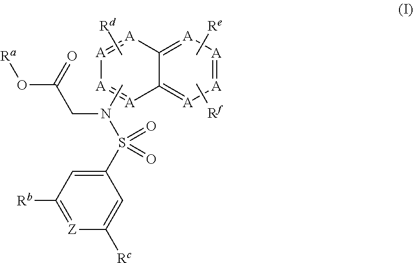

In the above formula (I)
R^{a} denotes H, a group of formula

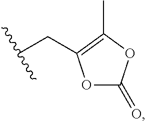

or a C₁₋₆-alkyl group, which may be substituted by
   C₁₋₆-alkyl-carbonyloxy, C₁₋₆-alkoxy-carbonyloxy, C₁₋₆-alkoxy, hydroxy,
   amino, aminocarbonyl or amino-C₂₋₃-alkyloxy, wherein in each case one or two of the hydrogen atoms present on the nitrogen may be replaced independently of one another by a C₁₋₃-alkyl group,
   heterocycloalkyl, heterocycloalkylcarbonyl, heterocycloalkyloxy or heterocycloalkyl-C₁₋₃-alkyloxy,
R^{b} and R^{c} each independently of one another denotes H, halogen, C₁₋₃-alkyl, C₂₋₃-alkenyl, C₂₋₃-alkynyl, C₁₋₃-perfluoroalkyl, C₁₋₃-alkoxy, C₁₋₃-perfluoroalkoxy, while in each case only one of the groups R^{b} and R^{c} may represent H,
A denotes CH or N, while a total of not more than four nitrogen atoms may be present in the bicyclic system,
Z denotes CH, CF or N,
R^{d} and R^{e} independently of one another denote H, halogen, cyano, hydroxy, nitro, C₁₋₆-alkyl, C₂₋₆-alkenyl, C₂₋₆-alkynyl, C₁₋₆-fluoroalkyl, C₁₋₆-perfluoroalkyl, C₃₋₇-cycloalkyl, heterocycloalkyl, aryl, heteroaryl, C₁₋₆-alkoxy, C₁₋₆-fluoroalkoxy, C₁₋₆-perfluoroalkoxy, C₃₋₇-cycloalkyloxy, heterocycloalkyloxy, aryloxy, heteroaryloxy, C₁₋₆-alkylsulphanyl, C₃₋₇-cycloalkylsulphanyl or a group selected from among R¹R²N, R¹R²N—CO, R¹R²N—CO—NR³, R¹R²N—SO, R¹R²N—SO₂, R¹R²N—SO₂—NR³, R⁴—CO, R⁴—CO—NR³, R⁵—SO, R⁵—SO—NR³, R⁵—SO₂, R⁵—SO₂—NR³— and R⁵—CO—O—, wherein
   R¹ denotes H, C₁₋₆-alkyl, C₃₋₇-cycloalkyl, heterocycloalkyl, aryl or heteroaryl,
   R² denotes H, C₁₋₆-alkyl, C₃₋₇-cycloalkyl, heterocycloalkyl, aryl or heteroaryl,
   R³ denotes H, C₁₋₆-alkyl or C₃₋₇-cycloalkyl,
   R⁴ denotes C₁₋₆-alkyl, C₃₋₇-cycloalkyl, heterocycloalkyl, aryl, heteroaryl, hydroxy, or C₁₋₆-alkyloxy and
   R⁵ denotes C₁₋₆-alkyl, C₃₋₇-cycloalkyl, heterocycloalkyl, aryl or heteroaryl,
      and
R^{f} denotes H, halogen, C₁₋₃-alkyl, C₂₋₃-alkenyl, C₂₋₃-alkynyl, C₁₋₃-perfluoroalkyl, C₁₋₃-alkoxy, C₁₋₃-perfluoroalkoxy or cyano,
   while the groups contained in the C₁₋₆-alkyl, C₂₋₆-alkenyl, C₂₋₆-alkynyl, C₃₋₇-cycloalkyl, C₁₋₆-alkyloxy and C₃₋₇-cycloalkyloxy groups mentioned hereinbefore for R^{d}, R^{e}, R^{f} as well as R¹ to R⁵ may each be di- or trisubstituted independently of one another in the carbon skeleton by a group selected from among
   cyano, hydroxy, C₃₋₇-cycloalkyl, heterocycloalkyl, aryl, heteroaryl, C₁₋₆-alkoxy, C₁₋₆-perfluoroalkoxy, C₃₋₇-cycloalkyloxy, heterocycloalkyloxy, aryloxy, heteroaryloxy
   and a group selected from among R⁶R⁷N, R⁶R⁷N—CO, R⁶R⁷N—CO—NR⁸, R⁶R⁷N—SO₂—NR⁸, R⁹—CO, R⁹—CO—NR⁸, R¹⁰—SO₂, R¹⁰—SO₂—NR⁸— and R¹⁰—CO—O, wherein
   R⁶ denotes H, C₁₋₄-alkyl, C₃₋₆-cycloalkyl, C₃₋₆-cycloalkyl-C₁₋₄-alkyl, heterocycloalkyl, heterocycloalkyl-C₁₋₄-alkyl, aryl, aryl-C₁₋₄-alkyl, heteroaryl or heteroaryl-C₁₋₄-alkyl,
   R⁷ denotes H, C₁₋₄-alkyl, C₃₋₆-cycloalkyl, C₃₋₆-cycloalkyl-C₁₋₄-alkyl, heterocycloalkyl, heterocycloalkyl-C₁₋₄-alkyl, aryl, aryl-C₁₋₄-alkyl, heteroaryl or heteroaryl-C₁₋₄-alkyl,
   R⁸ denotes H, C₁₋₄-alkyl, C₃₋₆-cycloalkyl or C₃₋₆-cycloalkyl-C₁₋₄-alkyl,
   R⁹ denotes C₁₋₄-alkyl, C₃₋₆-cycloalkyl, C₃₋₆-cycloalkyl-C₁₋₄-alkyl, heterocycloalkyl, heterocycloalkyl-C₁₋₄-alkyl, aryl, aryl-C₁₋₄-alkyl, heteroaryl, heteroaryl-C₁₋₄-alkyl, hydroxy or C₁₋₄-alkyloxy and
   R¹⁰ denotes C₁₋₄-alkyl, C₃₋₆-cycloalkyl, C₃₋₆-cycloalkyl-C₁₋₄-alkyl, heterocycloalkyl, heterocycloalkyl-C₁₋₄-alkyl, aryl, aryl-C₁₋₄-alkyl, heteroaryl or heteroaryl-C₁₋₄-alkyl,
   while the above-mentioned substituents must not be bound to a common carbon atom and heteroatoms must be separated from one another by at least two carbon atoms,
      and the aryl, heteroaryl, aryloxy and heteroaryloxy groups contained in the groups mentioned hereinbefore for R^{d}, R^{e} as well as R¹ to R⁵ may each be di- or trisubstituted independently of one another in the carbon skeleton by a group selected from among
   halogen, cyano, hydroxy, nitro, C₁₋₆-alkyl, C₂₋₆-alkenyl, C₂₋₆-alkynyl, C₃₋₇-cycloalkyl, C₃₋₇-cycloalkyl-C₁₋₄-alkyl, C₁₋₆-perchloroalkyl, C₁₋₆-fluoroalkyl, C₁₋₆-perfluoroalkyl, C₁₋₆-alkoxy, C₁₋₆-fluoroalkoxy, C₁₋₆-perfluoroalkoxy, C₃₋₇-cyclo-alkyloxy, C₃₋₇-cycloalkyl-C₁₋₄-alkyloxy, heterocycloalkyloxy, heterocycloalkyl-C₁₋₄-alkyloxy C₁₋₆-alkylsulphanyl, C₃₋₇-cycloalkylsulphanyl,
   and a group selected from among R⁶R⁷N, R⁶R⁷N—CO, R⁶R⁷N—CO—NR⁸, R⁶R⁷N—SO, R⁶R⁷N—SO₂, R⁶R⁷N—SO₂—NR⁸, R⁹—CO, R⁹—CO—NR⁸, R¹⁰—SO, R¹⁰—SO—NR⁸, R¹⁹—SO₂, R¹⁹—SO₂—NR⁸— and R¹⁰—CO—O, while R⁶ to R¹⁹ are as hereinbefore defined.

The invention also relates to the tautomers, stereoisomers, mixtures and salts, particularly the physiologically acceptable salts, of the compounds according to the invention.

The compounds of general formula (I) according to the invention and the physiologically acceptable salts thereof have valuable pharmacological properties, in particular they suppress the interaction of glycogen phosphorylase a with the G_{L}-subunit of glycogen-associated protein phosphatase 1 (PP1).

Therefore this invention also relates to the use of the compounds according to the invention, including the physiologically acceptable salts, as pharmaceutical compositions.

This invention further relates to pharmaceutical compositions containing at least one compound according to the invention or a physiologically acceptable salt according to the invention, optionally together with one or more inert carriers and/or diluents.

A further object of this invention is the use of at least one compound according to the invention or a physiologically acceptable salt of such a compound for preparing a pharmaceutical composition that is suitable for the treatment or prevention of diseases or conditions that can be influenced by suppressing the interaction of glycogen phosphorylase a with the G_{L}-subunit of glycogen-associated protein phosphatase 1 (PP1).

The invention also relates to the use of at least one compound according to the invention for preparing a pharmaceutical composition which is suitable for the treatment of metabolic disorders, for example type I or II diabetes mellitus.

The invention also relates to the use of at least one compound according to the invention for preparing a pharmaceutical composition for suppressing the interaction of glycogen phosphorylase a with the G_{L}-subunit of glycogen-associated protein phosphatase 1 (PP1).

A further object of this invention is a process for preparing a pharmaceutical composition according to the invention, characterised in that a compound according to the invention is incorporated in one or more inert carriers and/or diluents by a non-chemical method.

The present invention also relates to a process for preparing the compounds of general formula (I) according to the invention.

### DETAILED DESCRIPTION OF THE INVENTION

Unless stated otherwise, the groups, radicals and substituents, particularly R, R¹ to R⁴, X, Y, Z and A have the meanings given hereinbefore and hereinafter.

If groups, substituents or radicals occur more than once in a compound, they may have the same or different meanings.

Preferred compounds of the above general formula (I) are those wherein the bicyclic heteroaromatic group

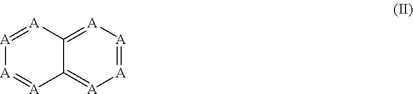

denotes naphthalene, quinoline, isoquinoline, quinazoline, quinoxaline, cinnoline, phthalazine, [1,5]naphthyridine, [1,8]naphthyridine, pyrido[3,2-d]pyrimidine, pyrimido[5,4-d]pyrimidine, or pteridine, and
R^{a} to R^{f}, R¹ to R¹⁰, A and Z are as hereinbefore defined, with the proviso that at least one of the groups R^{d} and R^{e} denotes H, halogen or C₁₋₃-alkyl.

Particularly preferred are those compounds of the above general formula (I), wherein the bicyclic heteroaromatic group

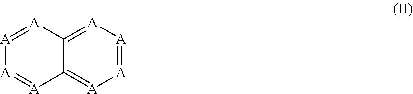

denotes naphthalene, quinoline, quinazoline, quinoxaline or cinnoline,
R^{a} denotes H, a group of formula

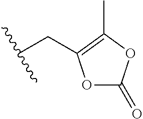

or a C₁₋₄-alkyl group, which may be substituted by C₁₋₄-alkoxy, hydroxy, di-(C₁₋₃-alkyl)-amino, pyrrolidin-1-yl, piperidin-1-yl, morpholin-4-yl, piperazin-1-yl or 4-methyl-piperazin-1-yl,
R^{b} and R^{c} independently of one another denote chlorine, bromine or C₁₋₂-alkyl,
Z denotes CH or N,
R^{d} denotes H, halogen, cyano, hydroxy, nitro, C₁₋₄-alkyl, C₂₋₄-alkenyl, C₂₋₄-alkynyl, aryl-C₂₋₃-alkynyl, C₁₋₄-fluoroalkyl, C₁₋₄-perfluoroalkyl, C₃₋₆-cycloalkyl, C₃₋₆-cycloalkyl-C₁₋₄-alkyl, heterocycloalkyl, heterocycloalkyl-C₁₋₄-alkyl, aryl, aryl-C₁₋₄-alkyl, heteroaryl, heteroaryl-C₁₋₄-alkyl, C₁₋₄-alkoxy, C₁₋₄-fluoroalkoxy, C₁₋₄-perfluoroalkoxy, C₃₋₆-cycloalkyloxy, C₃₋₆-cycloalkyl-C₁₋₄-alkyloxy, heterocycloalkyloxy, heterocycloalkyl-C₁₋₄-alkoxy, aryloxy, aryl-C₁₋₄-alkyloxy, heteroaryloxy, heteroaryl-C₁₋₄-alkyloxy, C₁₋₄-alkylsulphanyl or C₃₋₆-cyclo-alkylsulphanyl,
   while the aryl and heteroaryl groups contained in the groups mentioned hereinbefore for R^{d} may optionally be substituted by halogen, C₁₋₃-alkyl, trichloromethyl, phenyl, phenyl-C₁₋₃-alkyl, hydroxy, C₁₋₃-alkoxycarbonyl, phenyloxy-C₁₋₃-alkyl, phenylsulphonyl-C₁₋₃-alkyl, morpholin-4-yl-C₁₋₃-alkyl, cyano, amino, C₁₋₃-alkylamino, di-(C₁₋₃-alkyl)-amino, amino-C₁₋₃-alkylamino, C₁₋₃-alkylamino-C₁₋₃-alkylamino, di-(C₁₋₃-alkyl)-amino-C₁₋₃-alkylamino, N-(amino-C₁₋₃-alkyl)-N—(C₁₋₃-alkyl)amino, N—(C₁₋₃-alkylamino-C₁₋₃-alkyl)-N—(C₁₋₃-alkyl)-amino, N-[di-(C₁₋₃-alkyl)-amino-C₁₋₃-alkyl]-N—(C₁₋₃-alkyl)-amino, morpholin-4-yl, piperazin-1-yl or 4-(C₁₋₃-alkyl)-piperazin-1-yl,
   or a group selected from among R¹R²N, R¹R²N—CO, R¹R²N—CO—NR³, R¹R²N—SO, R¹R²N—SO₂, R¹R²N—SO₂—NR³, R⁴—CO, R⁴—CO—NR³, R⁵—SO, R⁵—SO—NR³, R⁵—SO₂— and R⁵—SO₂—NR³, wherein
   R¹ denotes H, C₁₋₄-alkyl, hydroxy-C₁₋₄-alkyl, C₃₋₆-cycloalkyl, C₃₋₆-cycloalkyl-C₁₋₄-alkyl, heterocycloalkyl, heterocycloalkyl-C₁₋₄-alkyl, aryl, aryl-C₁₋₄-alkyl, heteroaryl or heteroaryl-C₁₋₄-alkyl,
   R² denotes H, C₁₋₄-alkyl, hydroxy-C₁₋₄-alkyl, C₃₋₆-cycloalkyl, C₃₋₆-cycloalkyl-C₁₋₄-alkyl, heterocycloalkyl, heterocycloalkyl-C₁₋₄-alkyl, aryl, aryl-C₁₋₄-alkyl, heteroaryl or heteroaryl-C₁₋₄-alkyl,
   R³ denotes H, C₁₋₄-alkyl, C₃₋₆-cycloalkyl or C₃₋₆-cycloalkyl-C₁₋₄-alkyl,
   R⁴ denotes C₁₋₄-alkyl, C₃₋₆-cycloalkyl, C₃₋₆-cycloalkyl-C₁₋₄-alkyl, heterocycloalkyl, aryl, aryl-C₁₋₄-alkyl, heteroaryl, heteroaryl-C₁₋₄-alkyl, hydroxy or C₁₋₄-alkyloxy and
   R⁵ denotes C₁₋₄-alkyl, C₃₋₆-cycloalkyl, heterocycloalkyl, aryl, aryl-C₁₋₄-alkyl, heteroaryl or heteroaryl-C₁₋₄-alkyl,
      while the aryl and heteroaryl groups contained in the groups mentioned hereinbefore for R¹ to R⁵ may optionally be substituted by halogen, cyano, C₁₋₃-alkoxy, C₁₋₃-alkoxycarbonyl, carboxy, aminocarbonyl, C₁₋₃-alkylaminocarbonyl, di-(C₁₋₃-alkyl)aminocarbonyl, morpholin-4-ylcarbonyl, piperazin-1-ylcarbonyl, amino, C₁₋₃-alkylamino, di-(C₁₋₃-alkyl)amino, amino-C₁₋₃-alkyl, amino-C₁₋₃-alkylamino, C₁₋₃-alkylamino-C₁₋₃-alkyl-amino, di-(C₁₋₃-alkyl)amino-C₁₋₃-alkylamino, N-(amino-C₁₋₃-alkyl)-N—(C₁₋₃-alkyl)-amino, N—(C₁₋₃-alkylamino-C₁₋₃-alkyl)-N—(C₁₋₃-alkyl)amino or N-[di-(C₁₋₃-alkyl)amino-C₁₋₃-alkyl]-N—(C₁₋₃-alkyl)-amino,
R^{e} has the meaning given hereinbefore for R^{d}, with the proviso that at least one of the groups R^{d} and R^{e} must be H, halogen or C₁₋₃-alkyl, and
R^{f} denotes H or C₁₋₃-alkyl.

Particularly preferred are those compounds of the above general formula (I), wherein

the bicyclic heteroaromatic group of general formula (II) denotes naphthalene or quinoline,
R^{a} denotes H or a C₁₋₄-alkyl group optionally substituted by a di-(C₁₋₃-alkyl)-amino group,
R^{b} and R^{c} independently of one another denote chlorine, bromine or C₁₋₂-alkyl,
Z denotes CH,
R^{d} denotes H, or, if R^{e} denotes H, it may also denote a group selected from among
   fluorine, chlorine, bromine, cyano, C₁₋₃-alkoxy, 5-methyl-[1,2,4]oxadiazolyl,
   aminocarbonyl, wherein a hydrogen atom may be replaced by a C₁₋₃-alkyl group and the second hydrogen atom may be replaced independently thereof by a C₁₋₃-alkyl, phenyl or phenyl-C₁₋₃-alkyl group, and
   amino, wherein a hydrogen atom may be replaced by a C₁₋₃-alkyl group and the second hydrogen atom may be replaced independently thereof by a C₁₋₃-alkyl or a phenylsulphonyl group,
R^{e} denotes H, or, if R^{d} denotes H, it may also denote a group selected from among
   fluorine, chlorine, bromine, cyano, C₁₋₃-alkyl, C₁₋₃-alkoxy,
   furanyl, oxazolyl, isoxazolyl, which may be substituted in each case by one or two C₁₋₃-alkyl groups,
   [1,2,4]oxadiazolyl, which may be substituted by C₁₋₃-alkyl, trichloromethyl, phenyl, benzyl, hydroxy, C₁₋₃-alkoxycarbonyl, phenyloxymethyl, phenyl-sulphonylmethyl or morpholin-4-ylmethyl,
   5-oxo-4,5-dihydro-[1,2,4]oxadiazolyl, which may be substituted by C₁₋₃-alkyl,
   pyridinyl, pyrimidinyl, pyrazinyl, pyridazinyl, which may be substituted in each case by C₁₋₃-alkyl, cyano, amino, C₁₋₃-alkylamino, di-(C₁₋₃-alkyl)-amino, di-(C₁₋₃-alkyl)-amino-C₁₋₃-alkylamino, N-[di-(C₁₋₃-alkyl)-amino-C₁₋₃-alkyl]-N—(C₁₋₃-alkyl)-amino, morpholin-4-yl or piperazin-1-yl,
   pyrrolidin-1-ylcarbonyl, 3,4-dihydro-1H-isoquinolin-2-ylcarbonyl,
   and a group of formula n R¹R²N—CO, R¹R²N—CO—NR³ or R⁴CONR³, wherein
   R¹ denotes H, C₁₋₃-alkyl, hydroxy-C₁₋₃-alkyl, C₃₋₆-cycloalkyl-C₁₋₃-alkyl, phenyl, phenyl-C₁₋₃-alkyl, pyridinyl or pyridinyl-C₁₋₃-alkyl,
   R² denotes H or C₁₋₃-alkyl,
   R³ denotes H or C₁₋₃-alkyl,
   and
   R⁴ denotes C₁₋₃-alkyl, phenyl, phenyl-C₁₋₃-alkyl, pyridinyl or pyridinyl-C₁₋₃-alkyl,
      while the phenyl and pyridinyl groups contained in R¹ to R⁴ may optionally be substituted by chlorine, cyano, methoxy, carboxy, aminocarbonyl, C₁₋₃-alkylaminocarbonyl, di-(C₁₋₃-alkyl)-aminocarbonyl, morpholin-4-ylcarbonyl, piperazin-1-ylcarbonyl, amino, C₁₋₃-alkylamino, di-(C₁₋₃-alkyl)-amino, aminomethyl, di-(C₁₋₃-alkyl)-amino-C₁₋₃-alkylamino or N-[di-(C₁₋₃-alkyl)amino-C₁₋₃-alkyl]-N—(C₁₋₃-alkyl)-amino,
         and
R^{f} denotes H or C₁₋₃-alkyl.

Most particularly preferred are those compounds of the above general formula (I), wherein

the bicyclic heteroaromatic group of formula (II) is naphthalene or quinoline,
R^{a} denotes H,
R^{b} and R^{c} independently of one another denote chlorine, bromine or methyl,
Z denotes CH,
R^{d} denotes H, or, if R^{e} denotes H, it may also denote a group selected from among
   C₁₋₂-alkoxy, 5-methyl-[1,2,4]oxadiazole, N-phenylsulphonyl-N-methyl-amino, N-methyl-N-phenyl-aminocarbonyl, N-benzyl-aminocarbonyl and N-benzyl-N-methyl-aminocarbonyl,
R^{e} denotes H, or, if R^{d} denotes H, it may also denote a group selected from among
   methoxy, furanyl, oxazolyl, isoxazolyl, 3,5-dimethyl-isoxazole, 3-methyl-[1,2,4]oxadiazolyl, 5-methyl-[1,2,4]oxadiazolyl, 5-trichloromethyl-[1,2,4]oxadiazolyl, 5-isopropyl-[1,2,4]oxadiazolyl, 3-phenyl-[1,2,4]oxadiazolyl, 5-phenyl-[1,2,4]oxadiazolyl, 3-benzyl-[1,2,4]oxadiazolyl, 5-benzyl-[1,2,4]oxadiazolyl, 5-hydroxy-[1,2,4]oxadiazolyl, 3-ethoxycarbonyl-[1,2,4]oxadiazolyl, 3-phenyl-oxymethyl-[1,2,4]oxadiazolyl, 3-phenylsulphonylmethyl-[1,2,4]oxadiazolyl, 5-(morpholin-4-ylmethyl)-[1,2,4]oxadiazolyl, 5-oxo-4,5-dihydro-[1,2,4]oxadiazolyl, 4-methyl-5-oxo-4,5-dihydro-[1,2,4]oxadiazolyl, pyridinyl, pyrimidinyl, 4-(piperazin-1-yl)-pyrimidinyl, 2-(morpholin-4-yl)-pyrimidinyl, 4-(morpholin-4-yl)-pyrimidinyl, 4-(2-dimethylamino-ethylamino)-pyrimidinyl, 4-[N-(2-dimethylamino-ethyl)-N-methyl-amino]-pyrimidinyl, 5-(morpholin-4-yl)-pyrazin-2-yl, 5-(2-dimethylamino-ethylamino)-pyrazin-2-yl, 6-(morpholin-4-yl)-pyridazin-3-yl, 6-(2-dimethylamino-ethylamino)-pyridazin-3-yl, pyrrolidin-1-ylcarbonyl, 3,4-dihydro-1H-isoquinolin-2-ylcarbonyl,
   and a group of formula R¹R²N—CO, R¹R²N—CO—NR³ or R⁴CONR³, wherein
   R¹ denotes H, C₁₋₃-alkyl, hydroxyethyl, cyclohexylmethyl, phenyl, benzyl, 2-phenyl-ethyl, pyridinyl or pyridinylmethyl,
   R² denotes H or methyl,
   R³ denotes H
   and
   R⁴ denotes phenyl, benzyl, 2-phenyl-ethyl or pyridinyl,
      while the phenyl, benzyl and 2-phenyl-ethyl groups contained in R¹ and R⁴ may be substituted by a cyano, methoxy, carboxy, aminocarbonyl, methylaminocarbonyl, dimethylaminocarbonyl, morpholin-4-ylcarbonyl, piperazin-1-ylcarbonyl or aminomethyl group and
      the pyridinyl and pyridinylmethyl groups contained in R¹ and R⁴ may be substituted by a chlorine atom or a 2-dimethylamino-ethylamino or N-(2-dimethylamino-ethyl)-N-(methyl)-amino group,
         and
R^{f} denotes H,
   the enantiomers, the mixtures thereof and the salts thereof,
   but particularly the compounds of general formula

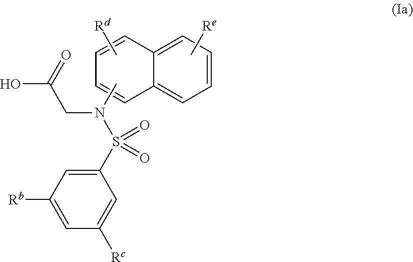

wherein
R^{b} and R^{c} each denote chlorine,
R^{d} denotes H, or, if R^{e} denotes H, it may also denote a group selected from among
   fluorine, chlorine, bromine, cyano, C₁₋₃-alkyl, C₁₋₃-alkoxy, 5-methyl-[1,2,4]oxadiazolyl,
   aminocarbonyl, wherein a hydrogen atom may be replaced by a C₁₋₃-alkyl group and the second hydrogen atom may be replaced independently thereof by a C₁₋₃-alkyl, phenyl or phenyl-C₁₋₃-alkyl group, and
   amino, wherein a hydrogen atom may be replaced by a C₁₋₃-alkyl group and the second hydrogen atom may be replaced independently thereof by a C₁₋₃-alkyl or a phenylsulphonyl group,
      and
R^{e} denotes H, or, if R^{d} denotes H, it may also denote a group selected from among
   fluorine, chlorine, bromine, cyano, C₁₋₃-alkyl, C₁₋₃-alkoxy,
   furanyl, oxazolyl, isoxazolyl, which may be substituted in each case by one or two C₁₋₃-alkyl groups,
   [1,2,4]oxadiazolyl, which may be substituted by C₁₋₃-alkyl, trichloromethyl, phenyl, benzyl, hydroxy, C₁₋₃-alkoxycarbonyl, phenyloxymethyl, phenyl-sulphonylmethyl or morpholin-4-ylmethyl,
   5-oxo-4,5-dihydro-[1,2,4]oxadiazolyl, which may be substituted by C₁₋₃-alkyl,
   pyridinyl, pyrimidinyl, pyrazinyl, pyridazinyl, which may be substituted in each case by C₁₋₃-alkyl, cyano, amino, C₁₋₃-alkylamino, di-(C₁₋₃-alkyl)-amino, di-(C₁₋₃-alkyl)-amino-C₁₋₃-alkylamino, N-[di-(C₁₋₃-alkyl)-amino-C₁₋₃-alkyl]-N—(C₁₋₃-alkyl)-amino, morpholin-4-yl or piperazin-1-yl,
   pyrrolidin-1-ylcarbonyl, 3,4-dihydro-1H-isoquinolin-2-ylcarbonyl,
   and a group of formula n R¹R²N—CO, R¹R²N—CO—NR³ or R⁴CONR³, wherein
   R¹ denotes H, C₁₋₃-alkyl, hydroxy-C₁₋₃-alkyl, C₃₋₆-cycloalkyl-C₁₋₃-alkyl, phenyl, phenyl-C₁₋₃-alkyl, pyridinyl or pyridinyl-C₁₋₃-alkyl,
   R² denotes H or C₁₋₃-alkyl,
   R³ denotes H or C₁₋₃-alkyl,
   and
   R⁴ denotes phenyl, phenyl-C₁₋₃-alkyl, pyridinyl or pyridinyl-C₁₋₃-alkyl,
      while the phenyl and pyridinyl groups contained in R¹ to R⁴ may optionally be substituted by chlorine, cyano, methoxy, carboxy, aminocarbonyl, C₁₋₃-alkylaminocarbonyl, di-(C₁₋₃-alkyl)-aminocarbonyl, morpholin-4-ylcarbonyl, piperazin-1-ylcarbonyl, amino, C₁₋₃-alkylamino, di-(C₁₋₃-alkyl)-amino, aminomethyl, di-(C₁₋₃-alkyl)-amino-C₁₋₃-alkylamino or N-[di-(C₁₋₃-alkyl)amino-C₁₋₃-alkyl]-N—(C₁₋₃-alkyl)-amino.

The following compounds may be mentioned by way of example:
(1) [[5-(4-aminocarbonyl-benzylaminocarbonyl)-naphthalen-2-yl]-(3,5-dichloro-phenylsulphonyl)-amino]-acetic acid,
(2) {(3,5-dichloro-phenylsulphonyl)-[6-(5-methyl-[1,2,4]oxadiazol-3-yl)-naphthalen-2-yl]-amino}-acetic acid,
(3) {(3,5-dichloro-phenylsulphonyl)-[6-(3-methyl-[1,2,4]oxadiazol-5-yl)-naphthalen-2-yl]-amino}-acetic acid,
(4) [(5-benzylaminocarbonyl-naphthalen-1-yl)-(3,5-dichloro-phenylsulphonyl)-amino]-acetic acid,
(5) [(3,5-dichloro-phenylsulphonyl)-(5-pyrimidin-2-yl-naphthalen-1-yl)-amino]-acetic acid,
(6) {(3,5-dichloro-phenylsulphonyl)-[5-(5-morpholin-4-ylmethyl-[1,2,4]oxadiazol-3-yl)naphthalen-2-yl]-amino}-acetic acid,
(7) ((3,5-dichloro-phenylsulphonyl)-{5-[(pyridin-3-ylmethyl)-aminocarbonyl]-naphthalen-1-yl}-amino)-acetic acid,
(8) {(3,5-dichloro-phenylsulphonyl)-[5-(3-phenyl-ureido)-naphthalen-1-yl]-amino}-acetic acid,
(9) [[5-(3-cyano-benzylaminocarbonyl)-naphthalen-1-yl]-(3,5-dichloro-phenylsulphonyl)-amino]-acetic acid,
(10) [[5-(2-cyano-benzylaminocarbonyl)-naphthalen-1-yl]-(3,5-dichloro-phenyl-sulphonyl)-amino]-acetic acid,
(11) ((3,5-dichloro-phenylsulphonyl)-{5-[4-(piperazin-1-ylcarbonyl)-benzylamino-carbonyl]-naphthalen-1-yl}-amino)acetic acid,
(12) {(3,5-dichloro-phenylsulphonyl)-[5-(4-methylaminocarbonyl-benzylamino-carbonyl)-naphthalen-1-yl]-amino}-acetic acid,
(13) {(3,5-dichloro-phenylsulphonyl)-[5-(3-methylaminocarbonyl-benzylamino-carbonyl)-naphthalen-1-yl]-amino}-acetic acid,
(14) {(3,5-dichloro-phenylsulphonyl)-[5-({2-N-[(2-dimethylamino-ethyl)-N-methyl-amino]-pyridin-4-ylmethyl}-aminocarbonyl)-naphthalen-1-yl]-amino}-acetic acid,
(15) ((3,5-dichloro-phenylsulphonyl)-{6-[5-(2-dimethylamino-ethylamino)-pyrazin-2-yl]-naphthalen-2-yl}-amino)-acetic acid,
(16) {(3,5-dichloro-phenylsulphonyl)-[6-(4-morpholin-4-yl-pyrimidin-2-yl)-naphthalen-2-yl]-amino}-acetic acid,
(17) [(3,5-dichloro-phenylsulphonyl)-quinolin-8-yl-amino]-acetic acid and
(18) [(3,5-dichloro-phenylsulphonyl)-(6-methoxy-quinolin-8-yl)-amino]-acetic acid.

### Terms and Definitions Used

Some terms used hereinbefore and hereinafter to describe the compounds according to the invention are defined more specifically below.

Unless otherwise stated, all the substituents are independent of one another. If for example there are a plurality of C₁₋₆-alkyl groups as substituents in one group, in the case of three C₁₋₆-alkyl substituents, independently of one another, one may represent methyl, one n-propyl and one tert-butyl.

Where a hyphen open on one side “-” is used in the structural formula of a substituent, this hyphen is to be understood as the linkage point to the remainder of the molecule. The substituent replaces the corresponding groups R^{a}, R^{b}, etc. If no hyphen open on one side is used in the structural formula of a substituent, the linkage point to the remainder of the molecule is clear from the name or the structural formula itself.

By the term “optionally substituted” is meant within the scope of the invention the above-mentioned group, optionally substituted by a lower-molecular group. Examples of lower-molecular groups regarded as chemically meaningful are groups consisting of 1-200 atoms. Preferably such groups have no negative effect on the pharmacological efficacy of the compounds.

The subject-matter of this invention also includes the compounds according to the invention, including the salts thereof, wherein one or more hydrogen atoms, for example one, two, three, four or five hydrogen atoms, are replaced by deuterium.

The term “halogen” within the scope of the present invention denotes fluorine, chlorine, bromine or iodine. Unless stated otherwise, fluorine, chlorine and bromine are regarded as preferred halogens.

By the term “C₁₋ₙ-alkyl” (including those which are part of other groups) are meant branched and unbranched alkyl groups with 1 to n carbon atoms. Examples include: methyl, ethyl, n-propyl, iso-propyl, n-butyl, iso-butyl, sec-butyl, tert-butyl, n-pentyl, iso-pentyl, neo-pentyl or hexyl. The abbreviations Me, Et, n-Pr, i-Pr, n-Bu, i-Bu, t-Bu, etc. Unless described otherwise, the definitions propyl, butyl, pentyl and hexyl include all the possible isomeric forms of the groups in question. Thus, for example, propyl includes n-propyl and iso-propyl, butyl includes iso-butyl, sec-butyl and tert-butyl etc.

By the term “C₁₋ₙ-fluoroalkyl” (including those which are part of other groups) are meant partly fluorinated, branched and unbranched alkyl groups with 1 to n carbon atoms, in which at least one hydrogen atom is replaced by fluorine. Examples of such partly fluorinated alkyl groups include difluoromethyl, trifluoroethyl and tetrafluoroethyl.

By the term “C₁₋ₙ-perfluoroalkyl” (including those which are part of other groups) is meant a F—(CF₂)ₙ group. Examples of such groups include trifluoromethyl, pentafluoroethyl, heptafluoro-n-propyl, heptafluoro-iso-propyl etc., but preferably trifluoromethyl and pentafluorethyl.

By the term “C₂₋ₙ-alkenyl” (including those which are part of other groups) are meant branched and unbranched alkenyl groups, with 2 to n carbon atoms, which contain one or more double bonds. Examples include: ethenyl or vinyl, propenyl, butenyl, pentenyl, or hexenyl. Unless described otherwise, the definitions propenyl, butenyl, pentenyl and hexenyl include all the possible isomeric forms of the groups in question. Thus, for example, propenyl includes 1-propenyl and 2-propenyl, butenyl includes 1 butenyl-, 2-butenyl, 3-butenyl, 1-methyl-1-propenyl and 1-methyl-2-propenyl etc.

By the term “C₂₋ₙ-alkynyl” (including those which are part of other groups) are meant branched and unbranched alkynyl groups, with 2 to n carbon atoms, which contain one or more triple bonds. Examples include: ethynyl, propynyl or butynyl. Unless described otherwise, the definitions propynyl and butynyl include all the possible isomeric forms of the groups in question. Thus, for example propynyl includes 1-propynyl and 2-propynyl, butynyl includes 1-butynyl, 2-butynyl, 3-butynyl, 1-methyl-1-propynyl and 1-methyl-2-propynyl etc.

By the term “C₃₋ₙ-cycloalkyl” (including those which are part of other groups) are meant saturated mono-, bi, tri or spirocyclic alkyl groups with 3 to n carbon atoms. Examples include: cyclopropyl, cyclobutyl, cyclopentyl, cyclohexyl, cycloheptyl, bicyclo[3,2,1]octyl, spiro[4,5]decyl, norpinyl, norbornyl, norcaryl, adamantyl. Preferably the term C₃₋₇-cycloalkyl includes monocyclic alkyl groups. Unless otherwise stated, the cyclic alkyl groups may be substituted by one or more groups selected from among methyl, ethyl, hydroxy, methoxy, amino, methylamino and dimethylamino.

By the term “aryl” (including those which are part of other groups) are meant aromatic ring systems with 6, 10 or 14 carbon atoms. Examples include: phenyl, naphthyl, anthracenyl or phenanthrenyl. Unless otherwise stated, the aromatic groups may be substituted by one or more groups selected from among methyl, ethyl, difluoromethyl, trifluoromethyl, cyano, hydroxy, methoxy, difluoromethoxy, trifluoromethoxy, amino, fluorine, chlorine, bromine and iodine. Preferred aryl groups are naphthyl and phenyl, of which phenyl is particularly preferred.

By the term “heteroaryl” are meant 5- to 10-membered mono- or bicyclic aromatic heterocycles, wherein up to three carbon atoms may be replaced by one or more heteroatoms selected from among oxygen, nitrogen and sulphur. Each of the above-mentioned heterocycles may optionally also be anellated to a benzene ring. The ring may be linked to the molecule through a carbon atom or, if present, through a nitrogen atom.

The following are examples of five- or six-membered heterocyclic aromatic groups:

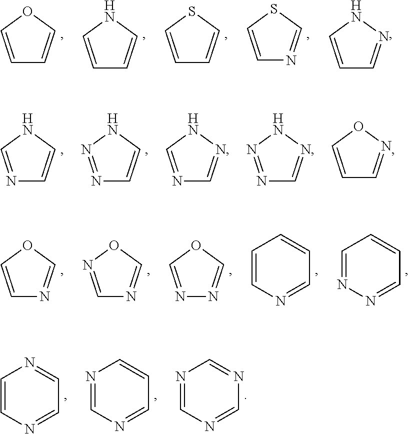

The following are mentioned as examples of 5-10-membered bicyclic heteroaryl rings: pyrrolizine, indole, indolizine, isoindole, indazole, purine, quinoline, isoquinoline, quinazoline, quinoxaline, cinnoline, phthalazine, naphthyridine, benzimidazole, benzofuran, benzothiophene, benzoxazole, benzothiazole, benzisothiazole, pyridopyrimidine, pteridine, pyrimidopyrimidine.

Unless otherwise stated, the heteroaromatic groups may be substituted by one or more groups selected from among methyl, ethyl, difluoromethyl, trifluoromethyl, cyano, hydroxy, methoxy, difluoromethoxy, trifluoromethoxy, amino, fluorine, chlorine, bromine and iodine.

Preferred heteroaryl groups are furanyl, thiophenyl, pyrrole, 1 H-imidazole, 1 H-pyrazole, oxazole, isoxazole, thiazole, [1,2,4]oxadiazole, pyridinyl, pyrimidinyl, pyrazinyl, pyridazinyl, benzoxazolyl and benzothiazolyl.

Particularly preferred heteroaryl groups are [1,2,4]oxadiazole, pyridinyl, pyrimidinyl, pyrazinyl and pyridazinyl.

By the term “heterocycloalkyl” are meant four- to seven-membered, preferably five- to six-membered, saturated heterocycles which contain one, two or three heteroatoms, selected from among oxygen, sulphur and nitrogen, preferably oxygen and nitrogen. The ring may be linked to the molecule via a carbon atom or, if present, via a nitrogen atom.

The following are mentioned by way of example:

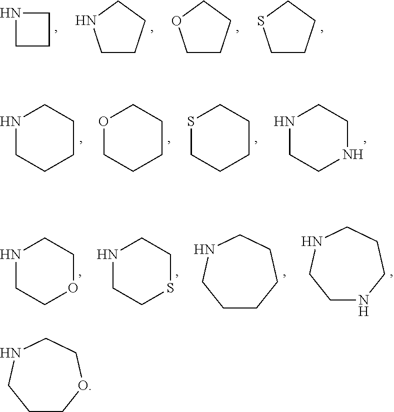

Unless otherwise mentioned, the heterocyclic group may be provided with one or more oxo groups. Examples include:

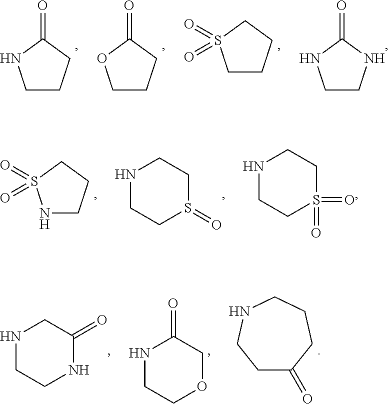

Unless otherwise mentioned, any nitrogen atoms contained in the ring may optionally be substituted by a methyl, ethyl, acetyl or methylsulphonyl group and the cyclic carbon atoms may be substituted by a methyl, ethyl, hydroxy, methoxy, amino, methylamino or dimethylamino group.

Preferred heterocycloalkyl groups are tetrahydrofuranyl, pyrrolidinyl, piperidinyl, morpholinyl, homomorpholinyl, piperazinyl, homopiperazinyl, 2-oxo-piperazinyl, 3-oxo-morpholinyl, 1,1-oxo-thiomorpholinyl and 1,1-dioxo-thiomorpholinyl.

Particularly preferred heterocycloalkyl groups are tetrahydrofuranyl, pyrrolidinyl, piperidinyl, morpholinyl and piperazinyl.

The term enantiomerically pure describes within the scope of the present invention compounds of formula (I), which are present in an enantiomerical purity of at least 85% ee, preferably at least 90% ee, particularly preferably >95% ee. The term ee (enantiomeric excess) is known in the art and describes the optical purity of chiral compounds.

The term “protective group” for the purposes of the present invention is to be understood as being a collective term for those organic groups with which certain functional groups of a molecule can be temporarily protected from attach by reagents, so that reactions can be carried out in a manner targeted on only the desired locations in the molecule. The protective groups should be capable of being introduced selectively under mild conditions and should be stable under the conditions of the planned reactions and cleaning operations, while racemisations and epimerisations should also be excluded. The protective groups should be cleavable under mild conditions selectively and ideally in a high yield. The choice of a suitable protective group, suitable conditions for its introduction (solvent, temperature, duration, etc.) as well as the possible ways of removing the protective group are known in the art (e.g. P. Kocienski, Protecting Groups, 3rd ed. 2004, THIEME, Stuttgart, ISBN: 3131370033).

By an “organic solvent” is meant, within the scope of the invention, an organic, low-molecular substance which can dissolve other organic substances by a physical method. To be suitable the prerequisite for the solvent is that neither the dissolving substance nor the dissolved substance should be chemically altered during the dissolving process, i.e. the components of the solution should be recoverable in their original form by physical separation processes such as distillation, crystallisation, sublimation, evaporation or adsorption. For various reasons, not only the pure solvents but also mixtures that combine the dissolving properties may be used.

Examples include:

alcohols, preferably methanol, ethanol, propanol, butanol, octanol and cyclohexanol;
glycols, preferably ethyleneglycol and diethyleneglycol;
ethers/glycolethers, preferably diethyl ether, tert-butyl-methylether, dibutylether, anisol, dioxane, tetrahydrofuran, and mono-, di- and tri-, polyethyleneglycol ethers;
ketones, preferably acetone, butanone and cyclohexanone;
esters, preferably acetic acid esters and glycolesters;
amides and other nitrogen compounds, preferably dimethylformamide, pyridine, N-methylpyrrolidone and acetonitrile;
sulphur compounds, preferably carbon disulphide, dimethylsulphoxide and sulpholane;
nitro compounds, preferably nitrobenzene;
halogenated hydrocarbons, preferably dichloromethane, chloroform, tetrachlormethane, trichloroethane, tetrachloroethane, 1,2-dichloroethane and chlorofluorocarbons;
aliphatic or alicyclic hydrocarbons, preferably benzines, petroleum ether, cyclohexane, methylcyclohexane, decaline and terpene-L.; and
aromatic hydrocarbons, preferably benzene, toluene, o-xylene, m-xylene and p-xylene;
and corresponding mixtures thereof.

Compounds of general formula (I) may contain acid groups, such as e.g. carboxylic acid or phosphonic acid groups and/or basic groups such as e.g. amino functions. Compounds of general formula (I) may therefore be present as internal salts, as salts with pharmaceutically useable inorganic acids such as hydrochloric acid, sulphuric acid, phosphoric acid, sulphonic acid or organic acids (such as for example maleic acid, fumaric acid, citric acid, tartaric acid or acetic acid) or as salts with pharmaceutically useable bases such as alkali metal or alkaline earth metal hydroxides or carbonates, zinc or ammonium hydroxides or organic amines such as e.g. diethylamine, triethylamine, triethanolamine, inter alia. For preparing the alkali metal and alkaline earth metal salts of the compound of formula (I), it is preferable to use the alkali metal and alkaline earth metal hydroxides and hydrides, while the hydroxides and hydrides of the alkali metals, particularly sodium and potassium are preferred, and sodium and potassium hydroxide are particularly preferred. (See also Pharmaceutical Salts, S. M. Birge et al., J. Pharm. Sci. 1977, 66, 1-19)

### Methods of Preparation

The compounds according to the invention may be obtained using methods of synthesis that are known in principle. Preferably the compounds are obtained by methods of preparation according to the invention that are described more fully hereinafter.

The preparation of compounds of general formula (I) may be carried out according to the process shown in Scheme 1 starting from a compound of general formula (III), wherein R^{a}, R^{b}, R^{c}, A and Z are as hereinbefore defined and R^{d′}, R^{e′} and R^{f′} either have the meanings given hereinbefore for R^{d}, R^{e} and R^{f} or denote groups that can be converted into R^{d}, R^{e} and R^{f} by known methods of synthesis.

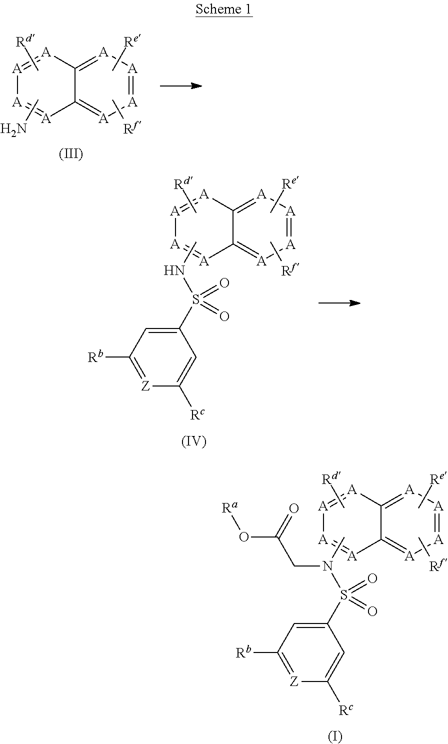

Compounds of general formula (IV) are obtained by sulphonylation of compounds of general formula (III).

The sulphonylation is carried out with aromatic sulphonyl chlorides in the presence of a base such as triethylamine, diisopropylethylamine, pyridine, or 4-dimethylamino-pyridine, but preferably pyridine. The reaction may be carried out in suitable solvents such as diethyl ether, tetrahydrofuran, toluene, pyridine, dichloromethane, or chloroform, but preferably dichloromethane. The temperature may be between 0° C. and 60° C., but preferably between 15° C. and 40° C. Examples of reactions of this kind are described in Example II.

Compounds of general formula (I) are obtained from compounds of general formula (IV) by alkylation.

Suitable alkylating agents are acetic acid ester derivatives which contain in the 2-position a leaving group such as chlorine, bromine, iodine, p-tolylsulphonate, methylsulphonate, or trifluoromethylsulphonate. The alkylation is carried out in a solvent such as dimethylformamide, dimethylacetamide, tetrahydrofuran, acetonitrile, N-methylpyrrolidone or dimethylsulphoxide, but preferably in dimethylformamide, in the presence of a base such as sodium carbonate, potassium carbonate or caesium carbonate, but preferably potassium carbonate, and at a temperature between 0° C. and 100° C., but preferably between 15° C. and 50° C.

Examples of reactions of this kind are described in Example I.

If acetic acid derivatives with a methyl or ethyl ester unit are used as alkylating agents, the esters obtained may then be cleaved to form the free carboxylic acid. This may take place hydrolytically in an aqueous solvent, e.g. in water, methanol/water, isopropanol/water, acetic acid/water, tetrahydrofuran/water or dioxane/water, but preferably in methanol/water, in the presence of an acid such as trifluoroacetic acid, hydrochloric acid or sulphuric acid or in the presence of an alkali metal base such as lithium hydroxide, sodium hydroxide or potassium hydroxide, but preferably sodium hydroxide, or aprotically, e.g. in the presence of iodotrimethylsilane, at temperatures between 0 and 120° C., preferably at temperatures between 10 and 100° C. Examples of reactions of this kind are described in Examples 1 and 2.

If acetic acid derivatives with a tert.-butyl ester unit are used as alkylating agents, compounds of general formula (I) are obtained wherein R^{a}=tert.-butyl. The cleaving of the tert.-butyl group is preferably carried out by treatment with an acid such as trifluoroacetic acid or hydrochloric acid or by treatment with iodotrimethylsilane optionally using a solvent such as methylene chloride, dioxane, methanol or diethyl ether.

Examples of reactions of this kind are described in Example 3.

Alternatively the intermediate compounds of general formula (IV) may also be prepared by the process shown in Scheme 2 according to the invention starting from a compound of general formula (V), wherein X denotes halogen or trifluoromethylsulphonate.

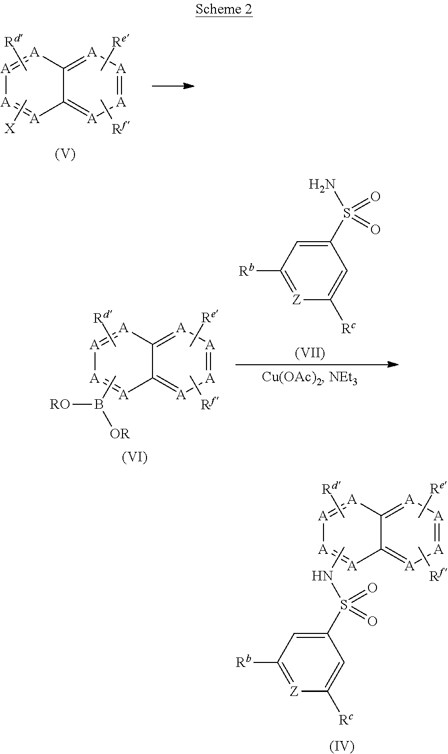

Compounds of general formula (V), wherein X denotes halogen, preferably bromine or iodine, are converted by metal-halogen exchange with a suitable reagent, e.g. n-butyllithium, tert.-butyllithium or phenylmagnesium bromide, intermediately into the corresponding organometallic compounds, which are then reacted with trialkylborates (cf. also Boronic Acids; Preparation and Applications in Organic Synthesis and Medicine, D. G. Hall ed., WILEY-VCH 2005, S. 28 ff). Examples of reactions of this kind are described in Example XXVI

Alternatively compounds of formula (V) wherein X is halogen or trifluoromethylsulphonate may be reacted with tetraalkoxydiboron compounds (RO)₂B—B(OR)₂ or dialkoxyboranes HB(OR)₂ in the presence of a suitable catalyst, for example PdCl₂(dppf), and a base to form the corresponding boron esters (VI) (see T. Ishiyama et al., J. Org. Chem. 1995, 60, 7508; M. Murata et al., J. Org. Chem. 1997; 62, 6458; N. Miyaura et al., Tetrahedron Lett. 1997, 38, 3447; M. Murata et al., J. Org. Chem. 2000; 65, 164).

After hydrolytic cleaving to obtain the free boric acid the boric acid esters (VI) thus obtained may then be reacted with sulphonamides of general formula (VII) to form the compounds of general formula (IV). This reaction is expediently carried out in the presence of copper(II) acetate and a tertiary amino base such as triethylamine or pyridine in a suitable solvent such as tetrahydrofuran or dichloromethane (D. M. T. Chan et al., Tetrahedron Lett. 1998, 39, 2933). Examples of reactions of this kind are described in Example XXIX.

The compounds of general formulae (III) to (VII) used as starting materials are known from the literature in some cases or may be prepared using methods known from the literature or those described hereinbefore, optionally with the additional introduction of protective groups (see Examples I to XLI).

In the reactions described hereinbefore, any reactive groups present such as carboxy, hydroxy, amino or alkylamino groups may be protected during the reaction by conventional protecting groups which are cleaved again after the reaction.

For example, a protecting group for a carboxy group may be a methyl, ethyl, tert.butyl or benzyl group.

For example, a protecting group for a hydroxy group may be an acetyl, benzyl or tetrahydropyranyl group.

Protecting groups for an amino or alkylamino may be a formyl, acetyl, trifluoroacetyl, ethoxycarbonyl, tert.butoxycarbonyl, benzyloxycarbonyl, benzyl, methoxybenzyl or 2,4-dimethoxybenzyl group.

A methoxy- or ethoxycarbonyl unit is cleaved for example by hydrolysis in an aqueous solvent, e.g. in water, methanol/water, isopropanol/water, acetic acid/water, tetrahydrofuran/water or dioxane/water, but preferably in methanol/water, in the presence of an acid such as trifluoroacetic acid, hydrochloric acid or sulphuric acid or in the presence of an alkali metal base such as lithium hydroxide, sodium hydroxide or potassium hydroxide, but preferably sodium hydroxide, or aprotically, e.g. in the presence of iodotrimethylsilane, at temperatures between 0 and 120° C., preferably at temperatures between 10 and 100° C.

A benzyl, methoxybenzyl or benzyloxycarbonyl group is advantageously cleaved by hydrogenolysis, e.g. with hydrogen in the presence of a catalyst such as palladium on charcoal in a suitable solvent such as methanol, ethanol, ethyl acetate or glacial acetic acid, optionally with the addition of an acid such as hydrochloric acid, at temperatures between 0 and 100° C., but preferably at temperatures between 20 and 60° C., and under a hydrogen pressure of 1 to 7 bar, but preferably 1 to 3 bar. However, a 2,4-dimethoxybenzyl group is preferably cleaved in trifluoroacetic acid in the presence of anisole.

A tert.-butyl or tert.-butyloxycarbonyl group is preferably cleaved by treating with an acid such as trifluoroacetic acid or hydrochloric acid or by treating with iodotrimethylsilane, optionally using a solvent such as methylene chloride, dioxane, methanol or diethyl ether.

Moreover, the compounds of general formula (I) obtained, or intermediate products from the synthesis of compounds of general formula (I), as already mentioned hereinbefore, may be resolved into their enantiomers and/or diastereomers. Thus, for example, cis/trans mixtures may be resolved into their cis and trans isomers, and compounds with at least one stereocentre may be resolved into their enantiomers.

Thus, for example, compounds of general formula (I), or intermediate products from the synthesis of compounds of general formula I, which occur as racemates may be separated by methods known per se (cf. N. L. Allinger and E. L. Eliel in “Topics in Stereochemistry”, Vol. 6, Wiley Interscience, 1971) into their optical antipodes. Compounds of general formula (I), or intermediate products from the synthesis of compounds of general formula (I), with at least 2 asymmetric carbon atoms may be resolved into their diastereomers on the basis of their physical-chemical differences using methods known per se, e.g. by chromatography and/or fractional crystallisation, and, if these compounds are obtained in racemic form, they may subsequently be resolved into the enantiomers as mentioned above.

The enantiomers are preferably separated by chromatography on chiral phases or by recrystallisation from an optically active solvent or by reacting with an optically active substance which forms salts or derivatives such as e.g. esters or amides with the racemic compound, and separating the diastereomeric mixture of salts or derivatives thus obtained, e.g. on the basis of their differences in solubility, whilst the free antipodes may be released from the pure diastereomeric salts or derivatives by the action of suitable agents. Examples of optically active substances include optically active acids and the activated derivatives or optically active alcohols thereof. Optically active acids in common use are e.g. the D- and L-forms of tartaric acid or dibenzoyltartaric acid, di-O-p-toluoyltartaric acid, malic acid, mandelic acid, camphorsulphonic acid, glutamic acid, aspartic acid or quinic acid. An optically active alcohol may be for example (+) or (−)-menthol and an optically active acyl group in amides, for example, may be a (+)- or (−)-menthyloxycarbonyl.

Furthermore, the compounds of formula (I) obtained, or intermediate products from the synthesis of compounds of general formula I, may be converted into the salts thereof, for pharmaceutical use in particular into the physiologically acceptable salts thereof with inorganic or organic acids. Acids which may be used for this purpose include for example hydrochloric acid, hydrobromic acid, sulphuric acid, methanesulphonic acid, phosphoric acid, fumaric acid, succinic acid, lactic acid, citric acid, tartaric acid or maleic acid.

Moreover, the new compounds of general formula (I) obtained, or intermediate products from the synthesis of compounds of general formula I, if they contain a carboxy group, may, if desired, be converted into the salts thereof with inorganic or organic bases, for pharmaceutical use particularly into the physiologically acceptable salts thereof. Suitable bases for this purpose include for example sodium hydroxide, potassium hydroxide, arginine, cyclohexylamine, ethanolamine, diethanolamine and triethanolamine.

### Biological Test

The compounds of general formula (I) are inhibitors of the interaction between human liver glycogen phosphorylase (HLGP) and the protein PPP1R3 (G_{L}-subunit of glycogen-associated protein phosphatase 1 (PP1)). The effect of the compounds on the binding of the protein PPP1R3 and the glycogen phosphorylase activated by phosphorylation is determined in a binding test based on SPA technology (Amersham Pharmacia). The binding of the substances inhibits the interaction of the glycogen phosphorylase with the protein PPP1R3B. PII measurements were made in triplicate in the 384-well format (Optiplate, Perkin Elmer). Human glycogen phosphorylase is recombinantly expressed in E. coli and purified. The isolated non-phosphorylated HLGP is radioactively labelled in a marking reaction with phosphorylase kinase (200-500 U/mg, P2014, Sigma) and ³³P-gamma ATP (110 TBq/mmol, Hartmann Analytic) (Ref.: Cohen et al., Methods Enzymol. 1988, Vol 159 pp 390). In a binding test, in a volume of 100 μl (test buffer: 50 mM Tris/HCl pH 7.0, 0.1 mM EGTA, 0.1% mercaptoethanol), different amounts of a test substance (final concentration: 1 nM to 30 μM) are incubated at ambient temperature for 16 hours with 100000 cpm of labelled HLGP, 375 μg streptavidin-SPA Beads (RPNQ 0007, Amersham Pharmacia), 0.1 μg GL-peptide (Biotin-FPEWPSYLGYEKLGPYY). After centrifuging for 5 minutes at 500 g the plate is measured (Topcount, Packard). The cpm values measured are used to calculate the IC₅₀ values specified. The basal value is determined in the absence of the peptide and the maximum value is determined in the absence of the test substance.

The compounds of general formula (I) described in the Examples have IC₅₀ values <5 μM.

### Indications

In view of their ability to suppress the interaction of glycogen phosphorylase a with the GL-subunit of glycogen-associated protein phosphatase 1 (PP1), the compounds of general formula (I) according to the invention and the corresponding pharmaceutically acceptable salts thereof are theoretically suitable for treating and/or preventatively treating all those conditions or diseases that can be influenced by inhibiting the interaction of glycogen phosphorylase a with the GL-subunit of glycogen-associated protein phosphatase 1 (PP1). Therefore the compounds according to the invention are particularly suitable for the prevention or treatment of diseases, particularly metabolic disorders, or conditions such as type 1 and type 2 diabetes mellitus, complications of diabetes (such as e.g. retinopathy, nephropathy or neuropathies, diabetic foot, ulcers, macroangiopathies), metabolic acidosis or ketosis, reactive hypoglycaemia, hyperinsulinaemia, glucose metabolic disorder, insulin resistance, metabolic syndrome, dyslipidaemias of different origins, atherosclerosis and related diseases, obesity, high blood pressure, chronic heart failure, oedema and hyperuricaemia. These substances are also suitable for preventing beta-cell degeneration such as e.g. apoptosis or necrosis of pancreatic beta cells. The substances are also suitable for improving or restoring the functionality of pancreatic cells, and also for increasing the number and size of pancreatic beta cells. The compounds according to the invention may also be used as diuretics or antihypertensives and are suitable for the prevention and treatment of acute renal failure.

In particular, the compounds according to the invention, including the physiologically acceptable salts thereof, are suitable for the prevention or treatment of diabetes, particularly type 1 and type 2 diabetes mellitus, and/or diabetic complications.

The dosage required to achieve the corresponding activity for treatment or prevention usually depends on the compound which is to be administered, the patient, the nature and gravity of the illness or condition and the method and frequency of administration and is for the patient's doctor to decide. Expediently, the dosage may be from 0.1 to 1000 mg, preferably 0.5 to 500 mg, by intravenous route, and 1 to 1000 mg, preferably 10 to 500 mg, by oral route, in each case administered 1 to 4 times a day. For this purpose, the compounds of formula (I) prepared according to the invention may be formulated, optionally together with other active substances, together with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof, to produce conventional galenic preparations such as plain or coated tablets, capsules, powders, suspensions or suppositories.

The compounds according to the invention may also be used in conjunction with other active substances, particularly for the treatment and/or prevention of the diseases and conditions mentioned above. Other active substances which are suitable for such combinations include in particular those which potentiate the therapeutic effect of an inhibitor of the interaction of glycogen phosphorylase a with the G_{L} subunit of glycogen-associated protein phosphatase 1 (PP1) according to the invention with respect to one of the indications mentioned and/or which allow the dosage of an inhibitor of the interaction of glycogen phosphorylase a with the GL subunit of glycogen-associated protein phosphatase 1 (PP1) according to the invention to be reduced. Therapeutic agents which are suitable for such a combination include, for example, antidiabetic agents such as metformin, sulphonylureas (e.g. glibenclamide, tolbutamide, glimepiride), nateglinide, repaglinide, thiazolidinediones (e.g. rosiglitazone, pioglitazone), PPAR-gamma-agonists (e.g. GI 262570) and antagonists, PPAR-gamma/alpha modulators (e.g. KRP 297), alpha-glucosidase inhibitors (e.g. Miglitol, acarbose, voglibose), DPPIV inhibitors (e.g. sitagliptine, vildagliptine), SGLT2-inhibitors, alpha2-antagonists, insulin and insulin analogues, GLP-1 and GLP-1 analogues (e.g. exendin-4) or amylin. Other active substances suitable as combination partners are inhibitors of protein tyrosinephosphatase 1, substances that affect deregulated glucose production in the liver, such as e.g. inhibitors of glucose-6-phosphatase, or fructose-1,6-bisphosphatase, glycogen phosphorylase, glucagon receptor antagonists and inhibitors of phosphoenol pyruvate carboxykinase, glycogen synthase kinase or pyruvate dehydrokinase, lipid lowering agents such as for example HMG-CoA-reductase inhibitors (e.g. simvastatin, atorvastatin), fibrates (e.g. bezafibrate, fenofibrate), nicotinic acid and the derivatives thereof, PPAR-alpha agonists, PPAR-delta agonists, ACAT inhibitors (e.g. avasimibe) or cholesterol absorption inhibitors such as, for example, ezetimibe, bile acid-binding substances such as, for example, cholestyramine, inhibitors of ileac bile acid transport, HDL-raising compounds such as CETP inhibitors or ABC1 regulators or active substances for treating obesity, such as sibutramine or tetrahydrolipostatin, dexfenfluramine, axokine, antagonists of the cannabinoid1 receptor, MCH-1 receptor antagonists, MC4 receptor agonists, NPY5 or NPY2 antagonists or R3-agonists such as SB-418790 or AD-9677 and agonists of the 5HT2c receptor.

Moreover, combinations with drugs for influencing high blood pressure, chronic heart failure or atherosclerosis such as e.g. P-II antagonists or ACE inhibitors, ECE inhibitors, diuretics, β-blockers, Ca-antagonists, centrally acting antihypertensives, antagonists of the alpha-2-adrenergic receptor, inhibitors of neutral endopeptidase, thrombocyte aggregation inhibitors and others or combinations thereof are suitable. Examples of angiotensin II receptor antagonists are candesartan cilexetil, potassium losartan, eprosartan mesylate, valsartan, telmisartan, irbesartan, EXP-3174, L-158809, EXP-3312, olmesartan, medoxomil, tasosartan, KT-3-671, GA-0113, RU-64276, EMD-90423, BR-9701, etc. Angiotensin II receptor antagonists are preferably used for the treatment or prevention of high blood pressure and complications of diabetes, often combined with a diuretic such as hydrochlorothiazide.

A combination with uric acid synthesis inhibitors or uricosurics is suitable for the treatment or prevention of gout.

A combination with GABA-receptor antagonists, Na-channel blockers, topiramat, protein-kinase C inhibitors, advanced glycation end product inhibitors or aldose reductase inhibitors may be used for the treatment or prevention of complications of diabetes.

The dosage for the combination partners mentioned above is usefully 1/5 of the lowest dose normally recommended up to 1/1 of the normally recommended dose.

Therefore, in another aspect, this invention relates to the use of a compound according to the invention or a physiologically acceptable salt of such a compound combined with at least one of the active substances described above as a combination partner, for preparing a pharmaceutical composition which is suitable for the treatment or prevention of diseases or conditions which can be affected by inhibiting the interaction of glycogen phosphorylase a with the G_{L} subunit of glycogen-associated protein phosphatase 1 (PP1). These are preferably metabolic diseases, particularly one of the diseases or conditions listed above, most particularly diabetes or diabetic complications.

The use of the compound according to the invention, or a physiologically acceptable salt thereof, in combination with another active substance may take place simultaneously or at staggered times, but particularly within a short space of time. If they are administered simultaneously, the two active substances are given to the patient together; if they are used at staggered times the two active substances are given to the patient within a period of less than or equal to 12 hours, but particularly less than or equal to 6 hours.

Consequently, in another aspect, this invention relates to a pharmaceutical composition which comprises a compound according to the invention or a physiologically acceptable salt of such a compound and at least one of the active substances described above as combination partners, optionally together with one or more inert carriers and/or diluents.

Thus, for example, a pharmaceutical composition according to the invention comprises a combination of a compound of formula (I) according to the invention or a physiologically acceptable salt of such a compound and at least one angiotensin II receptor antagonist optionally together with one or more inert carriers and/or diluents.

The compound according to the invention, or one of the physiologically acceptable salt thereof, and the additional active substance to be combined therewith may both be present together in one formulation, for example a tablet or capsule, or separately in two identical or different formulations, for example as a so-called kit-of-parts.

The Examples that follow are intended to illustrate the present invention without restricting it:

### Preparation of the Starting Compounds

### Example I

### Methyl [[5-(4-cyano-benzylaminocarbonyl)-naphthalen-2-yl]-(3,5-dichloro-phenylsulphonyl)-amino]-acetate

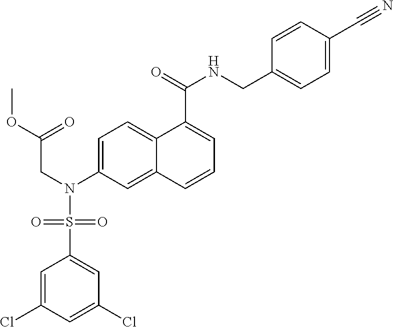

A mixture of 3.00 g 6-(3,5-dichloro-phenylsulphonylamino)-naphthalene-1-carboxylic acid-4-cyano-benzylamide, 0.63 ml methyl bromoacetate and 1.80 g potassium carbonate in 50 ml N,N-dimethylformamide is combined with 50 mg potassium iodide and stirred for 18 h at ambient temperature. Then another 50 μl methyl bromoacetate are added and the mixture is stirred for a further hour at ambient temperature. For working up 130 ml ice water are added. The precipitate formed is suction filtered, washed with water and dissolved in ethyl acetate. The aqueous phase is extracted with ethyl acetate. The combined organic phases are washed with saturated sodium chloride solution, dried on magnesium sulphate and dried and evaporated down. The crude product is purified by chromatography through a silica gel column with methylene chloride/ethyl acetate (90:10 to 85:15) as eluant.

Yield: 2.30 g (67% of theory)

R_{f} value: 0.50 (silica gel, methylene chloride/methanol=98:2)

Mass spectrum (ESI⁺): m/z=582, 584, 586 [M+H]⁺

The following compounds are obtained analogously to Example I:

### (1) methyl [[5-(benzylaminocarbonyl)-naphthalen-2-yl]-(3,5-dichloro-phenyl-sulphonyl)-amino]-acetate

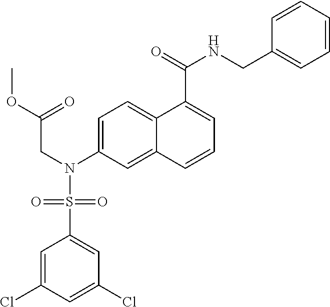

R_{f} value: 0.28 (silica gel, methylene chloride/methanol=99:1)

Mass spectrum (ESI⁺): m/z=557, 559, 561 [M+H]⁺

### (2) methyl [{5-[4-(tert-butoxycarbonylamino-methyl)-benzylaminocarbonyl]-naphthalen-2-yl}-(3,5-dichloro-phenylsulphonyl)-amino]-acetate

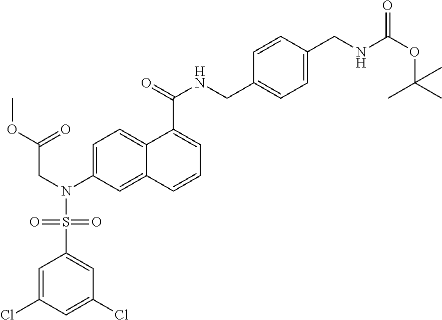

R_{f} value: 0.54 (silica gel, methylene chloride/methanol=95:5)

Mass spectrum (ESI⁺): m/z=686, 688, 690 [M+H]⁺

### (3) tert. Butyl [(3,5-dichloro-phenylsulphonyl)-(5-methylaminocarbonyl-naphthalen-2-yl)-amino]-acetate

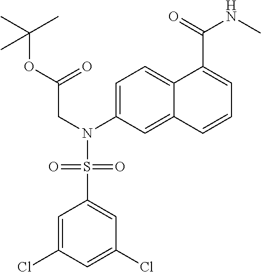

R_{f} value: 0.60 (silica gel, methylene chloride/methanol=95:5)

Mass spectrum (ESI⁺): m/z=523, 525, 527 [M+H]⁺

### (4) benzyl 6-[tert.-butoxycarbonylmethyl-(3,5-dichloro-phenylsulphonyl)-amino]-naphthalene-1-carboxylate

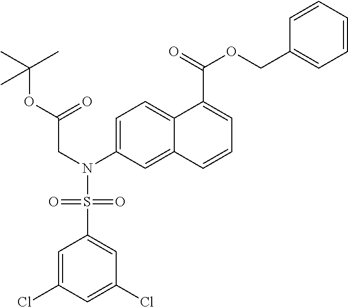

R_{f} value: 0.45 (silica gel, petroleum ether/ethyl acetate=8:2)

Mass spectrum (ESI⁺): m/z=617, 619, 621 [M+NH₄]⁺

### (5) tert. Butyl [(6-aminocarbonyl-naphthalen-2-yl)-(3,5-dichloro-phenylsulphonyl)-amino]-acetate

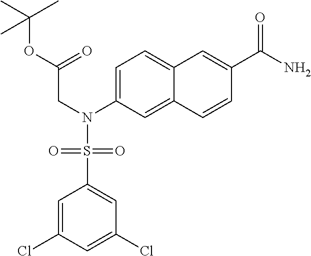

R_{f} value: 0.49 (silica gel, ethyl acetate/cyclohexane=4:1)

Mass spectrum (ESI⁺): m/z=509, 511, 513 [M+H]⁺

### (6) tert. Butyl [(6-cyano-naphthalen-2-yl)-(3,5-dichloro-phenylsulphonyl)-amino]-acetate

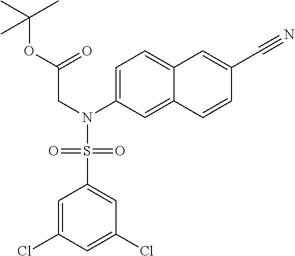

R_{f} value: 0.80 (silica gel, cyclohexane/ethyl acetate=3:1)

### (7) tert. Butyl {(3,5-dichloro-phenylsulphonyl)-[6-(2-hydroxy-ethylaminocarbonyl)-naphthalen-2-yl]-amino}acetate

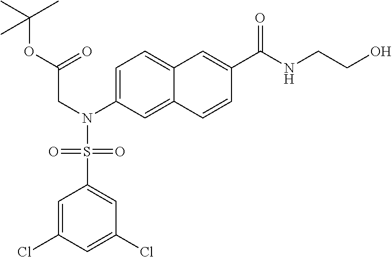

R_{f} value: 0.45 (silica gel, methylene chloride/methanol=9:1)

Mass spectrum (ESI⁻): m/z=551, 553, 555 [M−H]⁻

### (8 tert. Butyl) [(3,5-dichloro-phenylsulphonyl)-naphthalen-1-yl-amino]-acetate

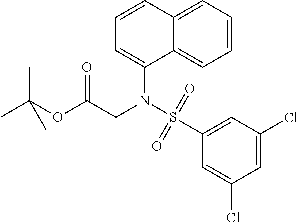

R_{f} value: 0.80 (silica gel, cyclohexane/ethyl acetate=3:1)

### (9) tert. Butyl [(6-benzylaminocarbonyl-naphthalen-2-yl)-(3,5-dichloro-phenylsulphonyl)-amino]-acetate

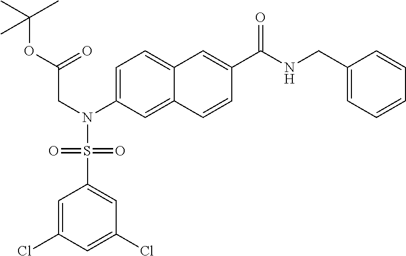

### (10) tert. Butyl [(5-benzylaminocarbonyl-naphthalen-2-yl)-(3,5-dimethyl-phenylsulphonyl)-amino]-acetate

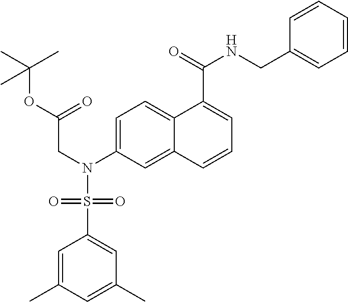

Mass spectrum (ESI⁺): m/z=559 [M+H]⁺

R_{f} value: 0.30 (silica gel, hexane/ethyl acetate=1:1)

### (11) methyl [(3,5-dichloro-phenylsulphonyl)-(6-phenylaminocarbonyl-naphthalen-2-yl)-amino]-acetate

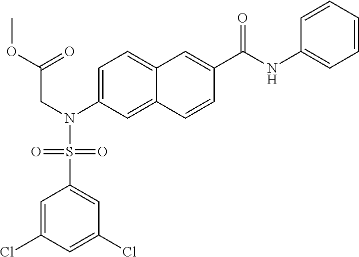

Mass spectrum (ESI⁺): m/z=543, 545, 547 [M+H]⁺

### (12) methyl [(3,5-dichloro-phenylsulphonyl)-(6-methylaminocarbonyl-naphthalen-2-yl)-amino]-acetate

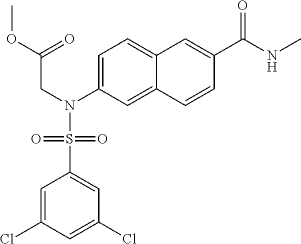

R_{f} value: 0.48 (silica gel, methylene chloride/methanol=95:5)

Mass spectrum (ESI⁺): m/z=481, 483, 485 [M+H]⁺

### (13) tert. Butyl [(6-benzylaminocarbonyl-naphthalen-2-yl)-(3,5-dimethyl-phenylsulphonyl)-amino]-acetate

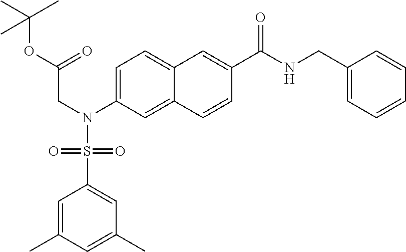

R_{f} value: 0.64 (silica gel, petroleum ether/ethyl acetate=1:1)

Mass spectrum (ESI⁺): m/z=559 [M+H]⁺

### (14) tert. Butyl [(7-benzoylamino-naphthalen-2-yl)-(3,5-dichloro-phenylsulphonyl)-amino]-acetate

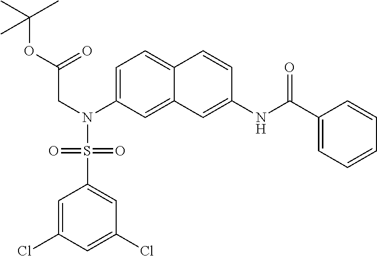

Mass spectrum (ESI⁺): m/z=585 [M+H]⁺

### (15) tert. Butyl [(7-amino-naphthalen-2-yl)-(3,5-dichloro-phenylsulphonyl)-amino]-acetate

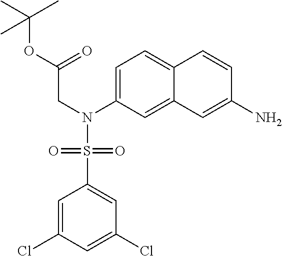

### (16) tert. Butyl [(3,5-dichlorophenylsulphonyl)-(6-trifluoromethanesulphonyloxy-naphthalen-2-yl)-amino]-acetate

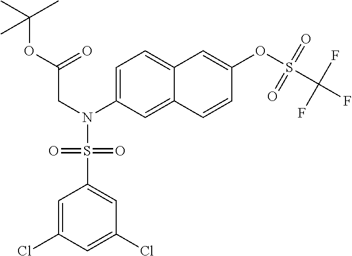

R_{f} value: 0.90 (silica gel, cyclohexane/ethyl acetate=1:1)

Mass spectrum (ESI⁻): m/z=648, 650, 652 [M+Cl]⁻

### (17) tert. Butyl [(7-cyano-naphthalen-2-yl)-(3,5-dichloro-phenylsulphonyl)-amino]-acetate

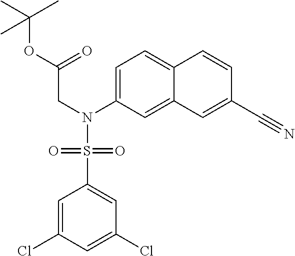

Mass spectrum (ESI⁺): m/z=508, 510, 512 [M+NH₄]⁺

### (18) tert. Butyl [(7-cyano-naphthalen-2-yl)-(3,5-dichloro-phenylsulphonyl)-amino]-acetate

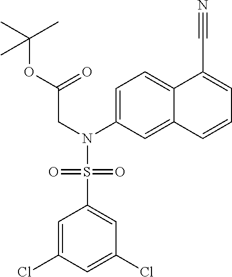

R_{f} value: 0.50 (silica gel, petroleum ether/ethyl acetate=3:1)

Mass spectrum (ESI⁺): m/z=508, 510, 512 [M+NH₄]⁺

### (19) tert. Butyl {(3,5-dichloro-phenylsulphonyl)-[5-(3-methyl-[1,2,4]oxadiazol-5-yl)-naphthalen-2-yl]-amino}-acetate

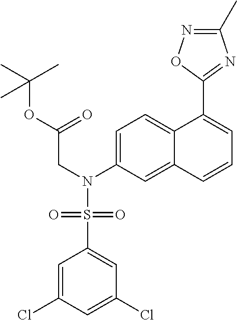

R_{f} value: 0.60 (silica gel, petroleum ether/ethyl acetate=3:1)

Mass spectrum (ESI⁺): m/z=548, 550, 552 [M+H]⁺

### (20) tert. Butyl {(3,5-dichloro-phenylsulphonyl)-[6-(3-methyl-[1,2,4]oxadiazol-5-yl)-naphthalen-2-yl]-amino}-acetate

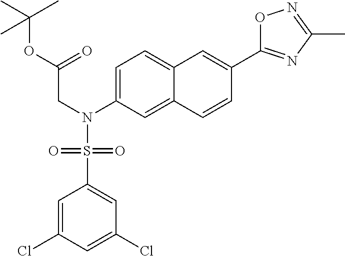

Mass spectrum (ESI⁺): m/z=548, 550, 552 [M+H]⁺

### (21) tert. Butyl {(3,5-dichloro-phenylsulphonyl)-[6-(3-phenyl-[1,2,4]oxadiazol-5-yl)-naphthalen-2-yl]-amino}-acetate

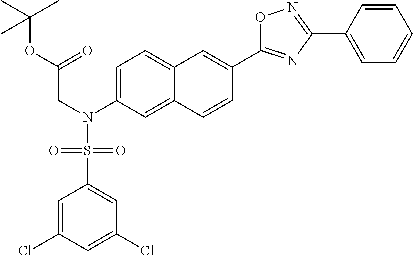

Mass spectrum (ESI⁺): m/z=610, 612, 614 [M+H]⁺

### (22) tert. Butyl {(3,5-dichloro-phenylsulphonyl)-[5-(3-ethoxycarbonyl-[1,2,4]oxadiazol-5-yl)-naphthalen-2-yl]-amino}-acetate

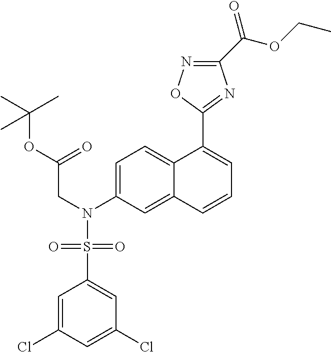

R_{f} value: 0.40 (silica gel, petroleum ether/ethyl acetate=3:1)

Mass spectrum (ESI⁺): m/z=606, 608, 610 [M+H]⁺

### (23) tert. Butyl {(3,5-dichloro-phenylsulphonyl)-[6-(5-methyl-[1,3,4]oxadiazol-2-yl)-naphthalen-2-yl]-amino}-acetate

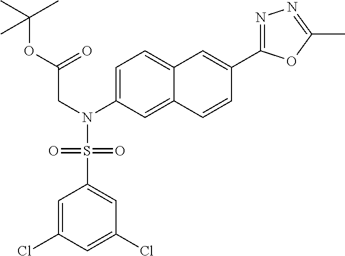

Mass spectrum (ESI⁺): m/z=548, 550, 552 [M+H]⁺

### (24) tert. Butyl {(3,5-dichloro-phenylsulphonyl)-[5-(3-benzyl-[1,2,4]oxadiazol-5-yl)-naphthalen-2-yl]-amino}-acetate

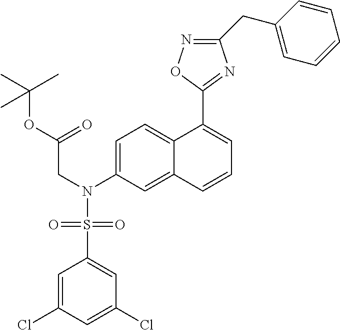

R_{f} value: 0.60 (silica gel, petroleum ether/ethyl acetate=3:1)

Mass spectrum (ESI⁺): m/z=624, 626, 628 [M+H]⁺

### (25) tert. Butyl {(3,5-dichloro-phenylsulphonyl)-[5-(3-phenyloxymethyl-[1,2,4]oxadiazol-5-yl)-naphthalen-2-yl]-amino}-acetate

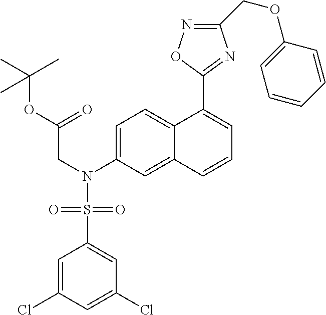

R_{f} value: 0.50 (silica gel, petroleum ether/ethyl acetate=3:1)

Mass spectrum (ESI⁺): m/z=640, 642, 644 [M+H]⁺

### (26) tert. Butyl {(3,5-dichloro-phenylsulphonyl)-[5-(3-phenylsulphonylmethyl-[1,2,4]oxadiazol-5-yl)-naphthalen-2-yl]-amino}-acetate

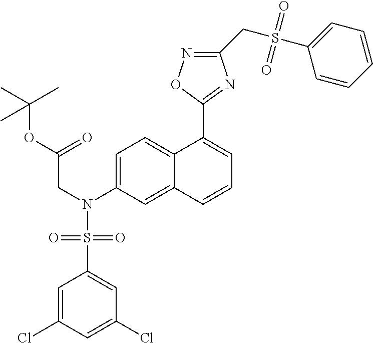

R_{f} value: 0.30 (silica gel, petroleum ether/ethyl acetate=3:1)

Mass spectrum (ESI⁺): m/z=688, 690, 692 [M+H]⁺

### (27) methyl [(3,5-dichloro-phenylsulphonyl)-(4-methoxy-naphthalen-2-yl)-amino]-acetate

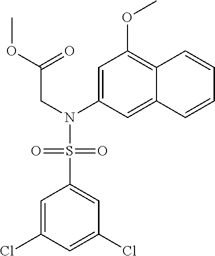

R_{f} value: 0.95 (silica gel, methylene chloride/methanol=98:2)

Mass spectrum (ESI⁺): m/z=454, 456, 458 [M+H]⁺

### (28) methyl [(5-benzylaminocarbonyl-naphthalen-1-yl)-(3,5-dichloro-phenylsulphonyl)-amino]-acetate

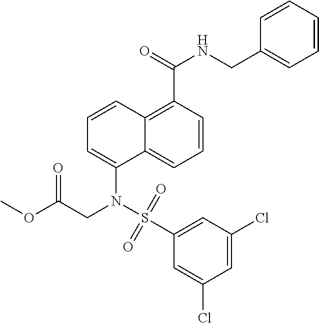

R_{f} value: 0.82 (silica gel, methylene chloride/methanol=95:5)

Mass spectrum (ESI⁺): m/z=557, 559, 561 [M+H]⁺

### (29) methyl [(3,5-dichloro-phenylsulphonyl)-(5-methylaminocarbonyl-naphthalen-1-yl)-amino]-acetate

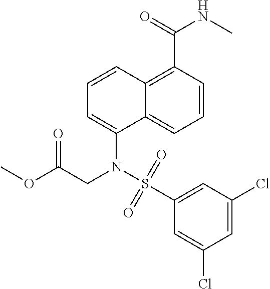

R_{f} value: 0.50 (silica gel, methylene chloride/methanol=95:5)

Mass spectrum (ESI⁺): m/z=481, 483, 485 [M+H]⁺

### (30) tert. Butyl [(5-amino-naphthalen-1-yl)-(3,5-dichloro-phenylsulphonyl)-amino]-acetate

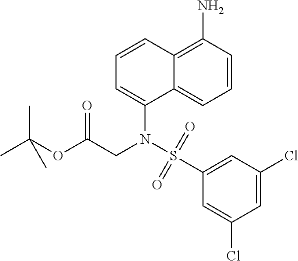

R_{f} value: 0.40 (silica gel, petroleum ether/ethyl acetate=2:1)

Mass spectrum (ESI⁺): m/z=481, 483, 485 [M+H]⁺

### (31) tert. Butyl [(3,5-dichloro-phenylsulphonyl)-(5-phenylaminocarbonyl-naphthalen-1-yl)-amino]-acetate

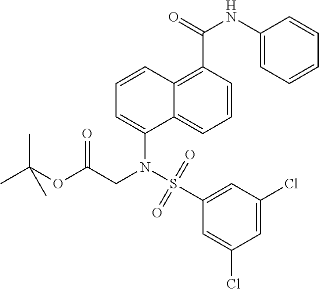

R_{f} value: 0.36 (silica gel, petroleum ether/ethyl acetate=5:1)

Mass spectrum (ESI⁻): m/z=583, 585, 587 [M−H]⁻

### (32) tert. Butyl [(5-cyano-naphthalen-1-yl)-(3,5-dichloro-phenylsulphonyl)-amino]-acetate

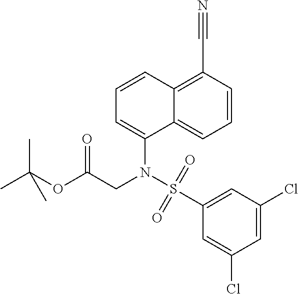

R_{f} value: 0.46 (silica gel, petroleum ether/ethyl acetate=5:1)

Mass spectrum (ESI⁺): m/z=508, 510, 512 [M+NH₄]⁺

### (32) tert. Butyl [(3,5-dichloro-phenylsulphonyl)-(6-pyrimidin-2-yl-naphthalen-2-yl)-amino]-acetate

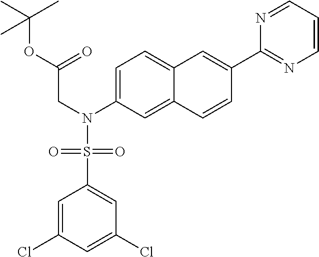

R_{f} value: 0.68 (silica gel, petroleum ether/ethyl acetate=1:1)

Mass spectrum (ESI⁺): m/z=544, 546, 548 [M+H]⁺

### (33) tert. Butyl [(3,5-dichloro-phenylsulphonyl)-(5-pyrimidin-2-yl-naphthalen-1-yl)-amino]-acetate

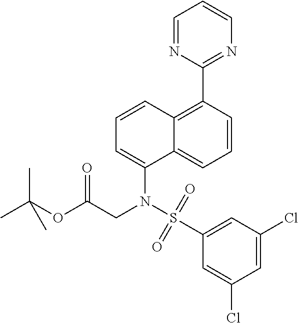

R_{f} value: 0.54 (silica gel, petroleum ether/ethyl acetate=1:1)

Mass spectrum (ESI⁺): m/z=544, 546, 548 [M+H]⁺

### (34) tert. Butyl [(3,5-dichloro-phenylsulphonyl)-(5-phenylethyl-aminocarbonyl-naphthalen-1-yl)-amino]-acetate

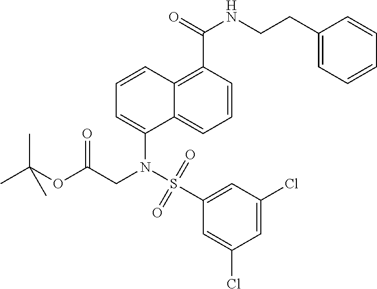

R_{f} value: 0.72 (silica gel, petroleum ether/ethyl acetate=1:1)

Mass spectrum (ESI⁺): m/z=613, 615, 617 [M+H]⁺

### (35) tert. Butyl [(3,5-dichloro-phenylsulphonyl)-(6-methoxy-naphthalen-1-yl)-amino]-acetate

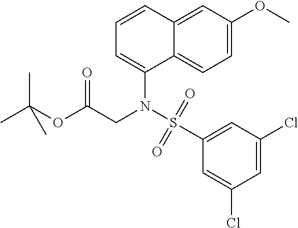

R_{f} value: 0.46 (silica gel, cyclohexane/ethyl acetate=4:1)

### (36) tert. Butyl {(3,5-dichloro-phenylsulphonyl)-[6-(3-ethoxycarbonyl-[1,2,4]oxadiazol-5-yl)-naphthalen-2-yl]-amino}-acetate

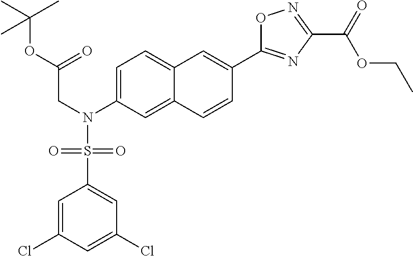

R_{f} value: 0.80 (silica gel, petroleum ether/ethyl acetate=1:1)

Mass spectrum (ESI⁺): m/z=606, 608, 610 [M+H]⁺

### (37) tert. Butyl {[6-(3-benzyl-[1,2,4]oxadiazol-5-yl)-naphthalen-2-yl]-(3,5-dichloro-phenylsulphonyl)-amino}-acetate

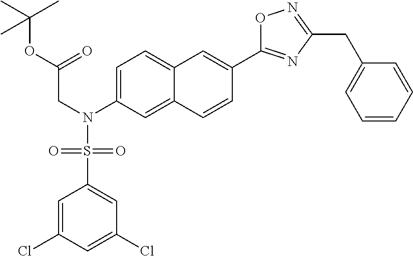

R_{f} value: 0.86 (silica gel, petroleum ether/ethyl acetate=1:1)

Mass spectrum (ESI⁺): m/z=624, 626, 628 [M+H]⁺

### (38) tert. Butyl {(3,5-dichloro-phenylsulphonyl)-[6-(3-phenyloxymethyl-[1,2,4]oxadiazol-5-yl)-naphthalen-2-yl]-amino}-acetate

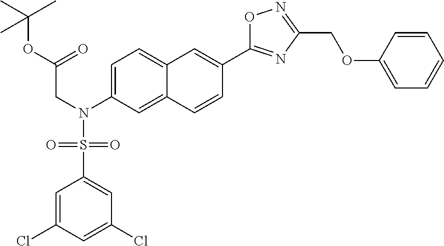

R_{f} value: 0.82 (silica gel, petroleum ether/ethyl acetate=1:1)

Mass spectrum (ESI⁺): m/z=640, 642, 644 [M+H]⁺

### (39) tert. Butyl {(3,5-dichloro-phenylsulphonyl)-[6-(3-phenylsulphonylmethyl-[1,2,4]oxadiazol-5-yl)-naphthalen-2-yl]-amino}-acetate

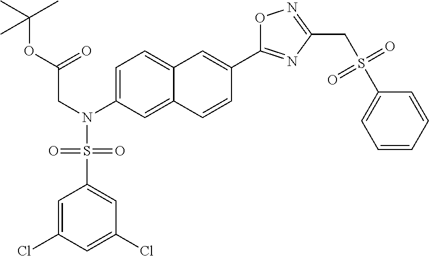

R_{f} value: 0.60 (silica gel, petroleum ether/ethyl acetate=1:1)

Mass spectrum (ESI⁺): m/z=688, 690, 692 [M+H]⁺

### (40) tert. Butyl {(3,5-dichloro-phenylsulphonyl)-[6-(5-methyl-[1,2,4]oxadiazol-3-yl)-naphthalen-1-yl]-amino}-acetate

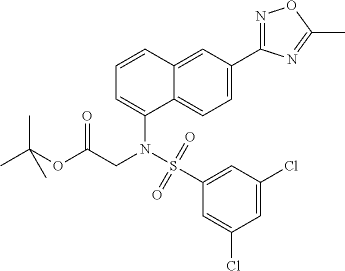

### (41) tert. Butyl ((3,5-dichloro-phenylsulphonyl)-{5-[(pyridin-4-ylmethyl)-aminocarbonyl]-naphthalen-1-yl}-amino)-acetate

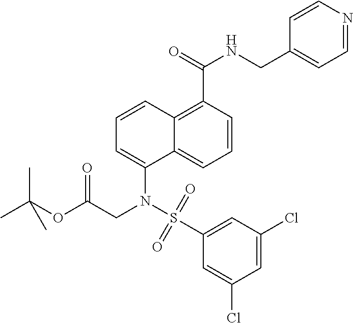

R_{f} value: 0.30 (silica gel, ethyl acetate)

Mass spectrum (ESI⁺): m/z=600, 602, 604 [M+H]⁺

### (42) (66) tert. Butyl [[5-(cyclohexylmethyl-aminocarbonyl)-naphthalen-1-yl]-(3,5-dichloro-phenylsulphonyl)-amino]-acetate

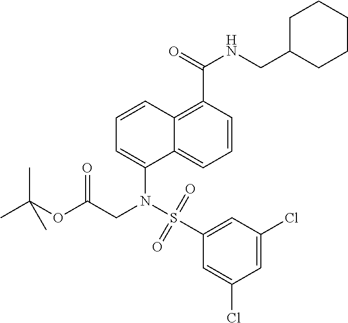

R_{f} value: 0.60 (silica gel, petroleum ether/ethyl acetate/acetic acid=70:30:0.1)

Mass spectrum (ESI⁺): m/z=605, 607, 609 [M+H]⁺

### (43) tert. Butyl ((3,5-dichloro-phenylsulphonyl)-{5-[(pyridin-2-ylmethyl)-aminocarbonyl]-naphthalen-1-yl}-amino)-acetate

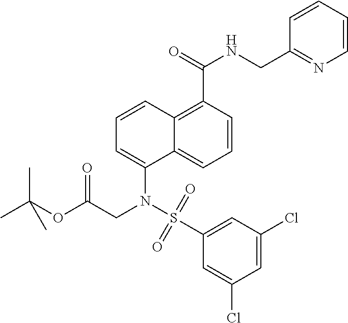

R_{f} value: 0.52 (silica gel, ethyl acetate)

Mass spectrum (ESI⁺): m/z=600, 602, 604 [M+H]⁺

### (44) tert. Butyl ((3,5-dichloro-phenylsulphonyl)-{5-[(pyridin-3-ylmethyl)-aminocarbonyl]-naphthalen-1-yl}-amino)-acetate

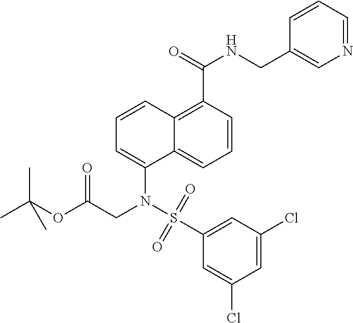

R_{f} value: 0.37 (silica gel, ethyl acetate)

Mass spectrum (ESI⁺): m/z=600, 602, 604 [M+H]⁺

### (45) tert. Butyl [(3,5-dibromo-phenylsulphonyl)-(naphthalen-1-yl)-amino]-acetate

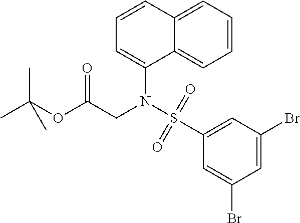

R_{f} value: 0.75 (silica gel, petroleum ether/ethyl acetate=7:3)

Mass spectrum (ESI⁺): m/z=571, 573, 575 [M+NH₄]⁺

### (46) tert. Butyl [(3-bromo-5-methyl-phenylsulphonyl)-(naphthalen-1-yl)-amino]-acetate

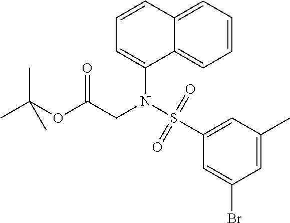

R_{f} value: 0.68 (silica gel, petroleum ether/ethyl acetate=7:3)

Mass spectrum (ESI⁺): m/z=507, 509 [M+NH₄]⁺

### (47) tert. Butyl [(3-bromo-5-chloro-phenylsulphonyl)-(naphthalen-1-yl)-amino]-acetate

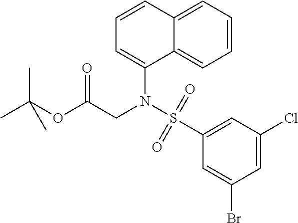

R_{f} value: 0.74 (silica gel, petroleum ether/ethyl acetate=7:3)

Mass spectrum (ESI⁺): m/z=527, 529, 531 [M+NH₄]⁺

### (48 tert. Butyl) [[5-(4-cyano-benzylaminocarbonyl)-naphthalen-1-yl]-(3,5-dichloro-phenylsulphonyl)-amino]-acetate

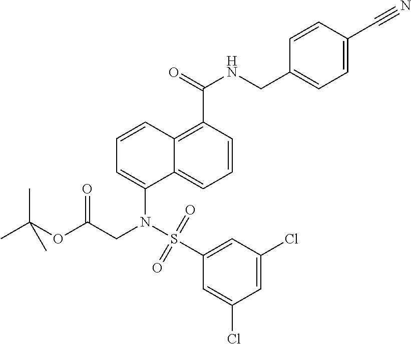

R_{f} value: 0.55 (silica gel, cyclohexane/ethyl acetate=1:1)

Mass spectrum (ESI⁺): m/z=624, 626, 628 [M+H]⁺

### (49) tert. Butyl [[5-(3-cyano-benzylaminocarbonyl)-naphthalen-1-yl]-(3,5-dichloro-phenylsulphonyl)-amino]-acetate

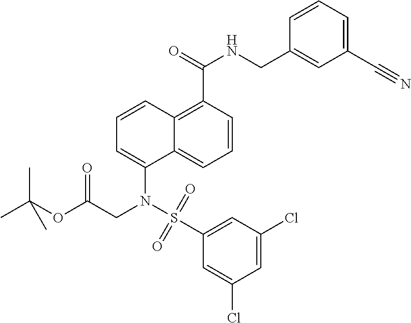

R_{f} value: 0.53 (silica gel, cyclohexane/ethyl acetate=1:1)

Mass spectrum (ESI⁺): m/z=624, 626, 628 [M+H]⁺

### (50) tert. Butyl [[5-(2-cyano-benzylaminocarbonyl)-naphthalen-1-yl]-(3,5-dichloro-phenylsulphonyl)-amino]-acetate

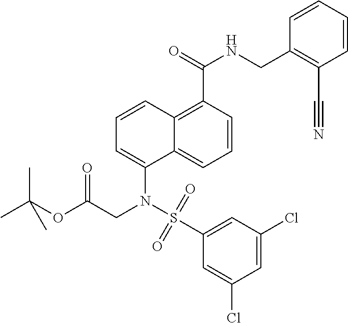

R_{f} value: 0.63 (silica gel, cyclohexane/ethyl acetate=1:1)

Mass spectrum (ESI⁻): m/z=622, 624, 626 [M−H]⁻

### (51) tert. Butyl [(2-cyano-naphthalen-1-yl)-(3,5-dichloro-phenylsulphonyl)-amino]-acetate

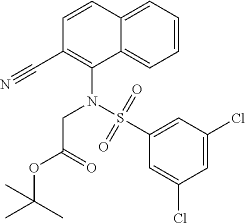

R_{f} value: 0.38 (silica gel, petroleum ether/ethyl acetate=5:1)

### (52) tert. Butyl {(3,5-dichloro-phenylsulphonyl)-[5-(2-methoxy-benzylcarbonylamino)-naphthalen-1-yl]-amino}-acetate

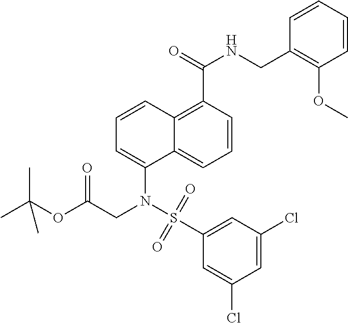

R_{f} value: 0.77 (silica gel, petroleum ether/ethyl acetate=1:1)

Mass spectrum (ESI⁺): m/z=629, 631, 633 [M+H]⁺

### (53) tert. Butyl {(3,5-dichloro-phenylsulphonyl)-[5-(3-methoxy-benzylcarbonylamino)-naphthalen-1-yl]-amino}-acetate

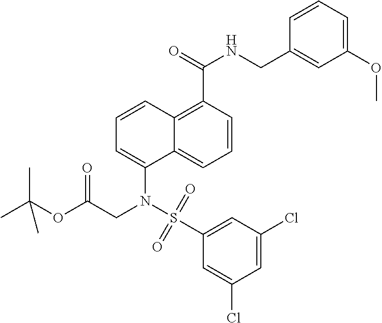

R_{f} value: 0.74 (silica gel, petroleum ether/ethyl acetate=1:1)

Mass spectrum (ESI⁺): m/z=629, 631, 633 [M+H]⁺

### (54) tert. Butyl {(3,5-dichloro-phenylsulphonyl)-[5-(4-methoxy-benzylcarbonylamino)-naphthalen-1-yl]-amino}-acetate

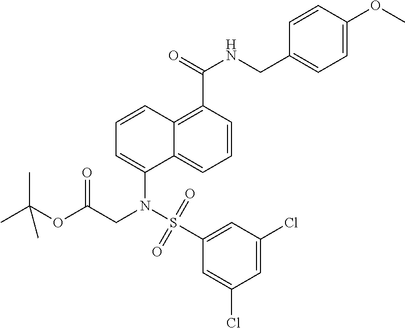

R_{f} value: 0.68 (silica gel, petroleum ether/ethyl acetate=1:1)

Mass spectrum (ESI⁺): m/z=629, 631, 633 [M+H]⁺

### (55) tert. Butyl [[3-(phenylsulphonyl-methyl-amino)-naphthalen-1-yl]-(3,5-dichloro-phenylsulphonyl)-amino]-acetate

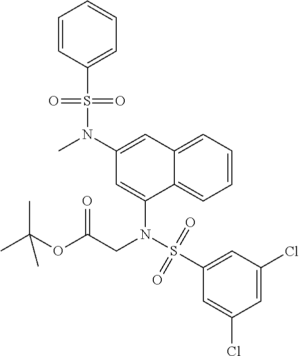

Mass spectrum (ESI⁺): m/z=652, 654, 656 [M+NH₄]⁺

### (56) N-(4-amino-naphthalen-2-yl)-N-methyl-phenylsulphonamide

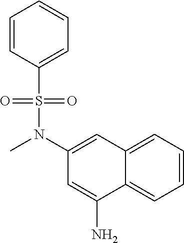

### (57) tert. Butyl [(4-benzylaminocarbonyl-naphthalen-1-yl)-(3,5-dichloro-phenylsulphonyl)-amino]-acetate

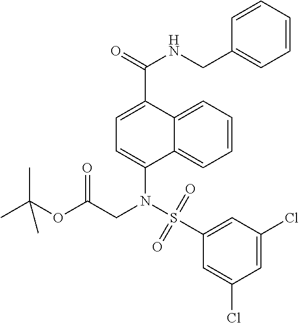

Mass spectrum (ESI⁺): m/z=599, 601, 603 [M+H]⁺

### (58) tert. Butyl [[5-(3-aminocarbonyl-benzylaminocarbonyl)-naphthalen-1-yl]-(3,5-dichloro-phenylsulphonyl)-amino]-acetate

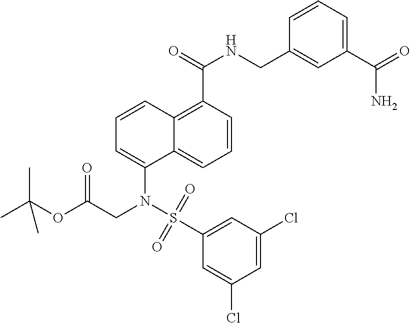

R_{f} value: 0.50 (silica gel, ethyl acetate)

Mass spectrum (ESI⁺): m/z=642, 644, 646 [M+H]⁺

### (59) tert. Butyl ((3,5-dichloro-phensulphonyl)-{5-[(pyridin-4-ylmethyl)-aminocarbonyl]-naphthalen-2-yl}-amino)-acetate

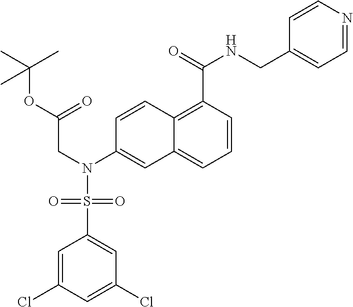

Mass spectrum (ESI⁺): m/z=600, 602, 604 [M+H]⁺

### (60) tert. Butyl ((3,5-dichloro-phensulphonyl)-{5-[(pyridin-3-ylmethyl)-aminocarbonyl]-naphthalen-2-yl}-amino)-acetate

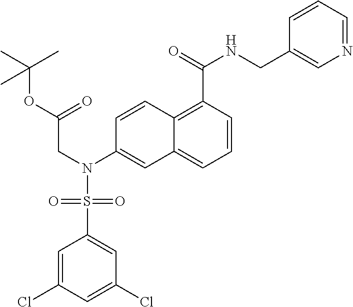

Mass spectrum (ESI⁺): m/z=600, 602, 604 [M+H]⁺

### (61) tert. Butyl ((3,5-dichloro-phenylsulphonyl)-{5-[4-(morpholin-4-ylcarbonyl)-benzylaminocarbonyl]-naphthalen-1-yl}-amino)-acetate

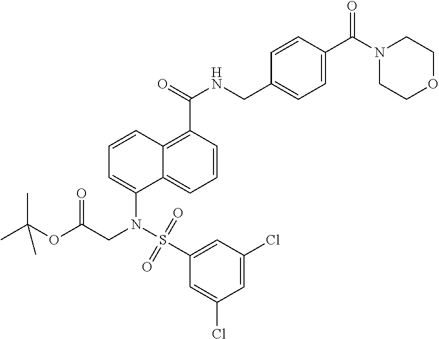

R_{f} value: 0.50 (silica gel, ethyl acetate)

Mass spectrum (ESI⁺): m/z=712, 714, 716 [M+H]⁺

### (62) tert. Butyl [[5-(4-aminocarbonyl-benzylaminocarbonyl)-naphthalen-1-yl]-(3,5-dichloro-phenylsulphonyl)-amino]-acetate

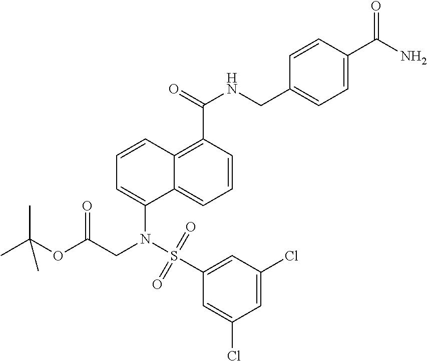

R_{f} value: 0.48 (silica gel, ethyl acetate)

Mass spectrum (ESI⁺): m/z=642, 644, 646 [M+H]⁺

### (63) tert.-butyl 4-{4-[({6-[tert.-butoxycarbonylmethyl-(3,5-dichloro-phenylsulphonyl)-amino]-naphthalene-1-carbonyl}-amino)-methyl]-benzoyl}-piperazine-1-carboxylate

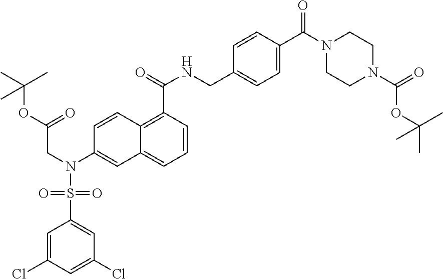

Mass spectrum (ESI⁺): m/z=811, 813, 815 [M+H]⁺

### (64) tert. Butyl ((3,5-dichloro-phenylsulphonyl)-{5-[4-(morpholin-4-ylcarbonyl)-benzylaminocarbonyl]-naphthalen-2-yl}-amino)-acetate

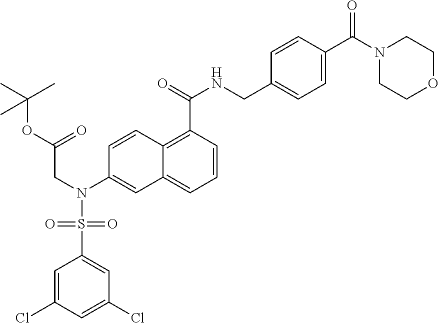

Mass spectrum (ESI⁺): m/z=712, 714, 716 [M+H]⁺

### (65) tert.-butyl 4-{4-[({5-[tert.-butoxycarbonylmethyl-(3,5-dichloro-phenylsulphonyl)-amino]-naphthalene-1-carbonyl}-amino)-methyl]-benzoyl}-piperazine-1-carboxylate

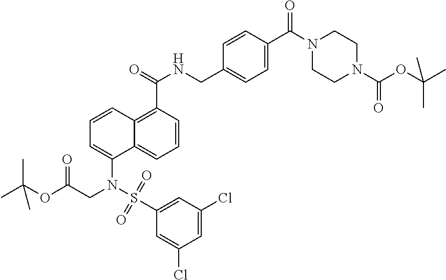

R_{f} value: 0.75 (silica gel, ethyl acetate)

Mass spectrum (ESI⁺): m/z=811, 813, 815 [M+H]⁺

### (66) tert. Butyl ((3,5-dichloro-phenylsulphonyl)-{5-[3-(morpholin-4-ylcarbonyl)-benzylaminocarbonyl]-naphthalen-1-yl}-amino)-acetate

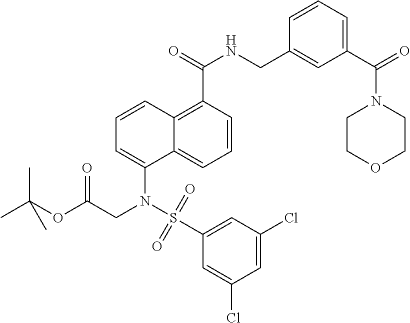

R_{f} value: 0.50 (silica gel, ethyl acetate)

Mass spectrum (ESI⁺): m/z=729, 731, 733 [M+NH₄]⁺

### (67) tert.-butyl 4-{3-[({5-[tert.-butoxycarbonylmethyl-(3,5-dichloro-phenylsulphonyl)-amino]-naphthalene-1-carbonyl}-amino)-methyl]-benzoyl}-piperazine-1-carboxylate

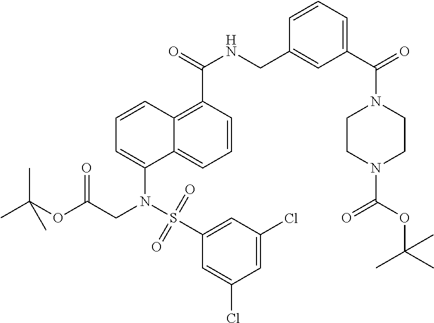

R_{f} value: 0.73 (silica gel, ethyl acetate)

Mass spectrum (ESI⁺): m/z=828, 830, 832 [M+NH₄]⁺

### (68) tert.-butyl 4-{2-[({6-[tert.-butoxycarbonylmethyl-(3,5-dichloro-phenylsulphonyl)-amino]-naphthalene-1-carbonyl}-amino)-methyl]-benzoyl}-piperazine-1-carboxylate

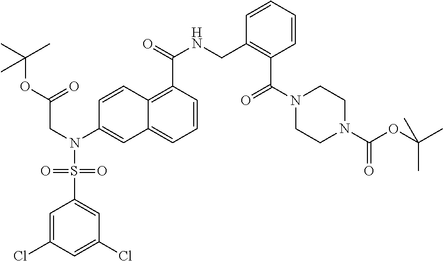

Mass spectrum (ESI⁺): m/z=811, 813, 815 [M+H]⁺

### (69) tert. Butyl ((3,5-dichloro-phenylsulphonyl)-{5-[2-(morpholin-4-ylcarbonyl)-benzylaminocarbonyl]-naphthalen-1-yl}-amino)acetate

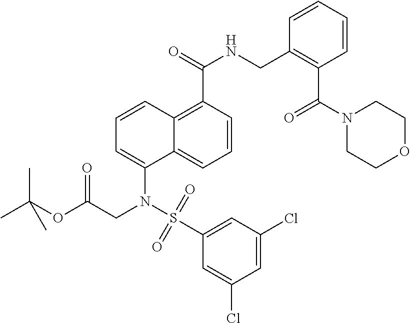

R_{f} value: 0.32 (silica gel, petroleum ether/ethyl acetate=1:2)

Mass spectrum (ESI⁺): m/z=712, 714, 716 [M+H]⁺

### (70) tert.-butyl 4-{2-[({5-[tert.-butoxycarbonylmethyl-(3,5-dichloro-phenylsulphonyl)-amino]-naphthalene-1-carbonyl}-amino)-methyl]-benzoyl}-piperazine-1-carboxylate

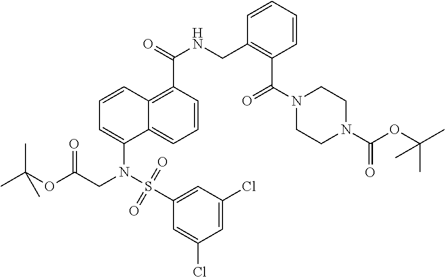

R_{f} value: 0.56 (silica gel, petroleum ether/ethyl acetate=1:2)

Mass spectrum (ESI⁺): m/z=811, 813, 815 [M+H]⁺

### (71) tert. Butyl {(3,5-dichloro-phenylsulphonyl)-[5-(3-dimethylaminocarbonyl-benzylaminocarbonyl)-naphthalen-1-yl]-amino}-acetate

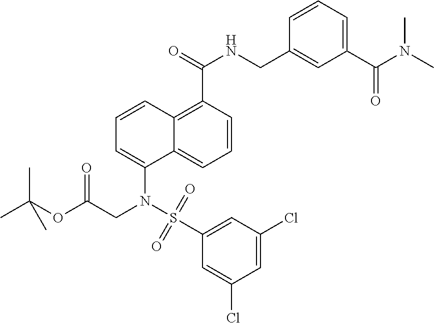

R_{f} value: 0.42 (silica gel, ethyl acetate)

Mass spectrum (ESI⁺): m/z=670, 672, 674 [M+H]⁺

### (72) tert. Butyl {(3,5-dichloro-phenylsulphonyl)-[5-(4-dimethylaminocarbonyl-benzylaminocarbonyl)-naphthalen-1-yl]-amino}-acetate

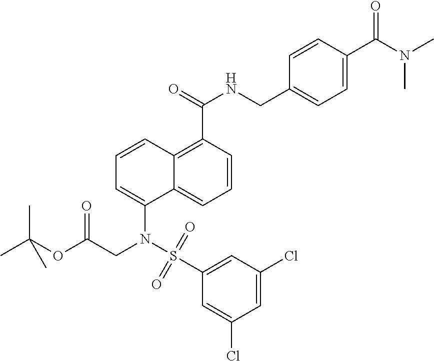

R_{f} value: 0.58 (silica gel, ethyl acetate)

Mass spectrum (ESI⁺): m/z=670, 672, 674 [M+H]⁺

### (73) tert. Butyl {[5-(2-aminocarbonyl-benzylaminocarbonyl)-naphthalen-1-yl]-(3,5-dichloro-phenylsulphonyl)-amino}-acetate

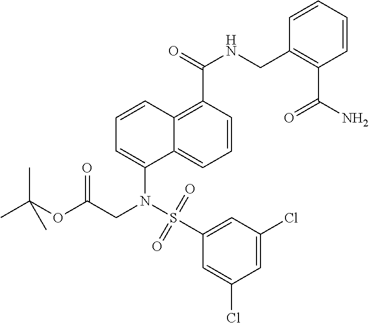

R_{f} value: 0.40 (silica gel, methylene chloride/methanol=95:5)

Mass spectrum (ESI⁺): m/z=642, 644, 646 [M+H]⁺

### (74) tert. Butyl {(3,5-dichloro-phenylsulphonyl)-[5-(4-methylaminocarbonyl-benzylaminocarbonyl)-naphthalen-1-yl]-amino}-acetate

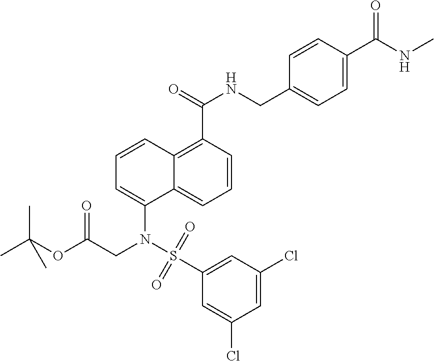

R_{f} value: 0.33 (silica gel, methylene chloride/methanol=95:5)

Mass spectrum (ESI⁺): m/z=656, 658, 660 [M+H]⁺

### (75) tert. Butyl {(3,5-dichloro-phenylsulphonyl)-[5-(3-methylaminocarbonyl-benzylaminocarbonyl)-naphthalen-1-yl]-amino}-acetate

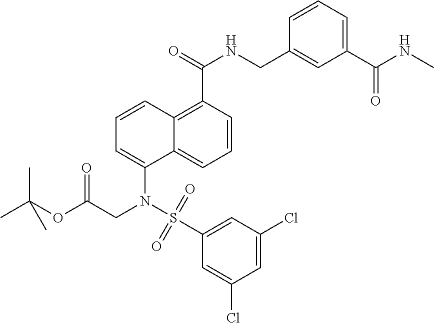

R_{f} value: 0.33 (silica gel, methylene chloride/methanol=95:5)

Mass spectrum (ESI⁺): m/z=656, 658, 660 [M+H]⁺

### (76) tert. Butyl {(3,5-dichloro-phenylsulphonyl)-[5-(4-dimethylaminocarbonyl-benzylaminocarbonyl)-naphthalen-2-yl]-amino}-acetate

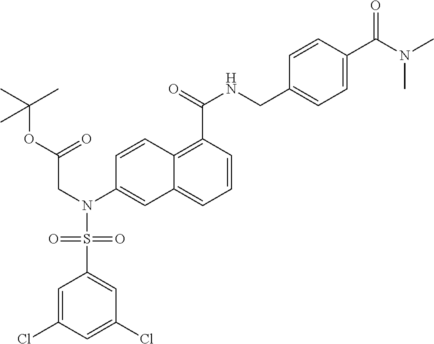

R_{f} value: 0.50 (silica gel, ethyl acetate)

Mass spectrum (ESI⁺): m/z=687, 689, 691 [M+NH₄]⁺

### (77) tert. Butyl {(3,5-dichloro-phenylsulphonyl)-[5-(3-dimethylaminocarbonyl-benzylaminocarbonyl)-naphthalen-2-yl]-amino}-acetate

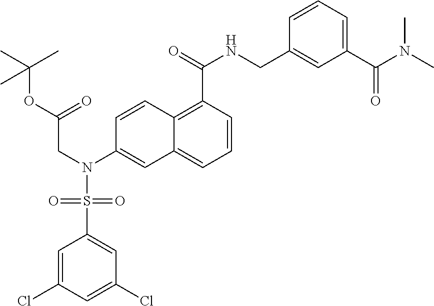

R_{f} value: 0.45 (silica gel, ethyl acetate)

Mass spectrum (ESI⁺): m/z=687, 689, 691 [M+NH₄]⁺

### (78) tert. Butyl [[3-(N-benzyl-N-methyl-aminocarbonyl)-naphthalen-1-yl]-(3,5-dichloro-phenylsulphonyl)-amino]-acetate

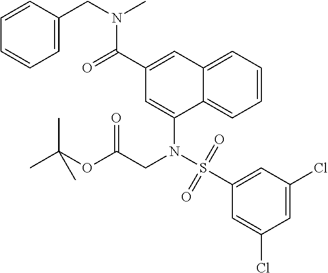

R_{f} value: 0.48 (silica gel, cyclohexane/ethyl acetate=7:3)

Mass spectrum (ESI⁺): m/z=630, 632, 634 [M+NH₄]⁺

### (79) tert. Butyl [(3,5-dichloro-phenylsulphonyl)-[3-(N-methyl-N-phenyl-aminocarbonyl)-naphthalen-1-yl]-acetate

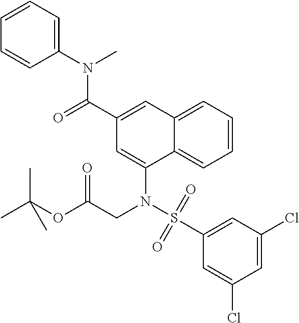

R_{f} value: 0.67 (silica gel, cyclohexane/ethyl acetate=1:1)

Mass spectrum (ESI⁺): m/z=616, 618, 620 [M+NH₄]⁺

### (80) benzyl 5-[tert.-butoxycarbonylmethyl-(3,5-dichloro-phenylsulphonyl)-amino]-naphthalene-1-carboxylate

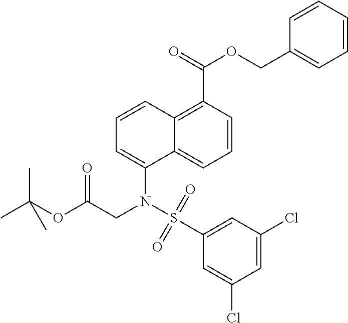

R_{f} value: 0.95 (silica gel, methylene chloride/methanol=98:2)

Mass spectrum (ESI⁺): m/z=617, 619, 621 [M+NH₄]⁺

### (81) tert. Butyl [(3,5-dichloro-phenylsulphonyl)-(5-trifluoromethanesulphonyloxy-naphthalen-1-yl)amino]-acetate

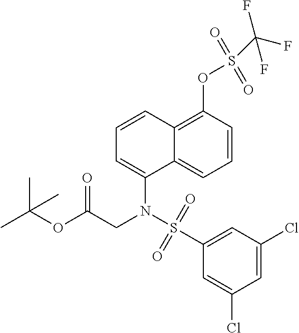

R_{f} value: 0.52 (silica gel, petroleum ether/ethyl acetate=5:1)

Mass spectrum (ESI⁺): m/z=631, 633, 635 [M+NH₄]⁺

### (82) tert. Butyl [(3,5-dichloro-phenylsulphonyl)-quinolin-8-yl-amino]-acetate

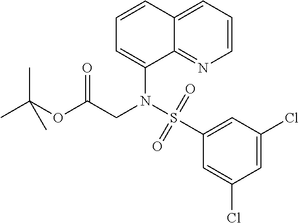

R_{f} value: 0.35 (silica gel, cyclohexane/ethyl acetate=4:1)

Mass spectrum (ESI⁺): m/z=467, 469, 471 [M+H]⁺

### (83) tert. Butyl [(3,5-dichloro-phenylsulphonyl)-(6-methoxy-quinolin-8-yl)-amino]-acetate

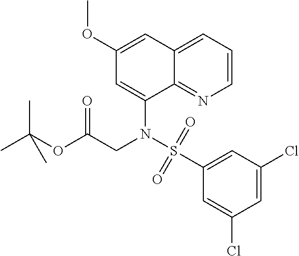

R_{f} value: 0.75 (silica gel, cyclohexane/ethyl acetate=1:1)

Mass spectrum (ESI⁺): m/z=497, 499, 501 [M+H]⁺

### Example II

### 6-(3,5-dichloro-phenylsulphonylamino)-naphthalene-1-carboxylic acid-4-cyano-benzylamide

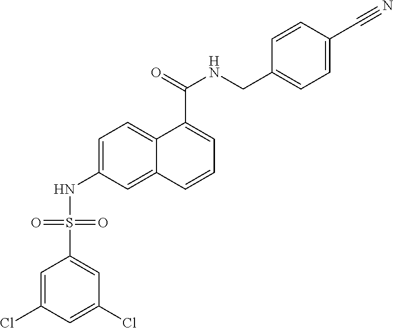

2.21 g 3,5-dichlorophenylsulphonyl chloride are added to a solution of 2.50 g 6-amino-naphthalene-1-carboxylic acid-4-cyano-benzylamide dissolved in 40 ml of pyridine while cooling with an ice bath. After one hour the ice bath is removed and the reaction mixture is stirred for another two hours at ambient temperature. Then the pyridine is distilled off in vacuo, the flask residue is taken up in methylene chloride, washed with 2N hydrochloric acid and water, dried on magnesium sulphate and evaporated down. The crude product is purified by chromatography through a silica gel column with methylene chloride/ethyl acetate (9:1 to 8:2) as eluant.

Yield: 3.53 g (83% of theory)

R_{f} value: 0.31 (silica gel, methylene chloride/ethyl acetate=9:1)

Mass spectrum (ESI⁺): m/z=510, 512, 514 [M+H]⁺

The following compounds are obtained analogously to Example II:

### (1) 6-(3,5-dichloro-phenylsulphonylamino)-naphthalene-1-carboxylic acid-benzylamide

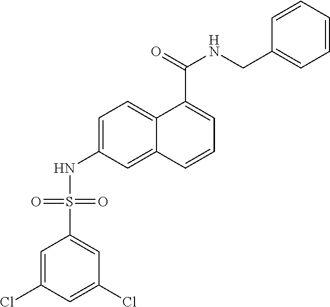

R_{f} value: 0.53 (silica gel, methylene chloride/methanol=95:5)

Mass spectrum (ESI⁺): m/z=485, 487, 489 [M+H]⁺

### (2) tert. Butyl [4-({[6-(3,5-dichloro-phenylsulphonylamino)-naphthalene-1-carbonyl]-amino}-methyl)-benzyl]-carbamate

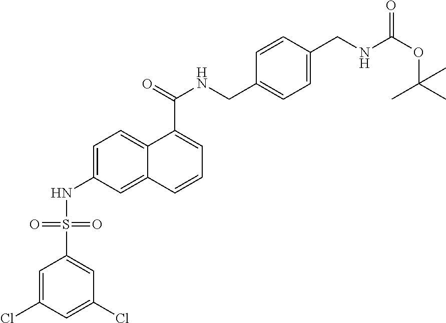

R_{f} value: 0.50 (silica gel, methylene chloride/methanol=95:5)

Mass spectrum (ESI⁻): m/z=612, 614, 616 [M−H]⁻

### (3) 6-(3,5-dichloro-phenylsulphonylamino)-naphthalene-1-carboxylic acid-methylamide

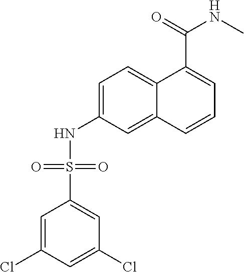

R_{f} value: 0.65 (silica gel, ethyl acetate)

Mass spectrum (ESI⁻): m/z=407, 409, 411 [M−H]⁻

### (4) benzyl 6-(3,5-dichloro-phenylsulphonylamino)-naphthalene-1-carboxylate

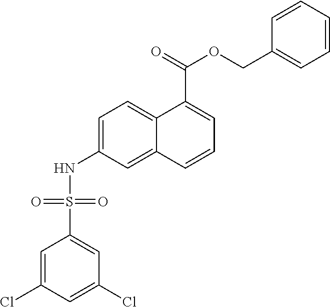

R_{f} value: 0.75 (silica gel, cyclohexane/ethyl acetate=1:1)

### (5) 6-(3,5-dichloro-phenylsulphonylamino)-naphthalene-2-carboxylic acid

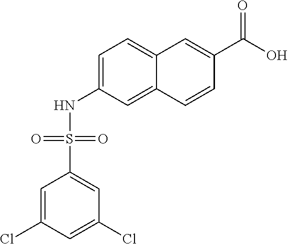

R_{f} value: 0.80 (silica gel, ethyl acetate)

Mass spectrum (ESI⁻): m/z=394, 396, 398 [M−H]⁻

### (6) 3,5-dichloro-N-naphthalen-1-yl-phenylsulphonamide

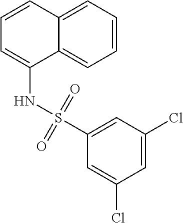

R_{f} value: 0.65 (silica gel, cyclohexane/ethyl acetate=3:1)

Mass spectrum (ESI⁻): m/z=350, 352, 354 [M−H]⁻

### (7) 6-(3,5-dichloro-phenylsulphonylamino)-naphthalene-2-carboxylic acid-benzylamide

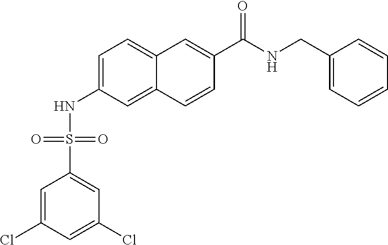

R_{f} value: 0.77 (silica gel, hexane/ethyl acetate=1:2)

### (8) 6-(3,5-dichloro-phenylsulphonylamino)-naphthalene-1-carboxylic acid

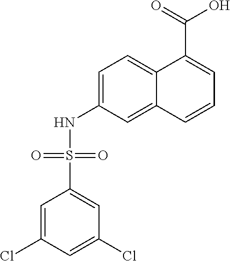

R_{f} value: 0.45 (silica gel, methylene chloride/methanol=9:1)

### (9) 6-(3,5-dimethyl-phenylsulphonylamino)-naphthalene-1-carboxylic acid-benzylamide

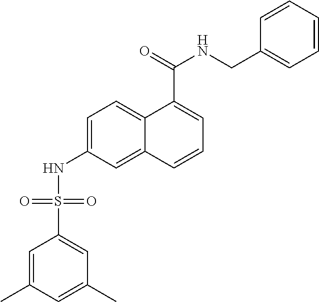

R_{f} value: 0.27 (silica gel, hexane/ethyl acetate=1:1)

### (10) 6-(3,5-dimethyl-phenylsulphonylamino)-naphthalene-2-carboxylic acid-benzylamide

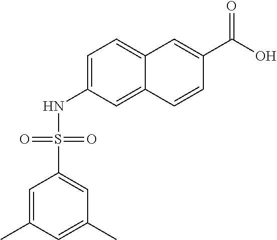

R_{f} value: 0.48 (silica gel, petroleum ether/ethyl acetate=1:1)

Mass spectrum (ESI⁺): m/z=356 [M+H]⁺

### (11) N-(7-amino-naphthalen-2-yl)-3,5-dichloro-phenylsulphonamide

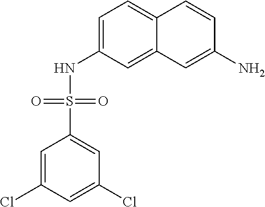

Mass spectrum (ESI⁻): m/z=365, 367, 369 [M−H]⁻

### (12) 3,5-dichloro-N-(6-hydroxy-naphthalen-2-yl)-phenylsulphonamide

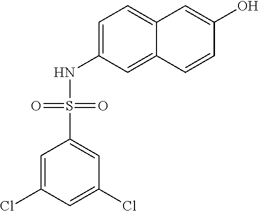

R_{f} value: 0.70 (silica gel, cyclohexane/ethyl acetate=1:1)

Mass spectrum (ESI⁺): m/z=368, 370, 372 [M+H]⁺

### (13) methyl 7-(3,5-dichloro-phenylsulphonylamino)-naphthalene-2-carboxylate

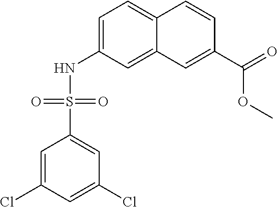

Mass spectrum (ESI⁺): m/z=410, 412, 414 [M+H]⁺

### (14) 3,5-dichloro-N-(4-methoxy-naphthalen-2-yl)-phenylsulphonamide

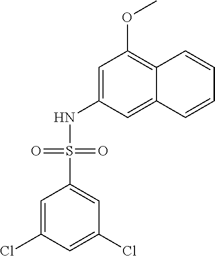

R_{f} value: 0.80 (silica gel, cyclohexane/ethyl acetate=1:1)

Mass spectrum (ESI⁺): m/z=382, 384, 386 [M+H]⁺

### (15) 5-(3,5-dichloro-phenylsulphonylamino)-naphthalene-1-carboxylic acid

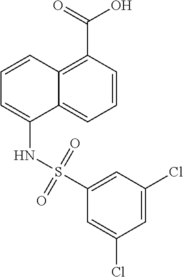

R_{f} value: 0.50 (silica gel, petroleum ether/ethyl acetate=1:1)

Mass spectrum (ESI⁻): m/z=394, 396, 398 [M−H]⁻

### (16) N-(5-amino-naphthalen-1-yl)-3,5-dichloro-phenylsulphonamide

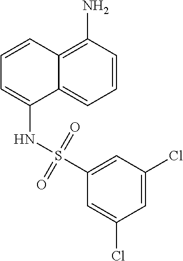

R_{f} value: 0.34 (silica gel, petroleum ether/ethyl acetate=2:1)

### (17) 3,5-dichloro-N-(6-pyrimidin-2-yl-naphthalen-2-yl)-phenylsulphonamide

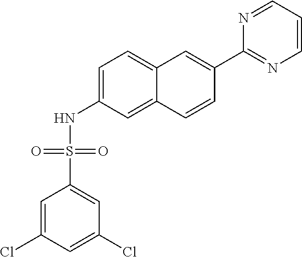

R_{f} value: 0.60 (silica gel, petroleum ether/ethyl acetate=1:1)

Mass spectrum (ESI⁺): m/z=430, 432, 434 [M+H]⁺

### (18) 3,5-dichloro-N-(5-pyrimidin-2-yl-naphthalen-1-yl)-phenylsulphonamide

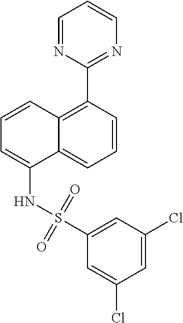

R_{f} value: 0.20 (silica gel, petroleum ether/ethyl acetate=2:1)

Mass spectrum (ESI⁺): m/z=430, 432, 434 [M+H]⁺

### (17) (3,5-dichloro-N-(6-methoxy-naphthalen-1-yl)-phenylsulphonamide

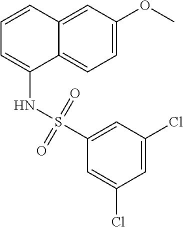

R_{f} value: 0.26 (silica gel, cyclohexane/ethyl acetate=4:1)

Mass spectrum (ESI⁺): m/z=382, 384, 386 [M+H]⁺

### (18) 3,5-dibromo-N-naphthalen-1-yl-phenylsulphonamide

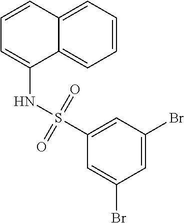

R_{f} value: 0.75 (silica gel, petroleum ether/ethyl acetate=3:2)

Mass spectrum (ESI⁻): m/z=438, 440, 442 [M−H]⁻

### (19) 3-bromo-5-methyl-N-naphthalen-1-yl-phenylsulphonamide

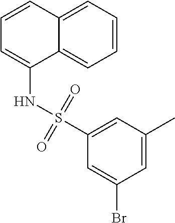

R_{f} value: 0.69 (silica gel, petroleum ether/ethyl acetate=3:2)

Mass spectrum (ESI⁻): m/z=374, 376 [M−H]⁻

### (19) 3-bromo-5-chloro-N-naphthalen-1-yl-phenylsulphonamide

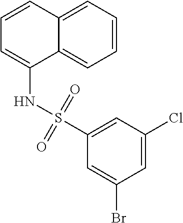

R_{f} value: 0.76 (silica gel, petroleum ether/ethyl acetate=3:2)

Mass spectrum (ESI⁻): m/z=394, 396, 398 [M−H]⁻

### (29) 3,5-dichloro-N-(2-cyano-naphthalen-1-yl)-phenylsulphonamide

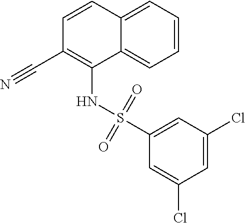

### (30) N-[3-(phenylsulphonyl-methyl-amino)-naphthalen-1-yl]-3,5-dichloro-phenylsulphonamide

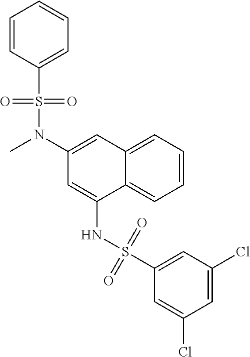

Mass spectrum (ESI⁻): m/z=519, 521, 523 [M−H]⁻

### (31) 4-(3,5-dichloro-phenylsulphonylamino)-naphthalene-1-carboxylic acid

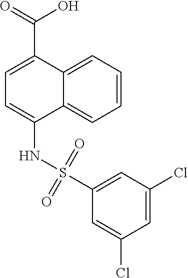

Mass spectrum (ESI⁻): m/z=394, 396, 398 [M−H]⁻

### (32) 4-(3,5-dichloro-phenylsulphonylamino)-naphthalene-2-carboxylic acid

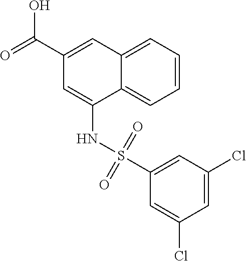

Mass spectrum (ESI⁺): m/z=413 [M+NH₄]⁺

### (33) 3,5-dichloro-N-(5-hydroxy-naphthalen-1-yl)-phenylsulphonamide

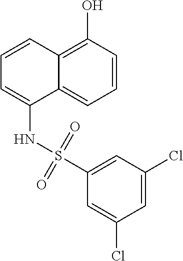

R_{f} value: 0.48 (silica gel, petroleum ether/ethyl acetate=2:1)

Mass spectrum (ESI⁻): m/z=366, 368, 370 [M−H]⁻

### (34) 3,5-dichloro-N-quinolin-8-yl-phenylsulphonamide

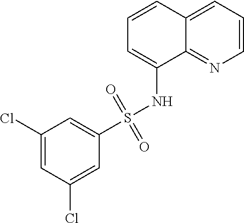

R_{f} value: 0.25 (silica gel, cyclohexane/ethyl acetate=3:1)

Mass spectrum (ESI⁺): m/z=353, 355, 357 [M+H]⁺

### (35) (3,5-dichloro-N-(6-methoxy-quinolin-8-yl)-phenylsulphonamide

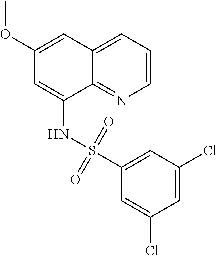

R_{f} value: 0.30 (silica gel, cyclohexane/ethyl acetate=1:1)

Mass spectrum (ESI⁺): m/z=383, 385, 387 [M+H]⁺

### Example III

### 6-amino-naphthalene-1-carboxylic acid-benzylamide

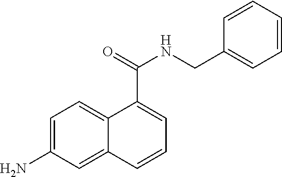

1.49 g 1-hydroxybenzotriazole, 3.53 g O-(benzotriazol-1-yl)-1,1,3,3-tetra-methyluronium-tetrafluoroborate, 6.93 ml triethylamine and 1.20 ml benzylamine are added under an argon atmosphere to 1.87 g 6-aminonaphthoic acid in a mixture of 20 ml of tetrahydrofuran and 20 ml N,N-dimethylformamide. The light brown solution is stirred for 3 h at ambient temperature. Then the solvent is distilled off using the rotary evaporator, the flask residue is slowly combined with ice water and extracted with ethyl acetate. The combined ethyl acetate extracts are washed with 2N citric acid and saturated sodium chloride solution. A precipitate is formed which is suction filtered, washed with water and ethyl acetate and dried. The filtrate is combined with a little methanol, the organic phase is separated off, dried on magnesium sulphate and evaporated down. The flask residue is combined with the suction filtered precipitate, stirred with ethyl acetate, suction filtered, washed with some ethyl acetate and diethyl ether and dried in the desiccator.

Yield: 1.81 g (66% of theory)

R_{f} value: 0.50 (silica gel, methylene chloride/methanol=95:5)

Mass spectrum (ESI⁺): m/z=277 [M+H]⁺

The following compounds are obtained analogously to Example III:

### (1) tert. Butyl (4-{[(6-amino-naphthalene-1-carbonyl)-amino]-methyl}-benzyl)-carbamate

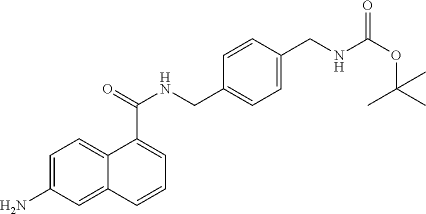

R_{f} value: 0.48 (silica gel, petroleum ether/ethyl acetate=1:2)

Mass spectrum (ESI⁺): m/z=406 [M+H]⁺

### (2) 6-amino-naphthalene-2-carboxylic acid-benzylamide

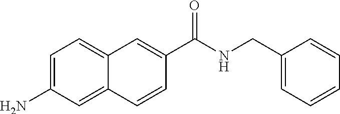

R_{f} value: 0.83 (silica gel, ethyl acetate)

### (3) tert. Butyl {(3,5-dichloro-phenylsulphonyl))-[5-(pyrrolidine-1-carbonyl)-naphthalen-2-yl]-amino}-acetate

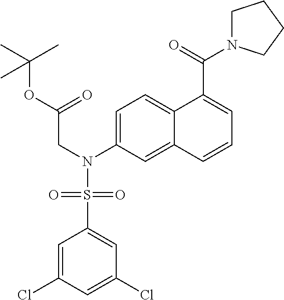

Mass spectrum (ESI⁺): m/z=563, 565, 567 [M+H]⁺

### (4) tert. Butyl [(3,5-dichloro-phenylsulphonyl)-(5-isopropylaminocarbonyl-naphthalen-2-yl)-amino]-acetate

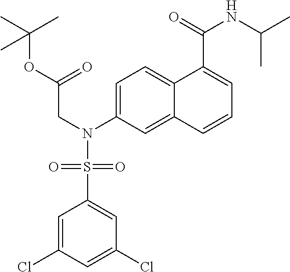

Mass spectrum (ESI⁺): m/z=551, 553, 555 [M+H]⁺

### (5) tert. Butyl {(3,5-dichloro-phenylsulphonyl)-[5-(2-hydroxy-ethylaminocarbonyl)-naphthalen-2-yl]-amino}-acetate

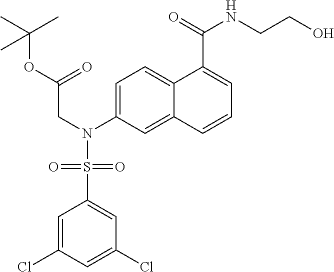

Mass spectrum (ESI⁺): m/z=553, 555, 557 [M+H]⁺

### Example IV

### Methyl [[5-(4-aminomethyl-benzylaminocarbonyl)-naphthalen-2-yl]-(3,5-dichloro-phenylsulphonyl)-amino]-acetate

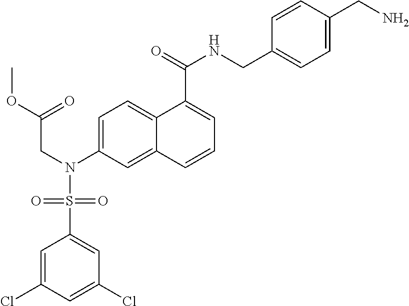

4 ml isopropanolic hydrochloric acid (5-6M) are added to 500 mg methyl [{5-[4-(tert.-butoxycarbonylamino-methyl)-benzylaminocarbonyl]-naphthalen-2-yl}-(3,5-dichloro-phenylsulphonyl)-amino]-acetate in 15 ml methylene chloride and the reaction mixture is stirred for 4 h at ambient temperature. For working up the reaction mixture is combined with some water and saturated sodium carbonate solution and extracted with methylene chloride. The combined extracts are washed with saturated sodium chloride solution, dried on magnesium sulphate and evaporated down. The flask residue is stirred with tert.-butylmethylether, suction filtered, washed with tert.-butylmethylether and dried in the desiccator.

Yield: 375 mg (88% of theory)

R_{f} value: 0.47 (silica gel, methylene chloride/methanol/conc. Aqueous ammonia=90:10:1)

Mass spectrum (ESI⁺): m/z=586, 588, 590 [M+H]⁺

### Example V

### 6-amino-naphthalene-1-carboxylic acid-methylamide

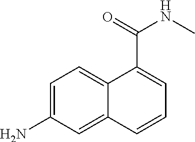

5.40 ml diisopropylethylamine and 5.30 g O-(benzotriazol-1-yl)-1,1,3,3-tetramethyluronium-tetrafluoroborate are added to 3.00 g 6-aminonaphthoic acid in 35 ml N,N-dimethylformamide. After ten minutes 12.02 ml methylamine solution (2M in tetrahydrofuran) are added and the reaction mixture is stirred overnight at ambient temperature. For working up the reaction mixture is diluted with 150 ml of ethyl acetate and washed with water. The organic phase is extracted with 1N hydrochloric acid. The combined aqueous extracts are made alkaline and extracted with ethyl acetate. The combined ethyl acetate-extracts are washed with saturated sodium chloride solution, dried on magnesium sulphate and evaporated down. The crude product is stirred with a little ethyl acetate, suction filtered, washed and dried.

Yield: 2.15 g (67% of theory)

R_{f} value: 0.40 (silica gel, ethyl acetate)

Mass spectrum (ESI⁺): m/z=201 [M+H]⁺

The following compounds are obtained analogously to Example V:

### (1) tert. Butyl [[5-(cyclohexylmethyl-aminocarbonyl)-naphthalen-2-yl]-(3,5-dichloro-phenylsulphonyl)-amino]-acetate

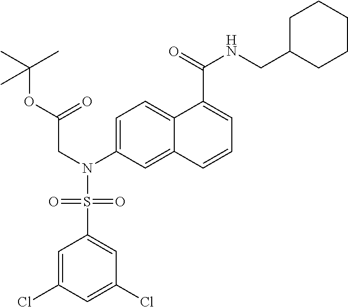

### (2) tert. Butyl [(3,5-dichloro-phenylsulphonyl)-(5-phenylaminocarbonyl-naphthalen-2-yl)-amino]-acetate

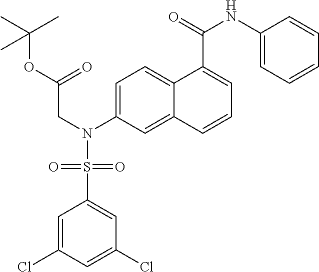

### (3) tert. Butyl {(3,5-dichloro-phenylsulphonyl)-[5-(3,4-dihydro-1H-isoquinoline-2-carbonyl)-naphthalen-2-yl]-amino}-acetate

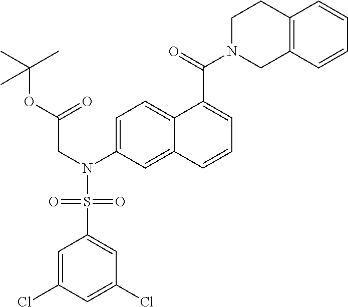

### (4) tert. Butyl [(3,5-dichloro-phenylsulphonyl)-(5-phenylethylaminocarbonyl-naphthalen-2-yl)-amino]-acetate

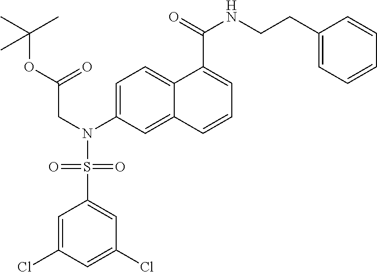

### (5) tert. Butyl [[5-(N-benzyl-N-methyl-aminocarbonyl)-naphthalen-2-yl]-(3,5-dichloro-phenylsulphonyl)-amino]-acetate

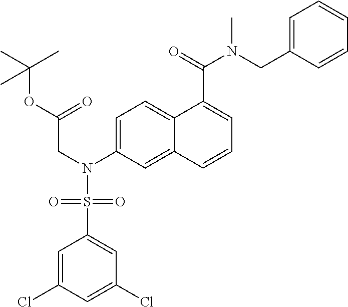

### (6) 6-(3,5-dichloro-phenylsulphonylamino)-naphthalene-2-carboxylic acid-(2-hydroxy-ethyl)-amide

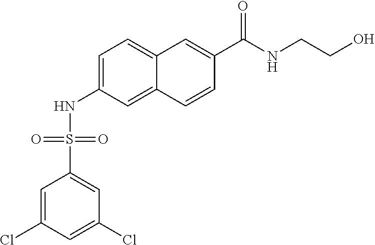

R_{f} value: 0.50 (silica gel, ethyl acetate)

### (7) 6-(3,5-dichloro-phenylsulphonylamino)-naphthalene-2-carboxylic acid-phenylamide

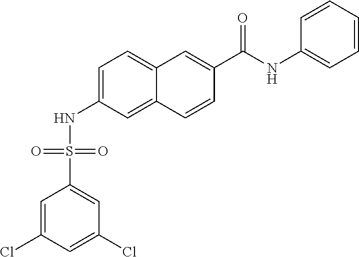

### (8) 6-(3,5-dichloro-phenylsulphonylamino)-naphthalene-2-carboxylic acid-methylamide

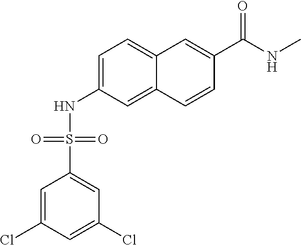

R_{f} value: 0.71 (silica gel, methylene chloride/methanol=95:5)

Mass spectrum (ESI⁺): m/z=409, 411, 413 [M+H]⁺

### (9) 6-(3,5-dimethyl-phenylsulphonylamino)-naphthalene-2-carboxylic acid-benzylamide

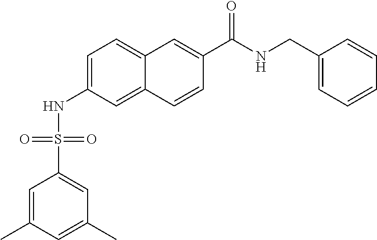

R_{f} value: 0.50 (silica gel, petroleum ether/ethyl acetate=1:1)

Mass spectrum (ESI⁺): m/z=445 [M+H]⁺

### (10) 5-(3,5-dichloro-phenylsulphonylamino)-naphthalene-1-carboxylic acid-benzylamide

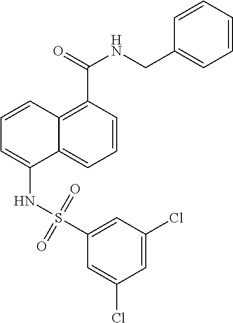

R_{f} value: 0.68 (silica gel, petroleum ether/ethyl acetate=1:1)

Mass spectrum (ESI⁺): m/z=485, 487, 489 [M+H]⁺

### (11) 5-(3,5-dichloro-phenylsulphonylamino)-naphthalene-1-carboxylic acid-methylamide

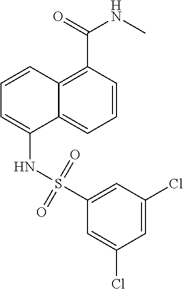

R_{f} value: 0.45 (silica gel, petroleum ether/ethyl acetate=1:2)

Mass spectrum (ESI⁺): m/z=409, 411, 413 [M+H]⁺

### (12) 5-(3,5-dichloro-phenylsulphonylamino)-naphthalene-1-carboxylic acid-phenylamide

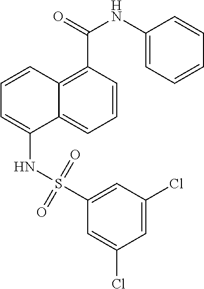

R_{f} value: 0.81 (silica gel, petroleum ether/ethyl acetate=1:1)

Mass spectrum (ESI⁺): m/z=471, 473, 475 [M+H]⁺

### (13) 5-(3,5-dichloro-phenylsulphonylamino)-naphthalene-1-carboxylic acid-phenylethylamide

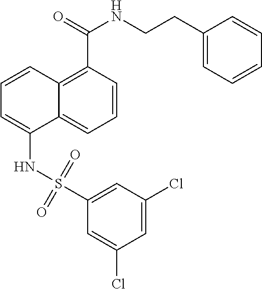

R_{f} value: 0.27 (silica gel, methylene chloride/methanol=95:5)

Mass spectrum (ESI⁺): m/z=499, 501, 503 [M+H]⁺

### (14) 5-(3,5-dichloro-phenylsulphonylamino)-naphthalene-1-carboxylic acid-2-methoxy-benzylamide

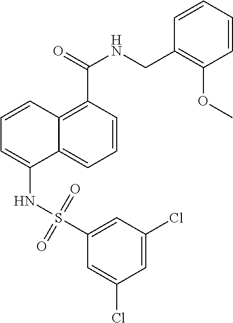

R_{f} value: 0.59 (silica gel, petroleum ether/ethyl acetate=1:1)

Mass spectrum (ESI⁺): m/z=515, 517, 519 [M+H]⁺

### (15) 5-(3,5-dichloro-phenylsulphonylamino)-naphthalene-1-carboxylic acid-3-methoxy-benzylamide

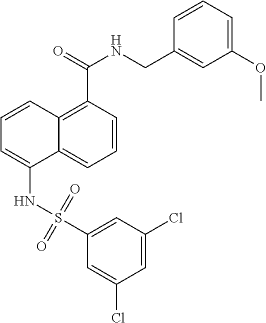

R_{f} value: 0.54 (silica gel, petroleum ether/ethyl acetate=1:1)

Mass spectrum (ESI⁺): m/z=515, 517, 519 [M+H]⁺

### (16) 5-(3,5-dichloro-phenylsulphonylamino)-naphthalene-1-carboxylic acid-4-methoxy-benzylamide

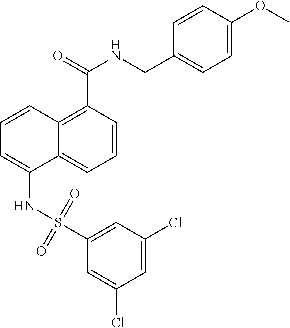

R_{f} value: 0.52 (silica gel, petroleum ether/ethyl acetate=1:1)

Mass spectrum (ESI⁺): m/z=515, 517, 519 [M+H]⁺

### (17) 4-(3,5-dichloro-phenylsulphonylamino)-naphthalene-1-carboxylic acid-benzylamide

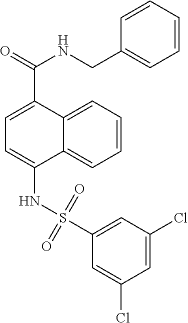

R_{f} value: 0.52 (silica gel, petroleum ether/ethyl acetate=1:1)

Mass spectrum (ESI⁻): m/z=483, 485, 487 [M−H]⁻

### (18) 5-(3,5-dichloro-phenylsulphonylamino)-naphthalene-1-carboxylic acid-3-aminocarbonyl-benzylamide

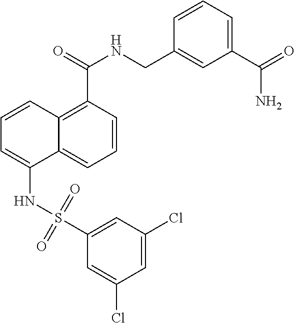

R_{f} value: 0.48 (silica gel, ethyl acetate)

Mass spectrum (ESI⁺): m/z=528, 530, 532 [M+H]⁺

### (19) 6-(3,5-dichloro-phenylsulphonylamino)-naphthalene-1-carboxylic acid-(pyridin-4-ylmethyl)-amide

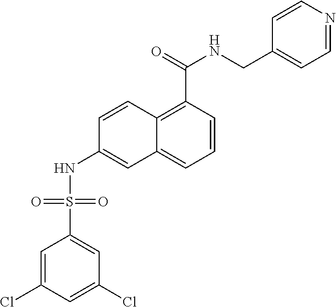

Mass spectrum (ESI⁺): m/z=486, 488, 490 [M+H]⁺

### (20) 6-(3,5-dichloro-phenylsulphonylamino)-naphthalene-1-carboxylic acid-(pyridin-3-ylmethyl)-amide

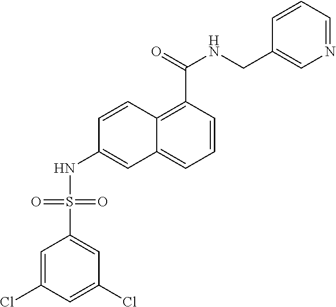

Mass spectrum (ESI⁺): m/z=486, 488, 490 [M+H]⁺

### (21) 5-(3,5-dichloro-phenylsulphonylamino)-naphthalene-1-carboxylic acid-4-(morpholine-4-carbonyl)-benzylamide

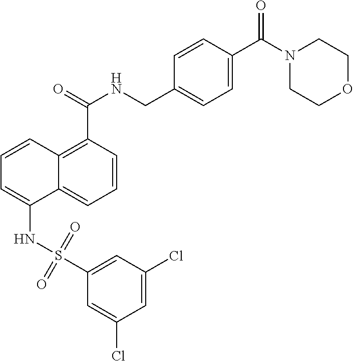

R_{f} value: 0.45 (silica gel, ethyl acetate)

Mass spectrum (ESI⁺): m/z=598, 600, 602 [M+H]⁺

### (22) ethyl 4-({[5-(3,5-dichloro-phenylsulphonylamino)-naphthalene-1-carbonyl]-amino}-methyl)-benzoate

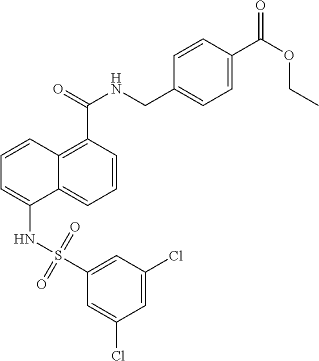

R_{f} value: 0.52 (silica gel, methylene chloride/methanol=95:5)

Mass spectrum (ESI⁺): m/z=557, 559, 561 [M+H]⁺

### (23) 5-(3,5-dichloro-phenylsulphonylamino)-naphthalene-1-carboxylic acid-4-aminocarbonyl-benzylamide

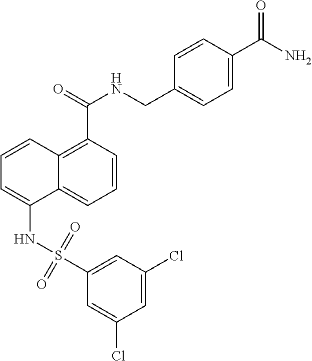

R_{f} value: 0.42 (silica gel, ethyl acetate)

Mass spectrum (ESI⁺): m/z=528, 530, 532 [M+H]⁺

### (24) tert.-butyl 4-[4-({[6-(3,5-dichloro-phenylsulphonylamino)-naphthalene-1-carbonyl]-amino}-methyl)benzoyl]-piperazine-1-carboxylate

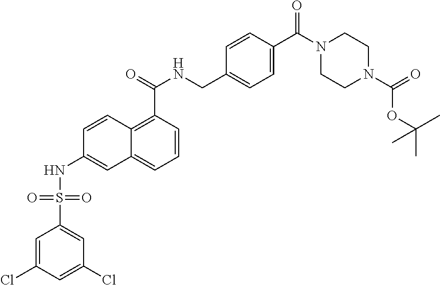

Mass spectrum (ESI⁺): m/z=697, 699, 701 [M+H]⁺

### (25) ethyl 4-({[6-(3,5-dichloro-phenylsulphonylamino)-naphthalene-1-carbonyl]-amino}-methyl)-benzoate

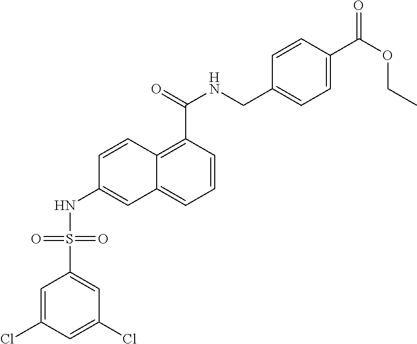

Mass spectrum (ESI⁺): m/z=557, 559, 561 [M+H]⁺

### (26) 6-(3,5-dichloro-phenylsulphonylamino)-naphthalene-1-carboxylic acid-4-(morpholine-4-carbonyl)-benzylamide

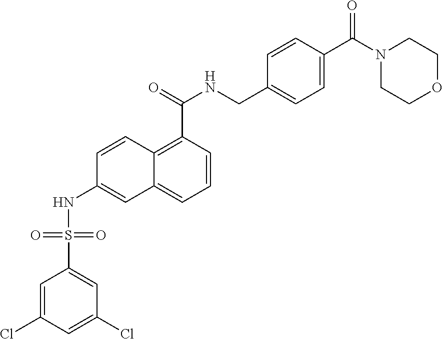

Mass spectrum (ESI⁺): m/z=598, 600, 602 [M+H]⁺

### (27) tert.-butyl 4-[4-({[5-(3,5-dichloro-phenylsulphonylamino)-naphthalene-1-carbonyl]-amino}methyl)benzoyl]-piperazine-1-carboxylate

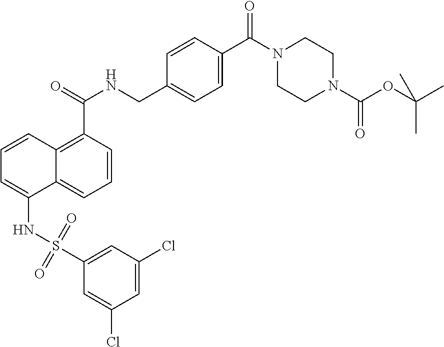

R_{f} value: 0.70 (silica gel, ethyl acetate)

Mass spectrum (ESI⁺): m/z=697, 699, 701 [M+H]⁺

### (28) 5-(3,5-dichloro-phenylsulphonylamino)-naphthalene-1-carboxylic acid-3-(morpholin-4-ylcarbonyl)-benzylamide

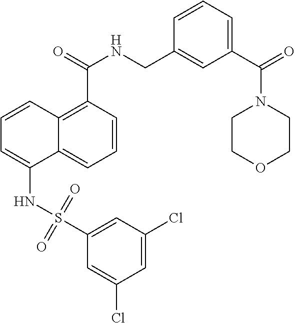

R_{f} value: 0.46 (silica gel, ethyl acetate)

Mass spectrum (ESI⁺): m/z=598, 600, 602 [M+H]⁺

### (29) methyl 3-({[5-(3,5-dichloro-phenylsulphonylamino)-naphthalene-1-carbonyl]-amino}-methyl)benzoate

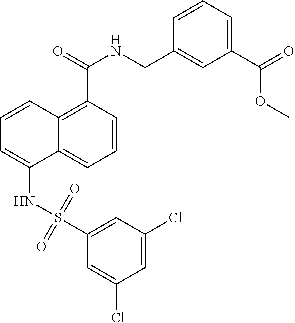

R_{f} value: 0.55 (silica gel, methylene chloride/methanol=95:5)

Mass spectrum (ESI⁺): m/z=543, 545, 547 [M+H]⁺

### (30) tert.-butyl 4-[3-({[5-(3,5-dichloro-phenylsulphonylamino)-naphthalene-1-carbonyl]-amino}-methyl)-benzoyl]-piperazine-1-carboxylate

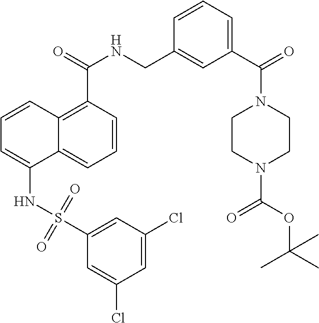

R_{f} value: 0.74 (silica gel, ethyl acetate)

Mass spectrum (ESI⁻): m/z=685, 697, 699 [M−H]⁻

### (31) tert.-butyl 4-[2-({[6-(3,5-dichloro-phenylsulphonylamino)-naphthalene-1-carbonyl]-amino}-methyl)-benzoyl]-piperazine-1-carboxylate

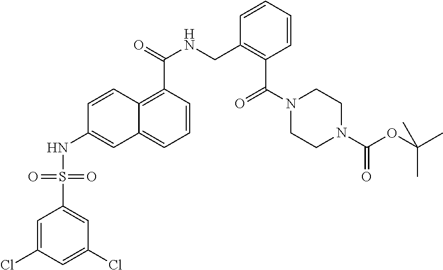

Mass spectrum (ESI⁺): m/z=697, 699, 701 [M+H]⁺

### (32) 5-(3,5-dichloro-phenylsulphonylamino)-naphthalene-1-carboxylic acid-2-(morpholin-4-ylcarbonyl)-benzylamide

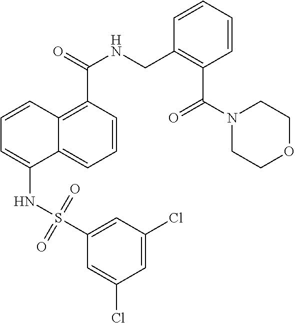

R_{f} value: 0.18 (silica gel, petroleum ether/ethyl acetate=1:2)

Mass spectrum (ESI⁺): m/z=598, 600, 602 [M+H]⁺

### (33) tert.-butyl 4-[2-({[5-(3,5-dichloro-phenylsulphonylamino)-naphthalene-1-carbonyl]-amino}-methyl)-benzoyl]-piperazine-1-carboxylate

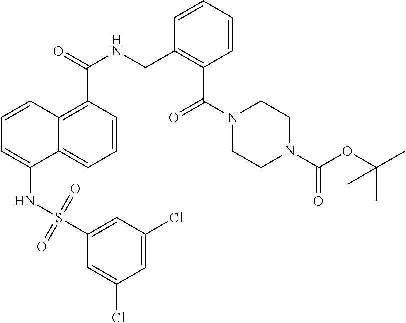

R_{f} value: 0.35 (silica gel, petroleum ether/ethyl acetate=1:2)

Mass spectrum (ESI⁺): m/z=697, 699, 701 [M+H]⁺

### (34) 5-(3,5-dichloro-phenylsulphonylamino)-naphthalene-1-carboxylic acid-3-dimethylaminocarbonyl-benzylamide

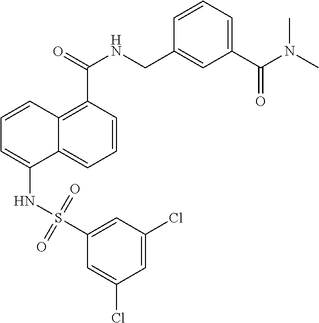

R_{f} value: 0.45 (silica gel, ethyl acetate)

Mass spectrum (ESI⁺): m/z=556, 558, 560 [M+H]⁺

### (35) 5-(3,5-dichloro-phenylsulphonylamino)-naphthalene-1-carboxylic acid-4-dimethylaminocarbonyl-benzylamide

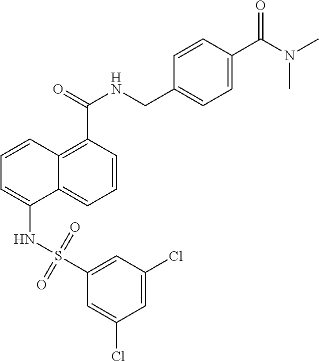

R_{f} value: 0.33 (silica gel, ethyl acetate)

Mass spectrum (ESI⁺): m/z=556, 558, 560 [M+H]⁺

### (36) 5-(3,5-dichloro-phenylsulphonylamino)-naphthalene-1-carboxylic acid-2-aminocarbonyl-benzylamide

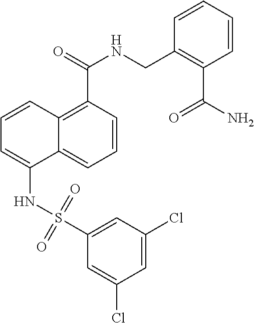

R_{f} value: 0.63 (silica gel, ethyl acetate)

Mass spectrum (ESI⁺): m/z=528, 530, 532 [M+H]⁺

### (37) 5-(3,5-dichloro-phenylsulphonylamino)-naphthalene-1-carboxylic acid-4-methylaminocarbonyl-benzylamide

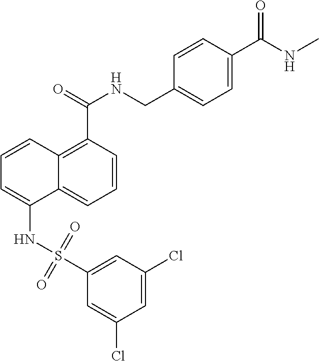

R_{f} value: 0.46 (silica gel, ethyl acetate)

Mass spectrum (ESI⁺): m/z=542, 544, 546 [M+H]⁺

### (38) 5-(3,5-dichloro-phenylsulphonylamino)-naphthalene-1-carboxylic acid-3-methylaminocarbonyl-benzylamide

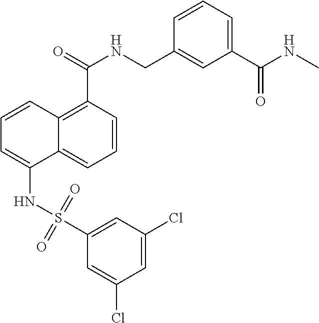

R_{f} value: 0.50 (silica gel, ethyl acetate)

Mass spectrum (ESI⁺): m/z=542, 544, 546 [M+H]⁺

### (39) 6-(3,5-dichloro-phenylsulphonylamino)-naphthalene-1-carboxylic acid-4-dimethylaminocarbonyl-benzylamide

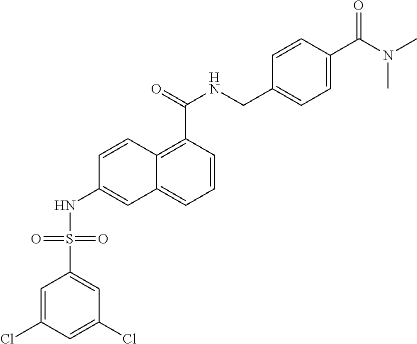

R_{f} value: 0.25 (silica gel, ethyl acetate)

Mass spectrum (ESI⁺): m/z=556, 558, 560 [M+H]⁺

### (40) 6-(3,5-dichloro-phenylsulphonylamino)-naphthalene-1-carboxylic acid-3-dimethylaminocarbonyl-benzylamide

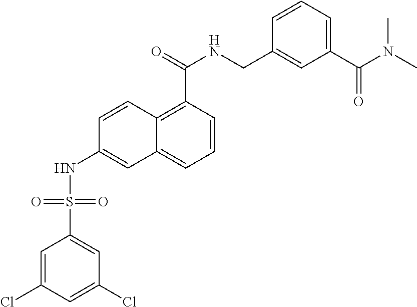

R_{f} value: 0.30 (silica gel, ethyl acetate)

Mass spectrum (ESI⁺): m/z=556, 558, 560 [M+H]⁺

### (41) 4-(3,5-dichloro-phenylsulphonylamino)-naphthalene-2-carboxylic acid-benzyl-methyl-amide

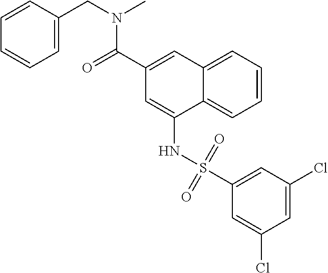

R_{f} value: 0.55 (silica gel, methylene chloride/methanol=95:5)

Mass spectrum (ESI⁺): m/z=516, 518, 520 [M+NH₄]⁺

### (42) 4-(3,5-dichloro-phenylsulphonylamino)-naphthalene-2-carboxylic acid-methyl-phenyl-amide

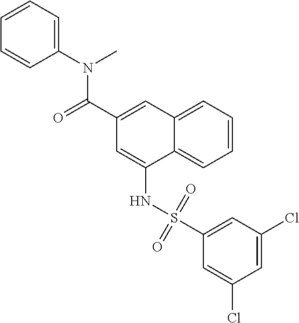

R_{f} value: 0.23 (silica gel, cyclohexane/ethyl acetate=7:3)

Mass spectrum (ESI⁺): m/z=485, 487, 489 [M+H]⁺

### (43) tert. Butyl [(3,5-dichloro-phenylsulphonyl)-(5-{[6-(2-dimethylamino-ethylamino)-pyridin-3-ylmethyl]-aminocarbonyl}-naphthalen-1-yl)amino]-acetate

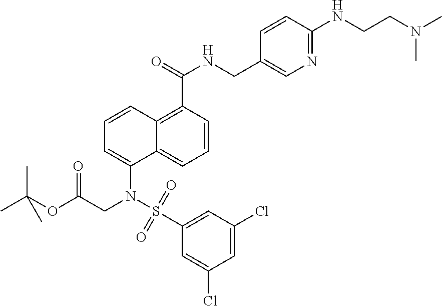

R_{f} value: 0.22 (silica gel, methylene chloride/methanol/conc. Aqueous ammonia=90:10:0.1)

Mass spectrum (ESI⁺): m/z=686, 688, 690 [M+H]⁺

### (44) tert. Butyl [(3,5-dichloro-phenylsulphonyl)-(5-{[2-(2-dimethylamino-ethylamino)-pyridin-4-ylmethyl]-aminocarbonyl}-naphthalen-1-yl)amino]-acetate

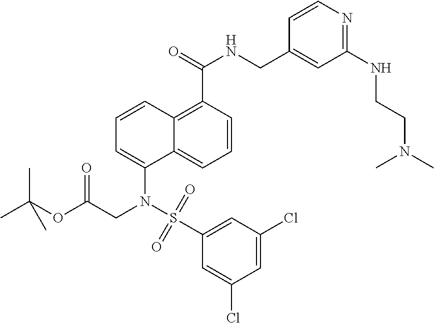

R_{f} value: 0.43 (silica gel, methylene chloride/methanol/conc. Aqueous ammonia=90:10:0.1)

Mass spectrum (ESI⁺): m/z=686, 688, 690 [M+H]⁺

### (45) tert. Butyl {(3,5-dichloro-phenylsulphonyl)-[5-({2-N-[(2-dimethylamino-ethyl)-N-methyl-amino]-pyridin-4-ylmethyl}-aminocarbonyl)-naphthalen-1-yl]-amino}-acetate

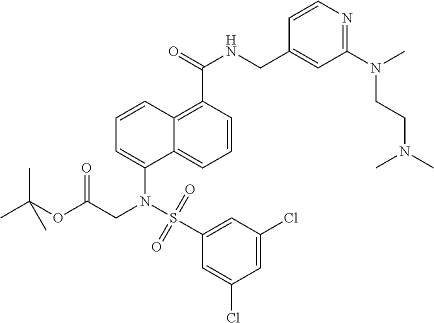

R_{f} value: 0.25 (silica gel, methylene chloride/methanol/conc. Aqueous ammonia=90:10:0.1)

Mass spectrum (ESI⁺): m/z=700, 702, 704 [M+H]⁺

### (46) tert. Butyl {(3,5-dichloro-phenylsulphonyl)-[5-({6-N-[(2-dimethylamino-ethyl)-N-methyl-amino]-pyridin-3-ylmethyl}-aminocarbonyl)-naphthalen-1-yl]-amino}-acetate

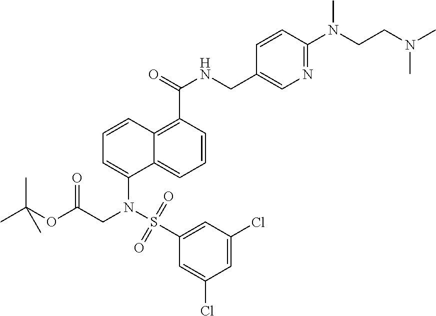

R_{f} value: 0.30 (silica gel, methylene chloride/methanol/conc. Aqueous ammonia=90:10:0.1)

Mass spectrum (ESI⁺): m/z=700, 702, 704 [M+H]⁺

### (47) tert. Butyl [(3,5-dichloro-phenylsulphonyl)-(5-{[6-(2-dimethylamino-ethylamino)-pyridin-3-ylmethyl]-aminocarbonyl}-naphthalen-2-yl)-amino]acetate

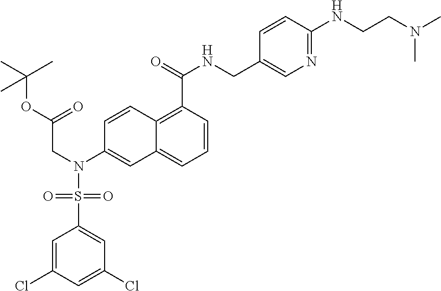

R_{f} value: 0.45 (silica gel, methylene chloride/methanol/conc. Aqueous ammonia=90:10:0.1)

Mass spectrum (ESI⁺): m/z=686, 688, 690 [M+H]⁺

### (48) tert. Butyl {(3,5-dichloro-phenylsulphonyl)-[5-({2-N-[(2-dimethylamino-ethyl)-N-methyl-amino]-pyridin-4-ylmethyl}-aminocarbonyl)-naphthalen-2-yl]-amino}-acetate

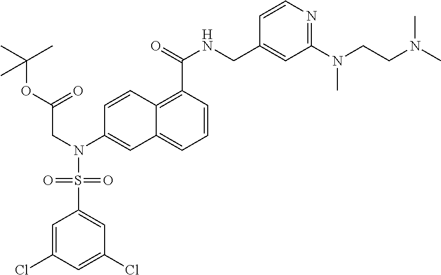

R_{f} value: 0.40 (silica gel, methylene chloride/methanol/conc. Aqueous ammonia=90:10:0.1)

Mass spectrum (ESI⁺): m/z=700, 702, 704 [M+H]⁺

### (49) tert. Butyl {(3,5-dichloro-phenylsulphonyl)-[5-({6-N-[(2-dimethylamino-ethyl)-N-methyl-amino]-pyridin-3-ylmethyl}-aminocarbonyl)-naphthalen-2-yl]-amino}-acetate

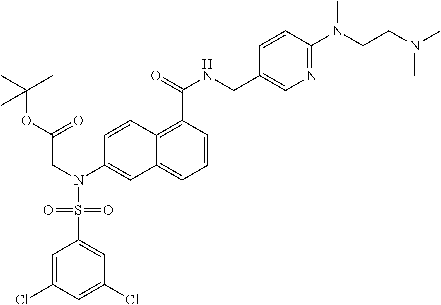

R_{f} value: 0.40 (silica gel, methylene chloride/methanol/conc. Aqueous ammonia=90:10:0.1)

Mass spectrum (ESI⁺): m/z=700, 702, 704 [M+H]⁺

### (50) tert. Butyl [(3,5-dichloro-phenylsulphonyl)-(5-{[2-(2-dimethylamino-ethylamino)-pyridin-4-ylmethyl]-carbamoyl}-naphthalen-2-yl)amino]-acetate

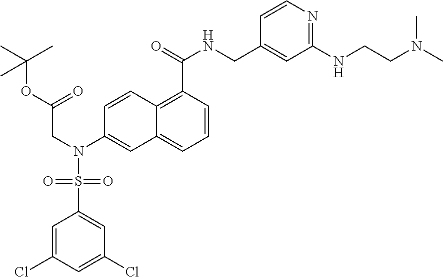

R_{f} value: 0.45 (silica gel, methylene chloride/methanol/conc. Aqueous ammonia=90:10:0.1)

Mass spectrum (ESI⁺): m/z=686, 688, 690 [M+H]⁺

### Example VI

### 6-[tert.-butoxycarbonylmethyl-(3,5-dichloro-phenylsulphonyl)-amino]-naphthalene-1-carboxylic acid

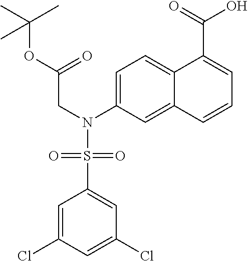

350 mg benzyl 6-[tert.-butoxycarbonylmethyl-(3,5-dichloro-phenylsulphonyl)-amino]-naphthalene-1-carboxylate are hydrogenated in 25 ml of tetrahydrofuran in the presence of 40 mg palladium on activated charcoal (10%) at ambient temperature and at a partial hydrogen pressure of 0.38 bar for 4.5 h. Then the catalyst is filtered off and the filtrate is evaporated down. The flask residue is dissolved slightly with a little tert-butylmethylether, diluted with petroleum ether, stirred, suction filtered, washed with petroleum ether and dried.

Yield: 291 mg (96% of theory)

R_{f} value: 0.30 (silica gel, methylene chloride/methanol=98:2)

Mass spectrum (ESI⁺): m/z=527, 529, 531 [M+NH₄]⁺

The following compounds are obtained analogously to Example VI:

### (1) 5-[tert.-butoxycarbonylmethyl-(3,5-dichloro-phenylsulphonyl)-amino]-naphthalene-1-carboxylic acid

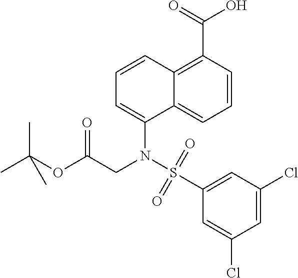

R_{f} value: 0.25 (silica gel, methylene chloride/methanol=98:2)

Mass spectrum (ESI⁺): m/z=527, 529, 531 [M+NH₄]⁺

### Example VII

Benzyl 6-amino-naphthalene-1-carboxylate

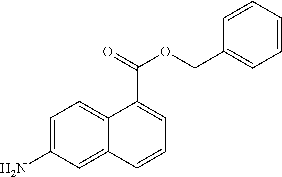

1.00 g 6-aminonaphthoic acid is suspended in benzylalcohol, combined with 920 mg anhydrous p-toluenesulphonic acid and stirred for a few minutes in the oil bath at 80° C. Then 1.23 g p-toluenesulphonic acid chloride are added and the reaction mixture is stirred for 4 h at 80° C.

A further 600 mg p-toluenesulphonic acid chloride are added and the reaction mixture is stirred for a further 5 h at 80-90° C. After cooling to ambient temperature the reaction mixture is diluted with 90 ml tert.-butylmethylether, the precipitate formed is suction filtered and washed with a little tert.-butylmethylether. The filter cake is taken up in tert.-butylmethylether and washed with dilute sodium carbonate solution, water and saturated sodium chloride solution, dried on magnesium sulphate and evaporated down.

Yield: 242 mg (16% of theory)

R_{f} value: 0.85 (silica gel, methylene chloride/methanol=95:5)

Mass spectrum (ESI⁺): m/z=278 [M+H]⁺

### Example VIII

### 6-(3,5-dichloro-phenylsulphonylamino)-naphthalene-2-carboxylic acid amide

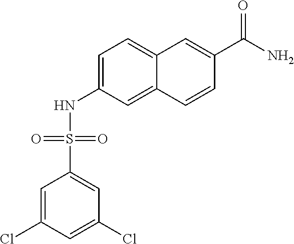

A mixture of 10.00 g 6-(3,5-dichloro-phenylsulphonylamino)-naphthalene-2-carboxylic acid, 24.30 g ammonium carbonate, 8.10 g O-(benzotriazol-1-yl)-1,1,3,3-tetramethyluronium-tetrafluoroborate and 3.52 ml triethylamine in 120 ml of tetrahydrofuran is stirred overnight at ambient temperature. For working up the reaction mixture is diluted with 150 ml of ethyl acetate and washed with 10% citric acid solution, 1N sodium hydroxide solution and saturated sodium chloride solution. The organic phase is dried on magnesium sulphate and evaporated down. The flask residue is stirred with diisopropylether, suction filtered and dried at 40° C. in the circulating air dryer. The sodium hydroxide solution phase is extracted with methylene chloride, during which time a precipitate is formed which is suction filtered and also dried in the circulating air dryer.

Yield: 7.13 g (72% of theory)

R_{f} value: 0.70 (silica gel, ethyl acetate)

Mass spectrum (ESI⁻): m/z=393, 395, 397 [M−H]⁻

The following compounds are obtained analogously to Example VIII:

### (1) tert. Butyl [(5-aminocarbonyl-naphthalen-2-yl)-(3,5-dichloro-phenylsulphonyl)-amino]-acetate

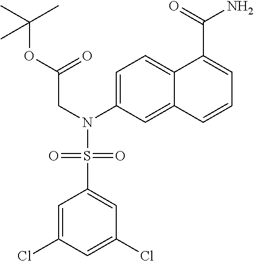

R_{f} value: 0.35 (silica gel, methylene chloride/methanol=95:5)

### (2) 7-(3,5-dichloro-phenylsulphonylamino)-naphthalene-2-carboxylic acid amide

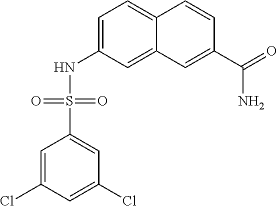

Mass spectrum (ESI⁺): m/z=395, 397, 399 [M+H]⁺

### Example IX

### tert. Butyl {(3,5-dichloro-phenylsulphonyl)-[6-(5-methyl-[1,2,4]oxadiazol-3-yl)-naphthalen-2-yl]-amino}-acetate

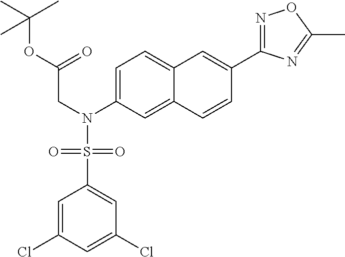

A mixture of 400 mg tert. Butyl {(3,5-dichloro-phenylsulphonyl)-[6-(N-hydroxy-carbamimidoyl)-naphthalen-2-yl]-amino}-acetate and 0.16 ml acetic anhydride in 5 ml acetonitrile is heated for five minutes in the microwave at 180° C. The reaction mixture is evaporated down, and the crude product is purified by chromatography through a silica gel column with cyclohexane/ethyl acetate (75:25 to 50:50) as eluant.

Yield: 250 mg (60% of theory)

R_{f} value: 0.90 (silica gel, cyclohexane/ethyl acetate=1:1)

The following compounds are obtained analogously to Example IX:

### (1) tert. Butyl {(3,5-dichloro-phenylsulphonyl)-[7-(5-methyl-[1,2,4]oxadiazol-3-yl)-naphthalen-2-yl]-amino}-acetate

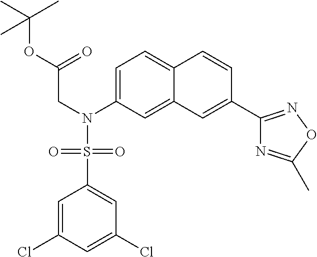

Mass spectrum (ESI⁺): m/z=565, 567, 569 [M+NH₄]⁺

### (2) tert. Butyl {(3,5-dichloro-phenylsulphonyl)-[5-(5-methyl-[1,2,4]oxadiazol-3-yl)-naphthalen-2-yl]-amino}-acetate

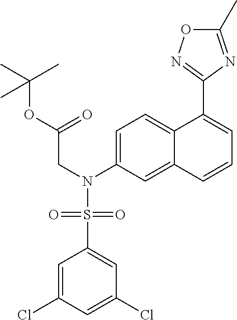

R_{f} value: 0.50 (silica gel, petroleum ether/ethyl acetate=3:1)

Mass spectrum (ESI⁺): m/z=565, 567, 569 [M+NH₄]⁺

### (3) tert. Butyl {(3,5-dichloro-phenylsulphonyl)-[5-(5-phenyl-[1,2,4]oxadiazol-3-yl)-naphthalen-2-yl]-amino}-acetate

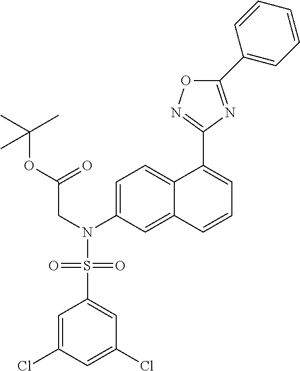

(reaction carried out with benzoyl chloride in 2,4,6-trimethylpyridine)

R_{f} value: 0.60 (silica gel, petroleum ether/ethyl acetate=3:1)

Mass spectrum (ESI⁺): m/z=610, 612, 614 [M+H]⁺

### (4) tert. Butyl {(3,5-dichloro-phenylsulphonyl)-[5-(5-benzyl-[1,2,4]oxadiazol-3-yl)-naphthalen-2-yl]-amino}-acetate

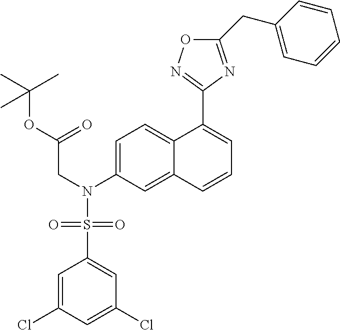

(reaction carried out with phenylacetyl chloride in 2,4,6-trimethylpyridine)

R_{f} value: 0.60 (silica gel, petroleum ether/ethyl acetate=3:1)

Mass spectrum (ESI⁺): m/z=624, 626, 628 [M+H]⁺

### (5) tert. Butyl {(3,5-dichloro-phenylsulphonyl)-[5-(5-isopropyl-[1,2,4]oxadiazol-3-yl)-naphthalen-2-yl]-amino}-acetate

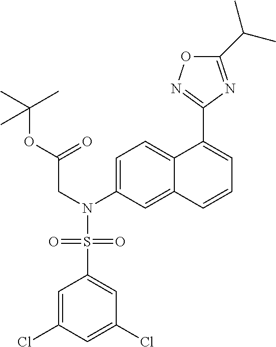

(reaction carried out with isobutyric acid chloride in 2,4,6-trimethylpyridine)

R_{f} value: 0.60 (silica gel, petroleum ether/ethyl acetate=3:1)

Mass spectrum (ESI⁺): m/z=576, 578, 580 [M+H]⁺

### (6) tert. Butyl {(3,5-dichloro-phenylsulphonyl)-[6-(5-phenyl-[1,2,4]oxadiazol-3-yl)-naphthalen-2-yl]-amino}-acetate

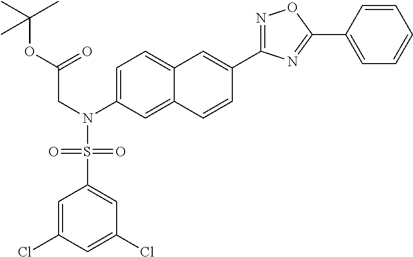

(reaction carried out with benzoyl chloride in 2,4,6-trimethylpyridine and methylene chloride)

R_{f} value: 0.76 (silica gel, petroleum ether/ethyl acetate=1:1)

Mass spectrum (ESI⁺): m/z=610, 612, 614 [M+H]⁺

### (7) tert. Butyl {(3,5-dichloro-phenylsulphonyl)-[6-(5-phenyl-[1,2,4]oxadiazol-3-yl)-naphthalen-2-yl]-amino}-acetate

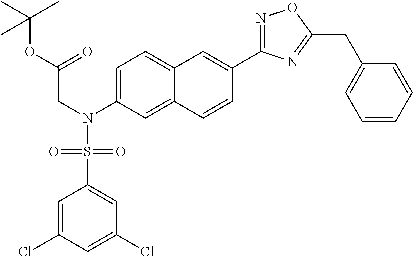

(reaction carried out with phenylacetyl chloride in 2,4,6-trimethylpyridine and methylene chloride)

R_{f} value: 0.75 (silica gel, petroleum ether/ethyl acetate=1:1)

Mass spectrum (ESI⁺): m/z=624, 626, 628 [M+H]⁺

### (8) tert. Butyl {(3,5-dichloro-phenylsulphonyl)-[6-(5-isopropyl-[1,2,4]oxadiazol-3-yl)-naphthalen-2-yl]-amino}-acetate

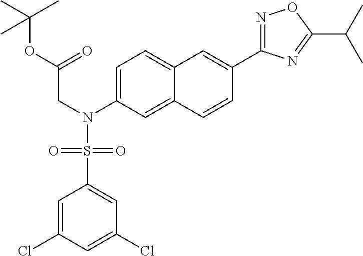

(reaction carried out with isobutyric acid chloride in 2,4,6-trimethylpyridine and methylene chloride)

R_{f} value: 0.71 (silica gel, petroleum ether/ethyl acetate=1:1)

Mass spectrum (ESI⁺): m/z=576, 578, 580 [M+H]⁺

### (9) tert. Butyl {(3,5-dichloro-phenylsulphonyl)-[5-(5-hydroxy-[1,2,4]oxadiazol-3-yl)-naphthalen-2-yl]-amino}-acetate

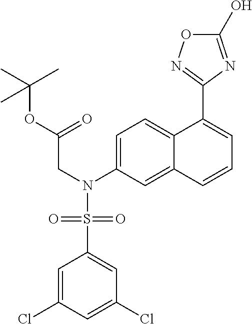

(formed as a by-product of the reaction with trichloroacetic acid chloride in 2,4,6-trimethylpyridine)

R_{f} value: 0.30 (silica gel, methylene chloride/methanol=20:1)

Mass spectrum (ESI⁻): m/z=548, 550, 552 [M−H]⁻

### (10) tert. Butyl {(3,5-dichloro-phenylsulphonyl)-[5-(5-trichloromethyl-[1,2,4]oxadiazol-3-yl)-naphthalen-2-yl]-amino}-acetate

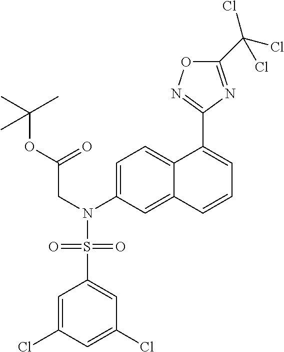

(reaction carried out with trichloroacetic acid chloride in 2,4,6-trimethylpyridine)

R_{f} value: 0.70 (silica gel, petroleum ether/ethyl acetate=3:1)

Mass spectrum (ESI⁺): m/z=667, 669, 671, 673 [M+NH₄]⁺

### (11) tert. Butyl {(3,5-dichloro-phenylsulphonyl)-[5-(5-methyl-[1,2,4]oxadiazol-3-yl)-naphthalen-1-yl]-amino}-acetate

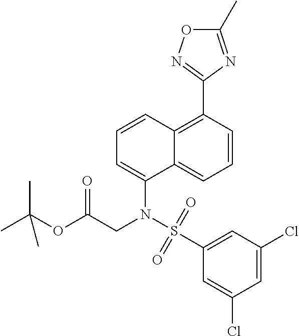

R_{f} value: 0.75 (silica gel, petroleum ether/ethyl acetate=1:1)

Mass spectrum (ESI⁺): m/z=565, 567, 569 [M+NH₄]⁺

### (12) tert. Butyl [[5-(5-chloromethyl-[1,2,4]oxadiazol-3-yl)-naphthalen-2-yl]-(3,5-dichloro-phenylsulphonyl)-amino]-acetate

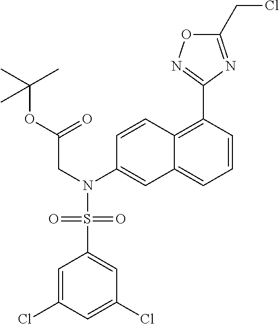

(reaction carried out with chloroacetyl chloride in 2,4,6-trimethylpyridine and methylene chloride)

R_{f} value: 0.50 (silica gel, petroleum ether/ethyl acetate=3:1)

### (13) 3-(5-bromo-naphthalen-2-yl)-5-methyl-[1,2,4]oxadiazole

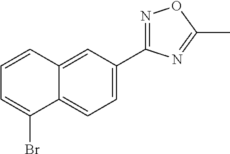

R_{f} value: 0.74 (silica gel, cyclohexane/ethyl acetate=1:1)

### (14) tert. Butyl {(3,5-dichloro-phenylsulphonyl)-[2-(5-methyl-[1,2,4]oxadiazol-3-yl)naphthalen-1-yl]-amino}-acetate

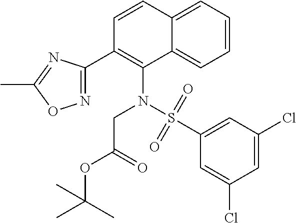

R_{f} value: 0.60 (silica gel, petroleum ether/ethyl acetate=5:1)

Mass spectrum (ESI⁺): m/z=548, 550, 52 [M+H]⁺

### Example X

### tert. Butyl {(3,5-dichloro-phenylsulphonyl)-[6-(N-hydroxycarbamimidoyl)-naphthalen-2-yl]-amino}-acetate

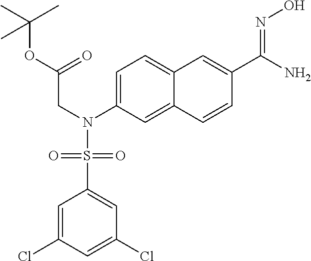

A mixture of 2.46 g tert butyl [(6-cyano-naphthalen-2-yl)-(3,5-dichloro-phenylsulphonyl)-amino]-acetate, 750 mg hydroxylamine-hydrochloride and 1.58 ml triethylamine in 25 ml absolute ethanol is refluxed for 3 h. The precipitate formed is suction filtered, washed with diethyl ether and dried at 50° C. in the circulating air dryer.

Yield: 1.57 g (60% of theory)

R_{f} value: 0.10 (silica gel, cyclohexane/ethyl acetate=3:1)

The following compounds are obtained analogously to Example X:

### (1) tert. Butyl {(3,5-dichloro-phenylsulphonyl)-[7-(N-hydroxycarbamimidoyl)-naphthalen-2-yl]-amino}-acetate

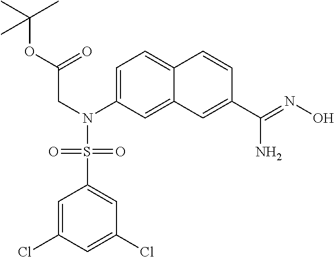

### (2) tert. Butyl {(3,5-dichloro-phenylsulphonyl)-[5-(N-hydroxycarbamimidoyl)-naphthalen-2-yl]-amino}-acetate

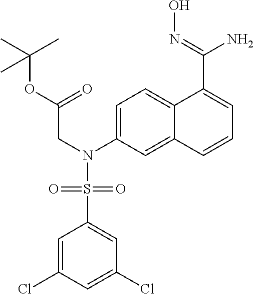

R_{f} value: 0.50 (silica gel, petroleum ether/ethyl acetate=1:1)

Mass spectrum (ESI⁺): m/z=524, 526, 528 [M+H]⁺

### (3) tert. Butyl {(3,5-dichloro-phenylsulphonyl)-[5-(N-hydroxycarbamimidoyl)-naphthalen-1-yl]-amino}-acetate

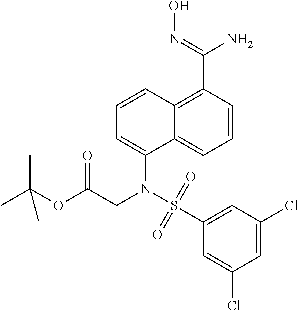

R_{f} value: 0.38 (silica gel, petroleum ether/ethyl acetate=1:1)

Mass spectrum (ESI⁺): m/z=524, 526, 528 [M+H]⁺

### (4) [(5-aminocarbonyl-naphthalen-1-yl)-(3,5-dichloro-phenylsulphonyl)-amino]-acetic acid

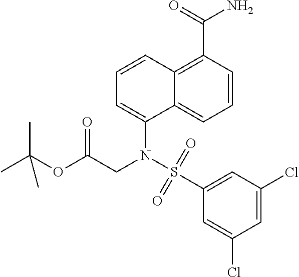

R_{f} value: 0.25 (silica gel, methylene chloride/methanol=9:1)

Mass spectrum (ESI⁺): m/z=509, 511, 513 [M+H]⁺

### (5) 5-bromo-N-hydroxy-naphthalene-2-carboxamidine

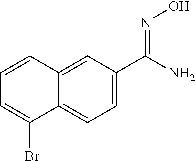

R_{f} value: 0.54 (silica gel, cyclohexane/ethyl acetate=1:1)

Mass spectrum (ESI⁺): m/z=265, 267 [M+H]⁺

### (6) tert. Butyl {(3,5-dichloro-phenylsulphonyl)-[2-(N-hydroxycarbamimidoyl)-naphthalen-1-yl]-amino}-acetate

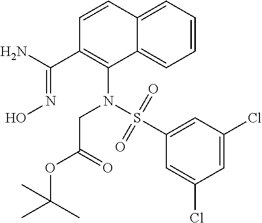

R_{f} value: 0.42 (silica gel, petroleum ether/ethyl acetate=2:1)

Mass spectrum (ESI⁺): m/z=524, 526, 528 [M+H]⁺

### Example XI

### 3,5-dichloro-N-(6-cyano-naphthalen-2-yl)-phenylsulphonamide

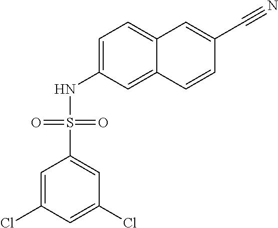

1.48 ml trifluoroacetic anhydride are added dropwise to 3.43 g 6-(3,5-dichloro-phenylsulphonylamino)-naphthalene-2-carboxylic acid amide and 3.64 ml triethylamine in 130 ml methylene chloride while cooling with an ice bath. The reaction mixture is stirred for 3 h at ambient temperature, then another 1.48 ml trifluoroacetic anhydride are added dropwise while cooling with an ice bath. After another two hours at ambient temperature a further 1.48 ml trifluoroacetic anhydride are added and the reaction mixture is stirred overnight at ambient temperature. For working up the reaction mixture is combined with 70 ml of water. The organic phase is separated off, washed with water and saturated sodium chloride solution, dried on magnesium sulphate and evaporated down. The flask residue is chromatographed through a silica gel column with cyclohexane/ethyl acetate (75:25 to 50:50) as eluant.

Yield: 2.29 g (70% of theory)

R_{f} value: 0.70 (silica gel, cyclohexane/ethyl acetate=3:1)

Mass spectrum (ESI⁻): m/z=375, 377, 379 [M−H]⁻

The following compounds are obtained analogously to Example XI:

### (1) 3,5-dichloro-N-(7-cyano-naphthalen-2-yl)-phenylsulphonamide

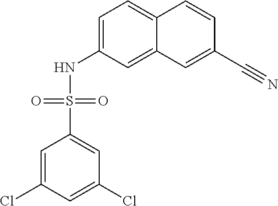

Mass spectrum (ESI⁺): m/z=394, 396, 398 [M+NH₄]⁺

### (2) 3,5-dichloro-N-(5-cyano-naphthalen-2-yl)-phenylsulphonamide

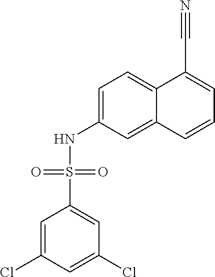

R_{f} value: 0.68 (silica gel, cyclohexane/ethyl acetate=1:1)

Mass spectrum (ESI⁻): m/z=375, 377, 379 [M−H]⁻

### (3) 3,5-dichloro-N-(5-cyano-naphthalen-1-yl)-phenylsulphonamide

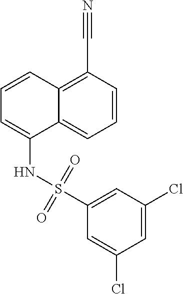

R_{f} value: 0.86 (silica gel, petroleum ether/ethyl acetate=1:1)

Mass spectrum (ESI⁻): m/z=375, 377, 379 [M−H]⁻

### Example XII

### tert. Butyl [(3,5-dichloro-phenylsulphonyl)-(6-oxazol-2-yl-naphthalen-2-yl)-amino]-acetate

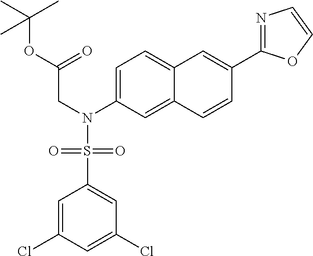

A mixture of 1.43 g tert. Butyl {(3,5-dichloro-phenylsulphonyl)-[6-(2-oxo-ethyl-aminocarbonyl)-naphthalen-2-yl]-amino}-acetate and 620 mg Burgess reagent in 10 ml of tetrahydrofuran is heated to 170° C. in the microwave for 5 min. The reaction mixture is evaporated down and chromatographed through a silica gel column with cyclohexane/ethyl acetate (80:20 to 66:34) as eluant.

Yield: 179 mg (13% of theory)

R_{f} value: 0.50 (silica gel, cyclohexane/ethyl acetate=3:1)

### Example XIII

### tert. Butyl {(3,5-dichloro-phenylsulphonyl)-[6-(2-oxo-ethylaminocarbonyl)-naphthalen-2-yl]-amino}-acetate

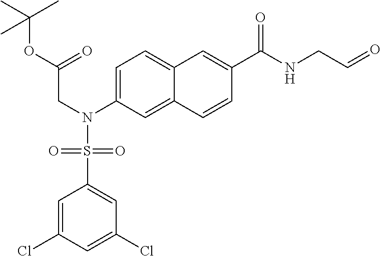

Prepared by oxidation of tert. Butyl {(3,5-dichloro-phenylsulphonyl)-[6-(2-hydroxy-ethylaminocarbonyl)-naphthalen-2-yl]-amino}-acetate with Dess-Martin reagent in methylene chloride at ambient temperature.

R_{f} value: 0.70 (silica gel, ethyl acetate)

### Example XIV

### 6-[tert.-butoxycarbonylmethyl-(3,5-dichloro-phenylsulphonyl)-amino]-naphthalene-1-carboxylic acid

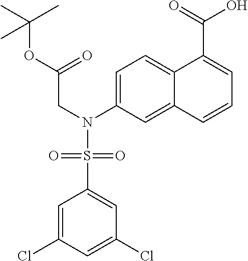

0.64 ml trimethylsilyl chloride are added to 1.00 g 6-(3,5-dichloro-phenylsulphonylamino)-naphthalene-1-carboxylic acid and 0.86 ml diisopropylethylamine in 10 ml of tetrahydrofuran while cooling with an ice bath. After ten minutes 2.10 g potassium-bis(trimethylsilyl)-amide are added batchwise, and the reaction mixture is heated to ambient temperature. After 6 h at this temperature the reaction mixture is combined with 0.20 ml tert. Butyl bromoacetate and stirred for a further 2 h at ambient temperature. For working up the reaction mixture is combined with 0.44 ml piperazine and 2 ml of methanol. After ten minutes 100 ml 1N hydrochloric acid are added and the mixture is extracted with ethyl acetate. The organic phase is separated off, dried on magnesium sulphate and evaporated down. The crude product is purified by chromatography through a silica gel column with methylene chloride/methanol (20:1 to 10:1) as eluant.

Yield: 730 mg (57% of theory)

R_{f} value: 0.60 (silica gel, methylene chloride/methanol=9:1)

### Example XV

### N-[7-(3,5-dichloro-phenylsulphonylamino)-naphthalen-2-yl]-benzamide

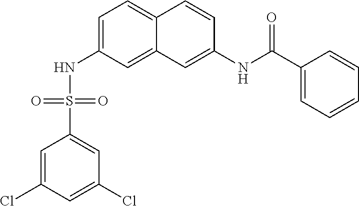

104 μl benzoyl chloride are added to 300 mg N-(7-amino-naphthalen-2-yl)-3,5-dichloro-phenylsulphonamide and 0.17 ml triethylamine in 5 ml methylene chloride while cooling with an ice bath. After heating to ambient temperature the reaction mixture is stirred for another 3 h. Then it is combined with dilute hydrochloric acid and extracted with ethyl acetate. The combined organic phases are washed with dilute hydrochloric acid and saturated sodium hydrogen carbonate solution, dried on magnesium sulphate and evaporated down. The flask residue is chromatographed through a silica gel column.

Yield: 310 mg (81% of theory)

Mass spectrum (ESI⁻): m/z=469, 471, 473 [M−H]⁻

The following compounds are obtained analogously to Example XV:

### (1) tert. Butyl [{7-[(4-chloro-pyridine-2-carbonyl)-amino]-naphthalen-2-yl}-(3,5-dichloro-phenylsulphonyl)-amino]-acetate

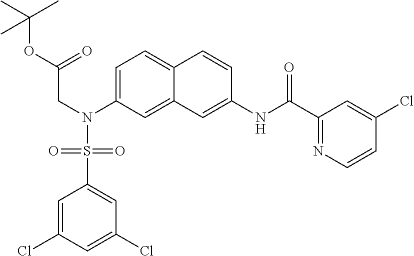

### (2) tert. Butyl [(5-benzoylamino-naphthalen-1-yl)-(3,5-dichloro-phenylsulphonyl)-amino]-acetate

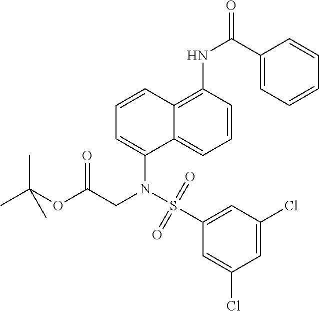

R_{f} value: 0.54 (silica gel, petroleum ether/ethyl acetate=2:1)

Mass spectrum (ESI⁻): m/z=583, 585, 587 [M−H]⁻

### (3) tert. Butyl [(3,5-dichloro-phenylsulphonyl)-(5-phenylacetylamino-naphthalen-1-yl)amino]-acetate

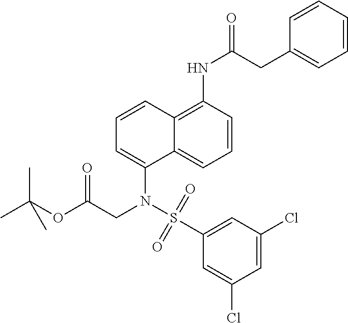

R_{f} value: 0.40 (silica gel, petroleum ether/ethyl acetate=2:1)

Mass spectrum (ESI⁻): m/z=597, 599, 601 [M−H]⁻

### (4) tert. Butyl {(3,5-dichloro-phenylsulphonyl)-[5-(3-phenyl-propionylamino)-naphthalen-1-yl]-amino}-acetate

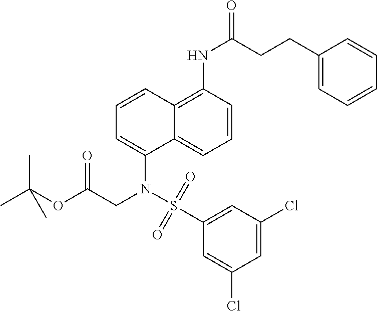

R_{f} value: 0.34 (silica gel, petroleum ether/ethyl acetate=2:1)

Mass spectrum (ESI⁻): m/z=611, 613, 615 [M−H]⁻

### (5) tert. Butyl [{5-[(4-chloro-pyridin-2-yl-carbonyl)-amino]-naphthalen-1-yl}-(3,5-dichloro-phenylsulphonyl)-amino]-acetate

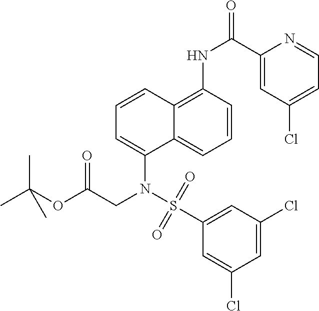

R_{f} value: 0.64 (silica gel, petroleum ether/ethyl acetate=2:1)

Mass spectrum (ESI⁺): m/z=620, 622, 624 [M+H]⁺

### Example XVI

### tert. Butyl [[7-(3-benzyl-ureido)-naphthalen-2-yl]-(3,5-dichloro-phenylsulphonyl)-amino]-acetate

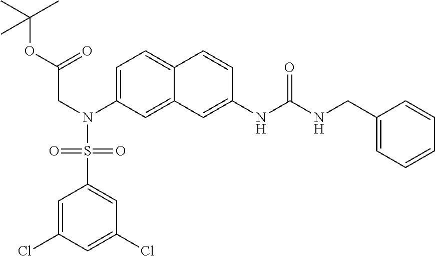

2 mg 4-dimethylaminopyridine and 18.5 μl benzylisocyanate are added to 60 mg tert. Butyl [(7-amino-naphthalen-2-yl)-(3,5-dichloro-phenylsulphonyl)-amino]-acetate in 4 ml methylene chloride. The reaction mixture is stirred overnight at ambient temperature and then evaporated down in vacuo. The crude product is purified by chromatography through a silica gel column.

Yield: 74 mg (97% of theory)

Mass spectrum (ESI⁻): m/z=658, 660, 662 [M+HCOO]⁻

The following compounds are obtained analogously to Example XVI:

### (1) tert. Butyl {(3,5-dichloro-phenylsulphonyl)-[5-(3-phenyl-ureido)-naphthalen-1-yl]-amino}-acetate

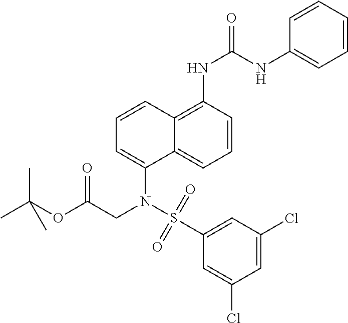

R_{f} value: 0.78 (silica gel, methylene chloride/methanol=95:5)

Mass spectrum (ESI⁺): m/z=600, 602, 604 [M+H]⁺

### (2) tert. Butyl [[5-(3-benzyl-ureido)-naphthalen-1-yl]-(3,5-dichloro-phenylsulphonyl)-amino]-acetate

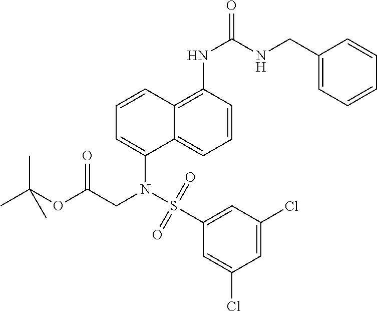

R_{f} value: 0.65 (silica gel, methylene chloride/methanol=95:5)

Mass spectrum (ESI⁺): m/z=614, 616, 618 [M+H]⁺

### Example XVII

### tert. Butyl [(3,5-dichloro-phenylsulphonyl)-(6-pyridin-3-yl-naphthalen-2-yl)-amino]-acetate

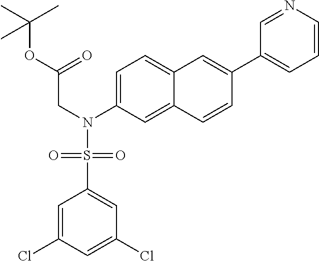

A mixture of 200 mg tert butyl [(3,5-dichlorophenylsulphonyl)-(6-trifluoromethanesulphonyloxy-naphthalen-2-yl)-amino]-acetate, 56 mg 3-pyridylboric acid and 69 mg sodium carbonate in 7 ml of toluene is combined with 0.80 ml of ethanol and 0.80 ml of water. The resulting solution is briefly evacuated twice and ventilated with argon. Then 56 mg of tetrakis-(triphenylphosphine)-palladium are added and the mixture is again evacuated and ventilated with argon. The reaction mixture is stirred for 7 h at 83° C. and then slowly cooled overnight to ambient temperature. For working up the reaction mixture is mixed with water and extracted with ethyl acetate. The organic phase is washed with water and saturated sodium chloride solution, dried on magnesium sulphate and evaporated down. The crude product is purified by chromatography through a silica gel column with petroleum ether/ethyl acetate (8:2 to 7:3).

Yield: 97 mg (55% of theory)

R_{f} value: 0.35 (silica gel, cyclohexane/ethyl acetate=1:1)

Mass spectrum (ESI⁺): m/z=543, 545, 547 [M+H]⁺

The following compounds are obtained analogously to Example XVII:

### (1) tert. Butyl [(3,5-dichloro-phenylsulphonyl)-(6-pyridin-4-yl-naphthalen-2-yl)-amino]-acetate

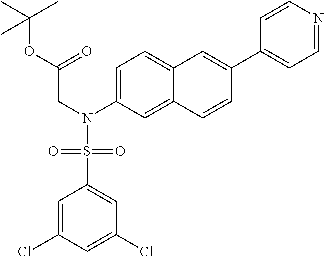

(The reaction is carried out with pinacolyl 4-pyridinylborate.)

R_{f} value: 0.33 (silica gel, cyclohexane/ethyl acetate=1:1)

Mass spectrum (ESI⁺): m/z=543, 545, 547 [M+H]⁺

### (2) tert. Butyl [(3,5-dichloro-phenylsulphonyl)-(6-furan-3-yl-naphthalen-2-yl)-amino]-acetate

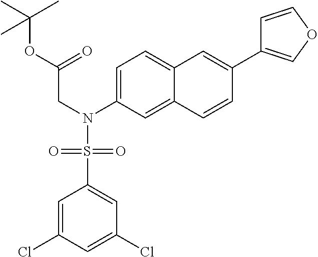

R_{f} value: 0.65 (silica gel, petroleum ether/ethyl acetate=8:2)

Mass spectrum (ESI⁺): m/z=549, 551, 553 [M+NH₄]⁺

### (3) tert. Butyl {(3,5-dichloro-phenylsulphonyl)-[6-(3,5-dimethyl-isoxazol-4-yl)-naphthalen-2-yl]-amino}-acetate

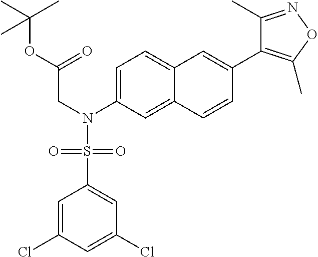

R_{f} value: 0.35 (silica gel, petroleum ether/ethyl acetate=8:2)

Mass spectrum (ESI⁺): m/z=578, 580, 582 [M+NH₄]⁺

### (4) tert. Butyl [(3,5-dichloro-phenylsulphonyl)-(6-furan-2-yl-naphthalen-2-yl)-amino]-acetate

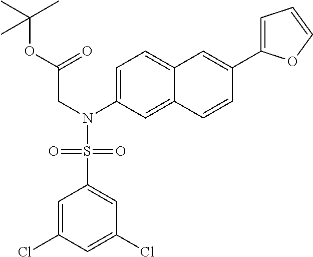

R_{f} value: 0.50 (silica gel, petroleum ether/ethyl acetate=8:2)

Mass spectrum (ESI⁺): m/z=549, 551, 553 [M+NH₄]⁺

### (5) tert. Butyl (6-pyrimidin-2-yl-naphthalen-2-yl)-carbamate

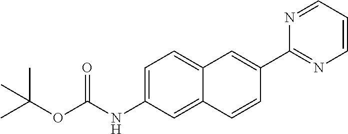

(The reaction takes place between 4-bromopyridine and 6-tert.-butoxycarbonylamino-naphthalen-2-yl-boric acid in a mixture of 1,4-dioxane and methanol.)

R_{f} value: 0.63 (silica gel, petroleum ether/ethyl acetate=1:1)

### (6) tert. Butyl (5-pyrimidin-2-yl-naphthalen-1-yl)-carbamate

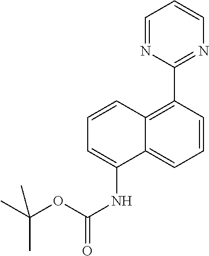

(The reaction takes place between 4-bromopyridine and 5-tert.-butoxycarbonylamino-naphthalen-1-yl-boric acid in a mixture of 1,4-dioxane and methanol.)

R_{f} value: 0.38 (silica gel, petroleum ether/ethyl acetate=1:1)

Mass spectrum (ESI⁺): m/z=322 [M+H]⁺

### (7) tert. Butyl [(3,5-dichloro-phenylsulphonyl)-(6-pyridin-2-yl-naphthalen-2-yl)-amino]-acetate

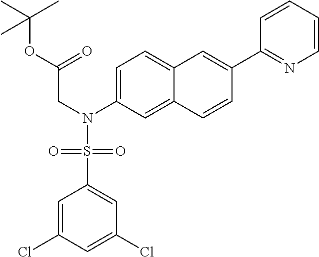

(The reaction takes place with diisopropyl 2-pyridinylborate.)

R_{f} value: 0.70 (silica gel, cyclohexane/ethyl acetate=1:1)

Mass spectrum (ESI⁺): m/z=543, 545, 547 [M+H]⁺

### (8) tert.-butyl 4-(2-{6-[tert.-butoxycarbonylmethyl-(3,5-dichloro-phenylsulphonyl)-amino]-naphthalen-2-yl}-pyrimidin-4-yl)-piperazine-1-carboxylate

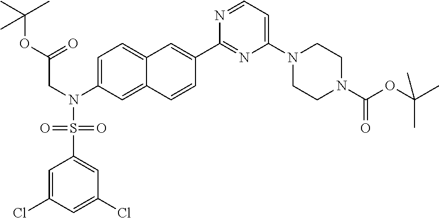

(The reaction takes place between tert.-butyl 4-(2-chloro-pyrimidin-4-yl)-piperazine-1-carboxylate and tert. Butyl {(3,5-dichloro-phenylsulphonyl)-[6-(4,4,5,5-tetramethyl-[1,3,2]dioxaborolan-2-yl)-naphthalen-2-yl]-amino}-acetate.)

R_{f} value: 0.55 (silica gel, cyclohexane/ethyl acetate=1:1)

Mass spectrum (ESI⁺): m/z=728, 730, 732 [M+H]⁺

### (9) tert.-butyl 4-(2-{5-[tert-butoxycarbonylmethyl-(3,5-dichloro-phenylsulphonyl)-amino]-naphthalen-1-yl}-pyrimidin-4-yl)piperazine-1-carboxylate

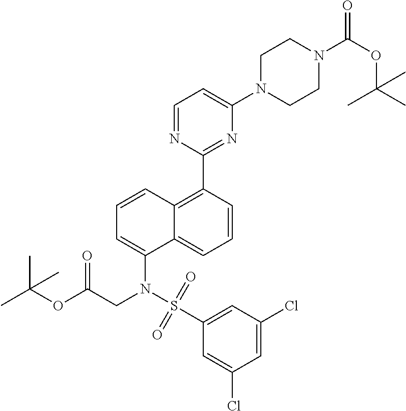

(The reaction takes place between tert.-butyl 4-(2-chloro-pyrimidin-4-yl)-piperazine-1-carboxylate and tert. Butyl {(3,5-dichloro-phenylsulphonyl)-[5-(4,4,5,5-tetramethyl-[1,3,2]dioxaborolan-2-yl)-naphthalen-1-yl]-amino}-acetate.)

R_{f} value: 0.65 (silica gel, tert.-butylmethylether/ethyl acetate=1:1)

Mass spectrum (ESI⁺): m/z=728, 730, 732 [M+H]⁺

### (10) tert. Butyl {(3,5-dichloro-phenylsulphonyl)-[6-(2-morpholin-4-yl-pyrimidin-4-yl)-naphthalen-2-yl]-amino}-acetate

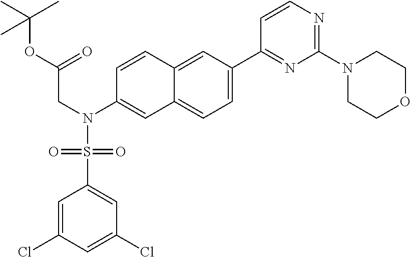

(The reaction takes place between 4-(4-chloro-pyrimidin-2-yl)-morpholine and tert. Butyl {(3,5-dichloro-phenylsulphonyl)-[6-(4,4,5,5-tetramethyl-[1,3,2]dioxaborolan-2-yl)-naphthalen-2-yl]-amino}-acetate.)

R_{f} value: 0.65 (silica gel, petroleum ether/ethyl acetate=1:1)

Mass spectrum (ESI⁺): m/z=629, 631, 633 [M+H]⁺

### (11) tert. Butyl [(3,5-dichloro-phenylsulphonyl)-(6-{4-[N-(2-dimethylamino-ethyl)-N-methyl-amino]-pyrimidin-2-yl}-naphthalen-2-yl)-amino]-acetate

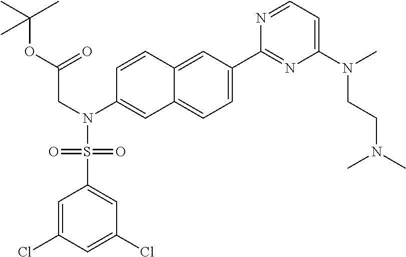

(The reaction takes place between N-(2-chloro-pyrimidin-4-yl)-N,N′,N′-trimethyl-ethane-1,2-diamine and tert. Butyl {(3,5-dichloro-phenylsulphonyl)-[6-(4,4,5,5-tetramethyl-[1,3,2]dioxaborolan-2-yl)-naphthalen-2-yl]-amino}-acetate.)

R_{f} value: 0.35 (silica gel, methylene chloride/methanol/conc. Aqueous ammonia=95:5:0.1)

Mass spectrum (ESI⁺): m/z=644, 646, 648 [M+H]⁺

### (12) tert. Butyl {(3,5-dichloro-phenylsulphonyl)-[5-(4-morpholin-4-yl-pyrimidin-2-yl)-naphthalen-1-yl]-amino}-acetate

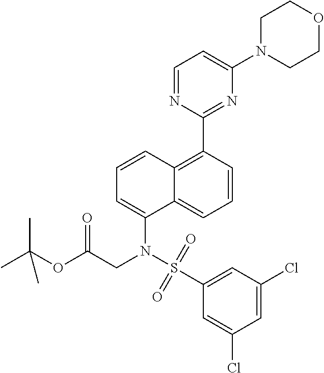

(The reaction takes place between 4-(2-chloro-pyrimidin-4-yl)-morpholine and tert. Butyl {(3,5-dichloro-phenylsulphonyl)-[5-(4,4,5,5-tetramethyl-[1,3,2]dioxaborolan-2-yl)-naphthalen-1-yl]-amino}-acetate.)

R_{f} value: 0.22 (silica gel, petroleum ether/ethyl acetate=1:1)

Mass spectrum (ESI⁺): m/z=629, 631, 633 [M+H]⁺

### (13) tert. Butyl ((3,5-dichloro-phenylsulphonyl)-{6-[4-(2-dimethylamino-ethylamino)-pyrimidin-2-yl]-naphthalen-2-yl}-amino)acetate

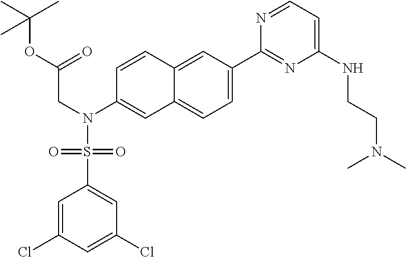

(The reaction takes place between N′-(2-chloro-pyrimidin-4-yl)-N,N-dimethyl-ethane-1,2-diamine and tert. Butyl {(3,5-dichloro-phenylsulphonyl)-[6-(4,4,5,5-tetramethyl-[1,3,2]dioxaborolan-2-yl)-naphthalen-2-yl]-amino}-acetate.)

R_{f} value: 0.28 (silica gel, methylene chloride/methanol/conc. Aqueous ammonia=90:10:0.1)

### (14) tert. Butyl [[5-(2-chloro-pyrimidin-4-yl)-naphthalen-1-yl]-(3,5-dichloro-phenylsulphonyl)-amino]-acetate

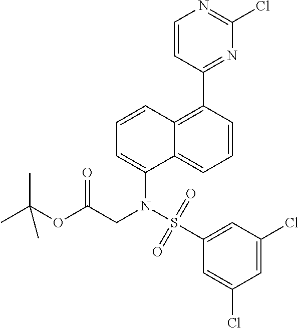

(The reaction takes place between 2,4-dichloropyrimidine and tert. Butyl {(3,5-dichloro-phenylsulphonyl)-[5-(4,4,5,5-tetramethyl-[1,3,2]dioxaborolan-2-yl)naphthalen-1-yl]-amino}-acetate.)

R_{f} value: 0.13 (silica gel, petroleum ether/ethyl acetate=5:1)

Mass spectrum (ESI⁺): m/z=578, 580, 582 [M+H]⁺

### (15) tert. Butyl {(3,5-dichloro-phenylsulphonyl)-[6-(6-morpholin-4-yl-pyridazin-3-yl)-naphthalen-2-yl]-amino}-acetate

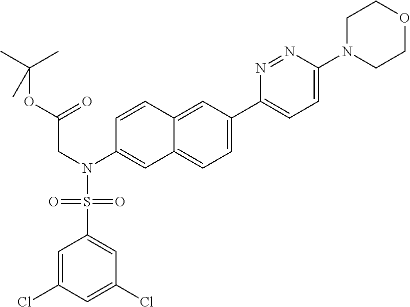

(The reaction takes place between 4-(6-iodo-pyridazin-3-yl)-morpholine and 6-[tert-butoxycarbonylmethyl-(3,5-dichloro-phenylsulphonyl)-amino]-naphthalene-2-boric acid.)

R_{f} value: 0.63 (silica gel, methylene chloride/methanol=95:5)

### (16) tert. Butyl {(3,5-dichloro-phenylsulphonyl)-[5-(2-morpholin-4-yl-pyrimidin-5-yl)-naphthalen-1-yl]-amino}-acetate

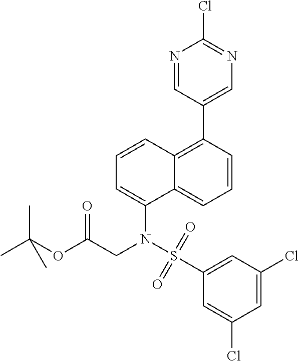

Mass spectrum (ESI⁺): m/z=578, 580, 582 [M+H]⁺

(The reaction takes place between 5-bromo-2-chloro-pyrimidine and tert. Butyl {(3,5-dichloro-phenylsulphonyl)-[5-(4,4,5,5-tetramethyl-[1,3,2]dioxaborolan-2-yl)naphthalen-1-yl]-amino}-acetate.)

### (17) tert. Butyl ((3,5-dichloro-phenylsulphonyl)-{6-[5-(2-dimethylamino-ethylamino)-pyrazin-2-yl]-naphthalen-2-yl}-amino)-acetate

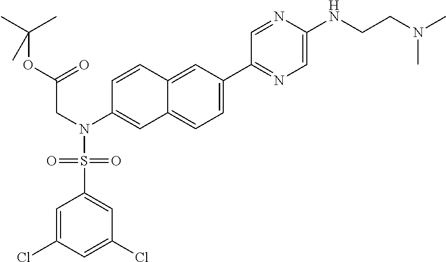

(The reaction takes place between N′-(5-bromo-pyrazin-2-yl)-N,N-dimethyl-ethane-1,2-diamine and 6-[tert-butoxycarbonylmethyl-(3,5-dichloro-phenylsulphonyl)-amino]-naphthalene-2-boric acid.)

R_{f} value: 0.52 (silica gel, methylene chloride/methanol/conc. Aqueous ammonia=90:10:1)

Mass spectrum (ESI⁺): m/z=630, 632, 634 [M+H]⁺

### (18) tert. Butyl ((3,5-dichloro-phenylsulphonyl)-{6-[6-(2-dimethylamino-ethylamino)-pyridazin-3-yl]-naphthalen-2-yl}-amino)-acetate

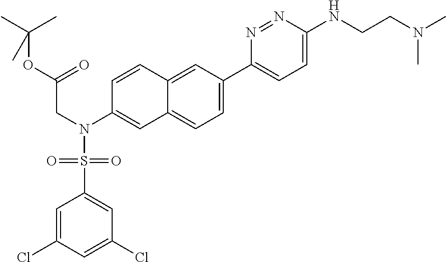

(The reaction takes place between N′-(6-bromo-pyridazin-3-yl)-N,N-dimethyl-ethane-1,2-diamine and 6-[tert-butoxycarbonylmethyl-(3,5-dichloro-phenyl-sulphonyl)-amino]-naphthalene-2-boric acid.)

R_{f} value: 0.46 (silica gel, methylene chloride/methanol/conc. Aqueous ammonia=90:10:1)

### (19) tert. Butyl {(3,5-dichloro-phenylsulphonyl)-[6-(4-morpholin-4-yl-pyrimidin-2-yl)-naphthalen-2-yl]-amino}-acetate

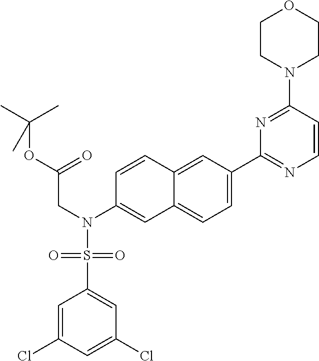

(The reaction takes place between 4-(2-chloro-4-pyrimidinyl)-morpholine and tert butyl {(3,5-dichloro-phenylsulphonyl)-[6-(4,4,5,5-tetramethyl-[1,3,2]dioxaborolan-2-yl)-naphthalen-2-yl]-amino}-acetate.)

R_{f} value: 0.30 (silica gel, cyclohexane/ethyl acetate=1:1)

Mass spectrum (ESI⁺): m/z=629, 631, 633 [M+H]⁺

### (20) tert. Butyl {(3,5-dichloro-phenylsulphonyl)-[6-(5-morpholin-4-yl-pyrazin-2-yl)-naphthalen-2-yl]-amino}-acetate

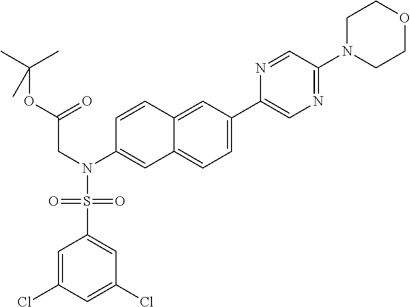

(The reaction takes place between 4-(5-bromo-pyrazin-2-yl)-morpholine and 6-[tert-butoxycarbonylmethyl-(3,5-dichloro-phenylsulphonyl)-amino]-naphthalene-2-boric acid.)

R_{f} value: 0.50 (silica gel, petroleum ether/ethyl acetate=1:1)

Mass spectrum (ESI⁺): m/z=629, 631, 633 [M+H]⁺

### Example XVIII

### 3,5-dichloro-N-(6-trifluoromethanesulphonyloxy-naphthalen-2-yl)-phenylsulphonamide

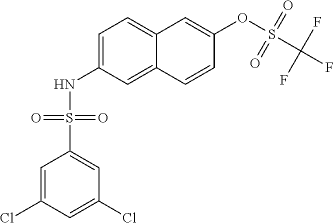

A solution of 0.36 ml trifluoromethanesulphonic acid anhydride in 5 ml methylene chloride is added dropwise to 760 mg of 3,5-dichloro-N-(6-hydroxy-naphthalen-2-yl)-phenylsulphonamide and 0.48 ml of pyridine in 25 ml methylene chloride while cooling with an ice bath, and the reaction mixture is slowly heated to ambient temperature. Then a further 0.20 ml of pyridine and 0.10 ml trifluoromethanesulphonic acid anhydride are added while cooling with an ice bath. For working up the reaction mixture is diluted with methylene chloride, washed with dilute sodium carbonate solution, dilute hydrochloric acid and water, dried on magnesium sulphate and evaporated down. The flask residue is stirred with petroleum ether, the precipitate formed is suction filtered and dried.

Yield: 808 mg (78% of theory)

R_{f} value: 0.85 (silica gel, cyclohexane/ethyl acetate=1:1)

Mass spectrum (ESI⁻): m/z=498, 500, 502 [M−H]⁻

The following compounds are obtained analogously to Example XVIII:

### (1) 3,5-dichloro-N-(5-trifluoromethanesulphonyloxy-naphthalen-1-yl)-phenylsulphonamide

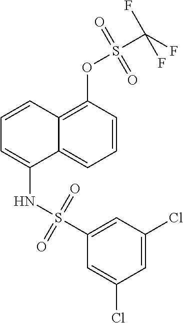

R_{f} value: 0.35 (silica gel, petroleum ether/ethyl acetate=5:1)

Mass spectrum (ESI⁺): m/z=517, 519, 521 [M+NH₄]⁺

### Example XIX

### 7-(3,5-dichloro-phenylsulphonylamino)-naphthalene-2-carboxylic acid

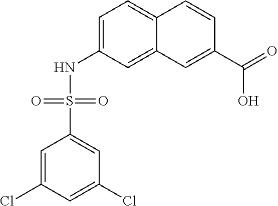

A mixture of 300 mg methyl 7-(3,5-dichloro-phenylsulphonylamino)-naphthalene-2-carboxylate, 10 ml of 2N sodium hydroxide solution and 10 ml of methanol is stirred for 6 h at ambient temperature, then the solvent is distilled off in vacuo. The flask residue is acidified with 2 N hydrochloric acid and extracted with ethyl acetate. The combined organic extracts are dried on magnesium sulphate and evaporated down. The crude product is extracted with diisopropylether, suction filtered and dried.

Yield: 277 mg (96% of theory)

Mass spectrum (ESI⁻): m/z=394, 396, 398 [M−H]⁻

The following compounds are obtained analogously to Example XIX:

### (1) 4-({[5-(3,5-dichloro-phenylsulphonylamino)-naphthalene-1-carbonyl]-amino}-methyl)-benzoic acid

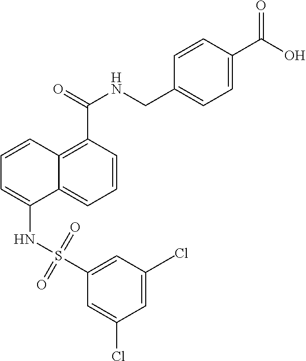

R_{f} value: 0.56 (silica gel, petroleum ether/ethyl acetate=1:2)

Mass spectrum (ESI⁺): m/z=529, 531, 533 [M+H]⁺

### (2) 4-({[5-(3,5-dichloro-phenylsulphonylamino)-naphthalene-2-carbonyl]-amino}-methyl)-benzoic acid

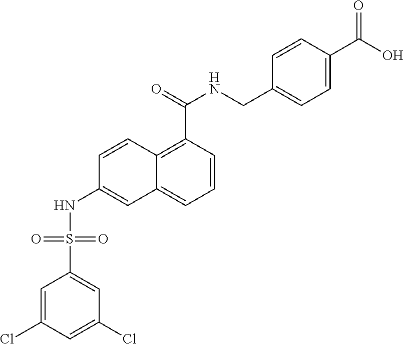

Mass spectrum (ESI⁺): m/z=529, 531, 533 [M+H]⁺

### (3) 3-({[5-(3,5-dichloro-phenylsulphonylamino)-naphthalene-1-carbonyl]-amino}-methyl)-benzoic acid

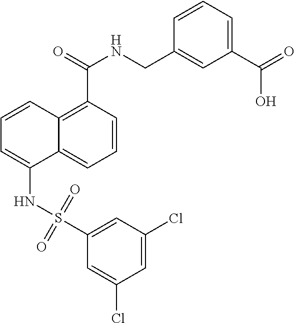

R_{f} value: 0.52 (silica gel, petroleum ether/ethyl acetate=1:2)

Mass spectrum (ESI⁺): m/z=529, 531, 533 [M+H]⁺

### Example XX

### 6-(3,5-dichloro-phenylsulphonylamino)-naphthalene-1-carboxylic acid amide

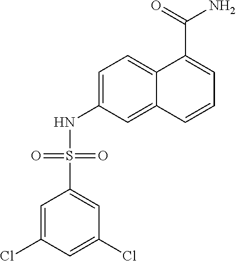

2.90 g carbonyldiimidazole are added to 7.20 g 6-(3,5-dichloro-phenylsulphonylamino)-naphthalene-1-carboxylic acid in 70 ml N,N-dimethylformamide and the reaction mixture is heated in a water bath. After cooling to ambient temperature the reaction mixture is stirred for another 5 h, combined with 20 ml concentrated ammonia and stirred overnight. Then the mixture is diluted with ethyl acetate, the organic phase is separated off and the aqueous phase is acidified with concentrated hydrochloric acid. A precipitate settles out, which is suction filtered and dried.

Yield: 4.50 g (63% of theory)

Mass spectrum (ESI⁺): m/z=395, 397, 399 [M+H]⁺

The following compounds are obtained analogously to Example XX:

### (1) 5-(3,5-dichloro-phenylsulphonylamino)-naphthalene-1-carboxylic acid amide

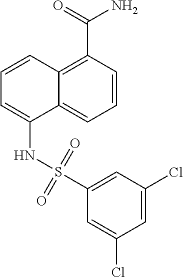

R_{f} value: 0.20 (silica gel, petroleum ether/ethyl acetate=1:1)

Mass spectrum (ESI⁺): m/z=395, 397, 399 [M+H]⁺

### (2) 5-(3,5-dichloro-phenylsulphonylamino)-naphthalene-1-carboxylic acid-(pyridin-4-ylmethyl)-amide

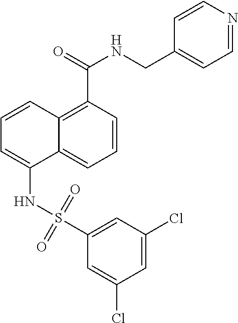

(The reaction takes place with 4-picolylamine in tetrahydrofuran.)

R_{f} value: 0.25 (silica gel, ethyl acetate)

Mass spectrum (ESI⁻): m/z=484, 486, 488 [M−H]⁻

### (3) 5-(3,5-dichloro-phenylsulphonylamino)-naphthalene-1-carboxylic acid-cyclohexylmethyl-amide

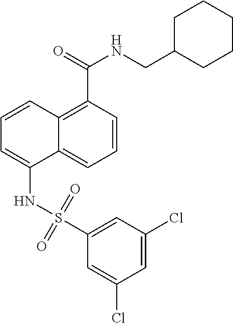

(The reaction takes place with aminomethylcyclohexane in tetrahydrofuran.)

R_{f} value: 0.80 (silica gel, cyclohexane/ethyl acetate=1:1)

Mass spectrum (ESI⁻): m/z=489, 491, 493 [M−H]⁻

### (4) 5-(3,5-dichloro-phenylsulphonylamino)-naphthalene-1-carboxylic acid-(pyridin-2-ylmethyl)-amide

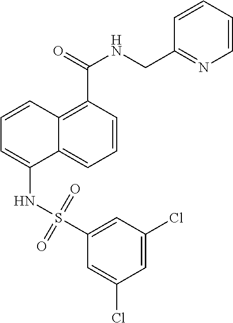

(The reaction takes place with 2-picolylamine in tetrahydrofuran.)

R_{f} value: 0.52 (silica gel, ethyl acetate)

Mass spectrum (ESI⁺): m/z=486, 488, 490 [M+H]⁺

### (5) 5-(3,5-dichloro-phenylsulphonylamino)-naphthalene-1-carboxylic acid-(pyridin-3-ylmethyl)-amide

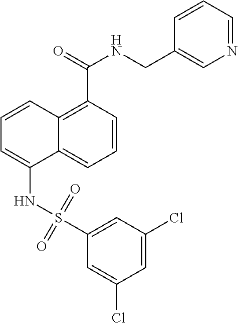

(The reaction takes place with 3-picolylamine in tetrahydrofuran.)

R_{f} value: 0.30 (silica gel, ethyl acetate)

Mass spectrum (ESI⁻): m/z=484, 486, 488 [M−H]⁻

### (6) 5-(3,5-dichloro-phenylsulphonylamino)-naphthalene-1-carboxylic acid-4-cyano-benzylamide

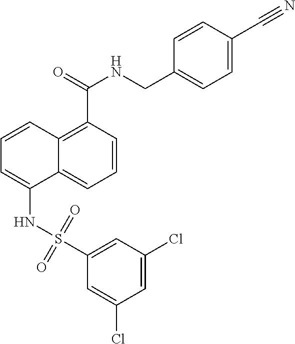

(The reaction takes place with 4-cyanobenzylamine in tetrahydrofuran.)

R_{f} value: 0.90 (silica gel, ethyl acetate)

Mass spectrum (ESI⁺): m/z=510, 512, 514 [M+H]⁺

### (7) 5-(3,5-dichloro-phenylsulphonylamino)-naphthalene-1-carboxylic acid-3-cyano-benzylamide

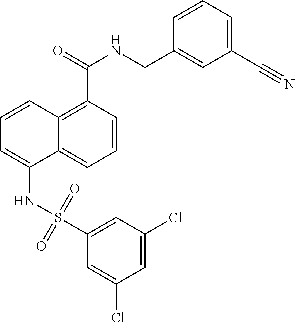

(The reaction takes place with 3-cyanobenzylamine in tetrahydrofuran.)

R_{f} value: 0.90 (silica gel, ethyl acetate)

Mass spectrum (ESI⁺): m/z=510, 512, 514 [M+H]⁺

### (8) 5-(3,5-dichloro-phenylsulphonylamino)-naphthalene-1-carboxylic acid-3-cyano-benzylamide

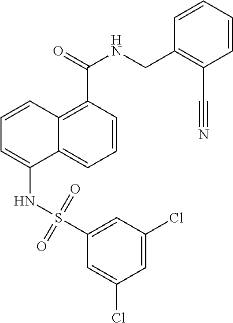

(The reaction takes place with 2-cyanobenzylamine in tetrahydrofuran.)

R_{f} value: 0.37 (silica gel, cyclohexane/ethyl acetate=1:1)

### Example XXI

### (3,5-dichloro-N-[5-(3-methyl-[1,2,4]oxadiazol-5-yl)-naphthalen-2-yl]-phenylsulphonamide

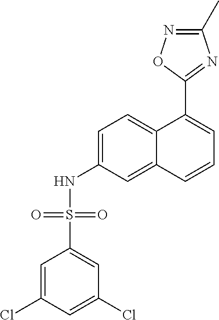

185 mg N-hydroxy-acetamidine are added to 1.00 g 6-(3,5-dichloro-phenylsulphonylamino)-naphthalene-1-carbonyl chloride in 2 ml 2,4,6-trimethylpyridine. The reaction mixture is heated to 110° C. and stirred for 6 h at this temperature. After cooling to ambient temperature the mixture is combined with ethyl acetate and water. The organic phase is separated off and washed with 1N hydrochloric acid, water and saturated sodium chloride solution, dried on magnesium sulphate and evaporated down. The flask residue stirred is with methylene chloride, suction filtered and dried.

Yield: 690 mg (66% of theory)

R_{f} value: 0.40 (silica gel, petroleum ether/ethyl acetate=3:1)

Mass spectrum (ESI⁻): m/z=432, 434, 436 [M−H]⁻

The following compounds are obtained analogously to Example XXI:

### (1) (3,5-dichloro-N-[6-(3-methyl-[1,2,4]oxadiazol-5-yl)-naphthalen-2-yl]-phenylsulphonamide

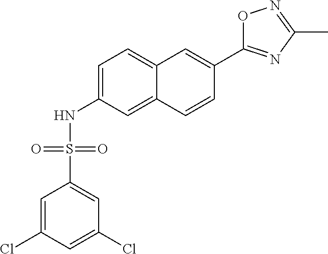

Mass spectrum (ESI⁺): m/z=434, 436, 438 [M+H]⁺

### (2) (3,5-dichloro-N-[6-(3-phenyl-[1,2,4]oxadiazol-5-yl)-naphthalen-2-yl]-phenylsulphonamide

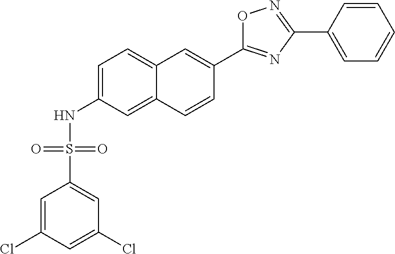

Mass spectrum (ESI⁺): m/z=496, 498, 500 [M+H]⁺

### (3) ethyl 5-[6-(3,5-dichloro-phenylsulphonylamino)-naphthalen-1-yl]-[1,2,4]oxadiazole-3-carboxylate

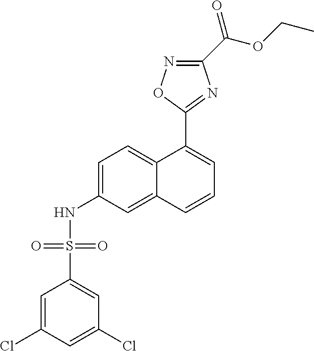

R_{f} value: 0.40 (silica gel, petroleum ether/ethyl acetate=3:1)

Mass spectrum (ESI⁺): m/z=492, 494, 496 [M+H]⁺

### (4) N-[5-(3-benzyl-[1,2,4]oxadiazol-5-yl)-naphthalen-2-yl]-3,5-dichloro-phenylsulphonamide

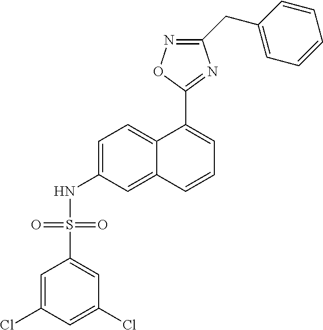

R_{f} value: 0.40 (silica gel, petroleum ether/ethyl acetate=3:1)

Mass spectrum (ESI⁺): m/z=510, 512, 514 [M+H]⁺

### (5) 3,5-dichloro-N-[5-(3-phenoxymethyl-[1,2,4]oxadiazol-5-yl)-naphthalen-2-yl]-phenylsulphonamide

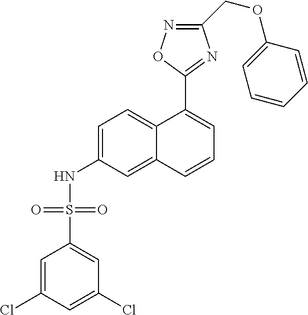

R_{f} value: 0.40 (silica gel, petroleum ether/ethyl acetate=3:1)

Mass spectrum (ESI⁺): m/z=526, 528, 530 [M+H]⁺

### (6) N-[5-(3-phenylsulphonylmethyl-[1,2,4]oxadiazol-5-yl)-naphthalen-2-yl]-3,5-dichloro-phenylsulphonamide

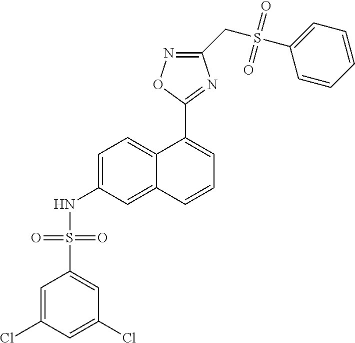

R_{f} value: 0.70 (silica gel, petroleum ether/ethyl acetate=1:1)

Mass spectrum (ESI⁻): m/z=572, 574, 576 [M−H]⁻

### (7) ethyl 5-[6-(3,5-dichloro-phenylsulphonylamino)-naphthalen-2-yl]-[1,2,4]oxadiazole-3-carboxylate

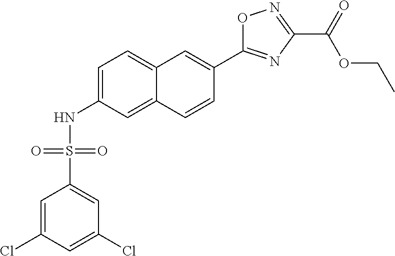

R_{f} value: 0.61 (silica gel, petroleum ether/ethyl acetate=1:1)

Mass spectrum (ESI⁻): m/z=490, 492, 494 [M−H]⁻

### (8) N-[6-(3-benzyl-[1,2,4]oxadiazol-5-yl)-naphthalen-2-yl]-3,5-dichloro-phenylsulphonamide

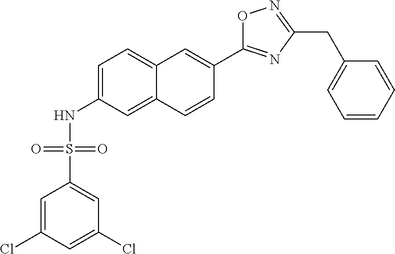

R_{f} value: 0.63 (silica gel, petroleum ether/ethyl acetate=1:1)

Mass spectrum (ESI⁺): m/z=510, 512, 514 [M+H]⁺

### (9) 3,5-dichloro-N-[6-(3-phenoxymethyl-[1,2,4]oxadiazol-5-yl)-naphthalen-2-yl]-phenylsulphonamide

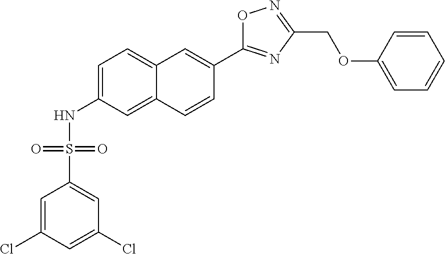

R_{f} value: 0.78 (silica gel, petroleum ether/ethyl acetate=1:1)

Mass spectrum (ESI⁺): m/z=526, 528, 530 [M+H]⁺

### (10) N-[6-(3-phenylsulphonylmethyl-[1,2,4]oxadiazol-5-yl)-naphthalen-2-yl]-3,5-dichloro-phenylsulphonamide

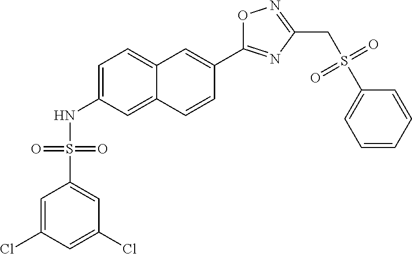

R_{f} value: 0.51 (silica gel, petroleum ether/ethyl acetate=1:1)

Mass spectrum (ESI⁻): m/z=572, 574, 576 [M−H]⁻

### Example XXII

### 6-(3,5-dichloro-phenylsulphonylamino)-naphthalene-1-carbonyl chloride

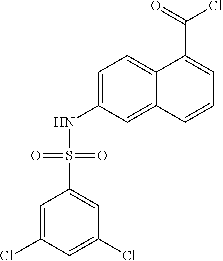

Prepared by reacting 6-(3,5-dichloro-phenylsulphonylamino)-naphthalene-1-carboxylic acid with thionyl chloride at reflux temperature.

The following compounds are obtained analogously to Example XXII:

### (1) 6-(3,5-dichloro-phenylsulphonylamino)-naphthalene-2-carbonyl chloride

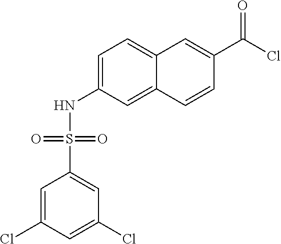

### Example XXIII

### 3,5-dichloro-N-[6-(5-methyl-[1.3.4]oxadiazol-2-yl)-naphthalen-2-yl]-phenylsulphonamide

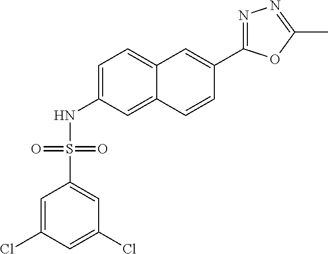

270 mg N-[6-(N′-acetyl-hydrazinocarbonyl)-naphthalen-2-yl]-3,5-dichloro-phenylsulphonamide are dissolved in 2 ml phosphorus oxychloride and stirred for 3 h at 80° C. Then the mixture is added to ice water, the precipitate formed is suction filtered, washed with water and dried. The crude product is stirred with ethanol, finely suspended in the ultrasound bath, suction filtered, washed with diethyl ether and dried.

Yield: 100 mg (39% of theory)

R_{f} value: 0.75 (silica gel, methylene chloride/methanol=9:1)

Mass spectrum (ESI⁺): m/z=434, 436, 438 [M+H]⁺

### Example XXIV

### N-[6-(N′-acetyl-hydrazinocarbonyl)-naphthalen-2-yl]-3,5-dichloro-phenylsulphonamide

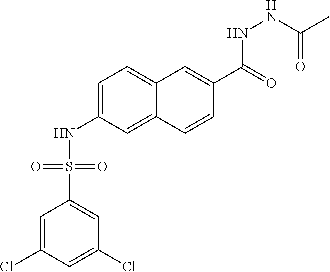

A mixture of 500 mg 6-(3,5-dichloro-phenylsulphonylamino)-naphthalene-2-carbonyl chloride, 250 mg acetic hydrazide and 3 ml 2,4,6-trimethylpyridine in 3 ml N,N-dimethylformamide is stirred overnight at 80° C. Then the mixture is diluted with ethyl acetate, washed with 2N hydrochloric acid and saturated sodium chloride solution, dried on magnesium sulphate and evaporated down. The residue is purified by chromatography through a silica gel column with methylene chloride/methanol (99:1 to 80:20) as eluant.

Yield: 270 mg (50% of theory)

R_{f} value: 0.58 (silica gel, methylene chloride/methanol=9:1)

Mass spectrum (ESI⁺): m/z=452, 454, 456 [M+H]⁺

### Example XXV

### 6-pyrimidin-2-yl-naphthalen-2-ylamine

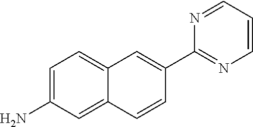

Prepared by treating tert butyl (6-pyrimidin-2-yl-naphthalen-2-yl)-carbamate with trifluoroacetic acid in methylene chloride at ambient temperature.

R_{f} value: 0.20 (silica gel, petroleum ether/ethyl acetate=2:1)

The following compounds are obtained analogously to Example XXV:

### (1) 5-pyrimidin-2-yl-naphthalen-1-ylamine

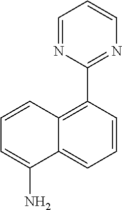

R_{f} value: 0.26 (silica gel, petroleum ether/ethyl acetate=1:1)

### Example XXVI

### 6-tert.-butoxycarbonylamino-naphthalen-2-yl-boric acid

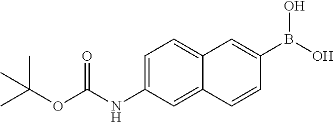

8.73 ml n-butyllithium solution (1.6M in hexane) are added dropwise to 1.50 g tert. Butyl (6-bromo-naphthalen-2-yl)-carbamate in 15 ml anhydrous tetrahydrofuran under an argon atmosphere at −70° C. The suspension is stirred for one hour at −50° C., then 1.61 ml triisopropyl borate, dissolved in 10 ml anhydrous tetrahydrofuran, are added dropwise within 5 minutes. The suspension is slowly heated to ambient temperature, combined with 10 ml 1N hydrochloric acid and stirred for 20 minutes at ambient temperature. The aqueous phase is saturated with common salt and the organic phase is separated off. The aqueous phase is extracted with ethyl acetate, and the combined organic phases are washed with saturated sodium hydrogen carbonate solution, dried on magnesium sulphate and evaporated down. The flask residue is chromatographed through a silica gel column with methylene chloride/methanol (98:2 to 95:5) as eluant.

Yield: 590 mg (44% of theory)

R_{f} value: 0.42 (silica gel, methylene chloride/methanol=95:5)

Mass spectrum (ESI⁺): m/z=288 [M+H]⁺

The following compounds are obtained analogously to Example XXVI:

### (1) 5-tert.-butoxycarbonylamino-naphthalen-1-yl-boric acid

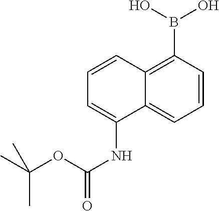

R_{f} value: 0.52 (silica gel, methylene chloride/methanol=95:5)

Mass spectrum (ESI⁺): m/z=288 [M+H]⁺

### (2) 6-(5-methyl-[1,2,4]oxadiazol-3-yl)-naphthalen-1-boric acid

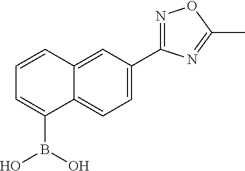

(carried out with tert.-butyllithium and trimethyl borate)

### Example XXVII

### tert. Butyl {(3,5-dichloro-phenylsulphonyl)-[6-(5-oxo-4,5-dihydro-[1,2,4]oxadiazol-3-yl)-naphthalen-2-yl]-amino}-acetate

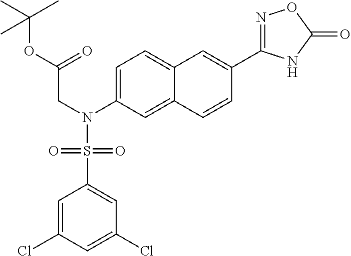

A solution of 400 mg bis-(trichloromethyl)-carbonate in 10 ml methylene chloride is added dropwise at −60° C. to 570 mg tert butyl {(3,5-dichloro-phenylsulphonyl)-[6-(N-hydroxycarbamimidoyl)-naphthalen-2-yl]-amino}-acetate and 0.35 ml triethylamine in 20 ml methylene chloride. After half an hour the cooling bath is removed and the reaction mixture is stirred overnight. Then the reaction mixture is diluted with ethyl acetate, washed successively with saturated ammonium chloride, sodium hydrogen carbonate and sodium chloride solution, dried on magnesium sulphate and evaporated down. The residue is chromatographed through a silica gel column with cyclohexane/ethyl acetate (9:1 to 0:10).

Yield: 480 mg (80% of theory)

R_{f} value: 0.70 (silica gel, petroleum ether/ethyl acetate=2:8)

Mass spectrum (ESI⁻): m/z=548, 550, 552 [M−H]⁻

Tert. Butyl {(3,5-dichloro-phenylsulphonyl)-[6-(4-methyl-5-oxo-4,5-dihydro-[1,2,4]oxadiazol-3-yl)-naphthalen-2-yl]-amino}-acetate washed obtained as a by-product.

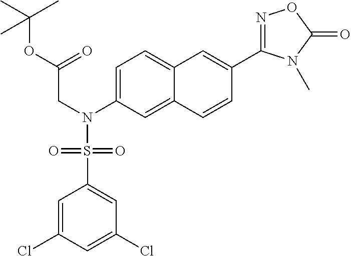

R_{f} value: 0.85 (silica gel, petroleum ether/ethyl acetate=2:8)

### Example XXVIII

### tert. Butyl {(3,5-dichloro-phenylsulphonyl)-[5-(5-morpholin-4-ylmethyl-[1,2,4]oxadiazol-3-yl)-naphthalen-2-yl]-amino}-acetate

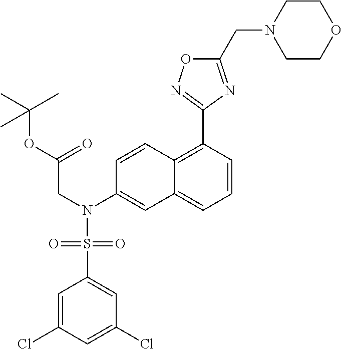

A mixture of 100 mg tert butyl [[5-(5-chloromethyl-[1,2,4]oxadiazol-3-yl)-naphthalen-2-yl]-(3,5-dichloro-phenylsulphonyl)-amino]-acetate, 20 μl morpholine and 29 mg potassium carbonate in 1 ml N,N-dimethylformamide is stirred overnight at ambient temperature. For working up the reaction mixture is diluted with ethyl acetate, washed with water and saturated sodium chloride solution, dried on magnesium sulphate and evaporated down. The flask residue is chromatographed through a silica gel column with petroleum ether/ethyl acetate (1:1) as eluant.

Yield: 73 mg (67% of theory)

R_{f} value: 0.40 (silica gel, petroleum ether/ethyl acetate=1:1)

Mass spectrum (ESI⁺): m/z=633, 635, 637 [M+H]⁺

### Example XXIX

### 3,5-dichloro-N-[6-(5-methyl-[1,2,4]oxadiazol-3-yl)-naphthalen-1-yl]-phenylsulphonamide

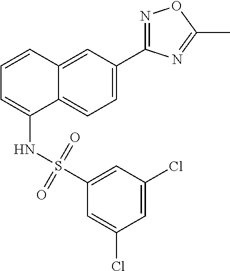

160 mg copper(II) acetate and 247 μl triethylamine are added to 405 mg 6-(5-methyl-[1,2,4]oxadiazol-3-yl)-naphthalene-1-boric acid, 200 mg of 3,5-dichloro-phenylsulphonamide and 100 mg molecular sieve (4 Å) in 15 ml methylene chloride and the reaction mixture is stirred for 24 h at ambient temperature. Then the mixture is diluted with ethyl acetate, washed with 1N sodium hydroxide solution, dried on magnesium sulphate and evaporated down. The crude product is purified by chromatography.

Yield: 120 mg (31% of theory)

### Example XXX

### 3-Bromo-5-chloro-phenylsulphonyl chloride

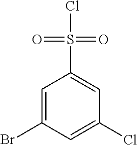

A solution of 400 mg sodium nitrite in 0.6 ml of water is added dropwise to 1.03 g 3-bromo-5-chloro-aniline in 2 ml concentrated hydrochloric acid while the mixture is cooled in a bath of ice/common salt. The reaction mixture is stirred for 15 minutes at 0° C. and then while being cooled it is added to a mixture of 4 ml of a saturated solution of sulphur dioxide in glacial acetic acid (approx. 30%) and 200 mg copper(II) chloride-dihydrate in 0.4 ml of water. The cooling bath is removed and the reaction mixture is stirred for 15 minutes at ambient temperature, then in the water bath with gentle heating until no further development of gas can be detected. Some ice water is then added while cooling with an ice bath. After 5 minutes the precipitate formed is suction filtered, washed with some ice water and dried in the desiccator. The sulphonyl chloride obtained is reacted further without any further purification.

Yield: 1.13 mg (78% of theory)

### Example XXXI

### tert.-butyl 4-(2-aminomethyl-benzoyl)-piperazine-1-carboxylate

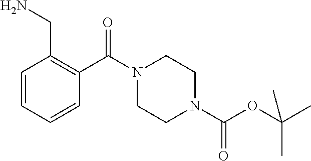

Prepared by hydrogenation of tert.-butyl 4-(2-cyanobenzoyl)-1-piperazine-carboxylate in ethanol/chloroform in the presence of platinum dioxide at ambient temperature and at a partial hydrogen pressure of 50 psi.

R_{f} value: 0.45 (silica gel, methylene chloride/methanol/conc. Aqueous ammonia=90:10:1)

Mass spectrum (ESI⁺): m/z=320 [M+H]⁺

The following compounds are obtained analogously to Example XXXI:

### (1) (2-aminomethyl-phenyl)-morpholin-4-yl-methanone

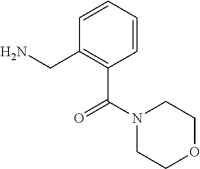

Mass spectrum (ESI⁺): m/z=221 [M+H]⁺

### Example XXXII

### Benzyl 5-(3,5-dichloro-phenylsulphonylamino)-naphthalene-1-carboxylate

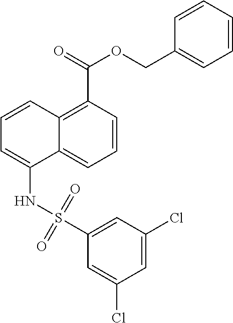

1.12 ml ethyldiisopropylamine and 610 mg 1-ethyl-3-(3-dimethylamino-propyl)-carbodiimide-hydrochloride are added to 1.05 g 5-(3,5-dichloro-phenylsulphonylamino)-naphthalene-1-carboxylic acid and 0.33 ml benzylalcohol in 20 ml acetonitrile and the reaction mixture is stirred for 1.5 h at ambient temperature. Then another 200 mg 1-ethyl-3-(3-dimethylaminopropyl)-carbodiimide-hydrochloride are added and the reaction mixture is stirred overnight at ambient temperature. Then the reaction mixture is evaporated down and the residue is distributed between tert-butylmethylether and dilute citric acid. The organic phase is washed with dilute sodium carbonate solution, water and saturated sodium chloride solution, dried on magnesium sulphate and evaporated down. The crude product is stirred with a little methanol, suction filtered, washed with a little methanol and dried.

Yield: 389 mg (30% of theory)

R_{f} value: 0.80 (silica gel, methylene chloride/methanol=95:5)

Mass spectrum (ESI⁻): m/z=484, 486, 488 [M−H]⁻

### Example XXXIII

### N-(4-aminomethyl-pyridin-2-yl)-N′,N′-dimethyl-ethane-1,2-diamine

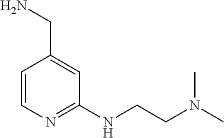

Prepared by treating 2-(2-dimethylamino-ethylamino)-isonicotinonitrile with hydrogen (50 psi) in the presence of Raney nickel in a mixture of methanol and methanolic ammonia solution at ambient temperature.

R_{f} value: 0.20 (silica gel, methylene chloride/methanol/conc. Aqueous ammonia=90:10:0.1)

Mass spectrum (ESI⁺): m/z=195 [M+H]⁺

The following compounds are obtained analogously to Example XXXIII:

### (1) N-(4-aminomethyl-pyridin-2-yl)-N,N′,N′-trimethyl-ethane-1,2-diamine

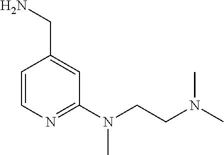

R_{f} value: 0.10 (silica gel, methylene chloride/methanol/conc. Aqueous ammonia=90:10:0.1)

Mass spectrum (ESI⁺): m/z=209 [M+H]⁺

### (2) N-(5-aminomethyl-pyridin-2-yl)-N,N′,N′-trimethyl-ethane-1,2-diamine

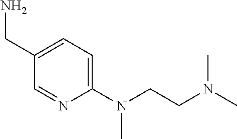

R_{f} value: 0.15 (silica gel, methylene chloride/methanol/conc. Aqueous ammonia=90:10:0.1)

Mass spectrum (ESI⁺): m/z=209 [M+H]⁺

### Example XXXIV

### 2-(2-dimethylamino-ethylamino)-isonicotinonitrile

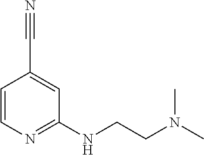

Prepared by reacting 2-chloro-4-cyano-pyridine with N,N-dimethyl-ethylenediamine in N,N-dimethylformamide in the presence of potassium carbonate at 100° C.

R_{f} value: 0.30 (silica gel, methylene chloride/methanol/conc. Aqueous ammonia=95:5:1)

Mass spectrum (ESI⁺): m/z=191 [M+H]⁺

The following compounds are obtained analogously to Example XXXIV:

### (1) 2-[(2-dimethylamino-ethyl)-methyl-amino]-isonicotinonitrile

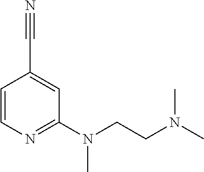

(The reaction is carried out in dimethylsulphoxide.)

R_{f} value: 0.20 (silica gel, methylene chloride/methanol/conc. Aqueous ammonia=95:5:0.1)

Mass spectrum (ESI⁺): m/z=205 [M+H]⁺

### (2) 6-[(2-dimethylamino-ethyl)-methyl-amino]-nicotinonitrile

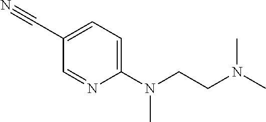

(The reaction is carried out in dimethylsulphoxide.)

R_{f} value: 0.40 (silica gel, methylene chloride/methanol/conc. Aqueous ammonia=95:5:0.1)

Mass spectrum (ESI⁺): m/z=205 [M+H]⁺

### Example XXXV

### Tert. Butyl {(3,5-dichloro-phenylsulphonyl)-[6-(4,4,5,5-tetramethyl-[1,3,2]dioxaborolan-2-yl)-naphthalen-2-yl]-amino}-acetate

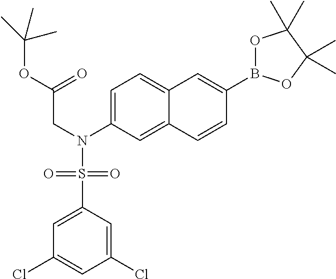

A mixture of 396 mg tert butyl [(3,5-dichloro-phenylsulphonyl)-(6-trifluoro-methanesulphonyloxy-naphthalen-2-yl)-amino]-}-acetate, 269 μl triethylamine and 26 mg [1,1′-bis-(diphenylphosphino)ferrocene]-dichloro-palladium(II) in 4 ml dioxane under an argon atmosphere is combined with the 327 mg of bis-(pinacolato)-diborane and gently refluxed for 5.5 h. After standing overnight at ambient temperature the reaction mixture is diluted with water and extracted with ethyl acetate. The combined ethyl acetate extracts are washed with water and saturated sodium chloride solution, dried on magnesium sulphate and evaporated down. The crude product is purified by chromatography through a silica gel column with petroleum ether/ethyl acetate (95:5 to 90:10) as eluant.

Yield: 216 mg (57% of theory)

R_{f} value: 0.35 (silica gel, methylene chloride)

Mass spectrum (ESI⁺): m/z=609, 611, 613 [M+NH₄]⁺

The following compounds are obtained analogously to Example XXXV:

### (1) tert. Butyl {(3,5-dichloro-phenylsulphonyl)-[5-(4,4,5,5-tetramethyl-[1,3,2]dioxaborolan-2-yl)-naphthalen-1-yl]-amino}-acetate

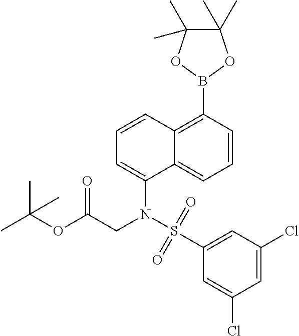

R_{f} value: 0.77 (silica gel, methylene chloride)

Mass spectrum (ESI⁺): m/z=609, 611, 613 [M+NH₄]⁺

### Example XXXVI

### N-(2-chloro-pyrimidin-4-yl)-N,N′,N′-trimethyl-ethane-1,2-diamine

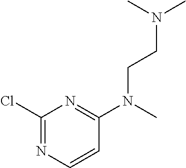

Prepared by reacting 2,4-dichloropyrimidine with N,N,N′-trimethyl-ethane-1,2-diamine in the presence of diisopropylethylamine in tetrahydrofuran at ambient temperature.

R_{f} value: 0.50 (silica gel, methylene chloride/methanol/conc. Aqueous ammonia=90:10:1)

Mass spectrum (ESI⁺): m/z=215 [M+H]⁺

The following compounds are obtained analogously to Example XXXVI:

### (1) N′-(2-chloro-pyrimidin-4-yl)-N,N-dimethyl-ethane-1,2-diamine

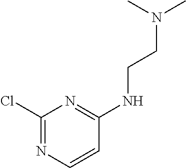

(The reaction takes place in N,N-dimethylformamide in the presence of potassium carbonate.)

Mass spectrum (ESI⁺): m/z=201 [M+H]⁺

### (2) 4-(6-iodo-pyridazin-3-yl)-morpholine

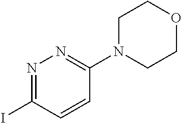

(The reaction takes place with 3,6-diiodopyridazine in dioxane in the presence of potassium carbonate.)

Mass spectrum (ESI⁺): m/z=292 [M+H]⁺

### (3) N′-(6-chloro-pyridazin-3-yl)-N,N-dimethyl-ethane-1,2-diamine

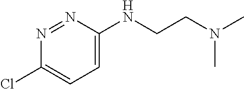

(The reaction takes place in dimethylsulphoxide in the presence of potassium carbonate.)

### Example XXXVII

### tert. Butyl {(3,5-dichloro-phenylsulphonyl)-[5-(2-morpholin-4-yl-pyrimidin-4-yl)-naphthalen-1-yl]-amino}-acetate

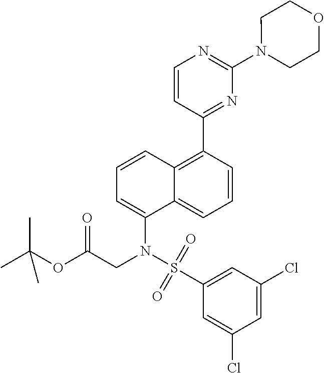

A mixture of 40 mg tert.butyl [[5-(2-chloro-pyrimidin-4-yl)-naphthalen-1-yl]-(3,5-dichloro-phenylsulphonyl)-amino]-acetate, 15 μl morpholine and 20 μl diisopropylethylamine in 1 ml isopropanol is heated to 120° C. in a microwaveable vessel for 10 minutes. The reaction mixture is evaporated down and chromatographed through a silica gel column with cyclohexane/ethyl acetate (85:15) as eluant.

Yield: 28 mg (64% of theory)

R_{f} value: 0.12 (silica gel, petroleum ether/ethyl acetate=5:1)

The following Examples are obtained analogously to Example XXXVII:

### (1) tert. Butyl {(3,5-dichloro-phenylsulphonyl)-[5-(2-morpholin-4-yl-pyrimidin-5-yl)-naphthalen-1-yl]-amino}-acetate

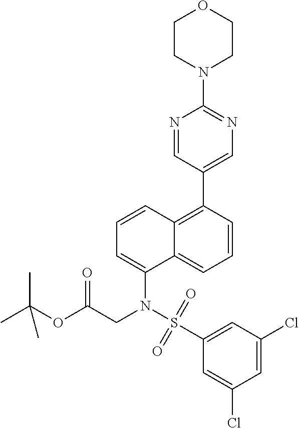

R_{f} value: 0.14 (silica gel, petroleum ether/ethyl acetate=5:1)

Mass spectrum (ESI⁺): m/z=629, 631, 633 [M+H]⁺

### Example XXXVIII

### 6-[tert-butoxycarbonylmethyl-(3,5-dichloro-phenylsulphonyl)-amino]-naphthalene-2-boric acid

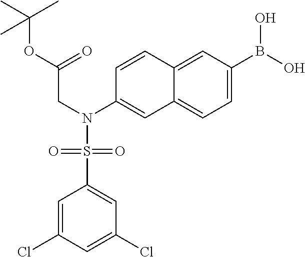

Prepared by treating tert.butyl {(3,5-dichloro-phenylsulphonyl)-[6-(4,4,5,5-tetramethyl-[1,3,2]dioxaborolan-2-yl)-naphthalen-2-yl]-amino}-acetate with sodium metaperiodate and ammonium acetate in acetone.

R_{f} value: 0.50 (silica gel, methylene chloride/methanol=95:5)

Mass spectrum (ESI⁺): m/z=527, 529, 531 [M+NH₄]⁺

### Example XXXIX

### N′-(5-bromo-pyrazin-2-yl)-N,N-dimethyl-ethane-1,2-diamine

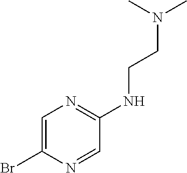

13.79 g sodium hydride (60% in mineral oil) are added batchwise to 24.00 g 5-bromo-pyrazin-2-ylamine and 19.87 g (2-chloro-ethyl)-dimethyl-amine-hydrochloride in 300 ml N,N-dimethylformamide at 0° C. After ten minutes the reaction mixture is heated to 80° C. for 2 h. After cooling to ambient temperature the reaction mixture is divided between semi-saturated sodium chloride solution and ethyl acetate. The aqueous phase is extracted with ethyl acetate and the combined organic phases are dried on magnesium sulphate and evaporated down. The flask residue is chromatographed through a silica gel column with methylene chloride/methanol (9:1 to 6:4) as eluant. The crude product is taken up in 150 ml of ethyl acetate and stirred with 12 g activated charcoal. After one hour the activated charcoal is filtered off, the filtrate is evaporated down, stirred with some petroleum ether and stored in the freezer for one hour. Then the petroleum ether is decanted off and the solid remaining is dried under a high vacuum.

Yield: 11.00 g (33% of theory)

Mass spectrum (ESI⁺): m/z=245, 247 [M+H]⁺

### Example XL

### N′-(6-bromo-pyridazin-3-yl)-N,N-dimethyl-ethane-1,2-diamine

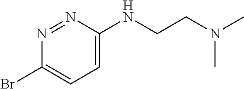

Prepared by treating N′-(6-chloro-pyridazin-3-yl)-N,N-dimethyl-ethane-1,2-diamine with 48% hydrobromic acid at 100° C.

Mass spectrum (ESI⁺): m/z=245, 247 [M+H]⁺

### Example XLI

### 4-(5-bromo-pyrazin-2-yl)-morpholine

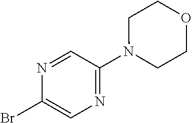

3.18 g N-bromosuccinimide are added at 0° C. to 2.95 g 4-pyrazin-2-yl-morpholine in 200 ml methylene chloride and the reaction mixture is heated to ambient temperature overnight with stirring. Then another 400 mg N-bromosuccinimide are added while cooling with an ice bath and the reaction mixture is stirred for a further 3 h. For working up the reaction mixture is washed with water and the organic phase is combined with magnesium sulphate and silica gel, stirred vigorously for 30 minutes and filtered. The filter cake is washed with a little methylene chloride followed by 200 ml of ethyl acetate. The combined filtrates are evaporated down. The flask residue is suction-filtered with tert.-butylmethylether, washed with a little tert.-butylmethylether and dried.

Yield: 3.05 g (69% of theory)

R_{f} value: 0.65 (silica gel, petroleum ether/ethyl acetate=1:1)

Mass spectrum (ESI⁺): m/z=244, 246 [M+H]⁺

### Preparation of the End Compounds

### Example 1

### [[5-(4-cyano-benzylaminocarbonyl)-naphthalen-2-yl]-(3,5-dichloro-phenyl-sulphonyl)-amino]-acetic acid

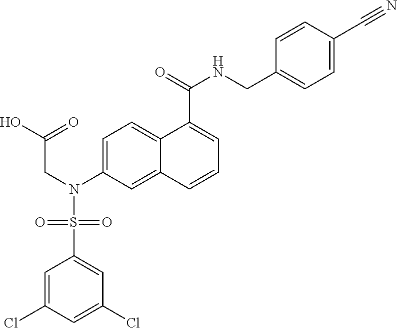

0.40 ml 1N sodium hydroxide solution are added to 100 mg methyl [[5-(4-cyano-benzylaminocarbonyl)-naphthalen-2-yl]-(3,5-dichloro-phenyl-sulphonyl)-amino]-acetate in 3 ml of methanol and the suspension is stirred overnight at ambient temperature. For working up 0.40 ml 1N hydrochloric acid are added dropwise and the mixture is diluted with approx. 15 ml of water. The precipitate formed is suction filtered and washed with water. The crude product is chromatographed through a silica gel column with ethyl acetate/methanol (100:0 to 98:2) as eluant. The product fractions are evaporated down and the flask residue is taken up in dilute sodium hydroxide solution. The product is precipitated by the addition of dilute hydrochloric acid, suction filtered, washed with water and dried.

Yield: 35 mg (36% of theory)

R_{f} value: 0.35 (silica gel, toluene/dioxane/ethanol/acetic acid=90:10:10:6)

Mass spectrum (ESI⁺): m/z=568, 570, 572 [M+H]⁺

The following compounds are obtained analogously to Example 1:

### (1) [(5-benzylaminocarbonyl-naphthalen-2-yl)-(3,5-dichloro-phenylsulphonyl)-amino]-acetic acid

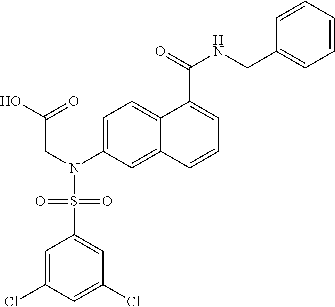

R_{f} value: 0.58 (silica gel, toluene/dioxane/ethanol/acetic acid=90:10:10:6)

Mass spectrum (ESI⁺): m/z=543, 545, 547 [M+H]⁺

### (2) [[5-(4-aminomethyl-benzylaminocarbonyl)-naphthalen-2-yl]-(3,5-dichloro-phenylsulphonyl)-amino]-acetic acid

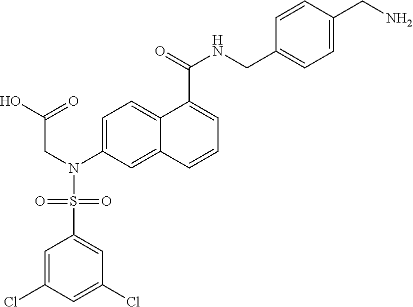

Mass spectrum (ESI⁺): m/z=572, 574, 576 [M+H]⁺

### (3) [(3,5-dichloro-phenylsulphonyl)-(6-phenylaminocarbonyl-naphthalen-2-yl)-amino]-acetic acid

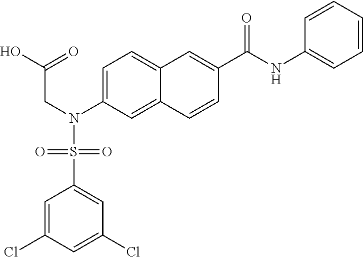

R_{f} value: 0.25 (silica gel, methylene chloride/methanol=95:5)

Mass spectrum (ESI⁺): m/z=529, 531, 533 [M+H]⁺

### (4) [(3,5-dichloro-phenylsulphonyl)-(6-methylaminocarbonyl-naphthalen-2-yl)-amino]-acetic acid

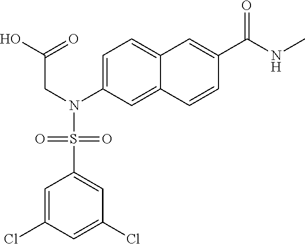

R_{f} value: 0.28 (silica gel, methylene chloride/methanol=95:5)

Mass spectrum (ESI⁺): m/z=467, 469, 471 [M+H]⁺

### (5) [(3,5-dichloro-phenylsulphonyl)-(4-methoxy-naphthalen-2-yl)-amino]-acetic acid

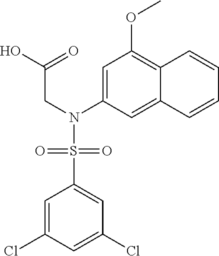

R_{f} value: 0.40 (silica gel, methylene chloride/methanol=95:5)

Mass spectrum (ESI⁺): m/z=440, 442, 444 [M+H]⁺

### (6) [(5-benzylaminocarbonyl-naphthalen-1-yl)-(3,5-dichloro-phenylsulphonyl)-amino]-acetic acid

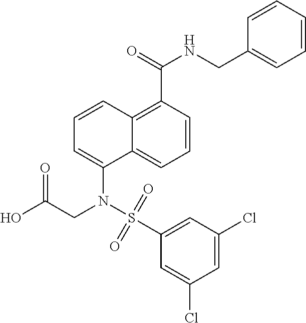

R_{f} value: 0.58 (silica gel, methylene chloride/methanol=9:1)

Mass spectrum (ESI⁺): m/z=543, 545, 547 [M+H]⁺

### (7) [(3,5-dichloro-phenylsulphonyl)-(5-methylaminocarbonyl-naphthalen-1-yl)-amino]-acetic acid

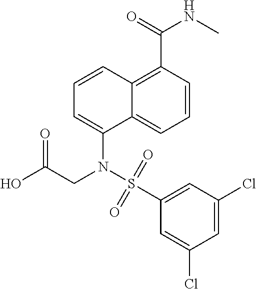

R_{f} value: 0.55 (silica gel, methylene chloride/methanol=9:1)

Mass spectrum (ESI⁺): m/z=467, 469, 471 [M+H]⁺

### (8) 4-[({5-[carboxymethyl-(3,5-dichloro-phenylsulphonyl)-amino]-naphthalen-1-ylcarbonyl}-amino)-methyl]-benzoic acid

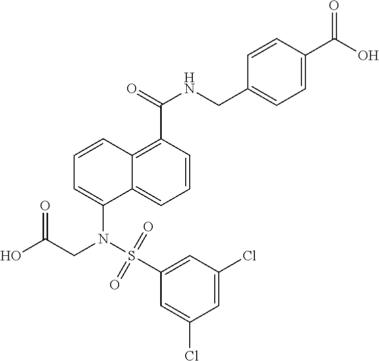

R_{f} value: 0.30 (silica gel, toluene/dioxane/ethanol/acetic acid=90:10:10:6)

Mass spectrum (ESI⁺): m/z=587, 589, 591 [M+H]⁺

### Example 2

### [[5-(4-aminocarbonyl-benzylaminocarbonyl)-naphthalen-2-yl]-(3,5-dichloro-phenylsulphonyl)-amino]-acetic acid

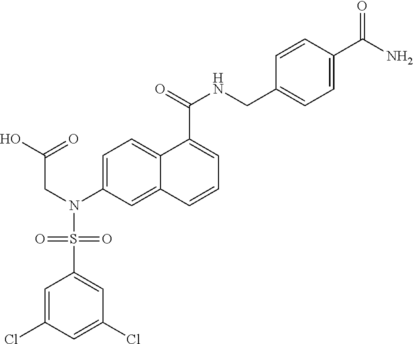

A mixture of 200 mg methyl [[5-(4-carbamimidoyl-benzylaminocarbonyl)-naphthalen-2-yl]-(3,5-dichloro-phenylsulphonyl)-amino]-acetate hydrochloride, 500 mg sodium carbonate, 2.5 ml of water and 5 ml of methanol is stirred overnight at ambient temperature and then refluxed for 8 h. After cooling to ambient temperature 1 ml of 1N sodium hydroxide solution is added. The reaction mixture is stirred overnight at ambient temperature, diluted with some water and acidified with 2N hydrochloric acid. The precipitate formed is suction filtered, washed with water and dried. The crude product thus obtained is stirred with methanol, suction filtered, washed with methanol and ether and dried.

Yield: 90 mg (49% of theory)

R_{f} value: 0.30 (silica gel, toluene/dioxane/ethanol/acetic acid=90:10:10:6)

Mass spectrum (ESI⁺): m/z=586, 588, 590 [M+H]⁺

### Example 3

### [(3,5-dichloro-phenylsulphonyl)-(5-methylaminocarbonyl-naphthalen-2-yl)-amino]-acetic acid

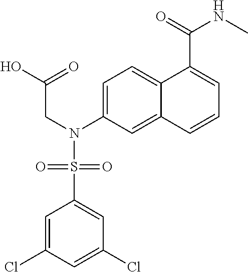

10 ml trifluoroacetic acid are added to 3.66 g tert.butyl [(3,5-dichloro-phenylsulphonyl)-(5-methylaminocarbonyl-naphthalen-2-yl)-amino]-acetate in 50 ml methylene chloride and the reaction mixture is stirred for 4.5 h at ambient temperature. Then the reaction mixture is evaporated down using the rotary evaporator and the flask residue is dissolved with dilute sodium hydroxide solution. This aqueous solution is washed with a little methylene chloride and acidified with 1N hydrochloric acid. The precipitate formed is suction filtered, washed with water and dried in the desiccator.

Yield: 3.04 g (Yield: 93% of theory)

R_{f} value: 0.30 (silica gel, toluene/dioxane/ethanol/acetic acid=90:10:10:6)

Mass spectrum (ESI⁻): m/z=465, 467, 469 [M−H]⁻

The following compounds are obtained analogously to Example 3:

### (1) [[5-(cyclohexylmethyl-aminocarbonyl)-naphthalen-2-yl]-(3,5-dichloro-phenylsulphonyl)-amino]-acetic acid

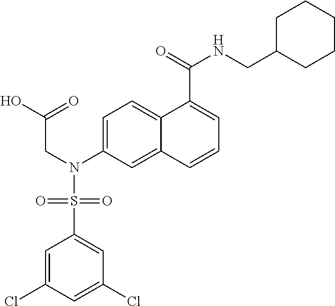

R_{f} value: 0.75 (silica gel, methylene chloride/methanol/conc. Aqueous ammonia=16:4:1)

Mass spectrum (ESI⁺): m/z=549, 551, 553 [M+H]⁺

### (2) [(3,5-dichloro-phenylsulphonyl)-(5-phenylaminocarbonyl-naphthalen-2-yl)-amino]-acetic acid

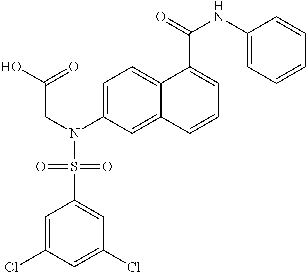

R_{f} value: 0.63 (silica gel, methylene chloride/methanol/conc. Aqueous ammonia=16:4:1)

Mass spectrum (ESI⁺): m/z=529, 531, 533 [M+H]⁺

### (3) {(3,5-dichloro-phenylsulphonyl)-[5-(3,4-dihydro-1 H-isoquinoline-2-carbonyl)-naphthalen-2-yl]-amino}-acetic acid

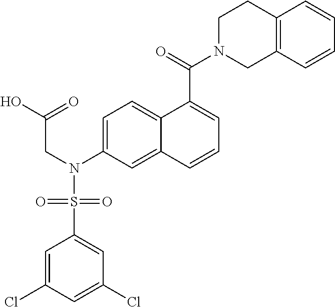

R_{f} value: 0.82 (silica gel, methylene chloride/methanol/conc. Aqueous ammonia=16:4:1)

Mass spectrum (ESI⁺): m/z=569, 571, 573 [M+H]⁺

### (4) [(3,5-dichloro-phenylsulphonyl)-(5-phenylethylaminocarbonyl-naphthalen-2-yl)-amino]-acetic acid

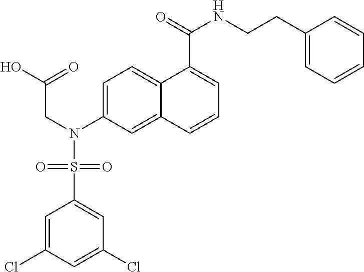

R_{f} value: 0.75 (silica gel, methylene chloride/methanol/conc. Aqueous ammonia=16:4:1)

Mass spectrum (ESI⁺): m/z=557, 559, 561 [M+H]⁺

### (5) [[5-(N-benzyl-N-methyl-aminocarbonyl)-naphthalen-2-yl]-(3,5-dichloro-phenylsulphonyl)-amino]-acetic acid

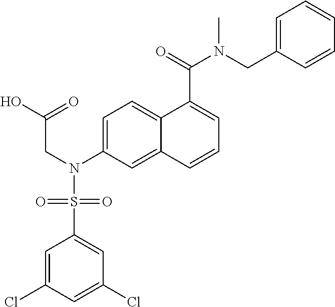

R_{f} value: 0.55 (silica gel, methylene chloride/methanol/conc. Aqueous ammonia=16:4:1)

Mass spectrum (ESI⁺): m/z=557, 559, 561 [M+H]⁺

### (6) [(6-aminocarbonyl-naphthalen-2-yl)-(3,5-dichloro-phenylsulphonyl)-amino]-acetic acid

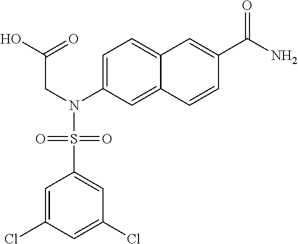

R_{f} value: 0.34 (Reversed phase ready-made TLC plate (E. Merck), acetonitrile/water/trifluoroacetic acid=50:50:1)

Mass spectrum (ESI⁺): m/z=453, 455, 457 [M+H]⁺

### (7) {(3,5-dichloro-phenylsulphonyl)-[6-(5-methyl-[1,2,4]oxadiazol-3-yl)-naphthalen-2-yl]-amino}-acetic acid

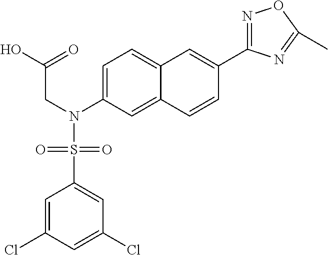

R_{f} value: 0.10 (Reversed phase ready-made TLC plate (E. Merck), acetonitrile/water/trifluoroacetic acid=50:50:1)

Mass spectrum (ESI⁻): m/z=490, 492, 494 [M−H]⁻

### (8) [(3,5-dichloro-phenylsulphonyl)-(6-oxazol-2-yl-naphthalen-2-yl)-amino]-acetic acid

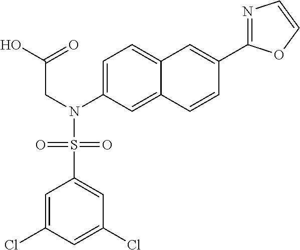

R_{f} value: 0.10 (Reversed phase ready-made TLC plate (E. Merck), acetonitrile/water/trifluoroacetic acid=50:50:1)

Mass spectrum (ESI⁻): m/z=475, 477, 479 [M−H]⁻

### (9) [(3,5-dichloro-phenylsulphonyl)-naphthalen-1-yl-amino]-acetic acid

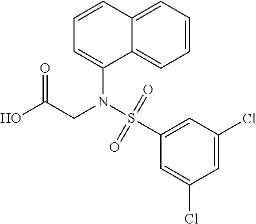

R_{f} value: 0.20 (Reversed phase ready-made TLC plate (E. Merck), acetonitrile/water/trifluoroacetic acid=50:50:1)

Mass spectrum (ESI⁻): m/z=408, 410, 412 [M−H]⁻

### (10) [(6-benzylaminocarbonyl-naphthalen-2-yl)-(3,5-dichloro-phenylsulphonyl)-amino]-acetic acid

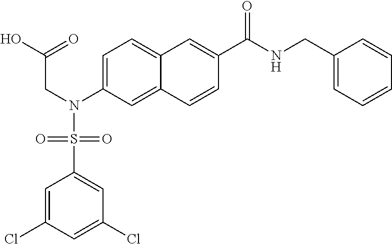

Mass spectrum (ESI⁺): m/z=543, 545, 547 [M+H]⁺

### (11) {(3,5-dichloro-phenylsulphonyl))-[5-(pyrrolidine-1-carbonyl)-naphthalen-2-yl]-amino}-acetic acid

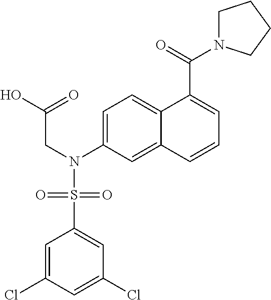

Mass spectrum (ESI⁺): m/z=507, 509, 511 [M+H]⁺

### (12) [(3,5-dichloro-phenylsulphonyl)-(5-isopropylaminocarbonyl-naphthalen-2-yl)-amino]-acetic acid

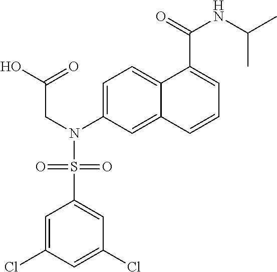

Mass spectrum (ESI⁺): m/z=495, 497, 499 [M+H]⁺

### (13) [(5-aminocarbonyl-naphthalen-2-yl)-(3,5-dichloro-phenylsulphonyl)-amino]-acetic acid

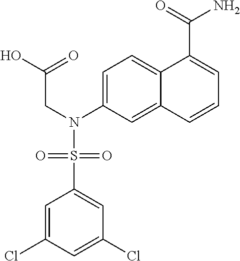

Mass spectrum (ESI⁺): m/z=470, 472, 474[M+N]⁺

### (14) {(3,5-dichloro-phenylsulphonyl)-[5-(2-hydroxy-ethylaminocarbonyl)-naphthalen-2-yl]-amino}-acetic acid

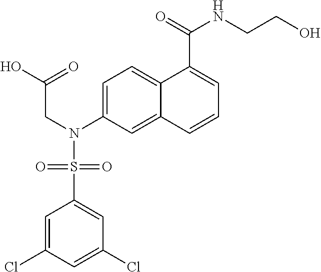

Mass spectrum (ESI⁺): m/z=497, 499, 501 [M+H]⁺

### (15) [(5-benzylaminocarbonyl-naphthalen-2-yl)-(3,5-dimethyl-phenylsulphonyl)-amino]acetic acid

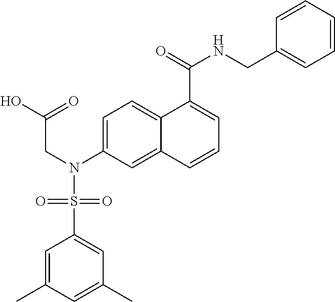

R_{f} value: 0.15 (silica gel, methylene chloride/methanol=9:1)

Mass spectrum (ESI⁺): m/z=503 [M+H]⁺

### (16) [(6-benzylaminocarbonyl-naphthalen-2-yl)-(3,5-dimethyl-phenylsulphonyl)-amino]-acetic acid

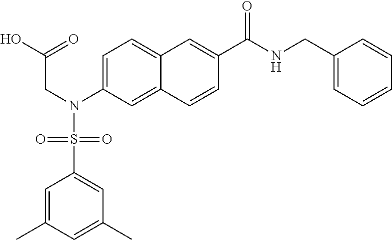

R_{f} value: 0.28 (silica gel, petroleum ether/ethyl acetate=1:2)

Mass spectrum (ESI⁺): m/z=503 [M+H]⁺

### (17) [(7-benzoylamino-naphthalen-2-yl)-(3,5-dichloro-phenylsulphonyl)-amino]-acetic acid

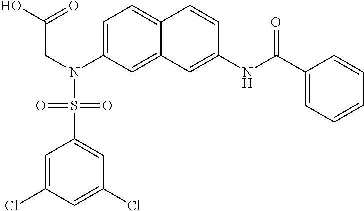

Mass spectrum (ESI⁻): m/z=527, 529, 531 [M−H]⁻

### (18) [{7-[(4-chloro-pyridin-2-carbonyl)-amino]-naphthalen-2-yl}-(3,5-dichloro-phenylsulphonyl)-amino]-acetic acid

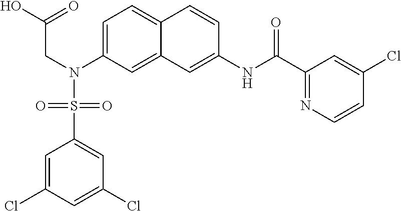

Mass spectrum (ESI⁻): m/z=562, 564, 566, 568 [M−H]⁻

### (19) [[7-(3-benzyl-ureido)-naphthalen-2-yl]-(3,5-dichloro-phenylsulphonyl)-amino]-acetic acid

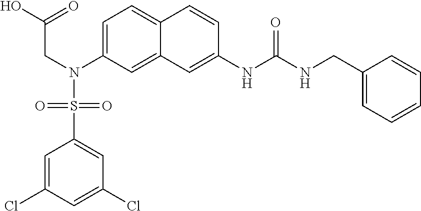

Mass spectrum (ESI⁻): m/z=556, 558, 560 [M−H]⁻

### (20) [(3,5-dichloro-phenylsulphonyl)-(6-pyridin-3-yl-naphthalen-2-yl)-amino]-acetic acid

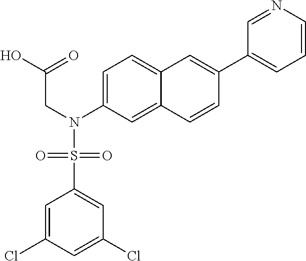

R_{f} value: 0.55 (silica gel, ethyl acetate/acetic acid=99:1)

Mass spectrum (ESI⁺): m/z=487, 489, 491 [M+H]⁺

### (21) {(3,5-dichloro-phenylsulphonyl)-[7-(5-methyl-[1,2,4]oxadiazol-3-yl)-naphthalen-2-yl]-amino}-acetic acid

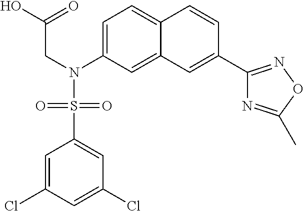

Mass spectrum (ESI⁻): m/z=490, 492, 494 [M−H]⁻

### (22) {(3,5-dichloro-phenylsulphonyl)-[5-(5-methyl-[1,2,4]oxadiazol-3-yl)-naphthalen-2-yl]-amino}-acetic acid

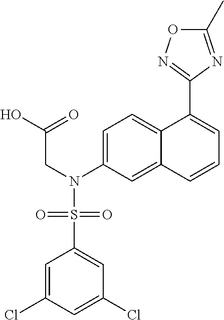

R_{f} value: 0.40 (silica gel, methylene chloride/methanol=10:1)

Mass spectrum (ESI⁺): m/z=492, 494, 496 [M+H]⁺

### (23) {(3,5-dichloro-phenylsulphonyl)-[5-(3-methyl-[1,2,4]oxadiazol-5-yl)-naphthalen-2-yl]-amino}-acetic acid

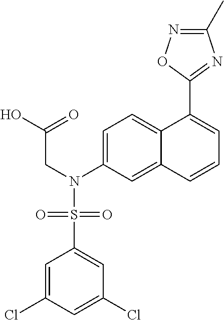

R_{f} value: 0.30 (silica gel, methylene chloride/methanol=10:1)

Mass spectrum (ESI⁺): m/z=492, 494, 496 [M+H]⁺

### (24) {(3,5-dichloro-phenylsulphonyl)-[6-(3-methyl-[1,2,4]oxadiazol-5-yl)-naphthalen-2-yl]-amino}-acetic acid

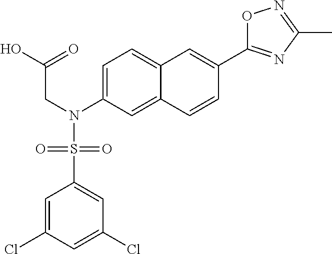

R_{f} value: 0.38 (silica gel, methylene chloride/methanol=9:1)

Mass spectrum (ESI⁺): m/z=492, 494, 496 [M+H]⁺

### (25) {(3,5-dichloro-phenylsulphonyl)-[6-(3-phenyl-[1,2,4]oxadiazol-5-yl)-naphthalen-2-yl]-amino}-acetic acid

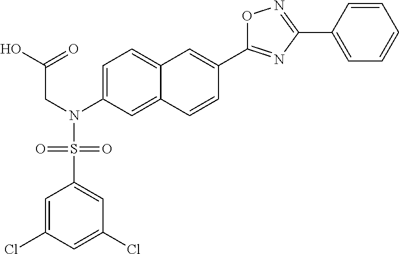

R_{f} value: 0.38 (silica gel, methylene chloride/methanol=9:1)

Mass spectrum (ESI⁺): m/z=554, 556, 558 [M+H]⁺

### (26) {(3,5-dichloro-phenylsulphonyl)-[5-(5-phenyl-[1,2,4]oxadiazol-3-yl)-naphthalen-2-yl]-amino}-acetic acid

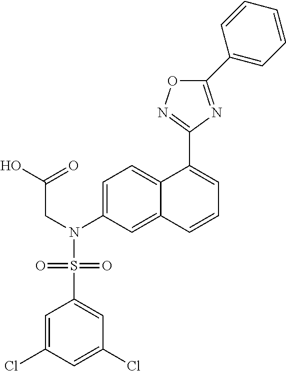

R_{f} value: 0.40 (silica gel, methylene chloride/methanol=10:1)

Mass spectrum (ESI⁺): m/z=554, 556, 558 [M+H]⁺

### (27) {(3,5-dichloro-phenylsulphonyl)-[5-(5-benzyl-[1,2,4]oxadiazol-3-yl)-naphthalen-2-yl]-amino}-acetic acid

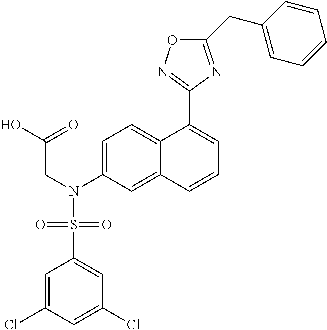

R_{f} value: 0.40 (silica gel, methylene chloride/methanol=10:1)

Mass spectrum (ESI⁺): m/z=568, 570, 572 [M+H]⁺

### (28) {(3,5-dichloro-phenylsulphonyl)-[5-(5-isopropyl-[1,2,4]oxadiazol-3-yl)-naphthalen-2-yl]-amino}-acetic acid

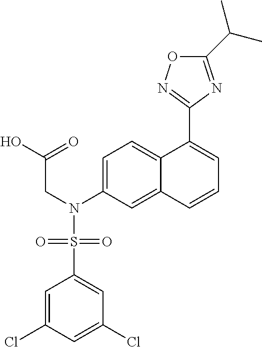

R_{f} value: 0.40 (silica gel, methylene chloride/methanol=10:1)

Mass spectrum (ESI⁺): m/z=520, 522, 524 [M+H]⁺

### (29) {(3,5-dichloro-phenylsulphonyl)-[6-(5-phenyl-[1,2,4]oxadiazol-3-yl)-naphthalen-2-yl]-amino}-acetic acid

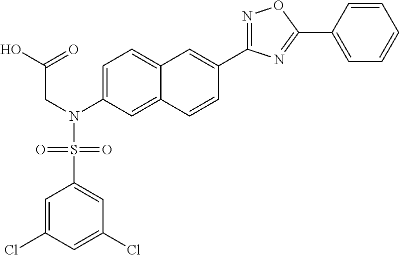

R_{f} value: 0.35 (silica gel, methylene chloride/methanol=9:1)

Mass spectrum (ESI⁺): m/z=554, 556, 558 [M+H]⁺

### (30) {(3,5-dichloro-phenylsulphonyl)-[6-(5-phenyl-[1,2,4]oxadiazol-3-yl)-naphthalen-2-yl]-amino}-acetic acid

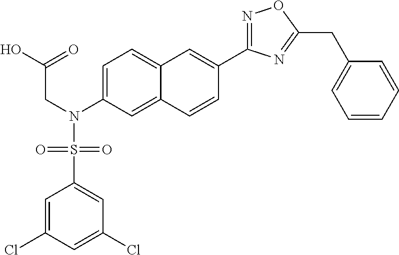

R_{f} value: 0.38 (silica gel, methylene chloride/methanol=9:1)

Mass spectrum (ESI⁺): m/z=568, 570, 572 [M+H]⁺

### (31) {(3,5-dichloro-phenylsulphonyl)-[6-(5-isopropyl-[1,2,4]oxadiazol-3-yl)-naphthalen-2-yl]-amino}-acetic acid

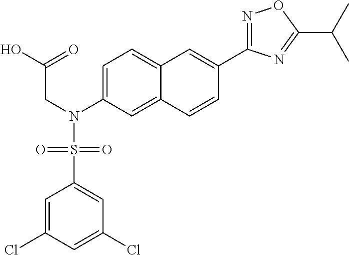

R_{f} value: 0.30 (silica gel, methylene chloride/methanol=9:1)

Mass spectrum (ESI⁺): m/z=520, 522, 524 [M+H]⁺

### (32) {(3,5-dichloro-phenylsulphonyl)-[5-(5-hydroxy-[1,2,4]oxadiazol-3-yl)-naphthalen-2-yl]-amino}-acetic acid

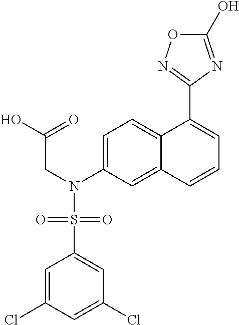

R_{f} value: 0.30 (silica gel, methylene chloride/methanol/acetic acid=5:1:0.1)

Mass spectrum (ESI⁺): m/z=494, 496, 498 [M+H]⁺

### (33) {(3,5-dichloro-phenylsulphonyl)-[5-(5-trichloromethyl-[1,2,4]oxadiazol-3-yl)-naphthalen-2-yl]-amino}-acetic acid

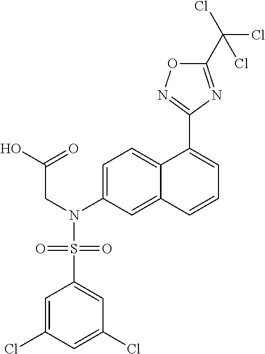

R_{f} value: 0.40 (silica gel, methylene chloride/methanol=10:1)

Mass spectrum (ESI⁺): m/z=594, 596, 598, 600 [M+H]⁺

### (34) {(3,5-dichloro-phenylsulphonyl)-[5-(3-ethoxycarbonyl-[1,2,4]oxadiazol-5-yl)-naphthalen-2-yl]-amino}-acetic acid

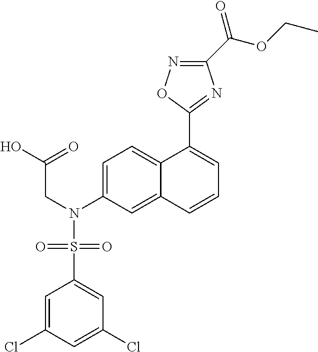

R_{f} value: 0.40 (silica gel, methylene chloride/methanol=10:1)

Mass spectrum (ESI⁺): m/z=550, 552, 554 [M+H]⁺

### (35) {(3,5-dichloro-phenylsulphonyl)-[6-(5-methyl-[1.3.4]oxadiazol-2-yl)-naphthalen-2-yl]-amino}-acetic acid

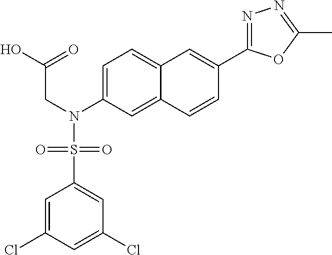

R_{f} value: 0.26 (silica gel, methylene chloride/methanol=9:1)

Mass spectrum (ESI⁺): m/z=492, 494, 496 [M+H]⁺

### (36) {(3,5-dichloro-phenylsulphonyl)-[5-(3-benzyl-[1,2,4]oxadiazol-5-yl)-naphthalen-2-yl]-amino}-acetic acid

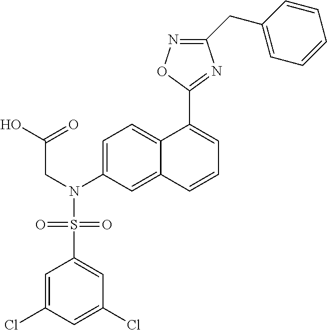

R_{f} value: 0.60 (silica gel, methylene chloride/methanol=5:1)

Mass spectrum (ESI⁺): m/z=568, 570, 572 [M+H]⁺

### (37) {(3,5-dichloro-phenylsulphonyl)-[5-(3-phenyloxymethyl-[1,2,4]oxadiazol-5-yl)-naphthalen-2-yl]-amino}-acetic acid

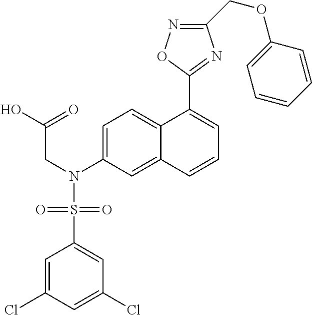

R_{f} value: 0.70 (silica gel, methylene chloride/methanol=5:1)

Mass spectrum (ESI⁻): m/z=582, 584, 586 [M−H]⁻

### (38) {(3,5-dichloro-phenylsulphonyl)-[5-(3-phenylsulphonylmethyl-[1,2,4]oxadiazol-5-yl)naphthalen-2-yl]-amino}-acetic acid

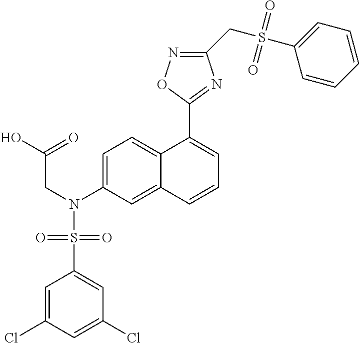

R_{f} value: 0.50 (silica gel, methylene chloride/methanol=5:1)

Mass spectrum (ESI⁻): m/z=630, 632, 634 [M−H]⁻

### (39) [(5-benzoylamino-naphthalen-1-yl)-(3,5-dichloro-phenylsulphonyl)-amino]-acetic acid

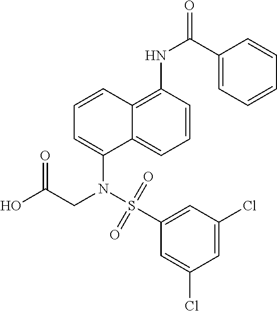

R_{f} value: 0.35 (silica gel, methylene chloride/methanol=95:5)

Mass spectrum (ESI⁻): m/z=527, 529, 531 [M−H]⁻

### (40) [(3,5-dichloro-phenylsulphonyl)-(5-phenylacetylamino-naphthalen-1-yl)-amino]-acetic acid

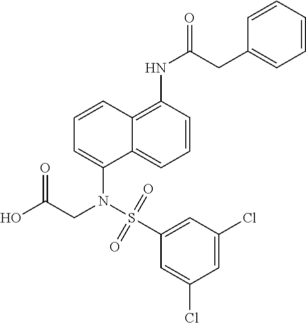

R_{f} value: 0.32 (silica gel, methylene chloride/methanol=95:5)

Mass spectrum (ESI⁻): m/z=541, 543, 545 [M−H]⁻

### (41) {(3,5-dichloro-phenylsulphonyl)-[5-(3-phenyl-propionylamino)-naphthalen-1-yl]-amino}-acetic acid

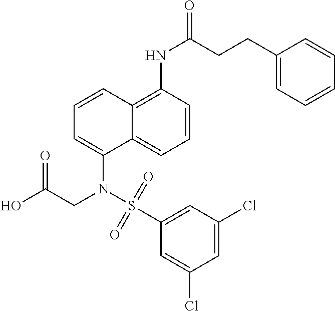

R_{f} value: 0.35 (silica gel, methylene chloride/methanol=95:5)

Mass spectrum (ESI⁻): m/z=555, 557, 559 [M−H]⁻

### (42) {(3,5-dichloro-phenylsulphonyl)-[5-(3-phenyl-ureido)-naphthalen-1-yl]-amino}-acetic acid

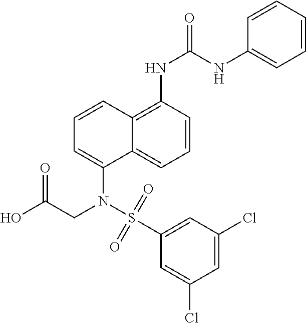

R_{f} value: 0.28 (silica gel, methylene chloride/methanol=95:5)

Mass spectrum (ESI⁻): m/z=542, 544, 546 [M−H]⁻

### (43) [[5-(3-benzyl-ureido)-naphthalen-1-yl]-(3,5-dichloro-phenylsulphonyl)-amino]-acetic acid

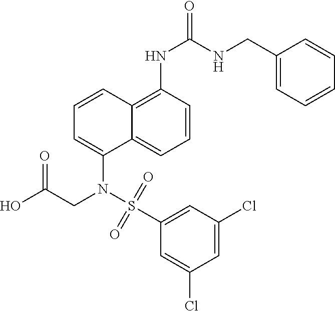

R_{f} value: 0.26 (silica gel, methylene chloride/methanol=95:5)

Mass spectrum (ESI⁻): m/z=556, 558, 560 [M−H]⁻

### (44) [{5-[(4-chloro-pyridin-2-yl-carbonyl)-amino]-naphthalen-1-yl}-(3,5-dichloro-phenylsulphonyl)-amino]-acetic acid

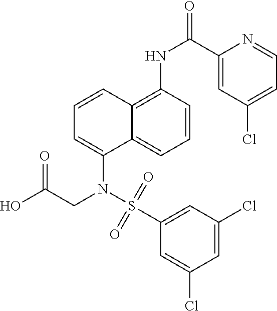

R_{f} value: 0.45 (silica gel, methylene chloride/methanol=95:5)

Mass spectrum (ESI⁺): m/z=564, 566, 568 [M+H]⁺

### (45) [(3,5-dichloro-phenylsulphonyl)-(5-phenylaminocarbonyl-naphthalen-1-yl)-amino]-acetic acid

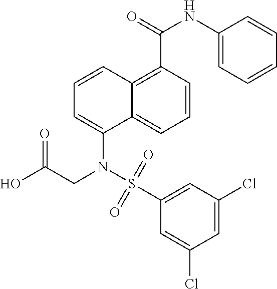

R_{f} value: 0.17 (silica gel, methylene chloride/methanol=95:5)

Mass spectrum (ESI⁺): m/z=529, 531, 533 [M+H]⁺

### (46) [(3,5-dichloro-phenylsulphonyl)-(6-pyridin-4-yl-naphthalen-2-yl)-amino]-acetic acid

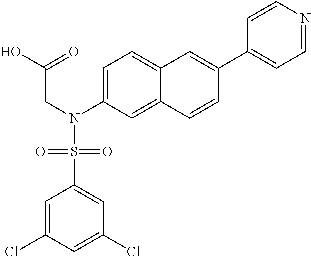

R_{f} value: 0.60 (silica gel, ethyl acetate/acetic acid=99:1)

Mass spectrum (ESI⁺): m/z=487, 489, 491 [M+H]⁺

### (47) [(3,5-dichloro-phenylsulphonyl)-(6-furan-3-yl-naphthalen-2-yl)-amino]-acetic acid

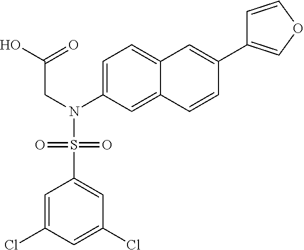

R_{f} value: 0.65 (silica gel, cyclohexane/ethyl acetate/acetic acid=50:50:1)

Mass spectrum (ESI⁻): m/z=474, 476, 478 [M−H]⁻

### (48) {(3,5-dichloro-phenylsulphonyl)-[6-(3,5-dimethyl-isoxazol-4-yl)-naphthalen-2-yl]-amino}-acetic acid

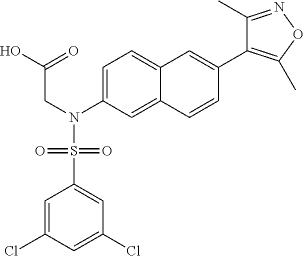

R_{f} value: 0.57 (silica gel, cyclohexane/ethyl acetate/acetic acid=50:50:1)

Mass spectrum (ESI⁻): m/z=503, 505, 507 [M−H]⁻

### (49) {(3,5-dichloro-phenylsulphonyl)-[5-(5-methyl-[1,2,4]oxadiazol-3-yl)-naphthalen-1-yl]-amino}-acetic acid

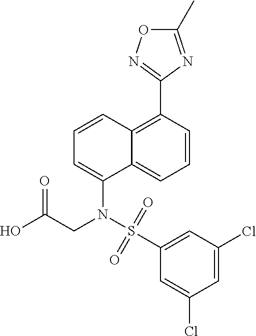

R_{f} value: 0.48 (silica gel, methylene chloride/methanol=95:5)

Mass spectrum (ESI⁻): m/z=490, 492, 494 [M−H]⁻

### (50) [(3,5-dichloro-phenylsulphonyl)-(6-furan-2-yl-naphthalen-2-yl)-amino]-acetic acid

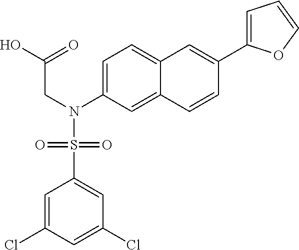

R_{f} value: 0.60 (silica gel, cyclohexane/ethyl acetate/acetic acid=50:50:1)

Mass spectrum (ESI⁻): m/z=474, 476, 478 [M−H]⁻

### (51) [(5-aminocarbonyl-naphthalen-1-yl)-(3,5-dichloro-phenylsulphonyl)-amino]-acetic acid

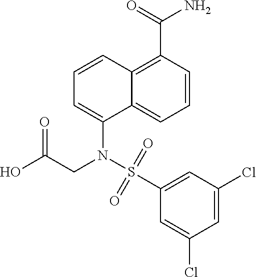

R_{f} value: 0.52 (silica gel, methylene chloride/methanol=9:1)

Mass spectrum (ESI⁺): m/z=453, 455, 457 [M+H]⁺

### (52) [(3,5-dichloro-phenylsulphonyl)-(6-pyrimidin-2-yl-naphthalen-2-yl)-amino]-acetic acid

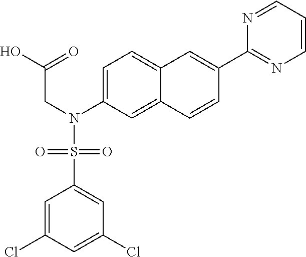

R_{f} value: 0.32 (silica gel, methylene chloride/methanol=95:5)

Mass spectrum (ESI⁺): m/z=488, 490, 492 [M+H]⁺

### (53) [(3,5-dichloro-phenylsulphonyl)-(5-pyrimidin-2-yl-naphthalen-1-yl)-amino]-acetic acid

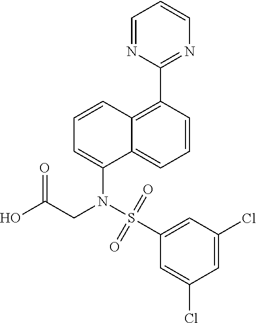

R_{f} value: 0.30 (silica gel, methylene chloride/methanol=95:5)

Mass spectrum (ESI⁺): m/z=488, 490, 492 [M+H]⁺

### (54) [(3,5-dichloro-phenylsulphonyl)-(6-pyridin-2-yl-naphthalen-2-yl)-amino]-acetic acid

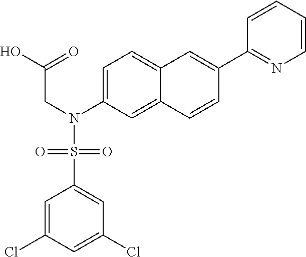

R_{f} value: 0.65 (silica gel, methylene chloride/methanol=9:1)

Mass spectrum (ESI⁺): m/z=487, 489, 491 [M+H]⁺

### (55) [(3,5-dichloro-phenylsulphonyl)-(5-phenylethyl-aminocarbonyl-naphthalen-1-yl)-amino]-acetic acid

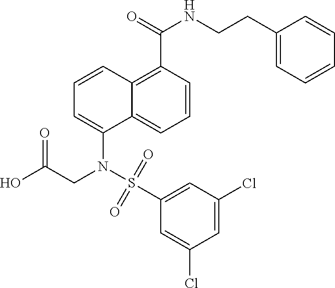

Mass spectrum (ESI⁺): m/z=557, 559, 561 [M+H]⁺

### (56) [(3,5-dichloro-phenylsulphonyl)-(6-methoxy-naphthalen-1-yl)-amino]-acetic acid

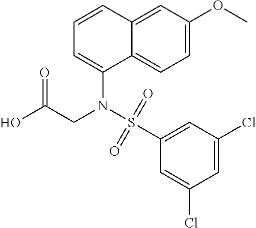

R_{f} value: 0.13 (Reversed phase ready-made TLC plate (E. Merck), acetonitrile/water/trifluoroacetic acid=50:50:1)

Mass spectrum (ESI⁻): m/z=438, 440, 442 [M−H]⁻

### (57) {(3,5-dichloro-phenylsulphonyl)-[6-(3-ethoxycarbonyl-[1,2,4]oxadiazol-5-yl)-naphthalen-2-yl]-amino}-acetic acid

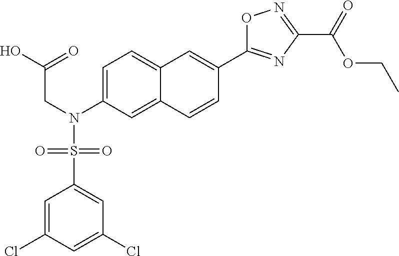

R_{f} value: 0.33 (silica gel, methylene chloride/methanol=9:1)

Mass spectrum (ESI⁺): m/z=550, 552, 564 [M+H]⁺

### (58) {[6-(3-benzyl-[1,2,4]oxadiazol-5-yl)-naphthalen-2-yl]-(3,5-dichloro-phenylsulphonyl)-amino}-acetic acid

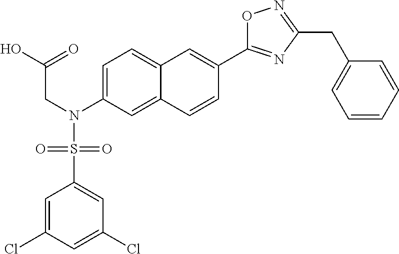

R_{f} value: 0.34 (silica gel, methylene chloride/methanol=9:1)

Mass spectrum (ESI⁺): m/z=568, 570, 572 [M+H]⁺

### (59) {(3,5-dichloro-phenylsulphonyl)-[6-(3-phenyloxymethyl-[1,2,4]oxadiazol-5-yl)-naphthalen-2-yl]-amino}-acetic acid

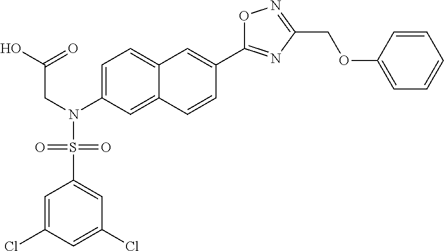

R_{f} value: 0.32 (silica gel, methylene chloride/methanol=9:1)

Mass spectrum (ESI⁻): m/z=582, 584, 586 [M−H]⁻

### (60) {(3,5-dichloro-phenylsulphonyl)-[6-(3-phenylsulphonylmethyl-[1,2,4]oxadiazol-5-yl)-naphthalen-2-yl]-amino}-acetic acid

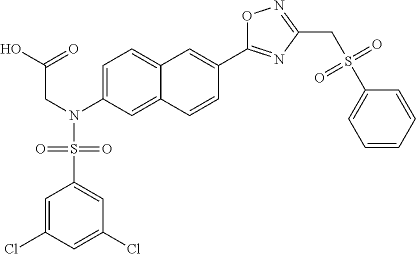

R_{f} value: 0.35 (silica gel, methylene chloride/methanol=9:1)

Mass spectrum (ESI⁻): m/z=630, 632, 634 [M−H]⁻

### (61) {(3,5-dichloro-phenylsulphonyl)-[6-(5-oxo-4,5-dihydro-[1,2,4]oxadiazol-3-yl)-naphthalen-2-yl]-amino}-acetic acid

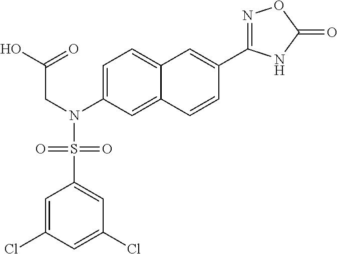

R_{f} value: 0.12 (silica gel, methylene chloride/methanol=9:1)

Mass spectrum (ESI⁻): m/z=492, 494, 496 [M−H]⁻

### (62) {(3,5-dichloro-phenylsulphonyl)-[6-(4-methyl-5-oxo-4,5-dihydro-[1,2,4]oxadiazol-3-yl)naphthalen-2-yl]-amino}-acetic acid

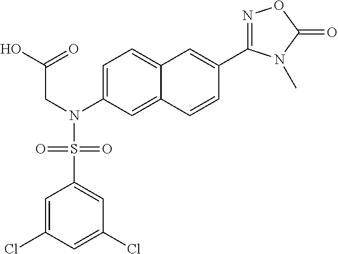

R_{f} value: 0.43 (silica gel, methylene chloride/methanol=9:1)

Mass spectrum (ESI⁻): m/z=506, 508, 510 [M−H]⁻

### (63) {(3,5-dichloro-phenylsulphonyl)-[5-(5-morpholin-4-ylmethyl-[1,2,4]oxadiazol-3-yl)-naphthalen-2-yl]-amino}-acetic acid

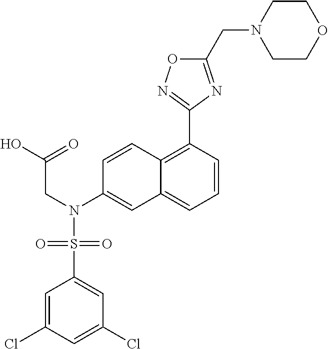

R_{f} value: 0.40 (silica gel, methylene chloride/methanol=9:1)

Mass spectrum (ESI⁺): m/z=577, 579, 581 [M+H]⁺

### (64) {(3,5-dichloro-phenylsulphonyl)-[6-(5-methyl-[1,2,4]oxadiazol-3-yl)-naphthalen-1-yl]-amino}-acetic acid

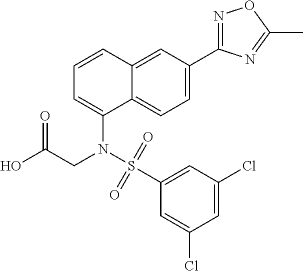

Mass spectrum (ESI⁻): m/z=490, 492, 494 [M−H]⁻

### (65) ((3,5-dichloro-phenylsulphonyl)-{5-[(pyridin-4-ylmethyl)-aminocarbonyl]-naphthalen-1-yl}-amino)-acetic acid

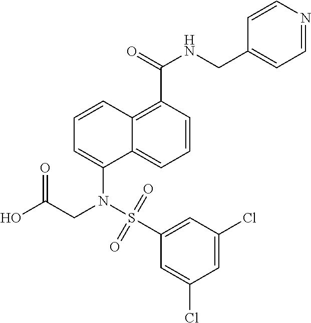

R_{f} value: 0.20 (silica gel, methylene chloride/methanol=9:1)

Mass spectrum (ESI⁺): m/z=544, 546, 548 [M+H]⁺

### (66) [[5-(cyclohexylmethyl-aminocarbonyl)-naphthalen-1-yl]-(3,5-dichloro-phenylsulphonyl)-amino]-acetic acid

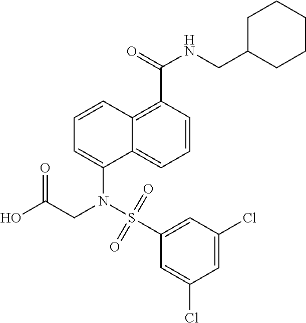

R_{f} value: 0.43 (silica gel, methylene chloride/methanol=95:5)

Mass spectrum (ESI⁺): m/z=549, 551, 553 [M+H]⁺

### (67) ((3,5-dichloro-phenylsulphonyl)-{5-[(pyridin-2-ylmethyl)-aminocarbonyl]-naphthalen-1-yl}-amino)-acetic acid

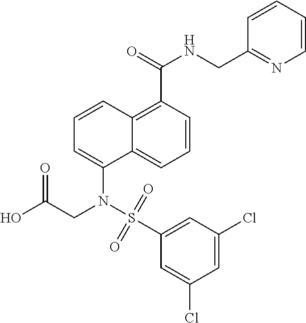

R_{f} value: 0.55 (silica gel, methylene chloride/methanol=9:1)

Mass spectrum (ESI⁺): m/z=544, 546, 548 [M+H]⁺

### (68) ((3,5-dichloro-phenylsulphonyl)-{5-[(pyridin-3-ylmethyl)-aminocarbonyl]-naphthalen-1-yl}-amino)-acetic acid

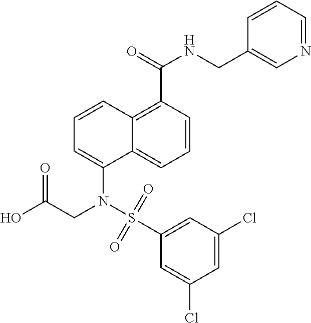

R_{f} value: 0.55 (silica gel, methylene chloride/methanol=9:1)

Mass spectrum (ESI⁺): m/z=544, 546, 548 [M+H]⁺

### (69) [(3,5-dibromo-phenylsulphonyl)-(naphthalen-1-yl)amino]-acetic acid

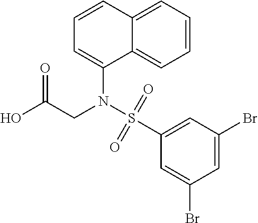

R_{f} value: 0.48 (silica gel, petroleum ether/ethyl acetate=2:3)

Mass spectrum (ESI⁺): m/z=498, 500, 502 [M+H]⁺

### (70) [(3-bromo-5-methyl-phenylsulphonyl)-(naphthalen-1-yl)-amino]-acetic acid

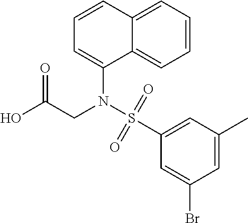

R_{f} value: 0.56 (silica gel, petroleum ether/ethyl acetate=2:3)

Mass spectrum (ESI⁺): m/z=434, 536 [M+H]⁺

### (71) [(3-bromo-5-chloro-phenylsulphonyl)-(naphthalen-1-yl)-amino]-acetic acid

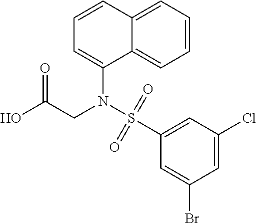

R_{f} value: 0.66 (silica gel, petroleum ether/ethyl acetate=2:3)

Mass spectrum (ESI⁻): m/z=452, 554, 556 [M−H]⁻

### (72) [[5-(4-cyano-benzylaminocarbonyl)-naphthalen-1-yl]-(3,5-dichloro-phenylsulphonyl)-amino]-acetic acid

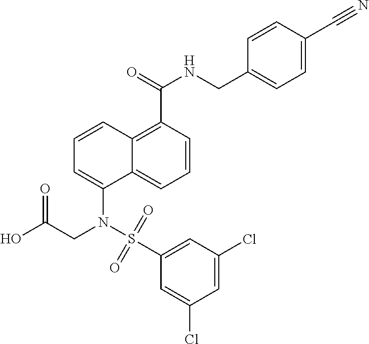

R_{f} value: 0.30 (silica gel, methylene chloride/methanol=95:5)

Mass spectrum (ESI⁺): m/z=568, 570, 572 [M+H]⁺

### (73) [[5-(3-cyano-benzylaminocarbonyl)-naphthalen-1-yl]-(3,5-dichloro-phenylsulphonyl)-amino]-acetic acid

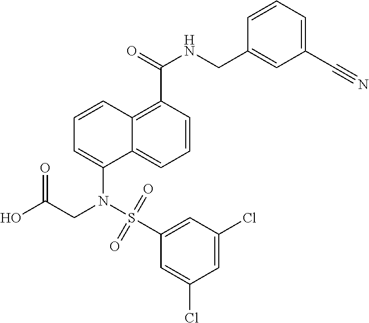

R_{f} value: 0.35 (silica gel, methylene chloride/methanol=95:5)

Mass spectrum (ESI⁻): m/z=566, 568, 570 [M−H]⁻

### (74) [[5-(2-cyano-benzylaminocarbonyl)-naphthalen-1-yl]-(3,5-dichloro-phenylsulphonyl)-amino]-acetic acid

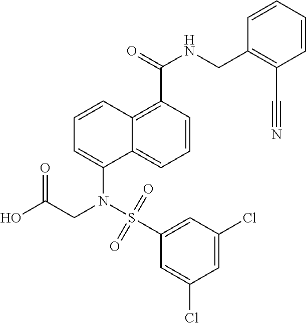

R_{f} value: 0.35 (silica gel, methylene chloride/methanol=95:5)

Mass spectrum (ESI⁺): m/z=568, 570, 572 [M+H]⁺

### (75) {(3,5-dichloro-phenylsulphonyl)-[2-(5-methyl-[1,2,4]oxadiazol-3-yl)-naphthalen-1-yl]-amino}-acetic acid

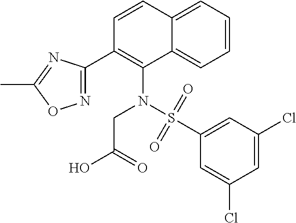

R_{f} value: 0.62 (silica gel, toluene/dioxane/ethanol/acetic acid=90:10:10:6)

Mass spectrum (ESI⁺): m/z=492, 494, 496 [M+H]⁺

### (76) {(3,5-dichloro-phenylsulphonyl)-[5-(2-methoxy-benzylcarbonylamino)-naphthalen-1-yl]-amino}-acetic acid

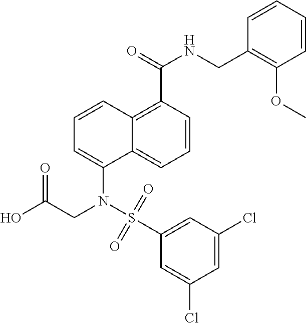

R_{f} value: 0.32 (silica gel, petroleum ether/ethyl acetate=1:1)

Mass spectrum (ESI⁺): m/z=573, 575, 577 [M+H]⁺

### (77) {(3,5-dichloro-phenylsulphonyl)-[5-(3-methoxy-benzylcarbonylamino)-naphthalen-1-yl]-amino}-acetic acid

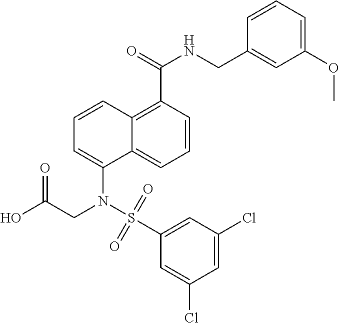

R_{f} value: 0.31 (silica gel, petroleum ether/ethyl acetate=1:1)

Mass spectrum (ESI⁺): m/z=573, 575, 577 [M+H]⁺

### (78) {(3,5-dichloro-phenylsulphonyl)-[5-(4-methoxy-benzylcarbonylamino)-naphthalen-1-yl]-amino}-acetic acid

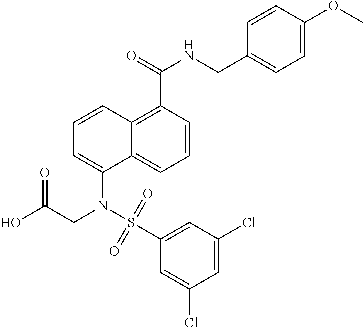

R_{f} value: 0.30 (silica gel, petroleum ether/ethyl acetate=1:1)

Mass spectrum (ESI⁺): m/z=573, 575, 577 [M+H]⁺

### (79) [[3-(phenylsulphonyl-methyl-amino)-naphthalen-1-yl]-(3,5-dichloro-phenylsulphonyl)-amino]-acetic acid

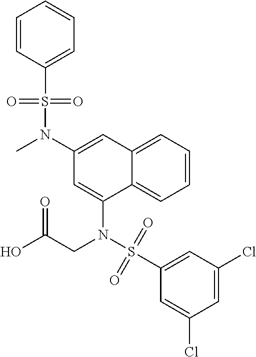

Mass spectrum (ESI⁻): m/z=577, 579, 581 [M−H]⁻

### (80) [(4-benzylaminocarbonyl-naphthalen-1-yl)-(3,5-dichloro-phenylsulphonyl)-amino]-acetic acid

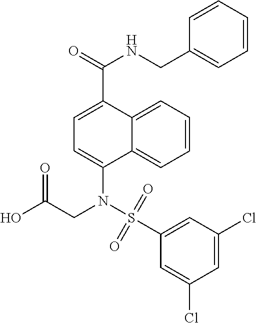

Mass spectrum (ESI⁺): m/z=543, 545, 547 [M+H]⁺

### (81) [[5-(3-aminocarbonyl-benzylaminocarbonyl)-naphthalen-1-yl]-(3,5-dichloro-phenylsulphonyl)-amino]-acetic acid

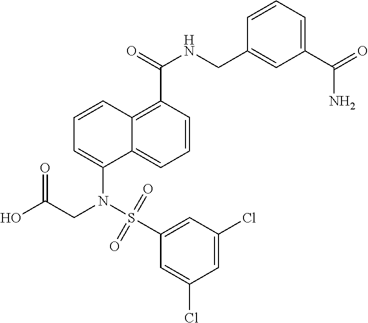

R_{f} value: 0.19 (silica gel, toluene/dioxane/ethanol/acetic acid=90:10:10:6)

Mass spectrum (ESI⁺): m/z=586, 588, 590 [M+H]⁺

### (82) ((3,5-dichloro-phensulphonyl)-{5-[(pyridin-4-ylmethyl)-aminocarbonyl]-naphthalen-2-yl}-amino)-acetic acid

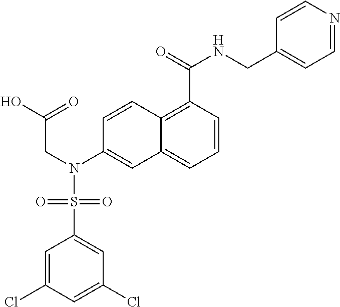

Mass spectrum (ESI⁺): m/z=544, 546, 548 [M+H]⁺

### (82) ((3,5-dichloro-phensulphonyl)-{5-[(pyridin-3-ylmethyl)-aminocarbonyl]-naphthalen-2-yl}-amino)-acetic acid

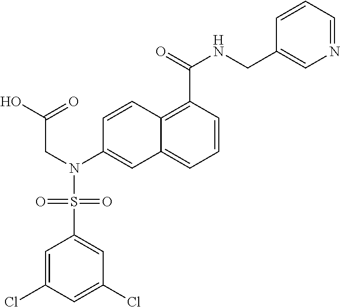

Mass spectrum (ESI⁺): m/z=544, 546, 548 [M+H]⁺

### (84) ((3,5-dichloro-phenylsulphonyl)-{5-[4-(morpholin-4-ylcarbonyl)-benzylaminocarbonyl]-naphthalen-1-yl}-amino)acetic acid

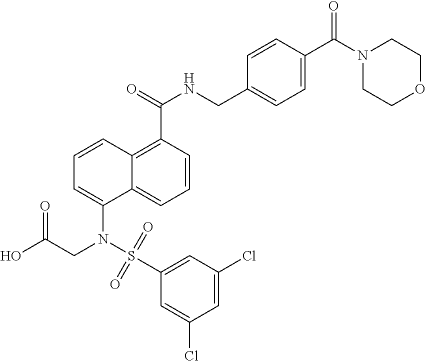

R_{f} value: 0.21 (silica gel, toluene/dioxane/ethanol/acetic acid=90:10:10:6)

Mass spectrum (ESI⁺): m/z=656, 658, 660 [M+H]⁺

### (85) [[5-(4-aminocarbonyl-benzylaminocarbonyl)-naphthalen-1-yl]-(3,5-dichloro-phenylsulphonyl)-amino]-acetic acid

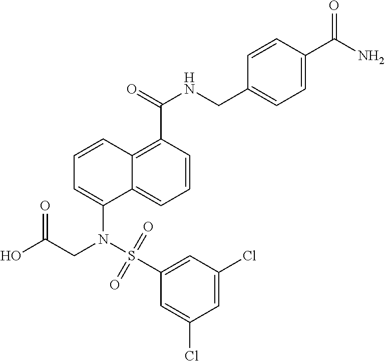

R_{f} value: 0.20 (silica gel, toluene/dioxane/ethanol/acetic acid=90:10:10:6)

Mass spectrum (ESI⁺): m/z=586, 588, 590 [M+H]⁺

### (86) ((3,5-dichloro-phenylsulphonyl)-{5-[4-(piperazin-1-ylcarbonyl)-benzylaminocarbonyl]-naphthalen-2-yl}-amino)-acetic acid

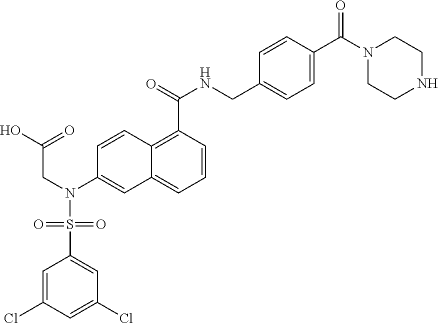

Mass spectrum (ESI⁺): m/z=655, 657, 659 [M+H]⁺

### (87) ((3,5-dichloro-phenylsulphonyl)-{5-[4-(morpholin-4-ylcarbonyl)-benzylaminocarbonyl]-naphthalen-2-yl}-amino)-acetic acid

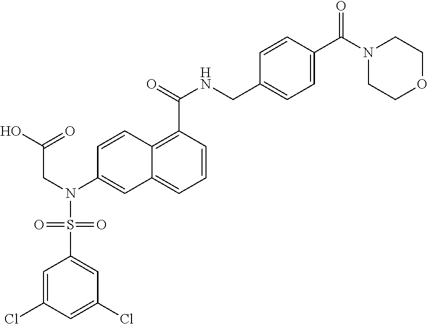

Mass spectrum (ESI⁺): m/z=656, 658, 660 [M+H]⁺

### (88) ((3,5-dichloro-phenylsulphonyl)-{5-[4-(piperazin-1-ylcarbonyl)-benzylaminocarbonyl]-naphthalen-1-yl}-amino)-acetic acid

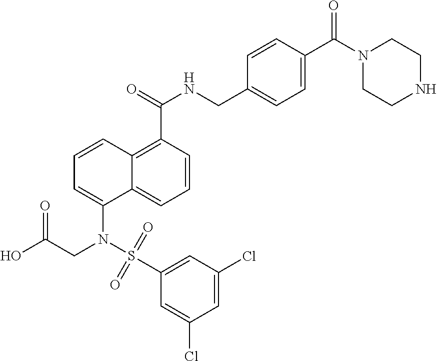

R_{f} value: 0.66 (Reversed phase ready-made TLC plate (E. Merck), acetonitrile/water/trifluoroacetic acid=60:40:1)

Mass spectrum (ESI⁺): m/z=655, 657, 659 [M+H]⁺

### (89) ((3,5-dichloro-phenylsulphonyl)-{5-[3-(morpholin-4-ylcarbonyl)-benzylaminocarbonyl]-naphthalen-1-yl}-amino)-acetic acid

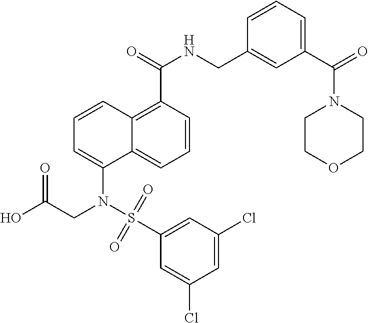

R_{f} value: 0.24 (silica gel, toluene/dioxane/ethanol/acetic acid=90:10:10:6)

Mass spectrum (ESI⁺): m/z=656, 658, 660 [M+H]⁺

### (90) ((3,5-dichloro-phenylsulphonyl)-{5-[3-(piperazin-1-ylcarbonyl)-benzylaminocarbonyl]-naphthalen-1-yl}-amino)-acetic acid

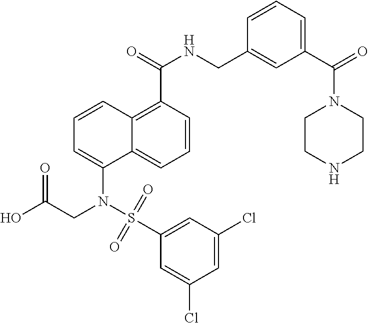

R_{f} value: 0.66 (Reversed phase ready-made TLC plate (E. Merck), acetonitrile/water/trifluoroacetic acid=60:40:1)

Mass spectrum (ESI⁺): m/z=655, 657, 659 [M+H]⁺

### (91) ((3,5-dichloro-phenylsulphonyl)-{5-[2-(piperazin-1-ylcarbonyl)-benzylaminocarbonyl]-naphthalen-2-yl}-amino)-acetic acid

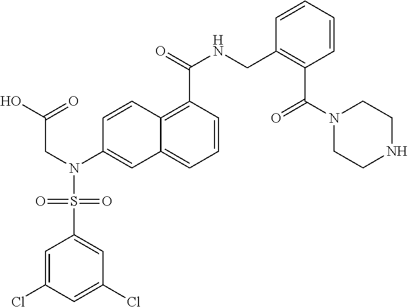

Mass spectrum (ESI⁺): m/z=655, 657, 659 [M+H]⁺

### (92) ((3,5-dichloro-phenylsulphonyl)-{5-[2-(morpholin-4-ylcarbonyl)-benzylaminocarbonyl]-naphthalen-1-yl}-amino)-acetic acid

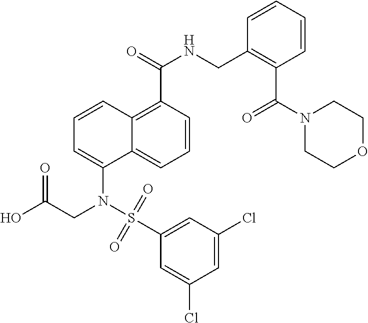

R_{f} value: 0.55 (Reversed phase ready-made TLC plate (E. Merck), acetonitrile/water/trifluoroacetic acid=60:40:1)

Mass spectrum (ESI⁺): m/z=656, 658, 660 [M+H]⁺

### (93) ((3,5-dichloro-phenylsulphonyl)-{5-[2-(piperazin-1-ylcarbonyl)-benzylaminocarbonyl]-naphthalen-1-yl}-amino)-acetic acid

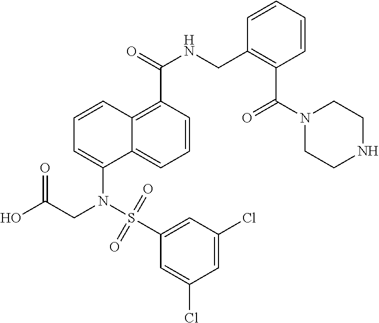

R_{f} value: 0.70 (Reversed phase ready-made TLC plate (E. Merck), acetonitrile/water/trifluoroacetic acid=60:40:1)

Mass spectrum (ESI⁺): m/z=655, 657, 659 [M+H]⁺

### (94) {(3,5-dichloro-phenylsulphonyl)-[5-(3-dimethylaminocarbonyl-benzylaminocarbonyl)-naphthalen-1-yl]-amino}-acetic acid

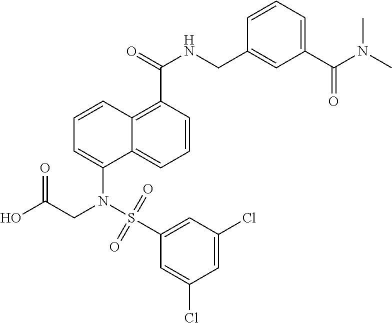

R_{f} value: 0.40 (silica gel, toluene/dioxane/ethanol/acetic acid=90:10:10:6)

Mass spectrum (ESI⁺): m/z=614, 616, 618 [M+H]⁺

### (95) {(3,5-dichloro-phenylsulphonyl)-[5-(4-dimethylaminocarbonyl-benzylaminocarbonyl)-naphthalen-1-yl]-amino}-acetic acid

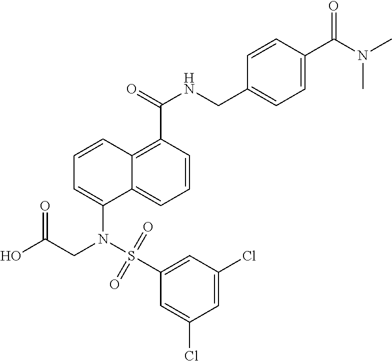

R_{f} value: 0.45 (silica gel, toluene/dioxane/ethanol/acetic acid=90:10:10:6)

Mass spectrum (ESI⁺): m/z=614, 616, 618 [M+H]⁺

### (96) {[5-(2-aminocarbonyl-benzylaminocarbonyl)-naphthalen-1-yl]-(3,5-dichloro-phenylsulphonyl)-amino}-acetic acid

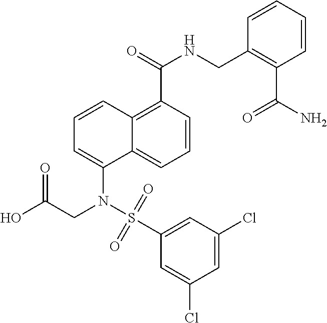

R_{f} value: 0.31 (silica gel, toluene/dioxane/ethanol/acetic acid=90:10:10:6)

Mass spectrum (ESI⁺): m/z=586, 588, 590 [M+H]⁺

### (97) {(3,5-dichloro-phenylsulphonyl)-[5-(4-methylaminocarbonyl-benzylaminocarbonyl)-naphthalen-1-yl]-amino}-acetic acid

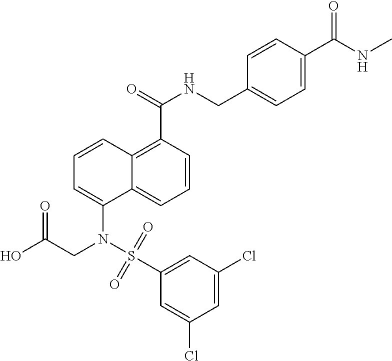

R_{f} value: 0.17 (silica gel, toluene/dioxane/ethanol/acetic acid=90:10:10:6)

Mass spectrum (ESI⁺): m/z=600, 602, 604 [M+H]⁺

### (98) {(3,5-dichloro-phenylsulphonyl)-[5-(3-methylaminocarbonyl-benzylaminocarbonyl)-naphthalen-1-yl]-amino}-acetic acid

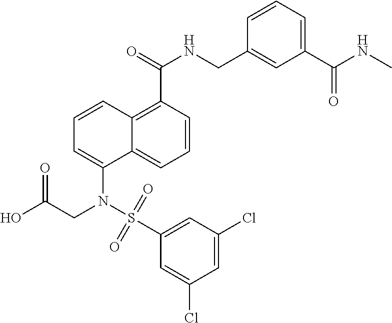

R_{f} value: 0.30 (silica gel, toluene/dioxane/ethanol/acetic acid=90:10:10:6)

Mass spectrum (ESI⁺): m/z=600, 602, 604 [M+H]⁺

### (99) {(3,5-dichloro-phenylsulphonyl)-[5-(4-dimethylaminocarbonyl-benzylaminocarbonyl)-naphthalen-2-yl]-amino}-acetic acid

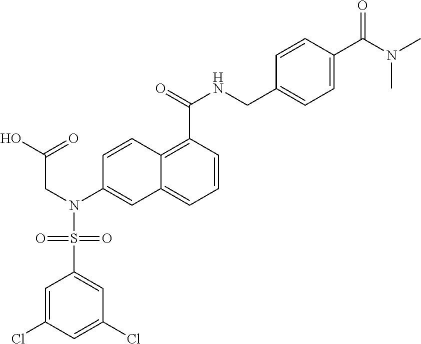

R_{f} value: 0.16 (silica gel, methylene chloride/methanol=95:5)

Mass spectrum (ESI⁺): m/z=614, 616, 618 [M+H]⁺

### (100) {(3,5-dichloro-phenylsulphonyl)-[5-(3-dimethylaminocarbonyl-benzylaminocarbonyl)-naphthalen-2-yl]-amino}-acetic acid

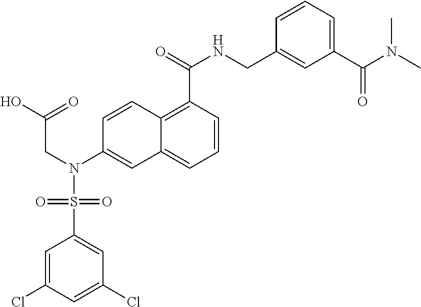

R_{f} value: 0.20 (silica gel, methylene chloride/methanol=95:5)

Mass spectrum (ESI⁺): m/z=614, 616, 618 [M+H]⁺

### (101) [[3-(N-benzyl-N-methyl-aminocarbonyl)-naphthalen-1-yl]-(3,5-dichloro-phenylsulphonyl)-amino]-acetic acid

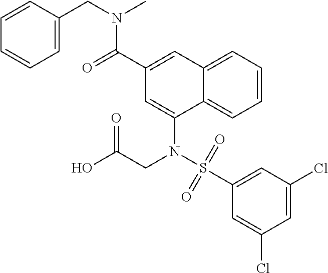

R_{f} value: 0.40 (silica gel, cyclohexane/ethyl acetate=1:1)

R_{f} value: 0.20 (silica gel, methylene chloride/methanol=95:5)

Mass spectrum (ESI⁺): m/z=574, 576, 578 [M+NH₄]⁺

### (102) [(3,5-dichloro-phenylsulphonyl)-[3-(N-methyl-N-phenyl-aminocarbonyl)-naphthalen-1-yl]-acetic acid

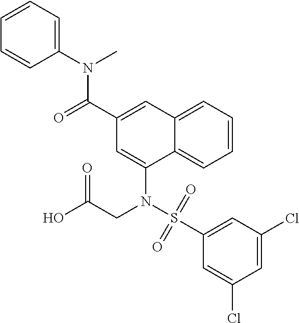

R_{f} value: 0.13 (silica gel, cyclohexane/ethyl acetate=1:1)

Mass spectrum (ESI⁺): m/z=543, 545, 547 [M+H]⁺

### (103) [(3,5-dichloro-phenylsulphonyl)-(5-{[6-(2-dimethylamino-ethylamino)-pyridin-3-ylmethyl]-aminocarbonyl}-naphthalen-1-yl)-amino]-acetic acid

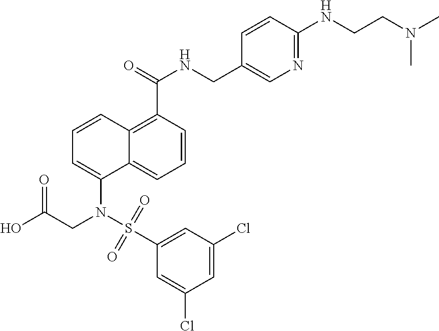

R_{f} value: 0.70 (Reversed phase ready-made TLC plate (E. Merck), acetonitrile/water/trifluoroacetic acid=60:40:1)

Mass spectrum (ESI⁺): m/z=630, 632, 634 [M+H]⁺

### (104) [(3,5-dichloro-phenylsulphonyl)-(5-{[2-(2-dimethylamino-ethylamino)-pyridin-4-ylmethyl]-aminocarbonyl}-naphthalen-1-yl)amino]-acetic acid

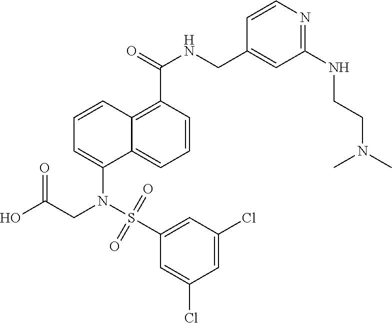

R_{f} value: 0.65 (Reversed phase ready-made TLC plate (E. Merck), acetonitrile/water/trifluoroacetic acid=60:40:1)

Mass spectrum (ESI⁺): m/z=630, 632, 634 [M+H]⁺

### (105) {(3,5-dichloro-phenylsulphonyl)-[5-({2-N-[(2-dimethylamino-ethyl)-N-methyl-amino]-pyridin-4-ylmethyl}-aminocarbonyl)-naphthalen-1-yl]-amino}-acetic acid

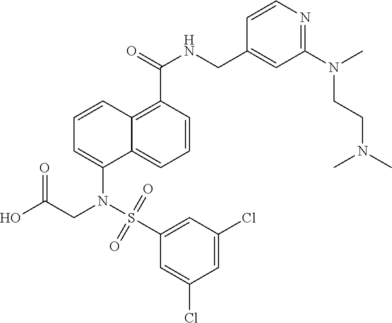

R_{f} value: 0.65 (Reversed phase ready-made TLC plate (E. Merck), acetonitrile/water/trifluoroacetic acid=60:40:1)

Mass spectrum (ESI⁺): m/z=644, 646, 648 [M+H]⁺

### (106) {(3,5-dichloro-phenylsulphonyl)-[5-({6-N-[(2-dimethylamino-ethyl)-N-methyl-amino]-pyridin-3-ylmethyl}-aminocarbonyl)-naphthalen-1-yl]-amino}-acetic acid

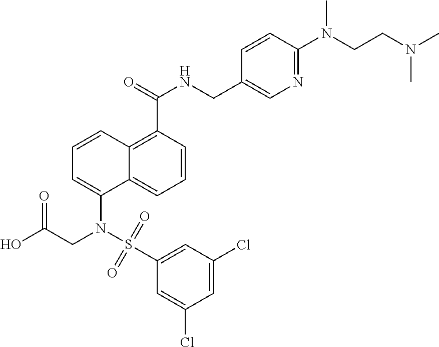

R_{f} value: 0.70 (Reversed phase ready-made TLC plate (E. Merck), acetonitrile/water/trifluoroacetic acid=60:40:1)

Mass spectrum (ESI⁺): m/z=644, 646, 648 [M+H]⁺

### (107) [(3,5-dichloro-phenylsulphonyl)-(5-{[6-(2-dimethylamino-ethylamino)-pyridin-3-ylmethyl]-aminocarbonyl}-naphthalen-2-yl)amino]-acetic acid

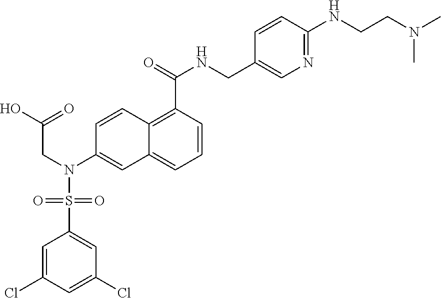

R_{f} value: 0.65 (Reversed phase ready-made TLC plate (E. Merck), acetonitrile/water/trifluoroacetic acid=60:40:1)

Mass spectrum (ESI⁺): m/z=630, 632, 634 [M+H]⁺

### (108) {(3,5-dichloro-phenylsulphonyl)-[5-({2-N-[(2-dimethylamino-ethyl)-N-methyl-amino]-pyridin-4-ylmethyl}-aminocarbonyl)-naphthalen-2-yl]-amino}-acetic acid

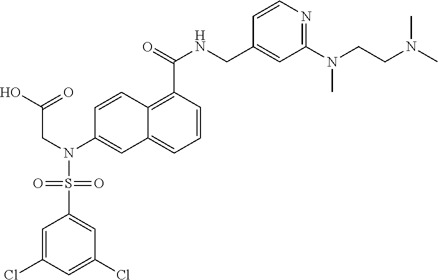

R_{f} value: 0.70 (Reversed phase ready-made TLC plate (E. Merck), acetonitrile/water/trifluoroacetic acid=60:40:1)

Mass spectrum (ESI⁺): m/z=644, 646, 648 [M+H]⁺

### (109) {(3,5-dichloro-phenylsulphonyl)-[5-({6-N-[(2-dimethylamino-ethyl)-N-methyl-amino]-pyridin-3-ylmethyl}-aminocarbonyl)-naphthalen-2-yl]-amino}-acetic acid

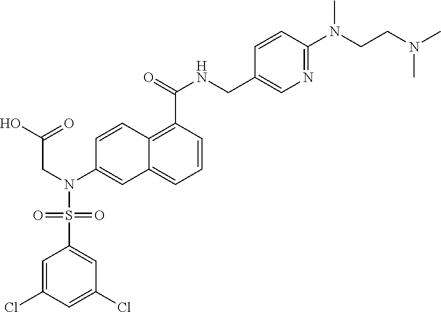

R_{f} value: 0.65 (Reversed phase ready-made TLC plate (E. Merck), acetonitrile/water/trifluoroacetic acid=60:40:1)

Mass spectrum (ESI⁺): m/z=644, 646, 648 [M+H]⁺

### (110) [(3,5-dichloro-phenylsulphonyl)-(5-{[2-(2-dimethylamino-ethylamino)-pyridin-4-ylmethyl]-carbamoyl}-naphthalen-2-yl)amino]-acetic acid

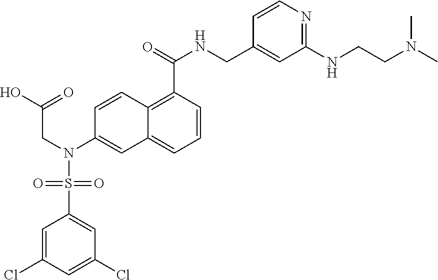

R_{f} value: 0.65 (Reversed phase ready-made TLC plate (E. Merck), acetonitrile/water/trifluoroacetic acid=60:40:1)

Mass spectrum (ESI⁺): m/z=630, 632, 634 [M+H]⁺

### (111) {(3,5-dichloro-phenylsulphonyl)-[6-(4-piperazin-1-yl-pyrimidin-2-yl)-naphthalen-2-yl]-amino}-acetic acid

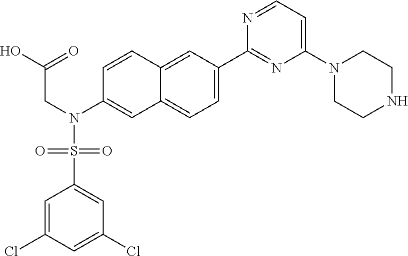

R_{f} value: 0.65 (Reversed phase ready-made TLC plate (E. Merck), acetonitrile/water/trifluoroacetic acid=60:40:1)

Mass spectrum (ESI⁺): m/z=572, 574, 576 [M+H]⁺

### (112) {(3,5-dichloro-phenylsulphonyl)-[5-(4-piperazin-1-yl-pyrimidin-2-yl)-naphthalen-1-yl]-amino}-acetic acid

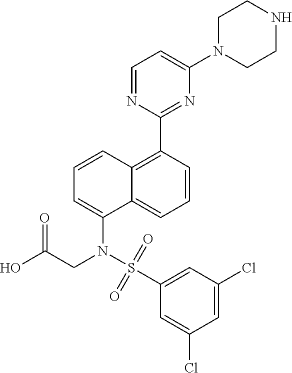

R_{f} value: 0.73 (Reversed phase ready-made TLC plate (E. Merck), acetonitrile/water/trifluoroacetic acid=60:40:1)

Mass spectrum (ESI⁺): m/z=572, 574, 576 [M+H]⁺

### (113) {(3,5-dichloro-phenylsulphonyl)-[6-(2-morpholin-4-yl-pyrimidin-4-yl)-naphthalen-2-yl]-amino}-acetic acid

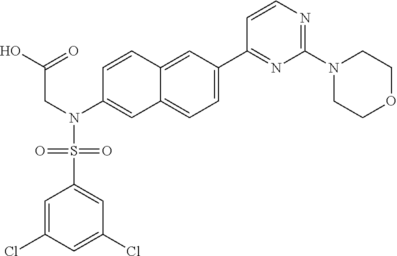

R_{f} value: 0.45 (Reversed phase ready-made TLC plate (E. Merck), acetonitrile/water/trifluoroacetic acid=60:40:1)

Mass spectrum (ESI⁻): m/z=571, 573, 575 [M−H]⁻

### (114) [(3,5-dichloro-phenylsulphonyl)-(6-{4-[N-(2-dimethylamino-ethyl)-N-methyl-amino]-pyrimidin-2-yl}-naphthalen-2-yl)-amino]-acetic acid

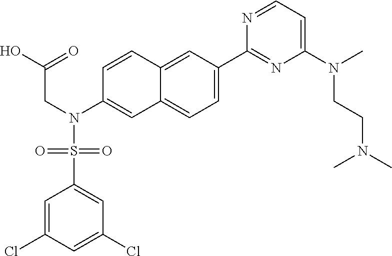

R_{f} value: 0.68 (Reversed phase ready-made TLC plate (E. Merck), acetonitrile/water/trifluoroacetic acid=60:40:1)

Mass spectrum (ESI⁺): m/z=588, 590, 592 [M+H]⁺

### (115) {(3,5-dichloro-phenylsulphonyl)-[5-(4-morpholin-4-yl-pyrimidin-2-yl)-naphthalen-1-yl]-amino}-acetic acid

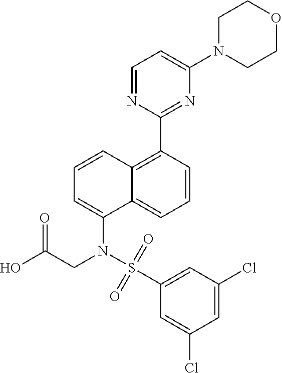

R_{f} value: 0.15 (silica gel, methylene chloride/methanol=95:5)

Mass spectrum (ESI⁺): m/z=573, 575, 577 [M+H]⁺

### (116) ((3,5-dichloro-phenylsulphonyl)-{6-[4-(2-dimethylamino-ethylamino)-pyrimidin-2-yl]-naphthalen-2-yl}-amino)-acetic acid

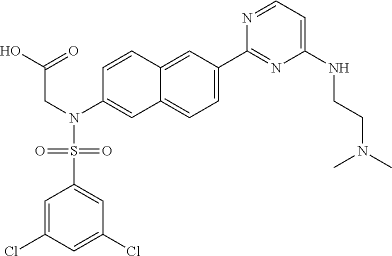

R_{f} value: 0.67 (Reversed phase ready-made TLC plate (E. Merck), acetonitrile/water/trifluoroacetic acid=60:40:1)

Mass spectrum (ESI⁺): m/z=574, 576, 578 [M+H]⁺

### (117) {(3,5-dichloro-phenylsulphonyl)-[5-(2-morpholin-4-yl-pyrimidin-4-yl)-naphthalen-1-yl]-amino}-acetic acid

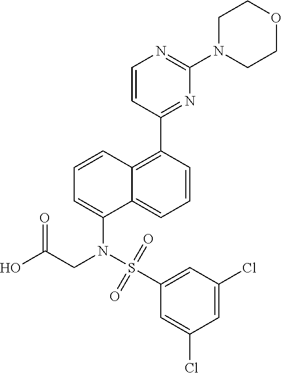

R_{f} value: 0.33 (silica gel, methylene chloride/methanol=95:5)

Mass spectrum (ESI⁺): m/z=573, 575, 577 [M+H]⁺

### (118) {(3,5-dichloro-phenylsulphonyl)-[6-(6-morpholin-4-yl-pyridazin-3-yl)-naphthalen-2-yl]-amino}-acetic acid

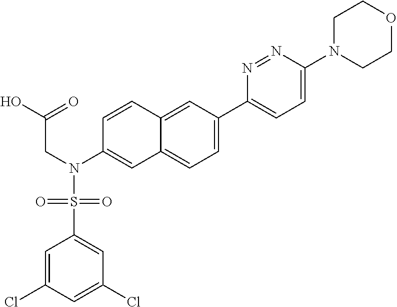

R_{f} value: 0.30 (silica gel, methylene chloride/methanol=95:5)

Mass spectrum (ESI⁺): m/z=573, 575, 577 [M+H]⁺

### (119) {(3,5-dichloro-phenylsulphonyl)-[5-(2-morpholin-4-yl-pyrimidin-5-yl)-naphthalen-1-yl]-amino}-acetic acid

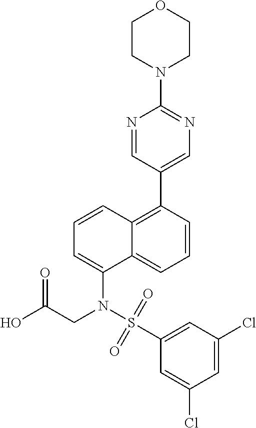

R_{f} value: 0.35 (silica gel, methylene chloride/methanol=95:5)

Mass spectrum (ESI⁺): m/z=573, 575, 577 [M+H]⁺

### (120) ((3,5-dichloro-phenylsulphonyl)-{6-[5-(2-dimethylamino-ethylamino)-pyrazin-2-yl]-naphthalen-2-yl}-amino)-acetic acid

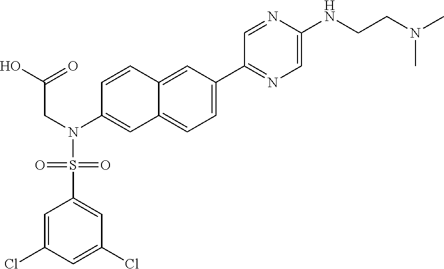

R_{f} value: 0.58 (Reversed phase ready-made TLC plate (E. Merck), acetonitrile/water/trifluoroacetic acid=60:40:1)

Mass spectrum (ESI⁺): m/z=574, 576, 578 [M+H]⁺

### (121) ((3,5-dichloro-phenylsulphonyl)-{6-[6-(2-dimethylamino-ethylamino)-pyridazin-3-yl]-naphthalen-2-yl}-amino)-acetic acid

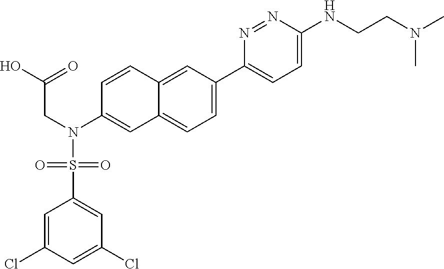

R_{f} value: 0.65 (Reversed phase ready-made TLC plate (E. Merck), acetonitrile/water/trifluoroacetic acid=60:40:1)

Mass spectrum (ESI⁺): m/z=574, 576, 578 [M+H]⁺

### (122) {(3,5-dichloro-phenylsulphonyl)-[6-(4-morpholin-4-yl-pyrimidin-2-yl)-naphthalen-2-yl]-amino}acetic acid

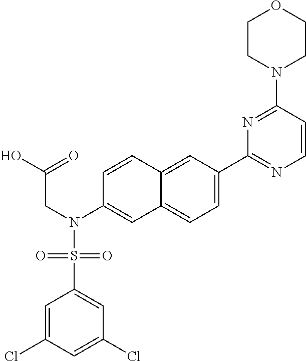

R_{f} value: 0.45 (Reversed phase ready-made TLC plate (E. Merck), acetonitrile/water/trifluoroacetic acid=60:40:1)

Mass spectrum (ESI⁺): m/z=573, 575, 577 [M+H]⁺

### (123) {(3,5-dichloro-phenylsulphonyl)-[6-(5-morpholin-4-yl-pyrazin-2-yl)-naphthalen-2-yl]-amino}-acetic acid

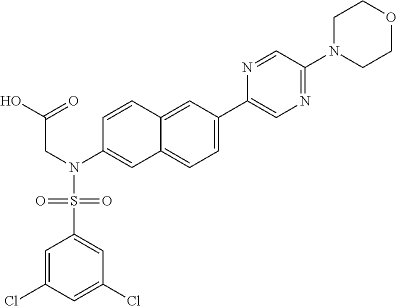

R_{f} value: 0.36 (silica gel, methylene chloride/methanol=95:5)

Mass spectrum (ESI⁺): m/z=573, 575, 577 [M+H]⁺

### (124) [(3,5-dichloro-phenylsulphonyl)-quinolin-8-yl-amino]-acetic acid

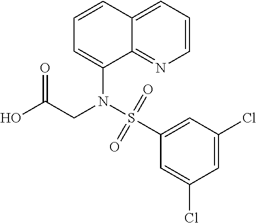

R_{f} value: 0.50 (silica gel, methylene chloride/methanol=15:1)

Mass spectrum (ESI⁺): m/z=411, 413, 415 [M+H]⁺

### (125) [(3,5-dichloro-phenylsulphonyl)-(6-methoxy-quinolin-8-yl)-amino]-acetic acid

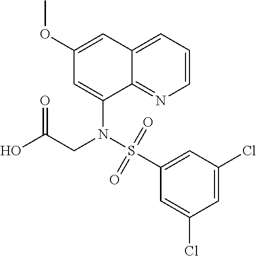

R_{f} value: 0.60 (Reversed phase ready-made TLC plate (E. Merck), acetonitrile/water/trifluoroacetic acid=60:40:1)

Mass spectrum (ESI⁺): m/z=441, 443, 445 [M+H]⁺

### Example 4

Coated tablets containing 75 mg of active substance

1 tablet core contains:

| | |
|---|---|
| active substance | 75.0 mg |
| calcium phosphate | 93.0 mg |
| corn starch | 35.5 mg |
| polyvinylpyrrolidone | 10.0 mg |
| hydroxypropylmethylcellulose | 15.0 mg |
| magnesium stearate | 1.5 mg |
| | 230.0 mg |

### Preparation:

The active substance is mixed with calcium phosphate, corn starch, polyvinylpyrrolidone, hydroxypropylmethylcellulose and half the specified amount of magnesium stearate. Blanks 13 mm in diameter are produced in a tablet-making machine and these are then rubbed through a screen with a mesh size of 1.5 mm using a suitable machine and mixed with the rest of the magnesium stearate. This granulate is compressed in a tablet-making machine to form tablets of the desired shape.
Weight of core: 230 mg
die: 9 mm, convex

The tablet cores thus produced are coated with a film consisting essentially of hydroxypropylmethylcellulose. The finished film-coated tablets are polished with beeswax.
Weight of coated tablet: 245 mg.

### Example 5

Tablets Containing 100 mg of active substance

### Composition:

1 tablet contains:

| | |
|---|---|
| active substance | 100.0 mg |
| lactose | 80.0 mg |
| corn starch | 34.0 mg |
| polyvinylpyrrolidone | 4.0 mg |
| magnesium stearate | 2.0 mg |
| | 220.0 mg |

### Method of Preparation:

The active substance, lactose and starch are mixed together and uniformly moistened with an aqueous solution of the polyvinylpyrrolidone. After the moist composition has been screened (2.0 mm mesh size) and dried in a rack-type drier at 50° C. it is screened again (1.5 mm mesh size) and the lubricant is added. The finished mixture is compressed to form tablets.
Weight of tablet: 220 mg
Diameter: 10 mm, biplanar, facetted on both sides and notched on one side.

### Example 6

Tablets containing 150 mg of active substance

### Composition:

1 tablet contains:

| | |
|---|---|
| active substance | 50.0 mg |
| powdered lactose | 89.0 mg |
| corn starch | 40.0 mg |
| colloidal silica | 10.0 mg |
| polyvinylpyrrolidone | 10.0 mg |
| magnesium stearate | 1.0 mg |
| | 300.0 mg |

### Preparation:

The active substance mixed with lactose, corn starch and silica is moistened with a 20% aqueous polyvinylpyrrolidone solution and passed through a screen with a mesh size of 1.5 mm. The granules, dried at 45° C., are passed through the same screen again and mixed with the specified amount of magnesium stearate. Tablets are pressed from the mixture.
Weight of tablet: 300 mg
die: 10 mm, flat

### Example 7

Hard gelatine capsules containing 150 mg of active substance

1 capsule contains:

| | | |
|---|---|---|
| active substance | 150.0 | mg |
| corn starch (dried | approx. 180.0 | mg |
| lactose (powdered) | approx. 87.0 | mg |
| magnesium stearate | 3.0 | mg |
| | approx. 420.0 | mg |

### Preparation:

The active substance is mixed with the excipients, passed through a screen with a mesh size of 0.75 mm and homogeneously mixed using a suitable apparatus. The finished mixture is packed into size 1 hard gelatine capsules.
Capsule filling: approx. 320 mg
Capsule shell: size 1 hard gelatine capsule.

### Example 8

Suppositories containing 150 mg of active substance

1 suppository contains:

| | |
|---|---|
| active substance | 150.0 mg |
| polyethyleneglycol 1500 | 550.0 mg |
| polyethyleneglycol 6000 | 460.0 mg |
| polyoxyethylene sorbitan monostearate | 840.0 mg |
| | 2,000.0 mg |

### Preparation:

After the suppository mass has been melted the active substance is homogeneously distributed therein and the melt is poured into chilled moulds.

### Example 9

Suspension containing 50 mg of active substance

100 ml of suspension contain:

| | | |
|---|---|---|
| active substance | 1.00 | g |
| carboxymethylcellulose-Na-salt | 0.10 | g |
| methyl p-hydroxybenzoate | 0.05 | g |
| propyl p-hydroxybenzoate | 0.01 | g |
| glucose | 10.00 | g |
| glycerol | 5.00 | g |
| 70% sorbitol solution | 20.00 | g |
| flavouring | 0.30 | g |
| dist. Water | ad 100 | ml |

### Preparation:

The distilled water is heated to 70° C. The methyl and propyl p-hydroxybenzoates together with the glycerol and sodium salt of carboxymethylcellulose are dissolved therein with stirring. The solution is cooled to ambient temperature and the active substance is added and homogeneously dispersed therein with stirring. After the sugar, the sorbitol solution and the flavouring have been added and dissolved, the suspension is evacuated with stirring to eliminate air.

5 ml of suspension contain 50 mg of active substance.

### Example 10

Ampoules containing 10 mg active substance

### Composition:

| active substance | 10.0 | mg |
|---|---|---|
| 0.01N hydrochloric acid | q.s. | |
| double-distilled water | ad 2.0 | ml |

### Preparation:

The active substance is dissolved in the necessary amount of 0.01 N HCl, made isotonic with common salt, filtered sterile and transferred into 2 ml ampoules.

### Example 11

Ampoules containing 50 mg of active substance

### Composition:

| active substance | 50.0 | mg |
|---|---|---|
| 0.01N hydrochloric acid | q.s. | |
| double-distilled water | ad 10.0 | ml |

### Preparation:

The active substance is dissolved in the necessary amount of 0.01 N HCl, made isotonic with common salt, filtered sterile and transferred into 10 ml ampoules.

1. A compound of general formula 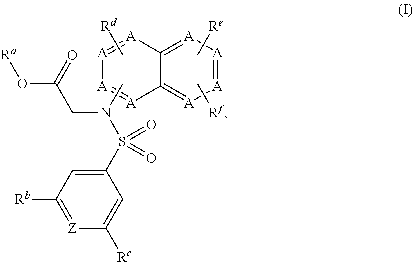
wherein
R^{a} denotes H, a group of formula 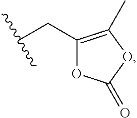
or a C₁₋₆-alkyl group, which may be substituted by
C₁₋₆-alkyl-carbonyloxy, C₁₋₆-alkoxy-carbonyloxy, C₁₋₆-alkoxy, hydroxy,
amino, aminocarbonyl or amino-C₂₋₃-alkyloxy, wherein in each case one or two of the hydrogen atoms present on the nitrogen may be replaced independently of one another by a C₁₋₃-alkyl group,
heterocycloalkyl, heterocycloalkylcarbonyl, heterocycloalkyloxy or heterocycloalkyl-C₁₋₃-alkyloxy,
R^{b} and R^{c} each independently of one another denotes H, halogen, C₁₋₃-alkyl, C₂₋₃-alkenyl, C₂₋₃-alkynyl, C₁₋₃-perfluoroalkyl, C₁₋₃-alkoxy, C₁₋₃-perfluoroalkoxy, while in each case only one of the groups R^{b} and R^{c} may represent H,
A denotes CH or N, while a total of not more than four nitrogen atoms may be present in the bicyclic system,
Z denotes CH, CF or N,
R^{d} and R^{e} independently of one another denote H, halogen, cyano, hydroxy, nitro, C₁₋₆-alkyl, C₂₋₆-alkenyl, C₂₋₆-alkynyl, C₁₋₆-fluoroalkyl, C₁₋₆-perfluoroalkyl, C₃₋₇-cycloalkyl, heterocycloalkyl, aryl, heteroaryl, C₁₋₆-alkoxy, C₁₋₆-fluoroalkoxy, C₁₋₆-perfluoroalkoxy, C₃₋₇-cycloalkyloxy, heterocycloalkyloxy, aryloxy, heteroaryloxy, C₁₋₆-alkylsulphanyl, C₃₋₇-cycloalkylsulphanyl
or a group selected from among R¹R²N, R¹R²N—CO, R¹R²N—CO—NR³, R¹R²N—SO, R¹R²N—SO₂, R¹R²N—SO₂—NR³, R⁴—CO, R⁴—CO—NR³, R⁵—SO, R⁵—SO—NR³, R⁵—SO₂, R⁵—SO₂—NR³— and R⁵—CO—O—, wherein
R¹ denotes H, C₁₋₆-alkyl, C₃₋₇-cycloalkyl, heterocycloalkyl, aryl or heteroaryl,
R² denotes H, C₁₋₆-alkyl, C₃₋₇-cycloalkyl, heterocycloalkyl, aryl or heteroaryl,
R³ denotes H, C₁₋₆-alkyl or C₃₋₇-cycloalkyl,
R⁴ denotes C₁₋₆-alkyl, C₃₋₇-cycloalkyl, heterocycloalkyl, aryl, heteroaryl, hydroxy, or C₁₋₆-alkyloxy and
R⁵ denotes C₁₋₆-alkyl, C₃₋₇-cycloalkyl, heterocycloalkyl, aryl or heteroaryl,
and
R^{f} denotes H, halogen, C₁₋₃-alkyl, C₂₋₃-alkenyl, C₂₋₃-alkynyl, C₁₋₃-perfluoroalkyl, C₁₋₃-alkoxy, C₁₋₃-perfluoroalkoxy or cyano,
while the groups contained in the C₁₋₆-alkyl, C₂₋₆-alkenyl, C₂₋₆-alkynyl, C₃₋₇-cycloalkyl, C₁₋₆-alkyloxy and C₃₋₇-cycloalkyloxy groups mentioned hereinbefore for R^{d}, R^{e}, R^{f} as well as R¹ to R⁵ may each be di- or trisubstituted independently of one another in the carbon skeleton by a group selected from among
cyano, hydroxy, C₃₋₇-cycloalkyl, heterocycloalkyl, aryl, heteroaryl, C₁₋₆-alkoxy, C₁₋₆-perfluoroalkoxy, C₃₋₇-cycloalkyloxy, heterocycloalkyloxy, aryloxy, heteroaryloxy
and a group selected from among R⁶R⁷N, R⁶R⁷N—CO, R⁶R⁷N—CO—NR⁸, R⁶R⁷N—SO₂—NR⁸, R⁹—CO, R⁹—CO—NR⁸, R¹⁰—SO₂, R¹⁰—SO₂—NR⁸— and R¹⁰—CO—O, wherein
R⁶ denotes H, C₁₋₄-alkyl, C₃₋₆-cycloalkyl, C₃₋₆-cycloalkyl-C₁₋₄-alkyl, heterocycloalkyl, heterocycloalkyl-C₁₋₄-alkyl, aryl, aryl-C₁₋₄-alkyl, heteroaryl or heteroaryl-C₁₋₄-alkyl,
R⁷ denotes H, C₁₋₄-alkyl, C₃₋₆-cycloalkyl, C₃₋₆-cycloalkyl-C₁₋₄-alkyl, heterocycloalkyl, heterocycloalkyl-C₁₋₄-alkyl, aryl, aryl-C₁₋₄-alkyl, heteroaryl or heteroaryl-C₁₋₄-alkyl,
R⁸ denotes H, C₁₋₄-alkyl, C₃₋₆-cycloalkyl or C₃₋₆-cycloalkyl-C₁₋₄-alkyl,
R⁹ denotes C₁₋₄-alkyl, C₃₋₆-cycloalkyl, C₃₋₆-cycloalkyl-C₁₋₄-alkyl, heterocycloalkyl, heterocycloalkyl-C₁₋₄-alkyl, aryl, aryl-C₁₋₄-alkyl, heteroaryl, heteroaryl-C₁₋₄-alkyl, hydroxy or C₁₋₄-alkyloxy and
R¹⁰ denotes C₁₋₄-alkyl, C₃₋₆-cycloalkyl, C₃₋₆-cycloalkyl-C₁₋₄-alkyl, heterocycloalkyl, heterocycloalkyl-C₁₋₄-alkyl, aryl, aryl-C₁₋₄-alkyl, heteroaryl or heteroaryl-C₁₋₄-alkyl,
while the above-mentioned substituents must not be bound to a common carbon atom and heteroatoms must be separated from one another by at least two carbon atoms,
and the aryl, heteroaryl, aryloxy and heteroaryloxy groups contained in the groups mentioned hereinbefore for R^{d}, R^{e} as well as R¹ to R⁵ may each be di- or trisubstituted independently of one another in the carbon skeleton by a group selected from among
halogen, cyano, hydroxy, nitro, C₁₋₆-alkyl, C₂₋₆-alkenyl, C₂₋₆-alkynyl, C₃₋₇-cycloalkyl, C₃₋₇-cycloalkyl-C₁₋₄-alkyl, C₁₋₆-perchloroalkyl, C₁₋₆-fluoroalkyl, C₁₋₆-perfluoroalkyl, C₁₋₆-alkoxy, C₁₋₆-fluoroalkoxy, C₁₋₆-perfluoroalkoxy, C₃₋₇-cycloalkyloxy, C₃₋₇-cycloalkyl-C₁₋₄-alkyloxy, heterocycloalkyloxy, heterocycloalkyl-C₁₋₄-alkyloxy C₁₋₆-alkylsulphanyl, C₃₋₇-cycloalkylsulphanyl,
and a group selected from among R⁶R⁷N, R⁶R⁷N—CO, R⁶R⁷N—CO—NR⁸, R⁶R⁷N—SO, R⁶R⁷N—SO₂, R⁶R⁷N—SO₂—NR⁸, R⁹—CO, R⁹—CO—NR⁸, R¹⁰—SO, R¹⁰—SO—NR⁸, R¹⁰—SO₂, R¹⁰—SO₂—NR⁸— and R¹⁰—CO—O, while R⁶ to R¹⁰ are as hereinbefore defined
as well as the physiologically acceptable salts thereof.

2. A compound of general formula (I) according to claim 1, wherein the bicyclic heteroaromatic group 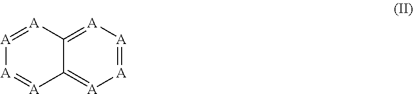
denotes naphthalene, quinoline, isoquinoline, quinazoline, quinoxaline, cinnoline, phthalazine, [1,5]naphthyridine, [1,8]naphthyridine, pyrido[3,2-d]pyrimidine, pyrimido[5,4-d]pyrimidine, or pteridine, and
R^{a} to R^{f}, R¹ to R¹⁰, A and Z are defined as in claim 1, with the proviso that at least one of the groups R^{d} and R^{e} denotes H, halogen or C₁₋₃-alkyl,
as well as the physiologically acceptable salts thereof.

3. A compound of general formula (I) according to claim 2, wherein the bicyclic heteroaromatic group 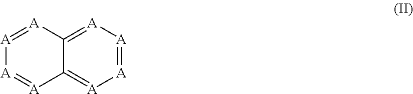
denotes naphthalene, quinoline, quinazoline, quinoxaline or cinnoline,
R^{a} denotes H, a group of formula 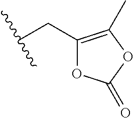
or a C₁₋₄-alkyl group, which may be substituted by C₁₋₄-alkoxy, hydroxy, di-(C₁₋₃-alkyl)-amino, pyrrolidin-1-yl, piperidin-1-yl, morpholin-4-yl, piperazin-1-yl or 4-methyl-piperazin-1-yl,
R^{b} and R^{c} independently of one another denote chlorine, bromine or C₁₋₂-alkyl,
Z denotes CH or N,
R^{d} denotes H, halogen, cyano, hydroxy, nitro, C₁₋₄-alkyl, C₂₋₄-alkenyl, C₂₋₄-alkynyl, aryl-C₂₋₃-alkynyl, C₁₋₄-fluoroalkyl, C₁₋₄-perfluoroalkyl, C₃₋₆-cycloalkyl, C₃₋₆-cycloalkyl-C₁₋₄-alkyl, heterocycloalkyl, heterocycloalkyl-C₁₋₄-alkyl, aryl, aryl-C₁₋₄-alkyl, heteroaryl, heteroaryl-C₁₋₄-alkyl, C₁₋₄-alkoxy, C₁₋₄-fluoroalkoxy, C₁₋₄-perfluoroalkoxy, C₃₋₆-cycloalkyloxy, C₃₋₆-cycloalkyl-C₁₋₄-alkyloxy, heterocycloalkyloxy, heterocycloalkyl-C₁₋₄-alkoxy, aryloxy, aryl-C₁₋₄-alkyloxy, heteroaryloxy, heteroaryl-C₁₋₄-alkyloxy, C₁₋₄-alkylsulphanyl or C₃₋₆-cycloalkylsulphanyl,
while the aryl and heteroaryl groups contained in the groups mentioned hereinbefore for R^{d} may optionally be substituted by halogen, C₁₋₃-alkyl, tri-chloromethyl, phenyl, phenyl-C₁₋₃-alkyl, hydroxy, C₁₋₃-alkoxycarbonyl, phenyloxy-C₁₋₃-alkyl, phenylsulphonyl-C₁₋₃-alkyl), morpholin-4-yl-C₁₋₃-alkyl, cyano, amino, C₁₋₃-alkylamino, di-(C₁₋₃-alkyl)-amino, amino-C₁₋₃-alkylamino, C₁₋₃-alkylamino-C₁₋₃-alkylamino, di-(C₁₋₃-alkyl)-amino-C₁₋₃-alkylamino, N-(amino-C₁₋₃-alkyl)-N—(C₁₋₃-alkyl)-amino, N—(C₁₋₃-alkylamino-C₁₋₃-alkyl)-N—(C₁₋₃-alkyl)-amino, N-[di-(C₁₋₃-alkyl)-amino-C₁₋₃-alkyl]-N—(C₁₋₃-alkyl)-amino, morpholin-4-yl, piperazin-1-yl or 4-(C₁₋₃-alkyl)-piperazin-1-yl,
or a group selected from among R¹R²N, R¹R²N—CO, R¹R²N—CO—NR³, R¹R²N—SO, R¹R²N—SO₂, R¹R²N—SO₂—NR³, R⁴—CO, R⁴—CO—NR³, R⁵—SO, R⁵—SO—NR³, R⁵—SO₂— and R⁵—SO₂—NR³, wherein
R¹ denotes H, C₁₋₄-alkyl, hydroxy-C₁₋₄-alkyl, C₃₋₆-cycloalkyl, C₃₋₆-cycloalkyl-C₁₋₄-alkyl, heterocycloalkyl, heterocycloalkyl-C₁₋₄-alkyl, aryl, aryl-C₁₋₄-alkyl, heteroaryl or heteroaryl-C₁₋₄-alkyl,
R² denotes H, C₁₋₄-alkyl, hydroxy-C₁₋₄-alkyl, C₃₋₆-cycloalkyl, C₃₋₆-cycloalkyl-C₁₋₄-alkyl, heterocycloalkyl, heterocycloalkyl-C₁₋₄-alkyl, aryl, aryl-C₁₋₄-alkyl, heteroaryl or heteroaryl-C₁₋₄-alkyl,
R³ denotes H, C₁₋₄-alkyl, C₃₋₆-cycloalkyl or C₃₋₆-cycloalkyl-C₁₋₄-alkyl,
R⁴ denotes C₁₋₄-alkyl, C₃₋₆-cycloalkyl, C₃₋₆-cycloalkyl-C₁₋₄-alkyl, heterocycloalkyl, aryl, aryl-C₁₋₄-alkyl, heteroaryl, heteroaryl-C₁₋₄-alkyl, hydroxy or C₁₋₄-alkyloxy and
R⁵ denotes C₁₋₄-alkyl, C₃₋₆-cycloalkyl, heterocycloalkyl, aryl, aryl-C₁₋₄-alkyl, hetero-aryl or heteroaryl-C₁₋₄-alkyl,
while the aryl and heteroaryl groups contained in the groups mentioned hereinbefore for R¹ to R⁵ may optionally be substituted by halogen, cyano, C₁₋₃-alkoxy, C₁₋₃-alkoxycarbonyl, carboxy, aminocarbonyl, C₁₋₃-alkylaminocarbonyl, di-(C₁₋₃-alkyl)-aminocarbonyl, morpholin-4-ylcarbonyl, piperazin-1-ylcarbonyl, amino, C₁₋₃-alkylamino, di-(C₁₋₃-alkyl)-amino, amino-C₁₋₃-alkyl, amino-C₁₋₃-alkylamino, C₁₋₃-alkylamino-C₁₋₃-alkylamino, di-(C₁₋₃-alkyl)-amino-C₁₋₃-alkylamino, N-(amino-C₁₋₃-alkyl)-N—(C₁₋₃-alkyl)-amino, N—(C₁₋₃-alkylamino-C₁₋₃-alkyl)-N—(C₁₋₃-alkyl)-amino or N-[di-(C₁₋₃-alkyl)-amino-C₁₋₃-alkyl]-N—(C₁₋₃-alkyl)-amino,
R^{e} has the meaning given hereinbefore for R^{d}, with the proviso that at least one of the groups R^{d} and R^{e} must be H, halogen or C₁₋₃-alkyl, and
R^{f} denotes H or C₁₋₃-alkyl,
as well as the physiologically acceptable salts thereof.

4. A compound of general formula (I) according to claim 3, wherein
the bicyclic heteroaromatic group of general formula (II) denotes naphthalene or quinoline,
R^{a} denotes H or a C₁₋₄-alkyl group optionally substituted by a di-(C₁₋₃-alkyl)-amino group,
R^{b} and R^{c} independently of one another denote chlorine, bromine or C₁₋₂-alkyl,
Z denotes CH,
R^{d} denotes H, or, if R^{e} denotes H, it may also denote a group selected from among
fluorine, chlorine, bromine, cyano, C₁₋₃-alkoxy, 5-methyl-[1,2,4]oxadiazolyl,
aminocarbonyl, wherein a hydrogen atom may be replaced by a C₁₋₃-alkyl group and the second hydrogen atom may be replaced independently thereof by a C₁₋₃-alkyl, phenyl or phenyl-C₁₋₃-alkyl group, and
amino, wherein a hydrogen atom may be replaced by a C₁₋₃-alkyl group and the second hydrogen atom may be replaced independently thereof by a C₁₋₃-alkyl or a phenyl-sulphonyl group,
R^{e} denotes H, or, if R^{d} denotes H, it may also denote a group selected from among
fluorine, chlorine, bromine, cyano, C₁₋₃-alkyl, C₁₋₃-alkoxy,
furanyl, oxazolyl, isoxazolyl, which may be substituted in each case by one or two C₁₋₃-alkyl groups,
[1,2,4]oxadiazolyl, which may be substituted by C₁₋₃-alkyl, trichloromethyl, phenyl, benzyl, hydroxy, C₁₋₃-alkoxycarbonyl, phenyloxymethyl, phenylsulphonylmethyl or morpholin-4-ylmethyl,
5-oxo-4,5-dihydro-[1,2,4]oxadiazolyl, which may be substituted by C₁₋₃-alkyl,
pyridinyl, pyrimidinyl, pyrazinyl, pyridazinyl, which may be substituted in each case by C₁₋₃-alkyl, cyano, amino, C₁₋₃-alkylamino, di-(C₁₋₃-alkyl)-amino, di-(C₁₋₃-alkyl)-amino-C₁₋₃-alkylamino, N-[di-(C₁₋₃-alkyl)-amino-C₁₋₃-alkyl]-N—(C₁₋₃-alkyl)-amino, morpholin-4-yl or piperazin-1-yl,
pyrrolidin-1-ylcarbonyl, 3,4-dihydro-1H-isoquinolin-2-ylcarbonyl,
and a group of formula R¹R²N—CO, R¹R²N—CO—NR³ or R⁴CONR³, wherein
R¹ denotes H, C₁₋₃-alkyl, hydroxy-C₁₋₃-alkyl, C₃₋₆-cycloalkyl-C₁₋₃-alkyl, phenyl, phenyl-C₁₋₃-alkyl, pyridinyl or pyridinyl-C₁₋₃-alkyl,
R² denotes H or C₁₋₃-alkyl,
R³ denotes H or C₁₋₃-alkyl,
and
R⁴ denotes C₁₋₃-alkyl, phenyl, phenyl-C₁₋₃-alkyl, pyridinyl or pyridinyl-C₁₋₃-alkyl,
while the phenyl and pyridinyl groups contained in R¹ to R⁴ may optionally be substituted by chlorine, cyano, methoxy, carboxy, aminocarbonyl, C₁₋₃-alkylaminocarbonyl, di-(C₁₋₃-alkyl)-aminocarbonyl, morpholin-4-ylcarbonyl, piperazin-1-ylcarbonyl, amino, C₁₋₃-alkylamino, di-(C₁₋₃-alkyl)-amino, aminomethyl, di-(C₁₋₃-alkyl)-amino-C₁₋₃-alkylamino or N-[di-(C₁₋₃-alkyl)-amino-C₁₋₃-alkyl]-N—(C₁₋₃-alkyl)-amino,
and
R^{f} denotes H or C₁₋₃-alkyl,
as well as the physiologically acceptable salts thereof.

5. A compound of general formula (I) according to claim 4, wherein
the bicyclic heteroaromatic group of formula (II) is naphthalene or quinoline,
R^{a} denotes H,
R^{b} and R^{c} independently of one another denote chlorine, bromine or methyl,
Z denotes CH,
R^{d} denotes H, or, if R^{e} denotes H, it may also denote a group selected from among
C₁₋₂-alkoxy, 5-methyl-[1,2,4]oxadiazole, N-phenylsulphonyl-N-methyl-amino, N-methyl-N-phenyl-aminocarbonyl, N-benzyl-aminocarbonyl and N-benzyl-N-methyl-aminocarbonyl,
R^{e} denotes H, or, if R^{d} denotes H, it may also denote a group selected from among
methoxy, furanyl, oxazolyl, isoxazolyl, 3,5-dimethyl-isoxazole, 3-methyl-[1,2,4]oxadiazolyl, 5-methyl-[1,2,4]oxadiazolyl, 5-trichloromethyl-[1,2,4]oxadiazolyl, 5-isopropyl-[1,2,4]oxadiazolyl, 3-phenyl-[1,2,4]oxadiazolyl, 5-phenyl-[1,2,4]oxadiazolyl, 3-benzyl-[1,2,4]oxadiazolyl, 5-benzyl-[1,2,4]oxadiazolyl, 5-hydroxy-[1,2,4]oxadiazolyl, 3-ethoxycarbonyl-[1,2,4]oxadiazolyl, 3-phenyloxymethyl-[1,2,4]oxadiazolyl, 3-phenylsulphonylmethyl-[1,2,4]oxadiazolyl, 5-(morpholin-4-ylmethyl)-[1,2,4]oxadiazolyl, 5-oxo-4,5-dihydro-[1,2,4]oxadiazolyl, 4-methyl-5-oxo-4,5-dihydro-[1,2,4]oxadiazolyl, pyridinyl, pyrimidinyl, 4-(piperazin-1-yl)-pyrimidinyl, 2-(morpholin-4-yl)-pyrimidinyl, 4-(morpholin-4-yl)-pyrimidinyl, 4-(2-dimethylamino-ethylamino)-pyrimidinyl, 4-[N-(2-dimethylamino-ethyl)-N-methyl-amino]-pyrimidinyl, 5-(morpholin-4-yl)-pyrazin-2-yl, 5-(2-dimethylamino-ethylamino)-pyrazin-2-yl, 6-(morpholin-4-yl)-pyridazin-3-yl, 6-(2-dimethylamino-ethylamino)-pyridazin-3-yl, pyrrolidin-1-ylcarbonyl, 3,4-dihydro-1H-isoquinolin-2-ylcarbonyl,
and a group of formula n R¹R²N—CO, R¹R²N—CO—NR³ or R⁴CONR³, wherein
R¹ denotes H, C₁₋₃-alkyl, hydroxyethyl, cyclohexylmethyl, phenyl, benzyl, 2-phenyl-ethyl, pyridinyl or pyridinylmethyl,
R² denotes H or methyl,
R³ denotes H
and
R⁴ denotes phenyl, benzyl, 2-phenyl-ethyl or pyridinyl,
while the phenyl, benzyl and 2-phenyl-ethyl groups contained in R¹ and R⁴ may be substituted by a cyano, methoxy, carboxy, aminocarbonyl, methylamino-carbonyl, dimethylaminocarbonyl, morpholin-4-ylcarbonyl, piperazin-1-ylcarbonyl or aminomethyl group and
the pyridinyl and pyridinylmethyl groups contained in R¹ and R⁴ may be substituted by a chlorine atom or a 2-dimethylamino-ethylamino or N-(2-dimethylamino-ethyl)-N-(methyl)-amino group,
and
R^{f} denotes H,
as well as the physiologically acceptable salts thereof.

6. A compound of general formula 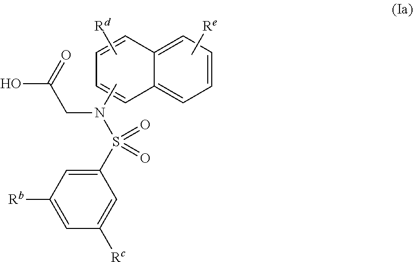
wherein
R^{b} and R^{c} each denote chlorine,
R^{d} denotes H, or, if R^{e} denotes H, it may also denote a group selected from among
fluorine, chlorine, bromine, cyano, C₁₋₃-alkyl, C₁₋₃-alkoxy, 5-methyl-[1,2,4]oxadiazolyl,
aminocarbonyl, wherein a hydrogen atom may be replaced by a C₁₋₃-alkyl group and the second hydrogen atom may be replaced independently thereof by a C₁₋₃-alkyl, phenyl or phenyl-C₁₋₃-alkyl group, and
amino, wherein a hydrogen atom may be replaced by a C₁₋₃-alkyl group and the second hydrogen atom may be replaced independently thereof by a C₁₋₃-alkyl or a phenyl-sulphonyl group,
and
R^{e} denotes H, or, if R^{d} denotes H, it may also denote a group selected from among
fluorine, chlorine, bromine, cyano, C₁₋₃-alkyl, C₁₋₃-alkoxy,
furanyl, oxazolyl, isoxazolyl, which may be substituted in each case by one or two C₁₋₃-alkyl groups,
[1,2,4]oxadiazolyl, which may be substituted by C₁₋₃-alkyl, trichloromethyl, phenyl, benzyl, hydroxy, C₁₋₃-alkoxycarbonyl, phenyloxymethyl, phenylsulphonylmethyl or morpholin-4-ylmethyl,
5-oxo-4,5-dihydro-[1,2,4]oxadiazolyl, which may be substituted by C₁₋₃-alkyl,
pyridinyl, pyrimidinyl, pyrazinyl, pyridazinyl, which may be substituted in each case by C₁₋₃-alkyl, cyano, amino, C₁₋₃-alkylamino, di-(C₁₋₃-alkyl)-amino, di-(C₁₋₃-alkyl)-amino-C₁₋₃-alkylamino, N-[di-(C₁₋₃-alkyl)-amino-C₁₋₃-alkyl]-N—(C₁₋₃-alkyl)-amino, morpholin-4-yl or piperazin-1-yl,
pyrrolidin-1-ylcarbonyl, 3,4-dihydro-1H-isoquinolin-2-ylcarbonyl,
and a group of formula n R¹R²N—CO, R¹R²N—CO—NR³ or R⁴CONR³, wherein
R¹ denotes H, C₁₋₃-alkyl, hydroxy-C₁₋₃-alkyl, C₃₋₆-cycloalkyl-C₁₋₃-alkyl, phenyl, phenyl-C₁₋₃-alkyl, pyridinyl or pyridinyl-C₁₋₃-alkyl,
R² denotes H or C₁₋₃-alkyl,
R³ denotes H or C₁₋₃-alkyl,
and
R⁴ denotes phenyl, phenyl-C₁₋₃-alkyl, pyridinyl or pyridinyl-C₁₋₃-alkyl,
while the phenyl and pyridinyl groups contained in R¹ to R⁴ may optionally be substituted by chlorine, cyano, methoxy, carboxy, aminocarbonyl, C₁₋₃-alkylaminocarbonyl, di-(C₁₋₃-alkyl)-aminocarbonyl, morpholin-4-ylcarbonyl, piperazin-1-ylcarbonyl, amino, C₁₋₃-alkylamino, di-(C₁₋₃-alkyl)-amino, aminomethyl, di-(C₁₋₃-alkyl)-amino-C₁₋₃-alkylamino or N-[di-(C₁₋₃-alkyl)-amino-C₁₋₃-alkyl]-N—(C₁₋₃-alkyl)-amino,
as well as the physiologically acceptable salts thereof.

7. A compound according to claim 1 selected from the group consisting of:
(1) [[5-(4-aminocarbonyl-benzylaminocarbonyl)-naphthalen-2-yl]-(3,5-dichloro-phenylsulphonyl)-amino]-acetic acid,
(2) {(3,5-dichloro-phenylsulphonyl)-[6-(5-methyl-[1,2,4]oxadiazol-3-yl)-naphthalen-2-yl]-amino}-acetic acid,
(3) {(3,5-dichloro-phenylsulphonyl)-[6-(3-methyl-[1,2,4]oxadiazol-5-yl)-naphthalen-2-yl]-amino}-acetic acid,
(4) [(5-benzylaminocarbonyl-naphthalen-1-yl)-(3,5-dichloro-phenylsulphonyl)-amino]-acetic acid,
(5) [(3,5-dichloro-phenylsulphonyl)-(5-pyrimidin-2-yl-naphthalen-1-yl)-amino]-acetic acid,
(6) {(3,5-dichloro-phenylsulphonyl)-[5-(5-morpholin-4-ylmethyl-[1,2,4]oxadiazol-3-yl)-naphthalen-2-yl]-amino}-acetic acid,
(7) ((3,5-dichloro-phenylsulphonyl)-{5-[(pyridin-3-ylmethyl)-aminocarbonyl]-naphthalen-1-yl}-amino)-acetic acid,
(8) {(3,5-dichloro-phenylsulphonyl)-[5-(3-phenyl-ureido)-naphthalen-1-yl]-amino}-acetic acid,
(9) [[5-(3-cyano-benzylaminocarbonyl)-naphthalen-1-yl]-(3,5-dichloro-phenylsulphonyl)-amino]-acetic acid,
(10) [[5-(2-cyano-benzylaminocarbonyl)-naphthalen-1-yl]-(3,5-dichloro-phenylsulphonyl)-amino]-acetic acid,
(11) ((3,5-dichloro-phenylsulphonyl)-{5-[4-(piperazin-1-ylcarbonyl)-benzylaminocarbonyl]-naphthalen-1-yl}-amino)-acetic acid,
(12) {(3,5-dichloro-phenylsulphonyl)-[5-(4-methylaminocarbonyl-benzylaminocarbonyl)-naphthalen-1-yl]-amino}-acetic acid,
(13) {(3,5-dichloro-phenylsulphonyl)-[5-(3-methylaminocarbonyl-benzylaminocarbonyl)-naphthalen-1-yl]-amino}-acetic acid,
(14) {(3,5-dichloro-phenylsulphonyl)-[5-({2-N-[(2-dimethylamino-ethyl)-N-methyl-amino]-pyridin-4-ylmethyl}-aminocarbonyl)-naphthalen-1-yl]-amino}-acetic acid,
(15) ((3,5-dichloro-phenylsulphonyl)-{6-[5-(2-dimethylamino-ethylamino)-pyrazin-2-yl]-naphthalen-2-yl}-amino)-acetic acid,
(16) {(3,5-dichloro-phenylsulphonyl)-[6-(4-morpholin-4-yl-pyrimidin-2-yl)-naphthalen-2-yl]-amino}-acetic acid,
(17) [(3,5-dichloro-phenylsulphonyl)-quinolin-8-yl-amino]-acetic acid and
(18) [(3,5-dichloro-phenylsulphonyl)-(6-methoxy-quinolin-8-yl)-amino]-acetic acid,
or a physiologically acceptable salt thereof.

8. Physiologically acceptable salt of a compound according to claim 1 with inorganic or organic acid or base.

9. A compound according to claim 1 for use as a pharmaceutical composition.

10. A method of treating a disease selected from the group consisting of type I and type II diabetes mellitus comprising administering an effective amount of a compound according to claim 1 or a physiologically acceptable salt of a compound according to claim 1 with inorganic or organic acids or bases to a patient in need thereof.

11. A pharmaceutical composition comprising a compound according to claim 1 or a physiologically acceptable salt of a compound according to claim 1 with inorganic or organic acids or bases optionally together with one or more inert carriers and/or diluents.

12. A method of treating a disease selected from the group consisting of type I and type II diabetes mellitus comprising administering to a patient an effective amount of a pharmaceutical composition according to claim 11.

13. Process for preparing a pharmaceutical composition comprising combining a compound according to claim 1 or a physiologically acceptable salt of a compound according to claim 1 with inorganic or organic acids or bases with one or more inert carriers and/or diluents by a non-chemical method.

14. Process for preparing the compounds of general formula (I) according to claim 1, characterised in that
a compound of general formula (IV) 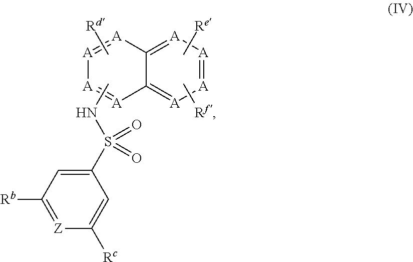
wherein R^{b}, R^{c}, Z and A are defined as in claim 1 and
R^{d}, R^{c}, and R^{f}, either have the meaning given for R^{d}, R^{e} and R^{f} in claim 1 or denote groups that can be converted into R^{d}, R^{e} and R^{f} by known methods of synthesis,
is alkylated by means of a suitable acetic acid ester derivative of general formula
R^{a′}—O—(CO)—CH₂—X,
wherein R^{a}′ either has the meaning given for R^{a} in claim 1 or denotes a group which may be converted into R^{a} by known methods of synthesis and
X denotes a leaving group,
and
if desired any protective group used to protect reactive groups during the reactions is cleaved afterwards or simultaneously and/or
a compound of general formula I thus obtained is converted into the salts thereof, particularly for pharmaceutical use into the physiologically acceptable salts thereof with an inorganic or organic acid or base.
